SEQUENCE LISTING (1259 sequences)

SEQ ID NO: 1 - protein, 13 aa, Human immunodeficiency virus type 1
GRKKRRQRRRPPQ

SEQ ID NO: 2 - protein, 16 aa, Artificial sequence
  Antennapedia penetratin cell-penetrating polypeptide
RQIKIWFQNRRMKWKK

SEQ ID NO: 3 - protein, 10 aa, Artificial sequence
  cell-penetrating polypeptide
KRRRGRKKRR

SEQ ID NO: 4 - protein, 27 aa, Artificial sequence
  cell-penetrating polypeptide
GWTLNSAGYLLGKINLKALAALAKKIL

SEQ ID NO: 5 - protein, 10 aa, Artificial sequence
  cell-penetrating polypeptide
RRGRKKRRKR

SEQ ID NO: 6 - protein, 10 aa, Artificial sequence
  cell-penetrating polypeptide
RGRKKRRKRR

SEQ ID NO: 7 - protein, 10 aa, Artificial sequence
  cell-penetrating polypeptide
GRKKRRKRRR

SEQ ID NO: 8 - protein, 10 aa, Artificial sequence
  cell-penetrating polypeptide
KRRRGRKKRR

SEQ ID NO: 9 - protein, 11 aa, Artificial sequence
  cell-penetrating polypeptide
YGRKKRRQRRR

SEQ ID NO: 10 - protein, 10 aa, Artificial sequence
  cell-penetrating polypeptide
RKKRRKRRRR

SEQ ID NO: 11 - protein, 10 aa, Artificial sequence
  cell-penetrating polypeptide
KKRRKRRRRK

SEQ ID NO: 12 - protein, 10 aa, Artificial sequence
  cell-penetrating polypeptide
KRRKRRRRKK

SEQ ID NO: 13 - protein, 10 aa, Artificial sequence
  cell-penetrating polypeptide
RRRGRKKRRK

SEQ ID NO: 14 - protein, 10 aa, Artificial sequence
  cell-penetrating polypeptide
RRKRRRRKKR

SEQ ID NO: 15 - protein, 10 aa, Artificial sequence
  cell-penetrating polypeptide
RKRRRRKKRR

SEQ ID NO: 16 - protein, 10 aa, Artificial sequence
  cell-penetrating polypeptide
KRRRRKKRRR

SEQ ID NO: 17 - protein, 10 aa, Artificial sequence
  cell-penetrating polypeptide
RRRRKKRRRR

SEQ ID NO: 18 - protein, 10 aa, Artificial sequence
  cell-penetrating polypeptide
ALKFGLKLAL

SEQ ID NO: 19 - protein, 10 aa, Artificial sequence
  cell-penetrating polypeptide
ALKLCLKLGL

SEQ ID NO: 20 - protein, 10 aa, Artificial sequence
  cell-penetrating polypeptide
CLKLALKLAL

SEQ ID NO: 21 - protein, 10 aa, Artificial sequence
  cell-penetrating polypeptide
GLKLALKFGL

SEQ ID NO: 22 - protein, 10 aa, Artificial sequence
  cell-penetrating polypeptide
KLALKLALKL

SEQ ID NO: 23 - protein, 10 aa, Artificial sequence
  cell-penetrating polypeptide
KLALKLGLKL

SEQ ID NO: 24 - protein, 10 aa, Artificial sequence
  cell-penetrating polypeptide
LGLKLALKLC

SEQ ID NO: 25 - protein, 10 aa, Artificial sequence
  cell-penetrating polypeptide
GQAGRARAAC

SEQ ID NO: 26 - protein, 33 aa, Artificial sequence
  cell-penetrating polypeptide
KLALKLGLKLALKLCLKLGLKLGLKLALKFGLK

SEQ ID NO: 27 - protein, 10 aa, Artificial sequence
  cell-penetrating polypeptide
RARAACKLAL

SEQ ID NO: 28 - protein, 10 aa, Artificial sequence
  cell-penetrating polypeptide
RAACKLALRL

SEQ ID NO: 29 - protein, 10 aa, Artificial sequence
  cell-penetrating polypeptide
QGARLRSARK

SEQ ID NO: 30 - protein, 10 aa, Artificial sequence
  cell-penetrating polypeptide
RLRSARKVLR

SEQ ID NO: 31 - protein, 10 aa, Artificial sequence
  cell-penetrating polypeptide
RKVLRATLKR

SEQ ID NO: 32 - protein, 31 aa, Artificial sequence
  cell-penetrating polypeptide
GDIMGEWGNEIFGAIAGFLGYGRKKRRQRRR

SEQ ID NO: 33 - protein, 15 aa, Artificial sequence
  cell-penetrating polypeptide
RKKRWFRRRRPKWKK

SEQ ID NO: 34 - protein, 20 aa, Artificial sequence
  cell-penetrating polypeptide
GLWRALWRLLRSLWRLLWRA

SEQ ID NO: 35 - protein, 8 aa, Artificial sequence
  cell-penetrating polypeptide
  MISC_FEATURE (1)..(1): Xaa is cyclohexylalanine
  MISC_FEATURE (2)..(2): Xaa is D-Arginine
  MISC_FEATURE (3)..(3): Xaa is cyclohexylalanine
  MISC_FEATURE (5)..(5): Xaa is cyclohexylalanine
  MISC_FEATURE (6)..(6): Xaa is D-Arginine
  MISC_FEATURE (7)..(7): Xaa is cyclohexylalanine
XXXKXXXK

SEQ ID NO: 36 - protein, 12 aa, Artificial sequence
  cell-penetrating polypeptide
PLILLRLLRGQF

SEQ ID NO: 37 - protein, 12 aa, Artificial sequence
  cell-penetrating polypeptide
RRILLQLLRGQF

SEQ ID NO: 38 - protein, 7 aa, Artificial sequence
  cell-penetrating polypeptide
  MISC_FEATURE (2)..(2): Xaa is L-2-naphthylalanine
FXRRRRQ

SEQ ID NO: 39 - protein, 8 aa, Artificial sequence
  cell-penetrating polypeptide
  MISC_FEATURE (3)..(3): Xaa is L-2-naphthylalanine
FFXRRRRQ

SEQ ID NO: 40 - protein, 10 aa, Artificial sequence
  cell-penetrating polypeptide; cyclization via a disulfide bond
CRRRRRRRRC

SEQ ID NO: 41 - protein, 5 aa, Artificial sequence
  cell-penetrating polypeptide; cyclo
RRRRR

SEQ ID NO: 42 - protein, 5 aa, Artificial sequence
  cell-penetrating polypeptide; Dodecanoyl-cyclo
RRRRR

SEQ ID NO: 43 - protein, 28 aa, Artificial sequence
  cell-penetrating polypeptide
LSTAADMQGVVTDGMASGLDKDYLKPDD

SEQ ID NO: 44 - protein, 18 aa, Artificial sequence
  cell-penetrating polypeptide
LSTAADMQGVVTDGMASG

SEQ ID NO: 45 - protein, 12 aa, Artificial sequence
  cell-penetrating polypeptide
VKKKKIKAEIKI

SEQ ID NO: 46 - protein, 17 aa, Artificial sequence
  cell-penetrating polypeptide
KGEGAAVLLPVLLAAPG

SEQ ID NO: 47 - protein, 11 aa, Artificial sequence
  cell-penetrating polypeptide
ACTGSTQHQCG

SEQ ID NO: 48 - protein, 7 aa, Artificial sequence
  cell-penetrating polypeptide
LCLRPVG

SEQ ID NO: 49 - protein, 9 aa, Artificial sequence
  cell-penetrating polypeptide
RKKRRQRRR

SEQ ID NO: 50 - protein, 10 aa, Artificial sequence
  cell-penetrating polypeptide
RRRKKRRRRR

SEQ ID NO: 51 - protein, 21 aa, Artificial sequence
  cell-penetrating polypeptide
KETWWETWWTEWSQPKKKRKV

SEQ ID NO: 52 - protein, 10 aa, Artificial sequence
  cell-penetrating polypeptide
VQRKRQKLMP

SEQ ID NO: 53 - protein, 10 aa, Artificial sequence
  cell-penetrating polypeptide
RRKKRRRRRG

SEQ ID NO: 54 - protein, 10 aa, Artificial sequence
  cell-penetrating polypeptide
RKKRRRRRGG

SEQ ID NO: 55 - protein, 11 aa, Artificial sequence
  FAM-labeled YARA peptide
YARAAARQARA

SEQ ID NO: 56 - protein, 12 aa, Artificial sequence
  cell-penetrating polypeptide
YARAAARQARAC

SEQ ID NO: 57 - protein, 13 aa, Artificial sequence
  N-terminal 5(6)-carboxyfluorescein-labeled peptide FAM-YARA-Cys
YARAAARQARAGC

SEQ ID NO: 58 - protein, 11 aa, Artificial sequence
  cell-penetrating polypeptide
KKIFKKILKFL

SEQ ID NO: 59 - protein, 10 aa, Artificial sequence
  cell-penetrating polypeptide
KKLFKKIVKY

SEQ ID NO: 60 - protein, 11 aa, Artificial sequence
  cell-penetrating polypeptide
KLFFKKILKYL

SEQ ID NO: 61 - protein, 13 aa, Artificial sequence
  cell-penetrating polypeptide
CYARAAARQARAC

SEQ ID NO: 62 - protein, 25 aa, Artificial sequence
  cell-penetrating polypeptide
KLIFKKILKYLKVFTISGKIILVGK

SEQ ID NO: 63 - protein, 13 aa, Artificial sequence
  cell-penetrating polypeptide
KRKRKKLFKKILK

SEQ ID NO: 64 - protein, 10 aa, Artificial sequence
  cell-penetrating polypeptide
SFATRFIPSP

SEQ ID NO: 65 - protein, 17 aa, Artificial sequence
  cell-penetrating polypeptide
YRQERRARRRRRRERER

SEQ ID NO: 66 - protein, 10 aa, Artificial sequence
  cell-penetrating polypeptide
ALKLALKLCL

SEQ ID NO: 67 - protein, 10 aa, Artificial sequence
  cell-penetrating polypeptide
ASISQLKRSF

SEQ ID NO: 68 - protein, 10 aa, Artificial sequence
  cell-penetrating polypeptide
CLKLGLKLGL

SEQ ID NO: 69 - protein, 10 aa, Artificial sequence
  cell-penetrating polypeptide
KLALKFGLKL

SEQ ID NO: 70 - protein, 10 aa, Artificial sequence
  cell-penetrating polypeptide
KLCLKLALKL

SEQ ID NO: 71 - protein, 10 aa, Artificial sequence
  cell-penetrating polypeptide
LALKLALKLA

SEQ ID NO: 72 - protein, 10 aa, Artificial sequence
  cell-penetrating polypeptide
LKLALKLALK

SEQ ID NO: 73 - protein, 10 aa, Artificial sequence
  cell-penetrating polypeptide
AGRARAACKL

SEQ ID NO: 74 - protein, 10 aa, Artificial sequence
  cell-penetrating polypeptide
GRARAACKLA

SEQ ID NO: 75 - protein, 10 aa, Artificial sequence
  cell-penetrating polypeptide
ARAACKLALR

SEQ ID NO: 76 - protein, 10 aa, Artificial sequence
  cell-penetrating polypeptide
RLNPGALRPA

SEQ ID NO: 77 - protein, 10 aa, Artificial sequence
  cell-penetrating polypeptide
GARLRSARKV

SEQ ID NO: 78 - protein, 10 aa, Artificial sequence
  cell-penetrating polypeptide
LRSARKVLRA

SEQ ID NO: 79 - protein, 10 aa, Artificial sequence
  cell-penetrating polypeptide
RKVLRAKLKR

SEQ ID NO: 80 - protein, 16 aa, Artificial sequence
  cell-penetrating polypeptide
GRKKRWFRRRRMKWKK

SEQ ID NO: 81 - protein, 15 aa, Artificial sequence
  cell-penetrating polypeptide
RIKRRFRRLRPKWKK

SEQ ID NO: 82 - protein, 13 aa, Artificial sequence
  cell-penetrating polypeptide
RRKKIWFRRLRMK

SEQ ID NO: 83 - protein, 8 aa, Artificial sequence
  cell-penetrating polypeptide
FRFKFRFK

SEQ ID NO: 84 - protein, 12 aa, Artificial sequence
  cell-penetrating polypeptide
PLIYLRLLRGQF

SEQ ID NO: 85 - protein, 12 aa, Artificial sequence
  cell-penetrating polypeptide
  MISC_FEATURE (1)..(12): all residues D-form
PLIYLRLLRGQF

SEQ ID NO: 86 - protein, 7 aa, Artificial sequence
  cell-penetrating polypeptide
  MISC_FEATURE (1)..(1): Xaa is D-phenylalanine
  MISC_FEATURE (2)..(2): Xaa is L-2-naphthylalanine
XXRRRRQ

SEQ ID NO: 87 - protein, 6 aa, Artificial sequence
  cell-penetrating polypeptide
  MISC_FEATURE (1)..(1): Xaa is L-Aspartic acid decylamine amide
XRRRRQ

SEQ ID NO: 88 - protein, 13 aa, Artificial sequence
  cell-penetrating polypeptide; cyclization via a disulfide bond
CYGRKKRRQRRRC

SEQ ID NO: 89 - protein, 6 aa, Artificial sequence
  cell-penetrating polypeptide; cyclo
RRRRRR

SEQ ID NO: 90 - protein, 6 aa, Artificial sequence
  cell-penetrating polypeptide; Dodecanoyl-cyclo
RRRRRR

SEQ ID NO: 91 - protein, 28 aa, Artificial sequence
  cell-penetrating polypeptide
SPANLDQIVSAKKPKIVQERLEKVIASA

SEQ ID NO: 92 - protein, 26 aa, Artificial sequence
  cell-penetrating polypeptide
SFEVHDKKNPTLEIPAGATVDVTFIN

SEQ ID NO: 93 - protein, 13 aa, Artificial sequence
  cell-penetrating polypeptide
GLFDIIKKIAESF

SEQ ID NO: 94 - protein, 5 aa, Artificial sequence
  cell-penetrating polypeptide
GFWFG

SEQ ID NO: 95 - protein, 86 aa, Human immunodeficiency virus type 1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 96 - protein, 378 aa, Drosophila melanogaster
MTMSTNNCESMTSYFTNSYMGADMHHGHYPGNGVTDLDAQQMHHYSQNANHQGNMPYPRFPPYDRMPYYNGQGMDQQQQHQVYSRPDSPSSQVGGVMPQAQTNGQLGVPQQQQQQQQQPSQNQQQQQAQQAPQQLQQQLPQVTQQVTHPQQQQQQPVVYASCKLQAAVGGLGMVPEGGSPPLVDQMSGHHMNAQMTLPHHMGHPQAQLGYTDVGVPDVTEVHQNHHNMGMYQQQSGVPPVGAPPQGMMHQGQGPPQMHQGHPGQHTPPSQNPNSQSSGMPSPLYPWMRSQFGKCQERKRGRQTYTRYQTLELEKEFHFNRYLTRRRRIEIAHALCLTERQIKIWFQNRRMKWKKENKTKGEPGSGGEGDEITPPNSPQ

SEQ ID NO: 97 - protein, 301 aa, herpes simplex virus type 1
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 98 - protein, 189 aa, Brome mosaic virus
MSTSGTGKMTRAQRRAAARRNRRTARVQPVIVEPLAAGQGKAIKAIAGYSISKWEASSDAITAKATNAMSITLPHELSSEKNKELKVGRVLLWLGLLPSVAGRIKACVAEKQAQAEAAFQVALAVADSSKEVVAAMYTDAFRGATLGDLLNLQIYLYASEAVPAKAVVVHLEVEHVRPTFDDFFTPVYR

SEQ ID NO: 99 - protein, 367 aa, Yersinia enterocolitica
MFINPRNVSNTFLQEPLRHSSDLTEMPVEAENVKSKAEYYNAWSEWERNAPPGNGEQRGMAVSRLRDCLDRQAHELELNNLGLSSLPELPPHLESLVASCNSLTELPELPQSLKSLQVDNNNLKALSDLPPLLEYLGAANNQLEELPELQNSSFLTSIDVDNNSLKTLPDLPPSLEFLAAGNNQLEELSELQNLPFLTAIYADNNSLKTLPDLPPSLKTLNVRENYLTDLPELPQSLTFLDVSDNIFSGLSELPPNLYNLNASSNEIRSLCDLPPSLVELDVRDNQLIELPALPPRLERLIASFNHLAEVPELPQNLKLLHVEYNALREFPDIPESVEDLRMDSERVIDPYEFAHETIDKLEDDVFE

SEQ ID NO: 100 - protein, 244 aa, Artificial sequence
  Artificial protein B1
MWFKREQGRGAVHRGGAHPGRAGRRRKRPQVQRVRRGRGRCHLRQADPEVHLHHRQAARALAHPRDHPDLRRAVLQPLPRPHEAARLLQVRHARRLRPGAHHLLQGRRQLQDPRRGEVRGRHPGEPHRAEGHRLQGGRQHPGAQAGVQLQQPQRLYHGRQAEERHQGELQDPPQHRGRQRAAHRPLPAEHPHRRRPRAAARQPLPEHPVRPEQRPQREARSHGPAGVRDRRRDHSRHGRGLNLE

SEQ ID NO: 101 - protein, 254 aa, Bombyx mori
MKPAIVILCLFVASLYAADSDVPNDILEEQLYNSVVVADYDSAVEKSKHLYEEKKSEVITNVVNKLIRNNKMNCMEYAYQLWLQGSKDIVRDCFPVEFRLIFAENAIKLMYKRDGLALTLSNDVQGDDGRPAYGKDKTSPRVSWKLIALWENNKVYFKILNTERNQYLVLGVGTNWNGDHMAFGVNSVDSFRAQWYLQPAKYDNDVLFYIYNREYSKALTLSRTVEPSGHRMAWGYNGRVIGSPEHYAWGIKAF

SEQ ID NO: 102 - protein, 248 aa, Artificial sequence
  Engineered +36 GFP
MGHHHHHHGGASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

SEQ ID NO: 103 - protein, 132 aa, Homo sapiens
MFTIAQGKGQKLCEIERIHFFLSKKKTDELRNLHKLLYNRPGTVSSLKKNVGQFSGFPFEKGSVQYKKKEEMLKKFRNAMLKSICEVLDLERSGVNSELVKRILNFLMHPKPSGKPLPKSKKTCSKGSKKER

SEQ ID NO: 104 - protein, 28 aa, Artificial sequence
  fusion peptide H
YARAAARQARAGGGGSVVIVGQIILSGR

SEQ ID NO: 105 - DNA, 22 bp, Artificial sequence
  oligomer
tcaacatcagtctgataagcta

SEQ ID NO: 106 - protein, 14 aa, Artificial sequence
  peptide inhibitor
GSVVIVGQIILSGR

SEQ ID NO: 107 - protein, 17 aa, Artificial sequence
  peptide cargo
GGGGSVVIVGQIILSGR

SEQ ID NO: 108 - protein, 6 aa, Artificial Sequence
  Synthetic CPP PAF95
AAAWFW

SEQ ID NO: 109 - protein, 27 aa, Artificial Sequence
  Synthetic CPP PN225
AAVACRICMRNFSTRQARRNHRRRHRR

SEQ ID NO: 110 - protein, 16 aa, Artificial Sequence
  Synthetic CPP MPS
AAVALLPAVLLALLAK

SEQ ID NO: 111 - protein, 26 aa, Artificial Sequence
  Synthetic CPP MPS-Galphai2
AAVALLPAVLLALLAKKNNLKDCGLF

SEQ ID NO: 112 - protein, 26 aa, Artificial Sequence
  Synthetic CPP MPS-Galphai3
AAVALLPAVLLALLAKKNNLKECGLY

SEQ ID NO: 113 - protein, 16 aa, Artificial Sequence
  Synthetic CPP MTS
AAVALLPAVLLALLAP

SEQ ID NO: 114 - protein, 41 aa, Artificial Sequence
  Synthetic CPP SKP
AAVALLPAVLLALLAPEILLPNNYNAYESYKYPGMFIALSK

SEQ ID NO: 115 - protein, 28 aa, Artificial Sequence
  Synthetic CPP PN227
AAVALLPAVLLALLAPRKKRRQRRRPPQ

SEQ ID NO: 116 - protein, 29 aa, Artificial Sequence
  Synthetic CPP PN27
AAVALLPAVLLALLAPRKKRRQRRRPPQC

SEQ ID NO: 117 - protein, 22 aa, Artificial Sequence
  Synthetic CPP PN365
AAVALLPAVLLALLAPRRRRRR

SEQ ID NO: 118 - protein, 28 aa, Artificial Sequence
  Synthetic CPP PN29
AAVALLPAVLLALLAPSGASGLDKRDYV

SEQ ID NO: 119 - protein, 26 aa, Artificial Sequence
  Synthetic CPP SN50
AAVALLPAVLLALLAPVQRKRQKLMP

SEQ ID NO: 120 - protein, 43 aa, Artificial Sequence
  Synthetic CPP Anti-BetaGamma
AAVALLPAVLLALLAVTDQLGEDFFAVDLEAFLQEFGLLPEKE

SEQ ID NO: 121 - protein, 17 aa, Artificial Sequence
  Synthetic CPP IA6d
ACGRGRGRCGRGRGRCG

SEQ ID NO: 122 - protein, 17 aa, Artificial Sequence
  Synthetic CPP IA6b
ACGRGRGRCRGRGRGCG

SEQ ID NO: 123 - protein, 17 aa, Artificial Sequence
  Synthetic CPP IA5_2H1W
ACHGRRWGCGRHRGRCG

SEQ ID NO: 124 - protein, 17 aa, Artificial Sequence
  Synthetic CPP kCA3
ACRDRFRNCPADEALCG

SEQ ID NO: 125 - protein, 17 aa, Artificial Sequence
  Synthetic CPP kCA4
ACRDRFRNCPADERLCG

SEQ ID NO: 126 - protein, 17 aa, Artificial Sequence
  Synthetic CPP kCA5
ACRDRFRRCPADERLCG

SEQ ID NO: 127 - protein, 17 aa, Artificial Sequence
  Synthetic CPP kCA6
ACRDRFRRCPADRRLCG

SEQ ID NO: 128 - protein, 17 aa, Artificial Sequence
  Synthetic CPP IA6a
ACRGRGRGCGRGRGRCG

SEQ ID NO: 129 - protein, 17 aa, Artificial Sequence
  Synthetic CPP CA3
ACRGRGRGCGSGSGSCG

SEQ ID NO: 130 - protein, 17 aa, Artificial Sequence
  Synthetic CPP CA4
ACRGRGRGCGSGSRSCG

SEQ ID NO: 131 - protein, 17 aa, Artificial Sequence
  Synthetic CPP IA6c
ACRGRGRGCRGRGRGCG

SEQ ID NO: 132 - protein, 17 aa, Artificial Sequence
  Synthetic CPP CA6
ACRGRGRRCGSGRRSCG

SEQ ID NO: 133 - protein, 17 aa, Artificial Sequence
  Synthetic CPP CA5
ACRGRGRRCGSGSRSCG

SEQ ID NO: 134 - protein, 17 aa, Artificial Sequence
  Synthetic CPP IA8a
ACRGRRRGCGRRRGRCG

SEQ ID NO: 135 - protein, 17 aa, Artificial Sequence
  Synthetic CPP IA4a
ACRGSGRGCGRGSGRCG

SEQ ID NO: 136 - protein, 17 aa, Artificial Sequence
  Synthetic CPP IA8b L (Linear variants)
ACRRSRRGCGRRSRRCG

SEQ ID NO: 137 - protein, 17 aa, Artificial Sequence
  Synthetic CPP kCA2 (Kallikrein inhibitor with internal arginines)
ACSDRFRNCPADEALCG

SEQ ID NO: 138 - protein, 25 aa, Artificial Sequence
  Synthetic CPP kEA1 8
ACSDRFRNCPADEALCGRRRRRRRR

SEQ ID NO: 139 - protein, 17 aa, Artificial Sequence
  Synthetic CPP IA4b
ACSGRGRGCGRGRGSCG

SEQ ID NO: 140 - protein, 17 aa, Artificial Sequence
  Synthetic CPP CA2 (Control internal arginine)
ACSGRGRGCGSGSGSCG

SEQ ID NO: 141 - protein, 17 aa, Artificial Sequence
  Synthetic CPP IA2
ACSGRGSGCGSGRGSCG

SEQ ID NO: 142 - protein, 17 aa, Artificial Sequence
  Synthetic CPP IA0 (Bicyclic) (integral arginine peptides)
ACSGSGSGCGSGSGSCG

SEQ ID NO: 143 - protein, 25 aa, Artificial Sequence
  Synthetic CPP EA1x8 L
ACSGSGSGCGSGSGSCGRRRRRRRR

SEQ ID NO: 144 - protein, 25 aa, Artificial Sequence
  Synthetic CPP EA8_4H (Histidine/tryptophan peptides)
ACSHSGHGCGHGSHSCGRRRRRRRR

SEQ ID NO: 145 - protein, 25 aa, Artificial Sequence
  Synthetic CPP EA8_2H2W
ACSHSGWGCGHGSWSCGRRRRRRRR

SEQ ID NO: 146 - protein, 11 aa, Artificial Sequence
  Synthetic CPP F4
ACSSSPSKHCG

SEQ ID NO: 147 - protein, 23 aa, Artificial Sequence
  Synthetic CPP B1
ACSSSPSKHCGGGGRRRRRRRRR

SEQ ID NO: 148 - protein, 30 aa, Artificial Sequence
  Synthetic CPP Inv9
ADVFDRGGPYLQRGVADLVPTATLLDTYSP

SEQ ID NO: 149 - protein, 16 aa, Artificial Sequence
  Synthetic CPP C11
AEAEAEAEAKAKAKAK

SEQ ID NO: 150 - protein, 28 aa, Artificial Sequence
  Synthetic CPP A9
AEAEAEAEAKAKAKAKAGGGHRRRRRRR

SEQ ID NO: 151 - protein, 26 aa, Artificial Sequence
  Synthetic CPP Inv5
AEKVDPVKLNLTLSAAAEALTGLGDK

SEQ ID NO: 152 - protein, 20 aa, Artificial Sequence
  Synthetic CPP TH peptide
AGYLLGHINLHHLAHLHHIL

SEQ ID NO: 153 - protein, 21 aa, Artificial Sequence
  Synthetic CPP TH peptide
AGYLLGHINLHHLAHLHHILC

SEQ ID NO: 154 - protein, 21 aa, Artificial Sequence
  Synthetic CPP Transportan 10 (TP10)
AGYLLGKINLKALAALAKKIL

SEQ ID NO: 155 - protein, 24 aa, Artificial Sequence
  Synthetic CPP Transportan 10
AGYLLGKINLKALAALAKKILGGC

SEQ ID NO: 156 - protein, 38 aa, Artificial Sequence
  Synthetic CPP Transportan-PKI
AGYLLGKINLKALAALAKKILTYADFIASGRTGRRNAI

SEQ ID NO: 157 - protein, 20 aa, Artificial Sequence
  Synthetic CPP TK peptide
AGYLLGKINLKKLAKLLLIL

SEQ ID NO: 158 - protein, 19 aa, Artificial Sequence
  Synthetic CPP TP14
AGYLLGKLKALAALAKKIL

SEQ ID NO: 159 - protein, 21 aa, Artificial Sequence
  Synthetic CPP NF1
AGYLLGKTNLKALAALAKKIL

SEQ ID NO: 160 - protein, 26 aa, Artificial Sequence
  Synthetic CPP pAntpHD
AHALCLTERQIKIWFQNRRMKWKKEN

SEQ ID NO: 161 - protein, 26 aa, Artificial Sequence
  Synthetic CPP pAntpHD 40P2
AHALCPPERQIKIWFQNRRMKWKKEN

SEQ ID NO: 162 - protein, 9 aa, Artificial Sequence
  Synthetic CPP TCTP(1-9) M1A subsetution mutant
AIIYRDLIS

SEQ ID NO: 163 - protein, 12 aa, Artificial Sequence
  Synthetic CPP Peptide 49
AIPNNQLGFPFK

SEQ ID NO: 164 - protein, 30 aa, Artificial Sequence
  Synthetic CPP 30 A-K
AKKAKAAKKAKAAKKAKAAKKAKAAKKAKA

SEQ ID NO: 165 - protein, 24 aa, Artificial Sequence
  Synthetic CPP 24 A-K
AKKKAAKAAKKKAAKAAKKKAAKA

SEQ ID NO: 166 - protein, 32 aa, Artificial Sequence
  Synthetic CPP 32 A-K
AKKKAAKAAKKKAAKAAKKKAAKAAKKKAAKA

SEQ ID NO: 167 - protein, 9 aa, Artificial Sequence
  Synthetic CPP Ala49 substitution mutant of Tat (49-57)
AKKRRQRRR

SEQ ID NO: 168 - protein, 18 aa, Artificial Sequence
  Synthetic CPP MTat2-Nat
AKKRRQRRRAKKRRQRRR

SEQ ID NO: 169 - protein, 34 aa, Artificial Sequence
  Synthetic CPP F3
AKVKDEPQRRSARLSAKPAPPKPEPKPKKAPAKK

SEQ ID NO: 170 - protein, 38 aa, Artificial Sequence
  Synthetic CPP D5
ALALALALALALALALKIKKIKKIKKIKKLAKLAKKIK

SEQ ID NO: 171 - protein, 18 aa, Artificial Sequence
  Synthetic CPP pVEC mutant
ALIILRRRIRKQAHAHSK

SEQ ID NO: 172 - protein, 13 aa, Artificial Sequence
  Synthetic CPP S4(13)
ALWKTLLKKVLKA

SEQ ID NO: 173 - protein, 20 aa, Artificial Sequence
  Synthetic CPP S4(13)-PV
ALWKTLLKKVLKAPKKKRKV

SEQ ID NO: 174 - protein, 15 aa, Artificial Sequence
  Synthetic CPP No.14-11
ALWMRWYSPTTRRYG

SEQ ID NO: 175 - protein, 28 aa, Artificial Sequence
  Synthetic CPP Dermaseptin S4
ALWMTLLKKVLKAAAKAALNAVLVGANA

SEQ ID NO: 176 - protein, 12 aa, Artificial Sequence
  Synthetic CPP CTP (cardiac targetting peptide)
APWHLSSQYSRT

SEQ ID NO: 177 - protein, 16 aa, Artificial Sequence
  Synthetic CPP Ala43 substitution mutant of pAntp (43-58)
AQIKIWFQNRRMKWKK

SEQ ID NO: 178 - protein, 19 aa, Artificial Sequence
  Synthetic CPP kEA2x1 (Kallikrein inhibitor with external arginines)
ARCSDRFRNCPADEALCGR

SEQ ID NO: 179 - protein, 19 aa, Artificial Sequence
  Synthetic CPP EA2x1 (External arginines)
ARCSGSGSGCGSGSGSCGR

SEQ ID NO: 180 - protein, 30 aa, Artificial Sequence
  Synthetic CPP 30 A-R
ARRARAARRARAARRARAARRARAARRARA

SEQ ID NO: 181 - protein, 21 aa, Artificial Sequence
  Synthetic CPP kEA2x2
ARRCSDRFRNCPADEALCGRR

SEQ ID NO: 182 - protein, 21 aa, Artificial Sequence
  Synthetic CPP EA2x2
ARRCSGSGSGCGSGSGSCGRR

SEQ ID NO: 183 - protein, 24 aa, Artificial Sequence
  Synthetic CPP 24 A-R
ARRRAARAARRRAARAARRRAARA

SEQ ID NO: 184 - protein, 32 aa, Artificial Sequence
  Synthetic CPP 32 A-R
ARRRAARAARRRAARAARRRAARAARRRAARA

SEQ ID NO: 185 - protein, 23 aa, Artificial Sequence
  Synthetic CPP kEA2x3
ARRRCSDRFRNCPADEALCGRRR

SEQ ID NO: 186 - protein, 23 aa, Artificial Sequence
  Synthetic CPP EA2x3
ARRRCSGSGSGCGSGSGSCGRRR

SEQ ID NO: 187 - protein, 25 aa, Artificial Sequence
  Synthetic CPP kEA2x4
ARRRRCSDRFRNCPADEALCGRRRR

SEQ ID NO: 188 - protein, 25 aa, Artificial Sequence
  Synthetic CPP EA2x4
ARRRRCSGSGSGCGSGSGSCGRRRR

SEQ ID NO: 189 - protein, 32 aa, Artificial Sequence
  Synthetic CPP Inv8
ARTINAQQAELDSALLAAAGFGNTTADVFDRG

SEQ ID NO: 190 - protein, 21 aa, Artificial Sequence
  Synthetic CPP FHV gamma peptide
ASMWERVKSIIKSSLAAASNI

SEQ ID NO: 191 - protein, 12 aa, Artificial Sequence
  Synthetic CPP Peptide 26
AVPAENALNNPF

SEQ ID NO: 192 - protein, 26 aa, Artificial Sequence
  Synthetic CPP pAntpHD 50A
AYALCLTERQIKIWFANRRMKWKKEN

SEQ ID NO: 193 - protein, 12 aa, Artificial Sequence
  Synthetic CPP TAT-cysteine peptide
AYGRKKRRQRRR

SEQ ID NO: 194 - protein, 20 aa, Artificial Sequence
  Synthetic CPP TP10
AYLLGKINLKALAALAKKIL

SEQ ID NO: 195 - protein, 20 aa, Artificial Sequence
  Synthetic CPP L1 (Ala32 substitution mutant of LALF (32-51))
AYRIKPTFRRLKWKYKGKFW

SEQ ID NO: 196 - protein, 9 aa, Artificial Sequence
  Synthetic CPP CAR
CARSKNKDC

SEQ ID NO: 197 - protein, 12 aa, Artificial Sequence
  Synthetic CPP Peptide 2
CASGQQGLLKLC

SEQ ID NO: 198 - protein, 13 aa, Artificial Sequence
  Synthetic CPP S-TAT
CAYGGQQGGQGGG

SEQ ID NO: 199 - protein, 13 aa, Artificial Sequence
  Synthetic CPP PTX-TAT-LP
CAYGRKKRRQRRR

SEQ ID NO: 200 - protein, 13 aa, Artificial Sequence
  Synthetic CPP TAT
CCTGRKKRRQRRR

SEQ ID NO: 201 - protein, 20 aa, Artificial Sequence
  Synthetic CPP Alexa488-Melan-A-polyLys (control peptide)
CELAGIGILTVKKKKKQKKK

SEQ ID NO: 202 - protein, 20 aa, Artificial Sequence
  Synthetic CPP Alexa488-Melan-A-TAT
CELAGIGILTVRKKRRQRRR

SEQ ID NO: 203 - protein, 23 aa, Artificial Sequence
  Synthetic CPP DPV15b
CGAYDLRRRERQSRLRRRERQSR

SEQ ID NO: 204 - protein, 36 aa, Artificial Sequence
  Synthetic CPP POD
CGGGARKKAAKAARKKAAKAARKKAAKAARKKAAKA

SEQ ID NO: 205 - protein, 16 aa, Artificial Sequence
  Synthetic CPP TAT
CGGGGYGRKKRRQRRR

SEQ ID NO: 206 - protein, 17 aa, Artificial Sequence
  Synthetic CPP sgRNA-CPP
CGGGRRRRRRRRRLLLL

SEQ ID NO: 207 - protein, 15 aa, Artificial Sequence
  Synthetic CPP AgNP-TAT
CGGGYGRKKRRQRRR

SEQ ID NO: 208 - protein, 30 aa, Artificial Sequence
  Synthetic CPP b-WT1-pTj
CGGKDCERRFSRSDQLKRHQRRHTGVKPFQ

SEQ ID NO: 209 - protein, 25 aa, Artificial Sequence
  Synthetic CPP M918(C-S)
CGGMVTVLFRRLRIRRASGPPRVRV

SEQ ID NO: 210 - protein, 7 aa, Artificial Sequence
  Synthetic CPP tLyp-1
CGNKRTR

SEQ ID NO: 211 - protein, 9 aa, Artificial Sequence
  Synthetic CPP Lyp-1
CGNKRTRGC

SEQ ID NO: 212 - protein, 20 aa, Artificial Sequence
  Synthetic CPP IX
CGRKKRAARQRAARAARPPQ

SEQ ID NO: 213 - protein, 16 aa, Artificial Sequence
  Synthetic CPP VI
CGRKKRAARQRRRPPQ

SEQ ID NO: 214 - protein, 20 aa, Artificial Sequence
  Synthetic CPP XIII
CGRKKRLLRQRLLRLLRPPQ

SEQ ID NO: 215 - protein, 16 aa, Artificial Sequence
  Synthetic CPP X
CGRKKRLLRQRRRPPQ

SEQ ID NO: 216 - protein, 16 aa, Artificial Sequence
  Synthetic CPP VIII
CGRKKRRQRAARRPPQ

SEQ ID NO: 217 - protein, 16 aa, Artificial Sequence
  Synthetic CPP XII
CGRKKRRQRLLRRPPQ

SEQ ID NO: 218 - protein, 16 aa, Artificial Sequence
  Synthetic CPP VII
CGRKKRRQRRAARPPQ

SEQ ID NO: 219 - protein, 16 aa, Artificial Sequence
  Synthetic CPP XI
CGRKKRRQRRLLRPPQ

SEQ ID NO: 220 - protein, 14 aa, Artificial Sequence
  Synthetic CPP C16NTD
CGRKKRRQRRRPPQ

SEQ ID NO: 221 - protein, 16 aa, Artificial Sequence
  Synthetic CPP III
CGRKKRRQRRWWRPPQ

SEQ ID NO: 222 - protein, 16 aa, Artificial Sequence
  Synthetic CPP IV
CGRKKRRQRWWRRPPQ

SEQ ID NO: 223 - protein, 16 aa, Artificial Sequence
  Synthetic CPP II
CGRKKRWWRQRRRPPQ

SEQ ID NO: 224 - protein, 20 aa, Artificial Sequence
  Synthetic CPP V
CGRKKRWWRQRWWRWWRPPQ

SEQ ID NO: 225 - protein, 15 aa, Artificial Sequence
  Synthetic CPP TAT
CGYGRKKRRQRRRGC

SEQ ID NO: 226 - protein, 8 aa, Artificial Sequence
  Synthetic CPP T7-LP
CHAIYPRH

SEQ ID NO: 227 - protein, 21 aa, Artificial Sequence
  Synthetic CPP HR9
CHHHHHRRRRRRRRRHHHHHC

SEQ ID NO: 228 - protein, 10 aa, Artificial Sequence
  Synthetic CPP CH2 R4 H2 C
CHHRRRRHHC

SEQ ID NO: 229 - protein, 26 aa, Artificial Sequence
  Synthetic CPP Melittin
CIGAVLKVLTTGLPALISWIKRKRQQ

SEQ ID NO: 230 - protein, 10 aa, Artificial Sequence
  Synthetic CPP TCTP-CPP 6
CIISRDLISH

SEQ ID NO: 231 - protein, 32 aa, Artificial Sequence
  Synthetic CPP F3 Peptide
CKDEPQRRSARLSAKPAPPKPEPKPKKAPAKK

SEQ ID NO: 232 - protein, 10 aa, Artificial Sequence
  Synthetic CPP ck9
CKKKKKKKKK

SEQ ID NO: 233 - protein, 13 aa, Artificial Sequence
  Synthetic CPP acFTAT
CKYGRKKRRQRRR

SEQ ID NO: 234 - protein, 31 aa, Artificial Sequence
  Synthetic CPP Dox-pVEC-gHo (Dox- gHoPe2)
CLLIILRRRIRKQAHAHSKNHQQQNPHQPPM

SEQ ID NO: 235 - protein, 20 aa, Artificial Sequence
  Synthetic CPP Mgpe-10
CLLYWFRRRHRHHRRRHRRC

SEQ ID NO: 236 - protein, 5 aa, Artificial Sequence
  Synthetic CPP NGR
CNGRC

SEQ ID NO: 237 - protein, 10 aa, Artificial Sequence
  Synthetic CPP Crot (27-39) derevative
CRFRFKCCKK

SEQ ID NO: 238 - protein, 10 aa, Artificial Sequence
  Synthetic CPP Crot (27-39) derevative
CRFRWKCCKK

SEQ ID NO: 239 - protein, 5 aa, Artificial Sequence
  Synthetic CPP RGD
CRGDC

SEQ ID NO: 240 - protein, 5 aa, Artificial Sequence
  Synthetic CPP CRGDK
CRGDK

SEQ ID NO: 241 - protein, 9 aa, Artificial Sequence
  Synthetic CPP iRGD
CRGDKGDPC

SEQ ID NO: 242 - protein, 9 aa, Artificial Sequence
  Synthetic CPP iRGD-CDD
CRGDKGPDC

SEQ ID NO: 243 - protein, 13 aa, Artificial Sequence
  Synthetic CPP D-TAT
CRKARYRGRKRQR

SEQ ID NO: 244 - protein, 9 aa, Artificial Sequence
  Synthetic CPP iNGR
CRNGRGPDC

SEQ ID NO: 245 - protein, 18 aa, Artificial Sequence
  Synthetic CPP Reduced linear penetratin
CRQIKIWFPNRRMKWKKC

SEQ ID NO: 246 - protein, 17 aa, Artificial Sequence
  Synthetic CPP Penetratin
CRQIKIWFQNRRMKWKK

SEQ ID NO: 247 - protein, 31 aa, Artificial Sequence
  Synthetic CPP KLA-Pen
CRQIKIWFQNRRMKWKKKLAKLAKKLAKLAK

SEQ ID NO: 248 - protein, 20 aa, Artificial Sequence
  Synthetic CPP Mgpe-9
CRRLRHLRHHYRRRWHRFRC

SEQ ID NO: 249 - protein, 9 aa, Artificial Sequence
  Synthetic CPP R8
CRRRRRRRR

SEQ ID NO: 250 - protein, 10 aa, Artificial Sequence
  Synthetic CPP Crot (27-39) derevative
CRWRFKCCKK

SEQ ID NO: 251 - protein, 10 aa, Artificial Sequence
  Synthetic CPP CyLoP-1
CRWRWKCCKK

SEQ ID NO: 252 - protein, 8 aa, Artificial Sequence
  Synthetic CPP Crot (27-39) derevative
CRWRWKCG

SEQ ID NO: 253 - protein, 11 aa, Artificial Sequence
  Synthetic CPP Crot (27-39) derevative
CRWRWKCGCKK

SEQ ID NO: 254 - protein, 10 aa, Artificial Sequence
  Synthetic CPP Crot (27-39) derevative
CRWRWKCSKK

SEQ ID NO: 255 - protein, 10 aa, Artificial Sequence
  Synthetic CPP Crot (27-39) derevative
CRWRWKSSKK

SEQ ID NO: 256 - protein, 17 aa, Artificial Sequence
  Synthetic CPP C105Y
CSIPPEVKFNKPFVYLI

SEQ ID NO: 257 - protein, 16 aa, Artificial Sequence
  Synthetic CPP C105Y
CSIPPEVKFNPFVYLI

SEQ ID NO: 258 - protein, 9 aa, Artificial Sequence
  Synthetic CPP CSK
CSKSSDYQC

SEQ ID NO: 259 - protein, 18 aa, Artificial Sequence
  Synthetic CPP 1A
CSSLDEPGRGGFSSESKV

SEQ ID NO: 260 - protein, 14 aa, Artificial Sequence
  Synthetic CPP LI
CTSTTAKRKKRKLK

SEQ ID NO: 261 - protein, 6 aa, Artificial Sequence
  Synthetic CPP Peptide 1-NTHS-delta
CTWLKY

SEQ ID NO: 262 - protein, 7 aa, Artificial Sequence
  Synthetic CPP Peptide 1-NTS-delta
CTWLKYH

SEQ ID NO: 263 - protein, 19 aa, Artificial Sequence
  Synthetic CPP DPV1048
CVKRGLKLRHVRPRVTRDV

SEQ ID NO: 264 - protein, 13 aa, Artificial Sequence
  Synthetic CPP S41
CVQWSLLRGYQPC

SEQ ID NO: 265 - protein, 15 aa, Artificial Sequence
  Synthetic CPP LMWP
CVSRRRRRRGGRRRR

SEQ ID NO: 266 - protein, 5 aa, Artificial Sequence
  Synthetic CPP AlkCWK3
CWKKK

SEQ ID NO: 267 - protein, 10 aa, Artificial Sequence
  Synthetic CPP AlkCWK8
CWKKKKKKKK

SEQ ID NO: 268 - protein, 15 aa, Artificial Sequence
  Synthetic CPP AlkCWK13
CWKKKKKKKKKKKKK

SEQ ID NO: 269 - protein, 20 aa, Artificial Sequence
  Synthetic CPP AlkCWK18
CWKKKKKKKKKKKKKKKKKK

SEQ ID NO: 270 - protein, 12 aa, Artificial Sequence
  Synthetic CPP PTX-N-TAT-LP
CYGRKKRRQRRR

SEQ ID NO: 271 - protein, 35 aa, Artificial Sequence
  Synthetic CPP EGFP-VP_22
DAATARGRGRSAASRPTERPRAPARSASRPRRPVD

SEQ ID NO: 272 - protein, 34 aa, Artificial Sequence
  Synthetic CPP VP22
DAATATRGRSAASRPTQRPRAPARSASRPRRPVE

SEQ ID NO: 273 - protein, 11 aa, Artificial Sequence
  Synthetic CPP Crot (27-39) derivative
DCRWRWKCCKK

SEQ ID NO: 274 - protein, 18 aa, Artificial Sequence
  Synthetic CPP hCT(1532)
DFNKFHTFPQTAIGVGAP

SEQ ID NO: 275 - protein, 12 aa, Artificial Sequence
  Synthetic CPP rV1aR (102-113a)
DITYRFRGPDWL

SEQ ID NO: 276 - protein, 12 aa, Artificial Sequence
  Synthetic CPP Peptide 52
DPATNPGPHFPR

SEQ ID NO: 277 - protein, 26 aa, Artificial Sequence
  Synthetic CPP VT5
DPKGDPKGVTVTVTVTVTGKGDPKPD

SEQ ID NO: 278 - protein, 15 aa, Artificial Sequence
  Synthetic CPP Secretory leukoprotease inhibitor derived PTD
DPVDTPNPTRRKPGK

SEQ ID NO: 279 - protein, 17 aa, Artificial Sequence
  Synthetic CPP Unknown
DRDDRDDRDDRDDRDDR

SEQ ID NO: 280 - protein, 10 aa, Artificial Sequence
  Synthetic CPP Unknown
DRDRDRDRDR

SEQ ID NO: 281 - protein, 17 aa, Artificial Sequence
  Synthetic CPP RSG 1.2 truncated
DRRRRGSRPSGAERRRR

SEQ ID NO: 282 - protein, 22 aa, Artificial Sequence
  Synthetic CPP RSG 1.2
DRRRRGSRPSGAERRRRRAAAA

SEQ ID NO: 283 - protein, 15 aa, Artificial Sequence
  Synthetic CPP 2
DSLKSYWYLQKFSWR

SEQ ID NO: 284 - protein, 22 aa, Artificial Sequence
  Synthetic CPP C45D18
DTWAGVEAIIRILQQLLFIHFR

SEQ ID NO: 285 - protein, 16 aa, Artificial Sequence
  Synthetic CPP GV1001
EARPALLTSRLRFIPK

SEQ ID NO: 286 - protein, 10 aa, Artificial Sequence
  Synthetic CPP Peptide 4
ECYPKKGQDP

SEQ ID NO: 287 - protein, 5 aa, Artificial Sequence
  Synthetic CPP Glu-Ala
EEEAA

SEQ ID NO: 288 - protein, 13 aa, Artificial Sequence
  Synthetic CPP Glu-Oct-6
EEEAAGRKRKKRT

SEQ ID NO: 289 - protein, 8 aa, Artificial Sequence
  Synthetic CPP Glu-Lys
EEEAAKKK

SEQ ID NO: 290 - protein, 23 aa, Artificial Sequence
  Synthetic CPP ACPP
EEEEEEEEPLGLAGRRRRRRRRN

SEQ ID NO: 291 - protein, 10 aa, Artificial Sequence
  Synthetic CPP Cyt 4-13
EKGKKIFIMK

SEQ ID NO: 292 - protein, 61 aa, Artificial Sequence
  Synthetic CPP Engrailed (454-513)
EKRPRTAFSSEQLARLKREFNENRYLTTERRRQQLSSELGLNEAQIKIWFQNKRAKIKKST

SEQ ID NO: 293 - protein, 18 aa, Artificial Sequence
  Synthetic CPP X
ELALELALEALEAALELA

SEQ ID NO: 294 - protein, 5 aa, Artificial Sequence
  Synthetic CPP Bip18
ELPVM

SEQ ID NO: 295 - protein, 12 aa, Artificial Sequence
  Synthetic CPP Peptide 65
EPDNWSLDFPRR

SEQ ID NO: 296 - protein, 14 aa, Artificial Sequence
  Synthetic CPP Unknown
ERERERERERERER

SEQ ID NO: 297 - protein, 8 aa, Artificial Sequence
  Synthetic CPP HATF3
ERKKRRRE

SEQ ID NO: 298 - protein, 20 aa, Artificial Sequence
  Synthetic CPP c-Myc-R11
ESGGGGSPGRRRRRRRRRRR

SEQ ID NO: 299 - protein, 12 aa, Artificial Sequence
  Synthetic CPP Peptide 34
FAPWDTASFMLG

SEQ ID NO: 300 - protein, 12 aa, Artificial Sequence
  Synthetic CPP Peptide 33
FDPFFWKYSPRD

SEQ ID NO: 301 - protein, 13 aa, Artificial Sequence
  Synthetic CPP Phe-Oct-6
FFFAAGRKRKKRT

SEQ ID NO: 302 - protein, 17 aa, Artificial Sequence
  Synthetic CPP F6R8 (Alexa)
FFFFFFGRRRRRRRRGC

SEQ ID NO: 303 - protein, 15 aa, Artificial Sequence
  Synthetic CPP F4R8 (Alexa)
FFFFGRRRRRRRRGC

SEQ ID NO: 304 - protein, 12 aa, Artificial Sequence
  Synthetic CPP F2R8 (Alexa)
FFGRRRRRRRGC

SEQ ID NO: 305 - protein, 27 aa, Artificial Sequence
  Synthetic CPP LAH4-X1F2
FFKKLALHALHLLALLWLHLAHLALKK

SEQ ID NO: 306 - protein, 15 aa, Artificial Sequence
  Synthetic CPP PEG-Pas-delta-PKR8(Alexa)
FFLIGRRRRRRRRGC

SEQ ID NO: 307 - protein, 17 aa, Artificial Sequence
  Synthetic CPP PasR8 (Alexa)
FFLIPKGRRRRRRRRGC

SEQ ID NO: 308 - protein, 16 aa, Artificial Sequence
  Synthetic CPP PR9
FFLIPKGRRRRRRRRR

SEQ ID NO: 309 - protein, 8 aa, Artificial Sequence
  Synthetic CPP F10
FHFHFRFR

SEQ ID NO: 310 - protein, 12 aa, Artificial Sequence
  Synthetic CPP TCTP-CPP 15
FIIFRIAASHKK

SEQ ID NO: 311 - protein, 6 aa, Artificial Sequence
  Synthetic CPP LR8DRIHF
FIRIGC

SEQ ID NO: 312 - protein, 16 aa, Artificial Sequence
  Synthetic CPP Tat (37-53)
FITKALGISYGRKKRR

SEQ ID NO: 313 - protein, 23 aa, Artificial Sequence
  Synthetic CPP Tat (37-60)
FITKALGISYGRKKRRQRRRPPQ

SEQ ID NO: 314 - protein, 7 aa, Artificial Sequence
  Synthetic CPP C.e SDC3
FKKFRKF

SEQ ID NO: 315 - protein, 26 aa, Artificial Sequence
  Synthetic CPP LAH4-X1F1
FKKLALHALHLLALLWLHLAHLALKK

SEQ ID NO: 316 - protein, 12 aa, Artificial Sequence
  Synthetic CPP PN285
FKQQQQQQQQQQ

SEQ ID NO: 317 - protein, 15 aa, Artificial Sequence
  Synthetic CPP M 511
FLGKKFKKYFLQLLK

SEQ ID NO: 318 - protein, 23 aa, Artificial Sequence
  Synthetic CPP G53-4
FLIFIRVICIVIAKLKANLMCKT

SEQ ID NO: 319 - protein, 19 aa, Artificial Sequence
  Synthetic CPP PF22
FLKLLKKFLKLFKKLLKLF

SEQ ID NO: 320 - protein, 19 aa, Artificial Sequence
  Synthetic CPP C1
FQFNFQFNGGGHRRRRRRR

SEQ ID NO: 321 - protein, 10 aa, Artificial Sequence
  Synthetic CPP pAntp (49-58)
FQNRRMKWKK

SEQ ID NO: 322 - protein, 12 aa, Artificial Sequence
  Synthetic CPP Peptide 32
FQPYDHPAEVSY

SEQ ID NO: 323 - protein, 16 aa, Artificial Sequence
  Synthetic CPP M4
FQWQRNMRKVRGPPVS

SEQ ID NO: 324 - protein, 8 aa, Artificial Sequence
  Synthetic CPP Single mitochondrial penetrating peptide
FRFKFRFK

SEQ ID NO: 325 - protein, 37 aa, Artificial Sequence
  Synthetic CPP ARF(1-37) scr
FRVPLRIRPCVVAPRLVMVRHTFGRIARWVAGPLETR

SEQ ID NO: 326 - protein, 9 aa, Artificial Sequence
  Synthetic CPP F8
FTFHFTFHF

SEQ ID NO: 327 - protein, 12 aa, Artificial Sequence
  Synthetic CPP Peptide 35
FTYKNFFWLPEL

SEQ ID NO: 328 - protein, 22 aa, Artificial Sequence
  Synthetic CPP ARF(1-22) scr
FVTRGCPRRLVARLIRVMVPRR

SEQ ID NO: 329 - protein, 14 aa, Artificial Sequence
  Synthetic CPP SFTI-M1
GACTKSIPPICFPD

SEQ ID NO: 330 - protein, 27 aa, Artificial Sequence
  Synthetic CPP MPG?
GALFLAFLAAALSLMGLWSQPKKKRKV

SEQ ID NO: 331 - protein, 27 aa, Artificial Sequence
  Synthetic CPP P(alpha)
GALFLAFLAAALSLMGLWSQPKKKRRV

SEQ ID NO: 332 - protein, 27 aa, Artificial Sequence
  Synthetic CPP MPG-beta
GALFLGFLGAAGSTMGAWSQPKKKRKV

SEQ ID NO: 333 - protein, 24 aa, Artificial Sequence
  Synthetic CPP EGFP-MPG
GALFLGWLGAAGSTMGAPKKKRKV

SEQ ID NO: 334 - protein, 27 aa, Artificial Sequence
  Synthetic CPP MPG-NLS
GALFLGWLGAAGSTMGAPKSKRKVGGC

SEQ ID NO: 335 - protein, 22 aa, Artificial Sequence
  Synthetic CPP DPV15b
GAYDLRRRERQSRLRRRERQSR

SEQ ID NO: 336 - protein, 16 aa, Artificial Sequence
  Synthetic CPP Tat
GCGGGYGRKKRRQRRR

SEQ ID NO: 337 - protein, 27 aa, Artificial Sequence
  Synthetic CPP Inv7
GDVYADAAPDLFDFLDSSVTTARTINA

SEQ ID NO: 338 - protein, 22 aa, Artificial Sequence
  Synthetic CPP
GEQIAQLIAGYIDIILKKKKSK

SEQ ID NO: 339 - protein, 24 aa, Artificial Sequence
  Synthetic CPP CF-Vim-TBS.58-81
GGAYVTRSSAVRLRSSVPGVRLLQ

SEQ ID NO: 340 - protein, 35 aa, Artificial Sequence
  Synthetic CPP POD
GGGARKKAAKAARKKAAKAARKKAAKAARKKAAKA

SEQ ID NO: 341 - protein, 17 aa, Artificial Sequence
  Synthetic CPP m9R
GGGGRRRRRRRRRLLLL

SEQ ID NO: 342 - protein, 19 aa, Artificial Sequence
  Synthetic CPP G3R6TAT
GGGRRRRRRYGRKKRRQRR

SEQ ID NO: 343 - protein, 11 aa, Artificial Sequence
  Synthetic CPP CTP
GGRRARRRRRR

SEQ ID NO: 344 - protein, 34 aa, Artificial Sequence
  Synthetic CPP MCoK6A mutant
GGVCPAILKKCRRDSDCPGACICRGNGYCGSGSD

SEQ ID NO: 345 - protein, 34 aa, Artificial Sequence
  Synthetic CPP MCoKKAA double mutant
GGVCPKILAACRRDSDCPGACICRGNGYCGSGSD

SEQ ID NO: 346 - protein, 34 aa, Artificial Sequence
  Synthetic CPP MCoK9A mutant
GGVCPKILAKCRRDSDCPGACICRGNGYCGSGSD

SEQ ID NO: 347 - protein, 34 aa, Artificial Sequence
  Synthetic CPP MCoK10A mutant
GGVCPKILKACRRDSDCPGACICRGNGYCGSGSD

SEQ ID NO: 348 - protein, 34 aa, Artificial Sequence
  Synthetic CPP MCoTI-M1
GGVCPKILKKCRRDSDCPGACICRGNGWCGSGSD

SEQ ID NO: 349 - protein, 34 aa, Artificial Sequence
  Synthetic CPP MCoTI-II
GGVCPKILKKCRRDSDCPGACICRGNGYCGSGSD

SEQ ID NO: 350 - protein, 34 aa, Artificial Sequence
  Synthetic CPP MCoTI-M3
GGVCPKILRRCRRDSDCPGACICRGNGWCGSGSD

SEQ ID NO: 351 - protein, 34 aa, Artificial Sequence
  Synthetic CPP MCoTI-M2
GGVCPKILRRCRRDSDCPGACICRGNGYCGSGSD

SEQ ID NO: 352 - protein, 34 aa, Artificial Sequence
  Synthetic CPP MCoTI-M4
GGVCPKILRRCRRDSDCPGACICRGNGYCGSGSR

SEQ ID NO: 353 - protein, 34 aa, Artificial Sequence
  Synthetic CPP MCoTI-M5
GGVCPRILRRCRRDSDCPGACICRGNGYCGSGSK

SEQ ID NO: 354 - protein, 42 aa, Artificial Sequence
  Synthetic CPP MG2A
GIGKFLHSAKKFGKAFVGEIMNSGGKKWKMRRNQFWVKVQRG

SEQ ID NO: 355 - protein, 23 aa, Artificial Sequence
  Synthetic CPP MG2d
GIGKFLHSAKKWGKAFVGQIMNC

SEQ ID NO: 356 - protein, 16 aa, Artificial Sequence
  Synthetic CPP Cyclin L ania-6a
GKHRHERGHHRDRRER

SEQ ID NO: 357 - protein, 16 aa, Artificial Sequence
  Synthetic CPP
GKINLKALAALAKKIL

SEQ ID NO: 358 - protein, 15 aa, Artificial Sequence
  Synthetic CPP GKK peptide
GKKALKLAAKLLKKC

SEQ ID NO: 359 - protein, 10 aa, Artificial Sequence
  Synthetic CPP Lys9
GKKKKKKKKK

SEQ ID NO: 360 - protein, 11 aa, Artificial Sequence
  Synthetic CPP TCF1-ALPHA
GKKKKRKREKL

SEQ ID NO: 361 - protein, 17 aa, Artificial Sequence
  Synthetic CPP beta Zip TF
GKKKRKLSNRESAKRSR

SEQ ID NO: 362 - protein, 17 aa, Artificial Sequence
  Synthetic CPP ABL-1
GKKTNLFSALIKKKKTA

SEQ ID NO: 363 - protein, 18 aa, Artificial Sequence
  Synthetic CPP GCN-4
GKRARNTEAARRSRARKL

SEQ ID NO: 364 - protein, 22 aa, Artificial Sequence
  Synthetic CPP HB-EGF
GKRKKKGKGLGKKRDPCLRKYK

SEQ ID NO: 365 - protein, 15 aa, Artificial Sequence
  Synthetic CPP DPV7
GKRKKKGKLGKKRDP

SEQ ID NO: 366 - protein, 17 aa, Artificial Sequence
  Synthetic CPP DPV7b
GKRKKKGKLGKKRPRSR

SEQ ID NO: 367 - protein, 15 aa, Artificial Sequence
  Synthetic CPP HEN2/NSLC2
GKRRRRATAKYRSAH

SEQ ID NO: 368 - protein, 18 aa, Artificial Sequence
  Synthetic CPP Thyroid A-1
GKRVAKRKLIEQNRERRR

SEQ ID NO: 369 - protein, 22 aa, Artificial Sequence
  Synthetic CPP Inv2
GKYVSLTTPKNPTKRRITPKDV

SEQ ID NO: 370 - protein, 36 aa, Artificial Sequence
  Synthetic CPP Peptide 599
GLFEAIEGFIENGWEGMIDGWYGGGGRRRRRRRRRK

SEQ ID NO: 371 - protein, 20 aa, Artificial Sequence
  Synthetic CPP JST-1
GLFEALLELLESLWELLLEA

SEQ ID NO: 372 - protein, 20 aa, Artificial Sequence
  Synthetic CPP ppTG1
GLFKALLKLLKSLWKLLLKA

SEQ ID NO: 373 - protein, 23 aa, Artificial Sequence
  Synthetic CPP ppTG
GLFKALLKLLKSLWKLLLKAGGC

SEQ ID NO: 374 - protein, 20 aa, Artificial Sequence
  Synthetic CPP EGFP-ppTG20
GLFRALLRLLRSLWRLLLRA

SEQ ID NO: 375 - protein, 34 aa, Artificial Sequence
  Synthetic CPP Inv6
GLGDKFGESIVNANTVLDDLNSRMPQSRHDIQQL

SEQ ID NO: 376 - protein, 22 aa, Artificial Sequence
  Synthetic CPP PN283
GLGSLLKKAGKKLKQPKSKRKV

SEQ ID NO: 377 - protein, 16 aa, Artificial Sequence
  Synthetic CPP Peptide 2C- GNS
GLKKLAELAHKLLKLG

SEQ ID NO: 378 - protein, 17 aa, Artificial Sequence
  Synthetic CPP EA
GLKKLAELAHKLLKLGC

SEQ ID NO: 379 - protein, 16 aa, Artificial Sequence
  Synthetic CPP TAMARA-peptide 1
GLKKLAELFHKLLKLG

SEQ ID NO: 380 - protein, 17 aa, Artificial Sequence
  Synthetic CPP EF
GLKKLAELFHKLLKLGC

SEQ ID NO: 381 - protein, 17 aa, Artificial Sequence
  Synthetic CPP RA
GLKKLARLAHKLLKLGC

SEQ ID NO: 382 - protein, 17 aa, Artificial Sequence
  Synthetic CPP RF
GLKKLARLFHKLLKLGC

SEQ ID NO: 383 - protein, 27 aa, Artificial Sequence
  Synthetic CPP N-E5L-Sc18
GLLEALAELLEGLRKRLRKFRNKIKEK

SEQ ID NO: 384 - protein, 12 aa, Artificial Sequence
  Synthetic CPP DSPE-PEG-CPP (CPP-Lp)
GLPRRRRRRRRR

SEQ ID NO: 385 - protein, 29 aa, Artificial Sequence
  Synthetic CPP kT20K mutant
GLPVCGETCVGGTCNTPGCKCSWPVCTRN

SEQ ID NO: 386 - protein, 29 aa, Artificial Sequence
  Synthetic CPP kV25K mutant
GLPVCGETCVGGTCNTPGCTCSWPKCTRN

SEQ ID NO: 387 - protein, 16 aa, Artificial Sequence
  Synthetic CPP CF-sC18
GLRKRLRKFRNKIKEK

SEQ ID NO: 388 - protein, 20 aa, Artificial Sequence
  Synthetic CPP CADY-1c
GLWRALWRALRSLWKLKRKV

SEQ ID NO: 389 - protein, 21 aa, Artificial Sequence
  Synthetic CPP CADY-2c
GLWRALWRALWRSLWKKKRKV

SEQ ID NO: 390 - protein, 21 aa, Artificial Sequence
  Synthetic CPP CADY-1b
GLWRALWRALWRSLWKLKRKV

SEQ ID NO: 391 - protein, 21 aa, Artificial Sequence
  Synthetic CPP CADY-2
GLWRALWRALWRSLWKLKWKV

SEQ ID NO: 392 - protein, 21 aa, Artificial Sequence
  Synthetic CPP CADY-2b
GLWRALWRALWRSLWKSKRKV

SEQ ID NO: 393 - protein, 20 aa, Artificial Sequence
  Synthetic CPP CADY-1e
GLWRALWRGLRSLWKKKRKV

SEQ ID NO: 394 - protein, 20 aa, Artificial Sequence
  Synthetic CPP CADY-1d
GLWRALWRGLRSLWKLKRKV

SEQ ID NO: 395 - protein, 20 aa, Artificial Sequence
  Synthetic CPP CAD-2 (des-acetyl, Lys19-CADY)
GLWRALWRLLRSLWRLLWKA

SEQ ID NO: 396 - protein, 27 aa, Artificial Sequence
  Synthetic CPP CADY-2e
GLWRALWRLLRSLWRLLWSQPKKKRKV

SEQ ID NO: 397 - protein, 24 aa, Artificial Sequence
  Synthetic CPP CADY-1
GLWWKAWWKAWWKSLWWRKRKRKA

SEQ ID NO: 398 - protein, 20 aa, Artificial Sequence
  Synthetic CPP CADY2
GLWWRLWWRLRSWFRLWFRA

SEQ ID NO: 399 - protein, 15 aa, Artificial Sequence
  Synthetic CPP HipC
GNYAHRVGAGAPVWL

SEQ ID NO: 400 - protein, 13 aa, Artificial Sequence
  Synthetic CPP 435B peptide
GPFHFYQFLFPPV

SEQ ID NO: 401 - protein, 14 aa, Artificial Sequence
  Synthetic CPP SFTI-M2
GRCTKSIPPICFPA

SEQ ID NO: 402 - protein, 14 aa, Artificial Sequence
  Synthetic CPP SFTI-1
GRCTKSIPPICFPD

SEQ ID NO: 403 - protein, 14 aa, Artificial Sequence
  Synthetic CPP SFTI-M3
GRCTKSIPPICWPD

SEQ ID NO: 404 - protein, 14 aa, Artificial Sequence
  Synthetic CPP SFTI-M4
GRCTKSIPPICWPK

SEQ ID NO: 405 - protein, 14 aa, Artificial Sequence
  Synthetic CPP SFTI-M5
GRCTRSIPPKCWPD

SEQ ID NO: 406 - protein, 24 aa, Artificial Sequence
  Synthetic CPP Pep3(Mutant)
GRGDGPRRKKKKGPRRKKKKGPRR

SEQ ID NO: 407 - protein, 8 aa, Artificial Sequence
  Synthetic CPP Pep1
GRGDSPRR

SEQ ID NO: 408 - protein, 24 aa, Artificial Sequence
  Synthetic CPP Pep3
GRGDSPRRKKKKSPRRKKKKSPRR

SEQ ID NO: 409 - protein, 12 aa, Artificial Sequence
  Synthetic CPP Pep2
GRGDSPRRSPRR

SEQ ID NO: 410 - protein, 12 aa, Artificial Sequence
  Synthetic CPP hPER3 NLS
GRKGKHKRKKLP

SEQ ID NO: 411 - protein, 14 aa, Artificial Sequence
  Synthetic CPP Ala substitution mutant of Tat (48-60)
GRKKRRQARAPPQC

SEQ ID NO: 412 - protein, 11 aa, Artificial Sequence
  Synthetic CPP Arg deletion mutant of Tat (48-60)
GRKKRRQPPQC

SEQ ID NO: 413 - protein, 14 aa, Artificial Sequence
  Synthetic CPP Ala substitution mutant of Tat (48-60)
GRKKRRQRARPPQC

SEQ ID NO: 414 - protein, 12 aa, Artificial Sequence
  Synthetic CPP Arg deletion mutant of Tat (48-60)
GRKKRRQRPPQC

SEQ ID NO: 415 - protein, 13 aa, Artificial Sequence
  Synthetic CPP Arg deletion mutant of Tat (48-60)
GRKKRRQRRPPQC

SEQ ID NO: 416 - protein, 10 aa, Artificial Sequence
  Synthetic CPP Tat (48-57)
GRKKRRQRRR

SEQ ID NO: 417 - protein, 11 aa, Artificial Sequence
  Synthetic CPP Pro deletion mutant of Tat (48-60)
GRKKRRQRRRC

SEQ ID NO: 418 - protein, 12 aa, Artificial Sequence
  Synthetic CPP Tat-CG
GRKKRRQRRRCG

SEQ ID NO: 419 - protein, 11 aa, Artificial Sequence
  Synthetic CPP TAT
GRKKRRQRRRG

SEQ ID NO: 420 - protein, 15 aa, Artificial Sequence
  Synthetic CPP TatsMTS (TMG)
GRKKRRQRRRMVSAL

SEQ ID NO: 421 - protein, 11 aa, Artificial Sequence
  Synthetic CPP TAT(47-57)
GRKKRRQRRRP

SEQ ID NO: 422 - protein, 12 aa, Artificial Sequence
  Synthetic CPP Tat (48-59)
GRKKRRQRRRPP

SEQ ID NO: 423 - protein, 13 aa, Artificial Sequence
  Synthetic CPP Tat (48-60)
GRKKRRQRRRPPQ

SEQ ID NO: 424 - protein, 14 aa, Artificial Sequence
  Synthetic CPP HIV-1 Tat (48-60)
GRKKRRQRRRPPQC

SEQ ID NO: 425 - protein, 39 aa, Artificial Sequence
  Synthetic CPP
GRKKRRQRRRPPQGRKKRRQRRRPPQGRKKRRQRRRPPQ

SEQ ID NO: 426 - protein, 14 aa, Artificial Sequence
  Synthetic CPP TAT
GRKKRRQRRRPPQK

SEQ ID NO: 427 - protein, 16 aa, Artificial Sequence
  Synthetic CPP Tat
GRKKRRQRRRPPQRKC

SEQ ID NO: 428 - protein, 30 aa, Artificial Sequence
  Synthetic CPP Tat-PKI
GRKKRRQRRRPPQTYADFIASGRTGRRNAI

SEQ ID NO: 429 - protein, 14 aa, Artificial Sequence
  Synthetic CPP Tat-Dex
GRKKRRQRRRPPQY

SEQ ID NO: 430 - protein, 12 aa, Artificial Sequence
  Synthetic CPP HIV-1 TAT peptide--Crystallins
GRKKRRQRRRPQ

SEQ ID NO: 431 - protein, 13 aa, Artificial Sequence
  Synthetic CPP TatP59W
GRKKRRQRRRPWQ

SEQ ID NO: 432 - protein, 18 aa, Artificial Sequence
  Synthetic CPP HME-1
GRKLKKKKNEKEDKRPRT

SEQ ID NO: 433 - protein, 8 aa, Artificial Sequence
  Synthetic CPP 6-Oct
GRKRKKRT

SEQ ID NO: 434 - protein, 17 aa, Artificial Sequence
  Synthetic CPP DPV6
GRPRESGKKRKRKRLKP

SEQ ID NO: 435 - protein, 16 aa, Artificial Sequence
  Synthetic CPP Erns3
GRQLRIAGKRLEGRSK

SEQ ID NO: 436 - protein, 16 aa, Artificial Sequence
  Synthetic CPP Erns6
GRQLRIAGKRLRGRSK

SEQ ID NO: 437 - protein, 16 aa, Artificial Sequence
  Synthetic CPP Erns7
GRQLRIAGRRLRGRSR

SEQ ID NO: 438 - protein, 16 aa, Artificial Sequence
  Synthetic CPP Erns9
GRQLRIAGRRLRRRSR

SEQ ID NO: 439 - protein, 16 aa, Artificial Sequence
  Synthetic CPP Erns8
GRQLRRAGRRLRGRSR

SEQ ID NO: 440 - protein, 16 aa, Artificial Sequence
  Synthetic CPP Erns10
GRQLRRAGRRLRRRSR

SEQ ID NO: 441 - protein, 18 aa, Artificial Sequence
  Synthetic CPP Nucleoplasmin X
GRRERNKMAAAKCRNRRR

SEQ ID NO: 442 - protein, 16 aa, Artificial Sequence
  Synthetic CPP hPER1- PTD (830-846) NLS
GRRHHCRSKAKRSRHH

SEQ ID NO: 443 - protein, 14 aa, Artificial Sequence
  Synthetic CPP HEN1/NSLC1
GRRRRATAKYRTAH

SEQ ID NO: 444 - protein, 12 aa, Artificial Sequence
  Synthetic CPP HNF3
GRRRRKRLSHRT

SEQ ID NO: 445 - protein, 10 aa, Artificial Sequence
  Synthetic CPP cAMP dependent TF
GRRRRRERNK

SEQ ID NO: 446 - protein, 10 aa, Artificial Sequence
  Synthetic CPP R9
GRRRRRRRRR

SEQ ID NO: 447 - protein, 13 aa, Artificial Sequence
  Synthetic CPP R9-TAT
GRRRRRRRRRPPQ

SEQ ID NO: 448 - protein, 14 aa, Artificial Sequence
  Synthetic CPP (42-38)-(9-1) Crot
GSGKKGGKKHCQKY

SEQ ID NO: 449 - protein, 14 aa, Artificial Sequence
  Synthetic CPP D form of (1-9)-(38-42) Crot
GSGKKGGKKICQKY

SEQ ID NO: 450 - protein, 13 aa, Artificial Sequence
  Synthetic CPP 439A peptide
GSPWGLQHHPPRT

SEQ ID NO: 451 - protein, 12 aa, Artificial Sequence
  Synthetic CPP Peptide 16
GSRHPSLIIPRQ

SEQ ID NO: 452 - protein, 15 aa, Artificial Sequence
  Synthetic CPP HSV-1 glycoprotein C gene (gC)--Crystallins
GSRVQIRCRFRNSTR

SEQ ID NO: 453 - protein, 16 aa, Artificial Sequence
  Synthetic CPP LMWP-EGFP
GSVSRRRRRRGGRRRR

SEQ ID NO: 454 - protein, 25 aa, Artificial Sequence
  Synthetic CPP Cyt C 71-101
GTKMIFVGIKKKEERADLIAYLKKA

SEQ ID NO: 455 - protein, 27 aa, Artificial Sequence
  Synthetic CPP TP5
GWTLNPAGYLLGKINLKALAALAKKIL

SEQ ID NO: 456 - protein, 27 aa, Artificial Sequence
  Synthetic CPP TP6
GWTLNPPGYLLGKINLKALAALAKKIL

SEQ ID NO: 457 - protein, 26 aa, Artificial Sequence
  Synthetic CPP TP4
GWTLNSAGYLLGKFLPLILRKIVTAL

SEQ ID NO: 458 - protein, 27 aa, Artificial Sequence
  Synthetic CPP Transportan
GWTLNSAGYLLGKINLKALAALAKKIL

SEQ ID NO: 459 - protein, 27 aa, Artificial Sequence
  Synthetic CPP TP2
GWTLNSAGYLLGKINLKALAALAKKLL

SEQ ID NO: 460 - protein, 27 aa, Artificial Sequence
  Synthetic CPP TP16
GWTLNSAGYLLGKINLKAPAALAKKIL

SEQ ID NO: 461 - protein, 25 aa, Artificial Sequence
  Synthetic CPP TP9
GWTLNSAGYLLGKLKALAALAKKIL

SEQ ID NO: 462 - protein, 30 aa, Artificial Sequence
  Synthetic CPP Galanin
GWTLNSAGYLLGPHAVGNHRSFSDKNGLTS

SEQ ID NO: 463 - protein, 21 aa, Artificial Sequence
  Synthetic CPP TP11
GWTLNSKINLKALAALAKKIL

SEQ ID NO: 464 - protein, 15 aa, Artificial Sequence
  Synthetic CPP No. 440
GYGNCRHFKQKPRRD

SEQ ID NO: 465 - protein, 23 aa, Artificial Sequence
  Synthetic CPP YM-3
GYGRKKRRGRRRTHRLPRRRRRR

SEQ ID NO: 466 - protein, 13 aa, Artificial Sequence
  Synthetic CPP Tat (47-57)
GYGRKKRRQRRRG

SEQ ID NO: 467 - protein, 38 aa, Artificial Sequence
  Synthetic CPP D4
GYGYGYGYGYGYGYGYKKRKKRKKRKKRKQQKQQKRRK

SEQ ID NO: 468 - protein, 28 aa, Artificial Sequence
  Synthetic CPP A8
HALAHKLKHLLHRLRHLLHRHLRHALAH

SEQ ID NO: 469 - protein, 20 aa, Artificial Sequence
  Synthetic CPP L2 (Ala33 substitution mutant of LALF (32-51))
HARIKPTFRRLKWKYKGKFW

SEQ ID NO: 470 - protein, 10 aa, Artificial Sequence
  Synthetic CPP Peptide 6
HATKSQNINF

SEQ ID NO: 471 - protein, 37 aa, Artificial Sequence
  Synthetic CPP GST-(HE)8EFG5YG(RG)6
HEHEHEHEHEHEHEHEEFGGGGGYGRGRGRGRGRGRG

SEQ ID NO: 472 - protein, 37 aa, Artificial Sequence
  Synthetic CPP GST-(HE)8EFG5YGR6G6
HEHEHEHEHEHEHEHEEFGGGGGYGRRRRRRGGGGGG

SEQ ID NO: 473 - protein, 41 aa, Artificial Sequence
  Synthetic CPP GST-(HE)10EFG5YG(RG)6
HEHEHEHEHEHEHEHEHEHEEFGGGGGYGRGRGRGRGRGRG

SEQ ID NO: 474 - protein, 41 aa, Artificial Sequence
  Synthetic CPP GST-(HE)10EFG5YGR6G6
HEHEHEHEHEHEHEHEHEHEEFGGGGGYGRRRRRRGGGGGG

SEQ ID NO: 475 - protein, 43 aa, Artificial Sequence
  Synthetic CPP GST-HE-MAP
HEHEHEHEHEHEHEHEHEHEGGGGGKLALKLALKALKAALKLA

SEQ ID NO: 476 - protein, 45 aa, Artificial Sequence
  Synthetic CPP GST-(HE)12EFG5YG(RG)6
HEHEHEHEHEHEHEHEHEHEHEHEEFGGGGGYGRGRGRGRGRGRG

SEQ ID NO: 477 - protein, 42 aa, Artificial Sequence
  Synthetic CPP GST-(HE)12EFG5-TAT
HEHEHEHEHEHEHEHEHEHEHEHEEFGGGGGYGRKKRRQRRR

SEQ ID NO: 478 - protein, 45 aa, Artificial Sequence
  Synthetic CPP GST-(HE)12EFG5YGR6G6
HEHEHEHEHEHEHEHEHEHEHEHEEFGGGGGYGRRRRRRGGGGGG

SEQ ID NO: 479 - protein, 12 aa, Artificial Sequence
  Synthetic CPP Peptide 29
HFAAWGGWSLVH

SEQ ID NO: 480 - protein, 26 aa, Artificial Sequence
  Synthetic CPP Foxp3-11R
HHHHHHESGGGGSPGRRRRRRRRRRR

SEQ ID NO: 481 - protein, 35 aa, Artificial Sequence
  Synthetic CPP STR-H20R8
HHHHHHHHHHHHHHHHHHHHRRRRRRRRRRRRRRR

SEQ ID NO: 482 - protein, 31 aa, Artificial Sequence
  Synthetic CPP H16R8
HHHHHHHHHHHHHHHHRRRRRRRRRRRRRRR

SEQ ID NO: 483 - protein, 27 aa, Artificial Sequence
  Synthetic CPP STR-H12R8
HHHHHHHHHHHHRRRRRRRRRRRRRRR

SEQ ID NO: 484 - protein, 16 aa, Artificial Sequence
  Synthetic CPP STR-H8R8
HHHHHHHHRRRRRRRR

SEQ ID NO: 485 - protein, 23 aa, Artificial Sequence
  Synthetic CPP H8R15
HHHHHHHHRRRRRRRRRRRRRRR

SEQ ID NO: 486 - protein, 15 aa, Artificial Sequence
  Synthetic CPP D9
HHHHHHRRRRRRRRR

SEQ ID NO: 487 - protein, 28 aa, Artificial Sequence
  Synthetic CPP Inv3.10
HHHHHHTKRRITPKDVIDVRSVTTEINT

SEQ ID NO: 488 - protein, 11 aa, Artificial Sequence
  Synthetic CPP 5-FAM-H3R8
HHHRRRRRRRR

SEQ ID NO: 489 - protein, 15 aa, Artificial Sequence
  Synthetic CPP D8
HHHRRRRRRRRRHHH

SEQ ID NO: 490 - protein, 14 aa, Artificial Sequence
  Synthetic CPP DNA-IL-PEI
HILPWKWPWWPWRR

SEQ ID NO: 491 - protein, 12 aa, Artificial Sequence
  Synthetic CPP Peptide 30
HIQLSPFSQSWR

SEQ ID NO: 492 - protein, 12 aa, Artificial Sequence
  Synthetic CPP Peptide 54
HPGSPFPPEHRP

SEQ ID NO: 493 - protein, 12 aa, Artificial Sequence
  Synthetic CPP Peptide 62
HQHKPPPLTNNW

SEQ ID NO: 494 - protein, 12 aa, Artificial Sequence
  Synthetic CPP Peptide 12
HRHIRRQSLIML

SEQ ID NO: 495 - protein, 27 aa, Artificial Sequence
  Synthetic CPP A7
HRLRHALAHLLHKLKHLLHALAHRLRH

SEQ ID NO: 496 - protein, 39 aa, Artificial Sequence
  Synthetic CPP VIP-TAT
HSDAVFTDNYTALRKQMAVKKYLNSILNYGRKKRRQRRR

SEQ ID NO: 497 - protein, 38 aa, Artificial Sequence
  Synthetic CPP PACAP
HSDGIFTDSYSRYRKQMAVKKYLAAVLGKRYKQRVKNK

SEQ ID NO: 498 - protein, 20 aa, Artificial Sequence
  Synthetic CPP L8 (Ala39 substitution mutant of LALF (32-51))
HYRIKPTARRLKWKYKGKFW

SEQ ID NO: 499 - protein, 20 aa, Artificial Sequence
  Synthetic CPP L12 (Ala43 substitution mutant of LALF (32-51))
HYRIKPTFRRLAWKYKGKFW

SEQ ID NO: 500 - protein, 20 aa, Artificial Sequence
  Synthetic CPP L20 (Ala51 substitution mutant of LALF (32-51))
HYRIKPTFRRLKWKYKGKFA

SEQ ID NO: 501 - protein, 17 aa, Artificial Sequence
  Synthetic CPP YTA4
IAWVKAFIRKLRKGPLG

SEQ ID NO: 502 - protein, 5 aa, Artificial Sequence
  Synthetic CPP Penetration
IGCRH

SEQ ID NO: 503 - protein, 4 aa, Artificial Sequence
  Synthetic CPP Xentry peptides
IIIR

SEQ ID NO: 504 - protein, 9 aa, Artificial Sequence
  Synthetic CPP TCTP (2-10) deletion mutant
IIYRDLISH

SEQ ID NO: 505 - protein, 38 aa, Artificial Sequence
  Synthetic CPP D7
IKIKIKIKIKIKIKIKKLAKLAKLAKLAKLAKLAKKIK

SEQ ID NO: 506 - protein, 14 aa, Artificial Sequence
  Synthetic CPP pAntp (45-58)
IKIWFQNRRMKWKK

SEQ ID NO: 507 - protein, 14 aa, Artificial Sequence
  Synthetic CPP TAM-MP
INLKALAALAKKIL

SEQ ID NO: 508 - protein, 5 aa, Artificial Sequence
  Synthetic CPP Bip14
IPALK

SEQ ID NO: 509 - protein, 8 aa, Artificial Sequence
  Synthetic CPP IPL
IPLVVPLC

SEQ ID NO: 510 - protein, 16 aa, Artificial Sequence
  Synthetic CPP RIPL peptide
IPLVVPLRRRRRRRRC

SEQ ID NO: 511 - protein, 5 aa, Artificial Sequence
  Synthetic CPP Bip10
IPMIK

SEQ ID NO: 512 - protein, 5 aa, Artificial Sequence
  Synthetic CPP Bip15
IPMLK

SEQ ID NO: 513 - protein, 15 aa, Artificial Sequence
  Synthetic CPP No.143
IPSRWKDQFWKRWHY

SEQ ID NO: 514 - protein, 7 aa, Artificial Sequence
  Synthetic CPP IRQ
IRQRRRR

SEQ ID NO: 515 - protein, 15 aa, Artificial Sequence
  Synthetic CPP NYAD-41
ISFDELLDYYGESGS

SEQ ID NO: 516 - protein, 12 aa, Artificial Sequence
  Synthetic CPP pAntp (47-58)
IWFQNRRMKWKK

SEQ ID NO: 517 - protein, 10 aa, Artificial Sequence
  Synthetic CPP Peptide 8
IWRYSLASQQ

SEQ ID NO: 518 - protein, 25 aa, Artificial Sequence
  Synthetic CPP P7-5
IYLATALAKWALKQGFGGRRRRRRR

SEQ ID NO: 519 - protein, 23 aa, Artificial Sequence
  Synthetic CPP P7-7
IYLATALAKWALKQGGRRRRRRR

SEQ ID NO: 520 - protein, 8 aa, Artificial Sequence
  Synthetic CPP TCTP (3-10) deletion mutant
IYRDLISH

SEQ ID NO: 521 - protein, 23 aa, Artificial Sequence
  Synthetic CPP KAFAK
KAFAKLAARLYRKALARQLGVAA

SEQ ID NO: 522 - protein, 18 aa, Artificial Sequence
  Synthetic CPP II
KALAALLKKLAKLLAALK

SEQ ID NO: 523 - protein, 18 aa, Artificial Sequence
  Synthetic CPP KLA8
KALAALLKKWAKLLAALK

SEQ ID NO: 524 - protein, 18 aa, Artificial Sequence
  Synthetic CPP KLA12
KALAKALAKLWKALAKAA

SEQ ID NO: 525 - protein, 18 aa, Artificial Sequence
  Synthetic CPP KLA10
KALKKLLAKWLAAAKALL

SEQ ID NO: 526 - protein, 18 aa, Artificial Sequence
  Synthetic CPP NAP
KALKLKLALALLAKLKLA

SEQ ID NO: 527 - protein, 10 aa, Artificial Sequence
  Synthetic CPP Crot (27-39) derevative
KCCKWRWRCK

SEQ ID NO: 528 - protein, 22 aa, Artificial Sequence
  Synthetic CPP rLF
KCFMWQEMLNKAGVPKLRCARK

SEQ ID NO: 529 - protein, 13 aa, Artificial Sequence
  Synthetic CPP M3
KCFQWQRNMRKVR

SEQ ID NO: 530 - protein, 19 aa, Artificial Sequence
  Synthetic CPP M1
KCFQWQRNMRKVRGPPVSC

SEQ ID NO: 531 - protein, 22 aa, Artificial Sequence
  Synthetic CPP hLF WT
KCFQWQRNMRKVRGPPVSCIKR

SEQ ID NO: 532 - protein, 22 aa, Artificial Sequence
  Synthetic CPP M2
KCFQWQRNMRKVRGPPVSSIKR

SEQ ID NO: 533 - protein, 14 aa, Artificial Sequence
  Synthetic CPP Crot (27-39) derevative
KCGCRWRWKCGCKK

SEQ ID NO: 534 - protein, 9 aa, Artificial Sequence
  Synthetic CPP ALPHA Virus nucelocapsid (311-320)
KCPSRRPKR

SEQ ID NO: 535 - protein, 11 aa, Artificial Sequence
  Synthetic CPP Crot (27-39) derevative
KCRWRWKCCKK

SEQ ID NO: 536 - protein, 28 aa, Artificial Sequence
  Synthetic CPP FITC-WT1-pTj
KDCERRFSRSDQLKRHQRRHTGVKPFQK

SEQ ID NO: 537 - protein, 12 aa, Artificial Sequence
  Synthetic CPP Crot (27-39) derevative
KDCRWRWKCCKK

SEQ ID NO: 538 - protein, 21 aa, Artificial Sequence
  Synthetic CPP Pep-2
KETWFETWFTEWSQPKKKRKV

SEQ ID NO: 539 - protein, 28 aa, Artificial Sequence
  Synthetic CPP PN183
KETWWETWWTEWSQPGRKKRRQRRRPPQ

SEQ ID NO: 540 - protein, 21 aa, Artificial Sequence
  Synthetic CPP EGFP-Pep-1
KETWWETWWTEWSQPKKKRKV

SEQ ID NO: 541 - protein, 22 aa, Artificial Sequence
  Synthetic CPP FP-lipo
KETWWETWWTEWSQPKKKRKVC

SEQ ID NO: 542 - protein, 10 aa, Artificial Sequence
  Synthetic CPP CPP-PNA
KFFKFFKFFK

SEQ ID NO: 543 - protein, 15 aa, Artificial Sequence
  Synthetic CPP hCT (1832)
KFHTFPQTAIGVGAP

SEQ ID NO: 544 - protein, 19 aa, Artificial Sequence
  Synthetic CPP IP-1
KFLNRFWHWLQLKPGQPMY

SEQ ID NO: 545 - protein, 9 aa, Artificial Sequence
  Synthetic CPP Cyt c (5-13)
KGKKIFIMK

SEQ ID NO: 546 - protein, 14 aa, Artificial Sequence
  Synthetic CPP q-NTD
KGRKKRRQRRRPPQ

SEQ ID NO: 547 - protein, 26 aa, Artificial Sequence
  Synthetic CPP Res4
KGRTPIKFGKADCDRPPKHSQNGMGK

SEQ ID NO: 548 - protein, 36 aa, Artificial Sequence
  Synthetic CPP PN509
KGSKKAVTKAQKKDGKKRKRSRKESYSVYVYKVLKQ

SEQ ID NO: 549 - protein, 26 aa, Artificial Sequence
  Synthetic CPP MMD45
KHHWHHVRLPPPVRLPPPGNHHHHHH

SEQ ID NO: 550 - protein, 25 aa, Artificial Sequence
  Synthetic CPP LAH6-X1
KHKALHALHLLALLWLHLAHLAKHK

SEQ ID NO: 551 - protein, 29 aa, Artificial Sequence
  Synthetic CPP (KH)9-Bp100
KHKHKHKHKHKHKHKHKHKKLFKKILKYL

SEQ ID NO: 552 - protein, 25 aa, Artificial Sequence
  Synthetic CPP LAH6-X1L-W
KHKLLHLLHLLALLWLHLLHLLKHK

SEQ ID NO: 553 - protein, 18 aa, Artificial Sequence
  Synthetic CPP KLA5
KIAAKSIAKIWKSILKIA

SEQ ID NO: 554 - protein, 20 aa, Artificial Sequence
  Synthetic CPP fGeT
KIAKLKAKIQKLKQKIAKLK

SEQ ID NO: 555 - protein, 18 aa, Artificial Sequence
  Synthetic CPP KLA11
KITLKLAIKAWKLALKAA

SEQ ID NO: 556 - protein, 13 aa, Artificial Sequence
  Synthetic CPP pAntp (46-58)
KIWFQNRRMKWKK

SEQ ID NO: 557 - protein, 17 aa, Artificial Sequence
  Synthetic CPP APP521
KKAAQIRSQVMTHLRVI

SEQ ID NO: 558 - protein, 26 aa, Artificial Sequence
  Synthetic CPP LAH4-L1
KKALLAHALHLLALLALHLAHALKKA

SEQ ID NO: 559 - protein, 25 aa, Artificial Sequence
  Synthetic CPP PN361
KKDGKKRKRSRKESYSVYVYKVLKQ

SEQ ID NO: 560 - protein, 16 aa, Artificial Sequence
  Synthetic CPP M867
KKICTRKPRFMSAWAQ

SEQ ID NO: 561 - protein, 16 aa, Artificial Sequence
  Synthetic CPP Cyt C 86-101
KKKEERADLIAYLKKA

SEQ ID NO: 562 - protein, 34 aa, Artificial Sequence
  Synthetic CPP CL22
KKKKKKGGFLGFWRGENGRKTRSAYERMCILKGK

SEQ ID NO: 563 - protein, 8 aa, Artificial Sequence
  Synthetic CPP K8-lip
KKKKKKKK

SEQ ID NO: 564 - protein, 9 aa, Artificial Sequence
  Synthetic CPP K9
KKKKKKKKK

SEQ ID NO: 565 - protein, 19 aa, Artificial Sequence
  Synthetic CPP Polylysine19
KKKKKKKKKKKKKKKKKKK

SEQ ID NO: 566 - protein, 15 aa, Artificial Sequence
  Synthetic CPP P1
KKKKKKNKKLQQRGD

SEQ ID NO: 567 - protein, 25 aa, Artificial Sequence
  Synthetic CPP LAH4-X1
KKLALHALHLLALLWLHLAHLALKK

SEQ ID NO: 568 - protein, 11 aa, Artificial Sequence
  Synthetic CPP CF-BP16
KKLFKKILKKL

SEQ ID NO: 569 - protein, 14 aa, Artificial Sequence
  Synthetic CPP RSV-A11
KKPGKKTTTKPTKK

SEQ ID NO: 570 - protein, 22 aa, Artificial Sequence
  Synthetic CPP RSV-A10
KKPGKKTTTKPTKKPTIKTTKK

SEQ ID NO: 571 - protein, 10 aa, Artificial Sequence
  Synthetic CPP RSV-A12
KKPTIKTTKK

SEQ ID NO: 572 - protein, 8 aa, Artificial Sequence
  Synthetic CPP Tat (50-57)
KKRRQRRR

SEQ ID NO: 573 - protein, 10 aa, Artificial Sequence
  Synthetic CPP RSV-A13
KKTTTKPTKK

SEQ ID NO: 574 - protein, 33 aa, Artificial Sequence
  Synthetic CPP MMD47
KKWALLALALHHLAHLALHLALALKKAHHHHHH

SEQ ID NO: 575 - protein, 19 aa, Artificial Sequence
  Synthetic CPP Pen7-9Arg
KKWKMRRGAGRRRRRRRRR

SEQ ID NO: 576 - protein, 16 aa, Artificial Sequence
  Synthetic CPP pAntpHD (58-43)
KKWKMRRNQFWIKIQR

SEQ ID NO: 577 - protein, 18 aa, Artificial Sequence
  Synthetic CPP KLA15
KLAAALLKKWKKLAAALL

SEQ ID NO: 578 - protein, 14 aa, Artificial Sequence
  Synthetic CPP KLA
KLAKLAKKLAKLAK

SEQ ID NO: 579 - protein, 23 aa, Artificial Sequence
  Synthetic CPP KLA-R7
KLAKLAKKLAKLAKGGRRRRRRR

SEQ ID NO: 580 - protein, 25 aa, Artificial Sequence
  Synthetic CPP KLA-TAT(47-57)
KLAKLAKKLAKLAKGRKKRRQRRRP

SEQ ID NO: 581 - protein, 24 aa, Artificial Sequence
  Synthetic CPP KLA-ECP(32-41)
KLAKLAKKLAKLAKNYRWRCKNQN

SEQ ID NO: 582 - protein, 18 aa, Artificial Sequence
  Synthetic CPP KLA3
KLALKAAAKAWKAAAKAA

SEQ ID NO: 583 - protein, 18 aa, Artificial Sequence
  Synthetic CPP KLA2
KLALKAALKAWKAAAKLA

SEQ ID NO: 584 - protein, 14 aa, Artificial Sequence
  Synthetic CPP IV
KLALKALKAALKLA

SEQ ID NO: 585 - protein, 14 aa, Artificial Sequence
  Synthetic CPP V
KLALKLALKALKAA

SEQ ID NO: 586 - protein, 16 aa, Artificial Sequence
  Synthetic CPP III
KLALKLALKALKAALK

SEQ ID NO: 587 - protein, 18 aa, Artificial Sequence
  Synthetic CPP I
KLALKLALKALKAALKLA

SEQ ID NO: 588 - protein, 20 aa, Artificial Sequence
  Synthetic CPP MAP
KLALKLALKALKAALKLAGC

SEQ ID NO: 589 - protein, 18 aa, Artificial Sequence
  Synthetic CPP VII
KLALKLALKALQAALQLA

SEQ ID NO: 590 - protein, 18 aa, Artificial Sequence
  Synthetic CPP KLA1
KLALKLALKAWKAALKLA

SEQ ID NO: 591 - protein, 18 aa, Artificial Sequence
  Synthetic CPP KLA13
KLALKLALKWAKLALKAA

SEQ ID NO: 592 - protein, 18 aa, Artificial Sequence
  Synthetic CPP VIII
KLALQLALQALQAALQLA

SEQ ID NO: 593 - protein, 28 aa, Artificial Sequence
  Synthetic CPP pepM
KLFMALVAFLRFLTIPPTAGILKRWGTI

SEQ ID NO: 594 - protein, 18 aa, Artificial Sequence
  Synthetic CPP VI
KLGLKLGLKGLKGGLKLG

SEQ ID NO: 595 - protein, 5 aa, Artificial Sequence
  Synthetic CPP Bip11
KLGVM

SEQ ID NO: 596 - protein, 13 aa, Artificial Sequence
  Synthetic CPP Res7
KLIKGRTPIKFGK

SEQ ID NO: 597 - protein, 25 aa, Artificial Sequence
  Synthetic CPP Res5
KLIKGRTPIKFGKADCDRPPKHSGK

SEQ ID NO: 598 - protein, 27 aa, Artificial Sequence
  Synthetic CPP Res3
KLIKGRTPIKFGKADCDRPPKHSQNGK

SEQ ID NO: 599 - protein, 27 aa, Artificial Sequence
  Synthetic CPP Res2
KLIKGRTPIKFGKADCDRPPKHSQNGM

SEQ ID NO: 600 - protein, 29 aa, Artificial Sequence
  Synthetic CPP Res1
KLIKGRTPIKFGKADCDRPPKHSQNGMGK

SEQ ID NO: 601 - protein, 25 aa, Artificial Sequence
  Synthetic CPP Res6
KLIKGRTPIKFGKARCRRPPKHSGK

SEQ ID NO: 602 - protein, 18 aa, Artificial Sequence
  Synthetic CPP KLA14
KLLAKAAKKWLLLALKAA

SEQ ID NO: 603 - protein, 18 aa, Artificial Sequence
  Synthetic CPP KLA9
KLLAKAALKWLLKALKAA

SEQ ID NO: 604 - protein, 18 aa, Artificial Sequence
  Synthetic CPP C5
KLLKLLLKLWKKLLKLLK

SEQ ID NO: 605 - protein, 28 aa, Artificial Sequence
  Synthetic CPP A6
KLLKLLLKLWKKLLKLLKGGGRRRRRRR

SEQ ID NO: 606 - protein, 19 aa, Artificial Sequence
  Synthetic CPP G55-9
KLPCRSNTFLNIFRRKKPG

SEQ ID NO: 607 - protein, 5 aa, Artificial Sequence
  Synthetic CPP Bip9
KLPVM

SEQ ID NO: 608 - protein, 5 aa, Artificial Sequence
  Synthetic CPP Bip12
KLPVT

SEQ ID NO: 609 - protein, 21 aa, Artificial Sequence
  Synthetic CPP CCMV GAG
KLTRAQRRAAARKNKRNTRGC

SEQ ID NO: 610 - protein, 15 aa, Artificial Sequence
  Synthetic CPP 7
KLWMRWWSPTTRRYG

SEQ ID NO: 611 - protein, 15 aa, Artificial Sequence
  Synthetic CPP No.14-2
KLWMRWYSATTRRYG

SEQ ID NO: 612 - protein, 15 aa, Artificial Sequence
  Synthetic CPP No.14
KLWMRWYSPTTRRYG

SEQ ID NO: 613 - protein, 15 aa, Artificial Sequence
  Synthetic CPP No.14-7
KLWMRWYSPWTRRYG

SEQ ID NO: 614 - protein, 16 aa, Artificial Sequence
  Synthetic CPP PN228
KLWSAWPSLWSSLWKP

SEQ ID NO: 615 - protein, 13 aa, Artificial Sequence
  Synthetic CPP Crot (27-39) derevative
KMDCRPRPKCCKK

SEQ ID NO: 616 - protein, 13 aa, Artificial Sequence
  Synthetic CPP Crot (27-39) derevative
KMDCRWRPKCCKK

SEQ ID NO: 617 - protein, 13 aa, Artificial Sequence
  Synthetic CPP Crot (27-39)
KMDCRWRWKCCKK

SEQ ID NO: 618 - protein, 12 aa, Artificial Sequence
  Synthetic CPP Crot (27-39) derevative
KMDCRWRWKCKK

SEQ ID NO: 619 - protein, 13 aa, Artificial Sequence
  Synthetic CPP Crot (27-39) derevative
KMDCRWRWKCSKK

SEQ ID NO: 620 - protein, 11 aa, Artificial Sequence
  Synthetic CPP Crot (27-39) derevative
KMDCRWRWKKK

SEQ ID NO: 621 - protein, 13 aa, Artificial Sequence
  Synthetic CPP Crot (27-39) derevative
KMDCRWRWKSCKK

SEQ ID NO: 622 - protein, 13 aa, Artificial Sequence
  Synthetic CPP Crot (27-39) derevative
KMDCRWRWKSSKK

SEQ ID NO: 623 - protein, 10 aa, Artificial Sequence
  Synthetic CPP Crot (27-39) derevative
KMDRWRWKKK

SEQ ID NO: 624 - protein, 13 aa, Artificial Sequence
  Synthetic CPP Crot (27-39) derevative
KMDSRWRWKCCKK

SEQ ID NO: 625 - protein, 13 aa, Artificial Sequence
  Synthetic CPP Crot (27-39) derevative
KMDSRWRWKCSKK

SEQ ID NO: 626 - protein, 13 aa, Artificial Sequence
  Synthetic CPP Crot (27-39) derevative
KMDSRWRWKSCKK

SEQ ID NO: 627 - protein, 13 aa, Artificial Sequence
  Synthetic CPP Crot (27-39) derevative
KMDSRWRWKSSKK

SEQ ID NO: 628 - protein, 10 aa, Artificial Sequence
  Synthetic CPP Cyt 79-88
KMIFVGIKKK

SEQ ID NO: 629 - protein, 14 aa, Artificial Sequence
  Synthetic CPP Cyt 79-92
KMIFVGIKKKEERA

SEQ ID NO: 630 - protein, 21 aa, Artificial Sequence
  Synthetic CPP BMV GAG
KMTRAQRRAAARRNRWTARGC

SEQ ID NO: 631 - protein, 15 aa, Artificial Sequence
  Synthetic CPP No. 2028
KNAWKHSSCHHRHQI

SEQ ID NO: 632 - protein, 12 aa, Artificial Sequence
  Synthetic CPP RSV-B3
KPRSKNPPKKPK

SEQ ID NO: 633 - protein, 24 aa, Artificial Sequence
  Synthetic CPP Yeast GCN 4 (231-252)
KRARNTEAARRSRARKLQRMKQGC

SEQ ID NO: 634 - protein, 12 aa, Artificial Sequence
  Synthetic CPP Peptide 2
KRIHPRLTRSIR

SEQ ID NO: 635 - protein, 12 aa, Artificial Sequence
  Synthetic CPP Peptide 1
KRIIQRILSRNS

SEQ ID NO: 636 - protein, 11 aa, Artificial Sequence
  Synthetic CPP RSV-A7
KRIPNKKPGKK

SEQ ID NO: 637 - protein, 12 aa, Artificial Sequence
  Synthetic CPP RSV-A6
KRIPNKKPGKKT

SEQ ID NO: 638 - protein, 19 aa, Artificial Sequence
  Synthetic CPP RSV-A5
KRIPNKKPGKKTTTKPTKK

SEQ ID NO: 639 - protein, 23 aa, Artificial Sequence
  Synthetic CPP RSV-A4
KRIPNKKPGKKTTTKPTKKPTIK

SEQ ID NO: 640 - protein, 27 aa, Artificial Sequence
  Synthetic CPP RSV-A3
KRIPNKKPGKKTTTKPTKKPTIKTTKK

SEQ ID NO: 641 - protein, 30 aa, Artificial Sequence
  Synthetic CPP RSV-A2
KRIPNKKPGKKTTTKPTKKPTIKTTKKDLK

SEQ ID NO: 642 - protein, 37 aa, Artificial Sequence
  Synthetic CPP RSV-A1
KRIPNKKPGKKTTTKPTKKPTIKTTKKDLKPQTTKPK

SEQ ID NO: 643 - protein, 10 aa, Artificial Sequence
  Synthetic CPP RSV-A8
KRIPNKKPKK

SEQ ID NO: 644 - protein, 7 aa, Artificial Sequence
  Synthetic CPP KW
KRKRWHW

SEQ ID NO: 645 - protein, 16 aa, Artificial Sequence
  Synthetic CPP Bipartite nucleoplasmin NLS (155-170)
KRPAAIKKAGQAKKKK

SEQ ID NO: 646 - protein, 15 aa, Artificial Sequence
  Synthetic CPP 44
KRPTMRFRYTWNPMK

SEQ ID NO: 647 - protein, 28 aa, Artificial Sequence
  Synthetic CPP Human c Fos (139-164)
KRRIRRERNKMAAAKSRNRRRELTDTGC

SEQ ID NO: 648 - protein, 7 aa, Artificial Sequence
  Synthetic CPP Tat (51-57)
KRRQRRR

SEQ ID NO: 649 - protein, 13 aa, Artificial Sequence
  Synthetic CPP hClock-(35-47)
KRVSRNKSEKKRR

SEQ ID NO: 650 - protein, 10 aa, Artificial Sequence
  Synthetic CPP Crot (27-39) derevative
KRWRWKCCKK

SEQ ID NO: 651 - protein, 18 aa, Artificial Sequence
  Synthetic CPP Retro-pVEC
KSHAHAQKRIRRRLIILL

SEQ ID NO: 652 - protein, 15 aa, Artificial Sequence
  Synthetic CPP RSV-B1
KSICKTIPSNKPKKK

SEQ ID NO: 653 - protein, 20 aa, Artificial Sequence
  Synthetic CPP KST
KSTGKANKITITNDKGRLSK

SEQ ID NO: 654 - protein, 12 aa, Artificial Sequence
  Synthetic CPP Peptide 64
KTIEAHPPYYAS

SEQ ID NO: 655 - protein, 11 aa, Artificial Sequence
  Synthetic CPP RSV-B2
KTIPSNKPKKK

SEQ ID NO: 656 - protein, 16 aa, Artificial Sequence
  Synthetic CPP E162
KTVLLRKLLKLLVRKI

SEQ ID NO: 657 - protein, 19 aa, Artificial Sequence
  Synthetic CPP MTpl-3
KWCFAVCYAGICYAACAGK

SEQ ID NO: 658 - protein, 19 aa, Artificial Sequence
  Synthetic CPP Tpl
KWCFRVCYRGICYRRCRGK

SEQ ID NO: 659 - protein, 15 aa, Artificial Sequence
  Synthetic CPP Pep-3
KWFETWFTEWPKKRK

SEQ ID NO: 660 - protein, 18 aa, Artificial Sequence
  Synthetic CPP Pep-3
KWFETWFTEWPKKRKGGC

SEQ ID NO: 661 - protein, 16 aa, Artificial Sequence
  Synthetic CPP PenetraMax
KWFKIQMQIRRWKNKR

SEQ ID NO: 662 - protein, 15 aa, Artificial Sequence
  Synthetic CPP MTpl-2
KWFRVYRGIYRRRGK

SEQ ID NO: 663 - protein, 19 aa, Artificial Sequence
  Synthetic CPP MTpl-1
KWSFRVSYRGISYRRSRGK

SEQ ID NO: 664 - protein, 25 aa, Artificial Sequence
  Synthetic CPP A11
LAELLAELLAELGGGGRRRRRRRRR

SEQ ID NO: 665 - protein, 18 aa, Artificial Sequence
  Synthetic CPP pVEC mutant
LAIILRRRIRKQAHAHSK

SEQ ID NO: 666 - protein, 36 aa, Artificial Sequence
  Synthetic CPP D9
LALALALALALALAKLAKLAKLAKLAKIKKIKKKIK

SEQ ID NO: 667 - protein, 38 aa, Artificial Sequence
  Synthetic CPP D8
LALALALALALALALAKIKKIKKIKKIKKLAKLAKKIK

SEQ ID NO: 668 - protein, 38 aa, Artificial Sequence
  Synthetic CPP D6
LALALALALALALALAKKLKKLKKLKKLKKLKKLKYAK

SEQ ID NO: 669 - protein, 35 aa, Artificial Sequence
  Synthetic CPP D10
LALALALALALALALAKLAKLAKLAKLAKLAKKIK

SEQ ID NO: 670 - protein, 25 aa, Artificial Sequence
  Synthetic CPP A12
LAQLLAQLLAQLGGGGRRRRRRRRR

SEQ ID NO: 671 - protein, 4 aa, Artificial Sequence
  Synthetic CPP Xentry peptides
LCLE

SEQ ID NO: 672 - protein, 4 aa, Artificial Sequence
  Synthetic CPP Xentry peptides
LCLH

SEQ ID NO: 673 - protein, 4 aa, Artificial Sequence
  Synthetic CPP Xentry peptides
LCLK

SEQ ID NO: 674 - protein, 4 aa, Artificial Sequence
  Synthetic CPP Xentry peptides
LCLN

SEQ ID NO: 675 - protein, 4 aa, Artificial Sequence
  Synthetic CPP Xentry peptides
LCLQ

SEQ ID NO: 676 - protein, 4 aa, Artificial Sequence
  Synthetic CPP Xentry peptides
LCLR

SEQ ID NO: 677 - protein, 12 aa, Artificial Sequence
  Synthetic CPP Peptide 45
LDITPFLSLTLP

SEQ ID NO: 678 - protein, 27 aa, Artificial Sequence
  Synthetic CPP Inv10
LDTYSPELFCTIRNFYDADRPDRGAAA

SEQ ID NO: 679 - protein, 18 aa, Artificial Sequence
  Synthetic CPP Tat (43-60)
LGISYGRKKRRQRRRPPQ

SEQ ID NO: 680 - protein, 18 aa, Artificial Sequence
  Synthetic CPP PN86
LGLLLRHLRHHSNLLANI

SEQ ID NO: 681 - protein, 24 aa, Artificial Sequence
  Synthetic CPP EGFP-hcT(9-32)
LGTYTQDFNKFHTFPQTAIGVGAP

SEQ ID NO: 682 - protein, 22 aa, Artificial Sequence
  Synthetic CPP B8
LHHLLHHLLHLLHHLLHHLHHL

SEQ ID NO: 683 - protein, 12 aa, Artificial Sequence
  Synthetic CPP TCTP-CPP 34
LIIFAIAASHKK

SEQ ID NO: 684 - protein, 12 aa, Artificial Sequence
  Synthetic CPP TCTP-CPP 35
LIIFAILISHKK

SEQ ID NO: 685 - protein, 12 aa, Artificial Sequence
  Synthetic CPP TCTP-CPP 16
LIIFRIAASHKK

SEQ ID NO: 686 - protein, 10 aa, Artificial Sequence
  Synthetic CPP TCTP-CPP 33
LIIFRILISH

SEQ ID NO: 687 - protein, 12 aa, Artificial Sequence
  Synthetic CPP TCTP-CPP 30
LIIFRILISHHH

SEQ ID NO: 688 - protein, 11 aa, Artificial Sequence
  Synthetic CPP TCTP-CPP 31
LIIFRILISHK

SEQ ID NO: 689 - protein, 12 aa, Artificial Sequence
  Synthetic CPP TCTP-CPP 27
LIIFRILISHKK

SEQ ID NO: 690 - protein, 11 aa, Artificial Sequence
  Synthetic CPP TCTP-CPP 32
LIIFRILISHR

SEQ ID NO: 691 - protein, 12 aa, Artificial Sequence
  Synthetic CPP TCTP-CPP 29
LIIFRILISHRR

SEQ ID NO: 692 - protein, 14 aa, Artificial Sequence
  Synthetic CPP TAM-rMP
LIKKALAALAKLNI

SEQ ID NO: 693 - protein, 15 aa, Artificial Sequence
  Synthetic CPP LILIR8 (Alexa)
LILIGRRRRRRRRGC

SEQ ID NO: 694 - protein, 38 aa, Artificial Sequence
  Synthetic CPP D11
LILILILILILILILIKRKKRKKRKKRKKRAKRAKHSK

SEQ ID NO: 695 - protein, 23 aa, Artificial Sequence
  Synthetic CPP EB1
LIRLWSHLIHIWFQNRRLKWKKK

SEQ ID NO: 696 - protein, 24 aa, Artificial Sequence
  Synthetic CPP EB1-Cys
LIRLWSHLIHIWFQNRRLKWKKKC

SEQ ID NO: 697 - protein, 26 aa, Artificial Sequence
  Synthetic CPP EB-1
LIRLWSHLIHIWFQNRRLKWKKKGGC

SEQ ID NO: 698 - protein, 15 aa, Artificial Sequence
  Synthetic CPP TAMARA-peptide 2
LKKLAELAHKLLKLG

SEQ ID NO: 699 - protein, 16 aa, Artificial Sequence
  Synthetic CPP LK-2
LKKLCKLLKKLCKLAG

SEQ ID NO: 700 - protein, 16 aa, Artificial Sequence
  Synthetic CPP LK-1
LKKLLKLLKKLLKLAG

SEQ ID NO: 701 - protein, 15 aa, Artificial Sequence
  Synthetic CPP [D]-K6L9
LKLLKKLLKKLLKLL

SEQ ID NO: 702 - protein, 35 aa, Artificial Sequence
  Synthetic CPP pepR
LKRWGTIKKSKAINVLRGFRKEIGRMLNILNRRRR

SEQ ID NO: 703 - protein, 18 aa, Artificial Sequence
  Synthetic CPP XI
LKTLATALTKLAKTLTTL

SEQ ID NO: 704 - protein, 18 aa, Artificial Sequence
  Synthetic CPP XIII
LKTLTETLKELTKTLTEL

SEQ ID NO: 705 - protein, 18 aa, Artificial Sequence
  Synthetic CPP pVEC mutant
LLAILRRRIRKQAHAHSK

SEQ ID NO: 706 - protein, 33 aa, Artificial Sequence
  Synthetic CPP PN202
LLETLLKPFQCRICMRNFSTRQARRNHRRRHRR

SEQ ID NO: 707 - protein, 38 aa, Artificial Sequence
  Synthetic CPP LL-37
LLGDFFRKSKEKIGKEFKRIVQRIKDFLRNLVPRTESC

SEQ ID NO: 708 - protein, 18 aa, Artificial Sequence
  Synthetic CPP TP8
LLGKINLKALAALAKKIL

SEQ ID NO: 709 - protein, 18 aa, Artificial Sequence
  Synthetic CPP S6KR
LLHILRRSIRKQAHAIRK

SEQ ID NO: 710 - protein, 18 aa, Artificial Sequence
  Synthetic CPP S6R
LLHILRRSIRRQAHAIRR

SEQ ID NO: 711 - protein, 18 aa, Artificial Sequence
  Synthetic CPP pVEC mutant
LLIALRRRIRKQAHAHSK

SEQ ID NO: 712 - protein, 18 aa, Artificial Sequence
  Synthetic CPP pVEC mutant
LLIIARRRIRKQAHAHSK

SEQ ID NO: 713 - protein, 18 aa, Artificial Sequence
  Synthetic CPP pVEC mutant
LLIILARRIRKQAHAHSK

SEQ ID NO: 714 - protein, 18 aa, Artificial Sequence
  Synthetic CPP pVEC mutant
LLIILRARIRKQAHAHSK

SEQ ID NO: 715 - protein, 18 aa, Artificial Sequence
  Synthetic CPP pVEC mutant
LLIILRRAIRKQAHAHSK

SEQ ID NO: 716 - protein, 18 aa, Artificial Sequence
  Synthetic CPP pVEC mutant
LLIILRRRARKQAHAHSK

SEQ ID NO: 717 - protein, 19 aa, Artificial Sequence
  Synthetic CPP pVEC mutant
LLIILRRRIARKQAHAHSK

SEQ ID NO: 718 - protein, 18 aa, Artificial Sequence
  Synthetic CPP pVEC mutant
LLIILRRRIRAQAHAHSK

SEQ ID NO: 719 - protein, 18 aa, Artificial Sequence
  Synthetic CPP pVEC mutant
LLIILRRRIRKAAHAHSK

SEQ ID NO: 720 - protein, 18 aa, Artificial Sequence
  Synthetic CPP pVEC mutant
LLIILRRRIRKQAAAHSK

SEQ ID NO: 721 - protein, 18 aa, Artificial Sequence
  Synthetic CPP pVEC mutant
LLIILRRRIRKQAHAASK

SEQ ID NO: 722 - protein, 18 aa, Artificial Sequence
  Synthetic CPP pVEC mutant
LLIILRRRIRKQAHAHAK

SEQ ID NO: 723 - protein, 18 aa, Artificial Sequence
  Synthetic CPP pVEC mutant
LLIILRRRIRKQAHAHSA

SEQ ID NO: 724 - protein, 18 aa, Artificial Sequence
  Synthetic CPP pVEC
LLIILRRRIRKQAHAHSK

SEQ ID NO: 725 - protein, 30 aa, Artificial Sequence
  Synthetic CPP FAM-pVEC-gHo (FAM- gHoPe2)
LLIILRRRIRKQAHAHSKNHQQQNPHQPPM

SEQ ID NO: 726 - protein, 18 aa, Artificial Sequence
  Synthetic CPP P9R
LLIILRRRIRRRARARSR

SEQ ID NO: 727 - protein, 16 aa, Artificial Sequence
  Synthetic CPP E165
LLKKRKVVRLIKFLLK

SEQ ID NO: 728 - protein, 19 aa, Artificial Sequence
  Synthetic CPP PF20
LLKLLKKLLKLLKKLLKLL

SEQ ID NO: 729 - protein, 18 aa, Artificial Sequence
  Synthetic CPP XII
LLKTTALLKTTALLKTTA

SEQ ID NO: 730 - protein, 18 aa, Artificial Sequence
  Synthetic CPP XIV
LLKTTELLKTTELLKTTE

SEQ ID NO: 731 - protein, 5 aa, Artificial Sequence
  Synthetic CPP Xentry peptides
LLLLR

SEQ ID NO: 732 - protein, 4 aa, Artificial Sequence
  Synthetic CPP Xentry peptides
LLLR

SEQ ID NO: 733 - protein, 5 aa, Artificial Sequence
  Synthetic CPP Xentry peptides
LLLRR

SEQ ID NO: 734 - protein, 15 aa, Artificial Sequence
  Synthetic CPP P6
LLRARWRRRRSRRFR

SEQ ID NO: 735 - protein, 18 aa, Artificial Sequence
  Synthetic CPP S9RH
LLRHLRRHIRRARRHIRR

SEQ ID NO: 736 - protein, 18 aa, Artificial Sequence
  Synthetic CPP S9R
LLRILRRSIRRARRAIRR

SEQ ID NO: 737 - protein, 18 aa, Artificial Sequence
  Synthetic CPP Mgpe-4
LLYWFRRRHRHHRRRHRR

SEQ ID NO: 738 - protein, 20 aa, Artificial Sequence
  Synthetic CPP TP13
LNSAGYLLGKALAALAKKIL

SEQ ID NO: 739 - protein, 24 aa, Artificial Sequence
  Synthetic CPP TP7
LNSAGYLLGKINLKALAALAKKIL

SEQ ID NO: 740 - protein, 19 aa, Artificial Sequence
  Synthetic CPP TP15
LNSAGYLLGKLKALAALAK

SEQ ID NO: 741 - protein, 21 aa, Artificial Sequence
  Synthetic CPP TP12
LNSAGYLLGKLKALAALAKIL

SEQ ID NO: 742 - protein, 12 aa, Artificial Sequence
  Synthetic CPP Peptide 44
LNVPPSWFLSQR

SEQ ID NO: 743 - protein, 12 aa, Artificial Sequence
  Synthetic CPP Peptide 46
LPHPVLHMGPLR

SEQ ID NO: 744 - protein, 29 aa, Artificial Sequence
  Synthetic CPP A4
LRHHLRHLLRHLRHLLRHLRHHLRHLLRH

SEQ ID NO: 745 - protein, 15 aa, Artificial Sequence
  Synthetic CPP D12
LRHLLRHLLRHLRHL

SEQ ID NO: 746 - protein, 29 aa, Artificial Sequence
  Synthetic CPP A3
LRHLLRHLLRHLRHLLRHLRHLLRHLLRH

SEQ ID NO: 747 - protein, 16 aa, Artificial Sequence
  Synthetic CPP DPV15
LRRERQSRLRRERQSR

SEQ ID NO: 748 - protein, 28 aa, Artificial Sequence
  Synthetic CPP p28
LSTAADMQGVVTDGMASGLDKDYLKPDD

SEQ ID NO: 749 - protein, 12 aa, Artificial Sequence
  Synthetic CPP Peptide 31
LTMPSDLQPVLW

SEQ ID NO: 750 - protein, 12 aa, Artificial Sequence
  Synthetic CPP Peptide 22
LTRNYEAWVPTP

SEQ ID NO: 751 - protein, 5 aa, Artificial Sequence
  Synthetic CPP X-Pep derivative
MAARL

SEQ ID NO: 752 - protein, 8 aa, Artificial Sequence
  Synthetic CPP X-Pep
MAARLCCQ

SEQ ID NO: 753 - protein, 15 aa, Artificial Sequence
  Synthetic CPP N-terminus of X-Pep
MAARLCCQLDPARDV

SEQ ID NO: 754 - protein, 20 aa, Artificial Sequence
  Synthetic CPP N-terminus of X-Pep
MAARLCCQLDPARDVLCLRP

SEQ ID NO: 755 - protein, 9 aa, Artificial Sequence
  Synthetic CPP TCTP(I-9) I2A subsetution mutant
MAIYRDLIS

SEQ ID NO: 756 - protein, 29 aa, Artificial Sequence
  Synthetic CPP CPPK
MAMPGEPRRANVMAHKLEPASLQLRNSCA

SEQ ID NO: 757 - protein, 28 aa, Artificial Sequence
  Synthetic CPP Human Prp (1-28)
MANLGCWMLVLFVATWSDLGLCKKRPKP

SEQ ID NO: 758 - protein, 28 aa, Artificial Sequence
  Synthetic CPP Mouse Prp (1-28)
MANLGYWLLALFVTMWTDVGLCKKRPKP

SEQ ID NO: 759 - protein, 29 aa, Artificial Sequence
  Synthetic CPP CPPL
MAPQRDTVGGRTTPPSWGPAKAQLRNSCA

SEQ ID NO: 760 - protein, 24 aa, Artificial Sequence
  Synthetic CPP LAMBDA N (1-22)
MDAQTRRRERRAEKQAQWKAANGC

SEQ ID NO: 761 - protein, 12 aa, Artificial Sequence
  Synthetic CPP Crot (27-39) derevative
MDCRWRWKCCKK

SEQ ID NO: 762 - protein, 23 aa, Artificial Sequence
  Synthetic CPP Peptide 2
MGLGLHLLVLAAALQGAKKKRKV

SEQ ID NO: 763 - protein, 27 aa, Artificial Sequence
  Synthetic CPP Peptide 1
MGLGLHLLVLAAALQGAWSQPKKKRKV

SEQ ID NO: 764 - protein, 12 aa, Artificial Sequence
  Synthetic CPP Peptide 6
MHKRPTTPSRKM

SEQ ID NO: 765 - protein, 9 aa, Artificial Sequence
  Synthetic CPP TCTP(1-9 I3A subsetution mutant
MIAYRDLIS

SEQ ID NO: 766 - protein, 9 aa, Artificial Sequence
  Synthetic CPP TCTP(1-9) Y4A subsetution mutant
MIIARDLIS

SEQ ID NO: 767 - protein, 12 aa, Artificial Sequence
  Synthetic CPP TCTP-CPP 26
MIIFAIAASHKK

SEQ ID NO: 768 - protein, 12 aa, Artificial Sequence
  Synthetic CPP TCTP-CPP 24
MIIFKIAASHKK

SEQ ID NO: 769 - protein, 12 aa, Artificial Sequence
  Synthetic CPP TCTP-CPP 14
MIIFRAAASHKK

SEQ ID NO: 770 - protein, 12 aa, Artificial Sequence
  Synthetic CPP TCTP-CPP 13
MIIFRALISHKK

SEQ ID NO: 771 - protein, 10 aa, Artificial Sequence
  Synthetic CPP TCTP-CPP 3
MIIFRDLISH

SEQ ID NO: 772 - protein, 12 aa, Artificial Sequence
  Synthetic CPP TCTP-CPP 12
MIIFRIAASHKK

SEQ ID NO: 773 - protein, 12 aa, Artificial Sequence
  Synthetic CPP TCTP-CPP 22
MIIFRIAATHKK

SEQ ID NO: 774 - protein, 12 aa, Artificial Sequence
  Synthetic CPP TCTP-CPP 20
MIIFRIAAYHKK

SEQ ID NO: 775 - protein, 12 aa, Artificial Sequence
  Synthetic CPP TCTP-CPP 28
MIIFRILISHKK

SEQ ID NO: 776 - protein, 10 aa, Artificial Sequence
  Synthetic CPP TCTP-CPP 9
MIIRRDLISE

SEQ ID NO: 777 - protein, 10 aa, Artificial Sequence
  Synthetic CPP TCTP-CPP 4
MIISRDLISH

SEQ ID NO: 778 - protein, 9 aa, Artificial Sequence
  Synthetic CPP TCTP(1-9) R5A subsetution mutant
MIIYADLIS

SEQ ID NO: 779 - protein, 10 aa, Artificial Sequence
  Synthetic CPP TCTP-CPP 11
MIIYARRAEE

SEQ ID NO: 780 - protein, 10 aa, Artificial Sequence
  Synthetic CPP TCTP-CPP 10
MIIYRAEISH

SEQ ID NO: 781 - protein, 9 aa, Artificial Sequence
  Synthetic CPP TCTP(1-9) D6A subsetution mutant
MIIYRALIS

SEQ ID NO: 782 - protein, 12 aa, Artificial Sequence
  Synthetic CPP TCTP-CPP 7
MIIYRALISHKK

SEQ ID NO: 783 - protein, 6 aa, Artificial Sequence
  Synthetic CPP TCTP (1-6) deletion mutant
MIIYRD

SEQ ID NO: 784 - protein, 9 aa, Artificial Sequence
  Synthetic CPP TCTP(1-9) L7A subsetution mutant
MIIYRDAIS

SEQ ID NO: 785 - protein, 10 aa, Artificial Sequence
  Synthetic CPP TCTP-CPP 2
MIIYRDKKSH

SEQ ID NO: 786 - protein, 7 aa, Artificial Sequence
  Synthetic CPP TCTP (1-7) deletion mutant
MIIYRDL

SEQ ID NO: 787 - protein, 9 aa, Artificial Sequence
  Synthetic CPP TCTP(1-9) I8A subsetution mutant
MIIYRDLAS

SEQ ID NO: 788 - protein, 8 aa, Artificial Sequence
  Synthetic CPP TCTP (1-8) deletion mutant
MIIYRDLI

SEQ ID NO: 789 - protein, 9 aa, Artificial Sequence
  Synthetic CPP TCTP(1-9) S9A subsetution mutant
MIIYRDLIA

SEQ ID NO: 790 - protein, 9 aa, Artificial Sequence
  Synthetic CPP TCTP (1-9) deletion mutant
MIIYRDLIS

SEQ ID NO: 791 - protein, 10 aa, Artificial Sequence
  Synthetic CPP TCTPPTD
MIIYRDLISH

SEQ ID NO: 792 - protein, 11 aa, Artificial Sequence
  Synthetic CPP TCTP-CPP 1
MIIYRDLISKK

SEQ ID NO: 793 - protein, 12 aa, Artificial Sequence
  Synthetic CPP TCTP-CPP 8
MIIYRIAASHKK

SEQ ID NO: 794 - protein, 10 aa, Artificial Sequence
  Synthetic CPP BagP
MLLLTRRRST

SEQ ID NO: 795 - protein, 30 aa, Artificial Sequence
  Synthetic CPP Bac-ELP-H1
MRRIRPRPPRLPRPRPRPLPFPRPGGCYPG

SEQ ID NO: 796 - protein, 12 aa, Artificial Sequence
  Synthetic CPP Peptide 56
MTPSSLSTLPWP

SEQ ID NO: 797 - protein, 30 aa, Artificial Sequence
  Synthetic CPP Bovine Prp (1-30)
MVKSKIGSWILVLFVAMWSDVGLCKKRPKP

SEQ ID NO: 798 - protein, 22 aa, Artificial Sequence
  Synthetic CPP ARF(1-22)
MVRRFLVTLRIRRACGPPRVRV

SEQ ID NO: 799 - protein, 37 aa, Artificial Sequence
  Synthetic CPP ARF(1-37)
MVRRFLVTLRIRRACGPPRVRVFVVHIPRLTGEWAAP

SEQ ID NO: 800 - protein, 22 aa, Artificial Sequence
  Synthetic CPP M918(R-K)
MVTVLFKRLRIRRACGPPRVKV

SEQ ID NO: 801 - protein, 22 aa, Artificial Sequence
  Synthetic CPP M918
MVTVLFRRLRIRRACGPPRVRV

SEQ ID NO: 802 - protein, 19 aa, Artificial Sequence
  Synthetic CPP P22 N
NAKTRRHERRRKLAIERGC

SEQ ID NO: 803 - protein, 12 aa, Artificial Sequence
  Synthetic CPP FAM-gHo
NHQQQNPHQPPM

SEQ ID NO: 804 - protein, 30 aa, Artificial Sequence
  Synthetic CPP FAM-gHo-pVEC (FAM- gHoPe3)
NHQQQNPHQPPMLLIILRRRIRKQAHAHSK

SEQ ID NO: 805 - protein, 12 aa, Artificial Sequence
  Synthetic CPP Peptide 50
NIENSTLATPLS

SEQ ID NO: 806 - protein, 8 aa, Artificial Sequence
  Synthetic CPP SRAM C105Y
NKPILVFY

SEQ ID NO: 807 - protein, 12 aa, Artificial Sequence
  Synthetic CPP Peptide 18
NKRILIRIMTRP

SEQ ID NO: 808 - protein, 13 aa, Artificial Sequence
  Synthetic CPP Asn-Oct-6
NNNAAGRKRKKRT

SEQ ID NO: 809 - protein, 11 aa, Artificial Sequence
  Synthetic CPP FHV-TA (39-49)
NRARRNRRRVR

SEQ ID NO: 810 - protein, 13 aa, Artificial Sequence
  Synthetic CPP E8
NRHFRFFFNFTNR

SEQ ID NO: 811 - protein, 8 aa, Artificial Sequence
  Synthetic CPP pAntp (51-58)
NRRMKWKK

SEQ ID NO: 812 - protein, 12 aa, Artificial Sequence
  Synthetic CPP Peptide 60
NSGTMQSASRAT

SEQ ID NO: 813 - protein, 9 aa, Artificial Sequence
  Synthetic CPP Peptide 1-S-delta
NTCTWLKYH

SEQ ID NO: 814 - protein, 10 aa, Artificial Sequence
  Synthetic CPP Peptide 1
NTCTWLKYHS

SEQ ID NO: 815 - protein, 10 aa, Artificial Sequence
  Synthetic CPP Peptide 1-C3G
NTGTWLKYHS

SEQ ID NO: 816 - protein, 10 aa, Artificial Sequence
  Synthetic CPP EDN(32-41)
NYQRRCKNQN

SEQ ID NO: 817 - protein, 10 aa, Artificial Sequence
  Synthetic CPP ECP(32-41)R3Q
NYQWRCKNQN

SEQ ID NO: 818 - protein, 10 aa, Artificial Sequence
  Synthetic CPP ECP(32-41)W4R
NYRRRCKNQN

SEQ ID NO: 819 - protein, 7 aa, Artificial Sequence
  Synthetic CPP ECP(32-38)
NYRWRCK

SEQ ID NO: 820 - protein, 8 aa, Artificial Sequence
  Synthetic CPP ECP(32-39)
NYRWRCKN

SEQ ID NO: 821 - protein, 9 aa, Artificial Sequence
  Synthetic CPP ECP(32-40)
NYRWRCKNQ

SEQ ID NO: 822 - protein, 10 aa, Artificial Sequence
  Synthetic CPP ECP(32-41)
NYRWRCKNQN

SEQ ID NO: 823 - protein, 12 aa, Artificial Sequence
  Synthetic CPP Peptide 48
NYTTYKSHFQDR

SEQ ID NO: 824 - protein, 11 aa, Artificial Sequence
  Synthetic CPP CTP501
PARAARRAARR

SEQ ID NO: 825 - protein, 6 aa, Artificial Sequence
  Synthetic CPP C105Y derivative
PFVYLI

SEQ ID NO: 826 - protein, 12 aa, Artificial Sequence
  Synthetic CPP Peptide 4
PIRRRKKLRRLK

SEQ ID NO: 827 - protein, 7 aa, Artificial Sequence
  Synthetic CPP SV40
PKKKRKV

SEQ ID NO: 828 - protein, 20 aa, Artificial Sequence
  Synthetic CPP PV-S4(13)
PKKKRKVALWKTLLKKVLKA

SEQ ID NO: 829 - protein, 18 aa, Artificial Sequence
  Synthetic CPP NS
PKKKRKVWKLLQQFFGLM

SEQ ID NO: 830 - protein, 12 aa, Artificial Sequence
  Synthetic CPP PreS2 (41-52)
PLSSIFSRIGDP

SEQ ID NO: 831 - protein, 5 aa, Artificial Sequence
  Synthetic CPP Bip5
PMLKE

SEQ ID NO: 832 - protein, 11 aa, Artificial Sequence
  Synthetic CPP Peptide 21
PNTRVRPDVSF

SEQ ID NO: 833 - protein, 12 aa, Artificial Sequence
  Synthetic CPP Peptide 14
PPHNRIQRRLNM

SEQ ID NO: 834 - protein, 15 aa, Artificial Sequence
  Synthetic CPP Secretory leukoprotease inhibitor derived PTD
PPKKSAQCLRYKKPE

SEQ ID NO: 835 - protein, 18 aa, Artificial Sequence
  Synthetic CPP Bac7-24
PPRLPRPRPRPLPFPRPG

SEQ ID NO: 836 - protein, 12 aa, Artificial Sequence
  Synthetic CPP Peptide 3
PPRLRKRRQLNM

SEQ ID NO: 837 - protein, 12 aa, Artificial Sequence
  Synthetic CPP Peptide 13
PQNRLQIRRHSK

SEQ ID NO: 838 - protein, 10 aa, Artificial Sequence
  Synthetic CPP Bac15-24
PRPLPFPRPG

SEQ ID NO: 839 - protein, 20 aa, Artificial Sequence
  Synthetic CPP Bac5-24
PRPPRLPRPRPRPLPFPRPG

SEQ ID NO: 840 - protein, 12 aa, Artificial Sequence
  Synthetic CPP Bac13-24
PRPRPLPFPRPG

SEQ ID NO: 841 - protein, 14 aa, Artificial Sequence
  Synthetic CPP Bac11-24
PRPRPRPLPFPRPG

SEQ ID NO: 842 - protein, 12 aa, Artificial Sequence
  Synthetic CPP Peptide 11
PSKRLLHNNLRR

SEQ ID NO: 843 - protein, 12 aa, Artificial Sequence
  Synthetic CPP PreS2 3S Mutant
PSSSSSSRIGDP

SEQ ID NO: 844 - protein, 12 aa, Artificial Sequence
  Synthetic CPP Peptide 61
QAASRVENYMHR

SEQ ID NO: 845 - protein, 10 aa, Artificial Sequence
  Synthetic CPP TCTP-CPP 5
QIISRDLISH

SEQ ID NO: 846 - protein, 15 aa, Artificial Sequence
  Synthetic CPP pAntp (44-58)
QIKIWFQNRRMKWKK

SEQ ID NO: 847 - protein, 18 aa, Artificial Sequence
  Synthetic CPP IX
QLALQLALQALQAALQLA

SEQ ID NO: 848 - protein, 5 aa, Artificial Sequence
  Synthetic CPP Bip17
QLPVM

SEQ ID NO: 849 - protein, 9 aa, Artificial Sequence
  Synthetic CPP pAntp (50-58)
QNRRMKWKK

SEQ ID NO: 850 - protein, 12 aa, Artificial Sequence
  Synthetic CPP Peptide 58
QPIIITSPYLPS

SEQ ID NO: 851 - protein, 15 aa, Artificial Sequence
  Synthetic CPP No. 2510
QQHLLIAINGYPRYN

SEQ ID NO: 852 - protein, 12 aa, Artificial Sequence
  Synthetic CPP Peptide 10
QRIRKSKISRTL

SEQ ID NO: 853 - protein, 12 aa, Artificial Sequence
  Synthetic CPP Peptide 28
QSPTDFTFPNPL

SEQ ID NO: 854 - protein, 15 aa, Artificial Sequence
  Synthetic CPP Lambda-N (48-62)
QTRRRERRAEKQAQW

SEQ ID NO: 855 - protein, 10 aa, Artificial Sequence
  Synthetic CPP M6
QWQRNMRKVR

SEQ ID NO: 856 - protein, 19 aa, Artificial Sequence
  Synthetic CPP M5
QWQRNMRKVRGPPVSCIKR

SEQ ID NO: 857 - protein, 17 aa, Artificial Sequence
  Synthetic CPP Buforin-II
RAGLQFPVGRVHRLLRK

SEQ ID NO: 858 - protein, 16 aa, Artificial Sequence
  Synthetic CPP Ala44 substitution mutant of pAntp (43-58)
RAIKIWFQNRRMKWKK

SEQ ID NO: 859 - protein, 9 aa, Artificial Sequence
  Synthetic CPP Ala50 substitution mutant of Tat (49-57)
RAKRRQRRR

SEQ ID NO: 860 - protein, 32 aa, Artificial Sequence
  Synthetic CPP 32 RA
RARARARARARARARARARARARARARARARA

SEQ ID NO: 861 - protein, 15 aa, Artificial Sequence
  Synthetic CPP No.14-12
RAWMRWYSPTTRRYG

SEQ ID NO: 862 - protein, 15 aa, Artificial Sequence
  Synthetic CPP E3
RFTFHFRFEFTFHFE

SEQ ID NO: 863 - protein, 25 aa, Artificial Sequence
  Synthetic CPP A10
RFTFHFRFEFTFHFEGGGRRRRRRR

SEQ ID NO: 864 - protein, 5 aa, Artificial Sequence
  Synthetic CPP cRGD
RGDFK

SEQ ID NO: 865 - protein, 15 aa, Artificial Sequence
  Synthetic CPP P2
RGDGPRRRPRKRRGR

SEQ ID NO: 866 - protein, 19 aa, Artificial Sequence
  Synthetic CPP PD1
RGDRGDRRDLRLDRGDLRC

SEQ ID NO: 867 - protein, 19 aa, Artificial Sequence
  Synthetic CPP PD2
RGDRLDRRDLRLDRRDLRC

SEQ ID NO: 868 - protein, 19 aa, Artificial Sequence
  Synthetic CPP PE1
RGERGERRELRLERGELRC

SEQ ID NO: 869 - protein, 19 aa, Artificial Sequence
  Synthetic CPP PE2
RGERLERRELRLERRELRC

SEQ ID NO: 870 - protein, 17 aa, Artificial Sequence
  Synthetic CPP SynB5
RGGRLAYLRRRWAVLGR

SEQ ID NO: 871 - protein, 18 aa, Artificial Sequence
  Synthetic CPP SynB1
RGGRLSYSRRRFSTSTGR

SEQ ID NO: 872 - protein, 19 aa, Artificial Sequence
  Synthetic CPP SynB1-ELP-H1
RGGRLSYSRRRFSTSTGRA

SEQ ID NO: 873 - protein, 15 aa, Artificial Sequence
  Synthetic CPP P7
RGPRRQPRRHRRPRR

SEQ ID NO: 874 - protein, 36 aa, Artificial Sequence
  Synthetic CPP PN404
RGSRRAVTRAQRRDGRRRRRSRRESYSVYVYRVLRQ

SEQ ID NO: 875 - protein, 11 aa, Artificial Sequence
  Synthetic CPP F3
RHHLRHLRRHL

SEQ ID NO: 876 - protein, 22 aa, Artificial Sequence
  Synthetic CPP B5
RHHLRHLRRHLRHLLRHLRHHL

SEQ ID NO: 877 - protein, 33 aa, Artificial Sequence
  Synthetic CPP A1
RHHLRHLRRHLRHLLRHLRHHLRHLRRHLRHLL

SEQ ID NO: 878 - protein, 22 aa, Artificial Sequence
  Synthetic CPP B6
RHHRRHHRRHRRHHRRHHRHHR

SEQ ID NO: 879 - protein, 16 aa, Artificial Sequence
  Synthetic CPP PDX -1-PTD
RHIKIWFQNRRMKWKK

SEQ ID NO: 880 - protein, 14 aa, Artificial Sequence
  Synthetic CPP E7
RHNFRFFFNFRTNR

SEQ ID NO: 881 - protein, 10 aa, Artificial Sequence
  Synthetic CPP Peptide 5
RHVYHVLLSQ

SEQ ID NO: 882 - protein, 6 aa, Artificial Sequence
  Synthetic CPP LR8DHFRI
RIFIGC

SEQ ID NO: 883 - protein, 10 aa, Artificial Sequence
  Synthetic CPP LR15DL
RIFIHFRIGC

SEQ ID NO: 884 - protein, 8 aa, Artificial Sequence
  Synthetic CPP LR8DHF
RIFIRIGC

SEQ ID NO: 885 - protein, 30 aa, Artificial Sequence
  Synthetic CPP Human c Jun (252-279)
RIKAERKRMRNRIAASKSRKRKLERIARGC

SEQ ID NO: 886 - protein, 11 aa, Artificial Sequence
  Synthetic CPP LR11
RILQQLLFIHF

SEQ ID NO: 887 - protein, 15 aa, Artificial Sequence
  Synthetic CPP LR15
RILQQLLFIHFRIGC

SEQ ID NO: 888 - protein, 17 aa, Artificial Sequence
  Synthetic CPP LR17
RILQQLLFIHFRIGCRH

SEQ ID NO: 889 - protein, 20 aa, Artificial Sequence
  Synthetic CPP LR20
RILQQLLFIHFRIGCRHSRI

SEQ ID NO: 890 - protein, 13 aa, Artificial Sequence
  Synthetic CPP DS4.3
RIMRILRILKLAR

SEQ ID NO: 891 - protein, 12 aa, Artificial Sequence
  Synthetic CPP Peptide 8
RIRMIQNLIKKT

SEQ ID NO: 892 - protein, 9 aa, Artificial Sequence
  Synthetic CPP Ala51 substitution mutant of Tat (49-57)
RKARRQRRR

SEQ ID NO: 893 - protein, 6 aa, Artificial Sequence
  Synthetic CPP PAF96
RKKAAA

SEQ ID NO: 894 - protein, 9 aa, Artificial Sequence
  Synthetic CPP Ala52 substitution mutant of Tat (49-57)
RKKARQRRR

SEQ ID NO: 895 - protein, 10 aa, Artificial Sequence
  Synthetic CPP hBCPP
RKKNPNCRRH

SEQ ID NO: 896 - protein, 9 aa, Artificial Sequence
  Synthetic CPP Ala53 substitution mutant of Tat (49-57)
RKKRAQRRR

SEQ ID NO: 897 - protein, 9 aa, Artificial Sequence
  Synthetic CPP Ala54 substitution mutant of Tat (49-57)
RKKRRARRR

SEQ ID NO: 898 - protein, 9 aa, Artificial Sequence
  Synthetic CPP Ala55 substitution mutant of Tat (49-57)
RKKRRQARR

SEQ ID NO: 899 - protein, 7 aa, Artificial Sequence
  Synthetic CPP Tat (49-55)
RKKRRQR

SEQ ID NO: 900 - protein, 9 aa, Artificial Sequence
  Synthetic CPP Ala56 substitution mutant of Tat (49-57)
RKKRRQRAR

SEQ ID NO: 901 - protein, 8 aa, Artificial Sequence
  Synthetic CPP Tat (49-56)
RKKRRQRR

SEQ ID NO: 902 - protein, 9 aa, Artificial Sequence
  Synthetic CPP Ala57 substitution mutant of Tat (49-57)
RKKRRQRRA

SEQ ID NO: 903 - protein, 9 aa, Artificial Sequence
  Synthetic CPP Tat (49-57)
RKKRRQRRR

SEQ ID NO: 904 - protein, 11 aa, Artificial Sequence
  Synthetic CPP Tat-Cys
RKKRRQRRRGC

SEQ ID NO: 905 - protein, 12 aa, Artificial Sequence
  Synthetic CPP Tat
RKKRRQRRRGGG

SEQ ID NO: 906 - protein, 27 aa, Artificial Sequence
  Synthetic CPP TatLK15
RKKRRQRRRGGGKLLKLLLKLLLKLLK

SEQ ID NO: 907 - protein, 15 aa, Artificial Sequence
  Synthetic CPP dTAT
RKKRRQRRRHRRKKR

SEQ ID NO: 908 - protein, 29 aa, Artificial Sequence
  Synthetic CPP PN28
RKKRRQRRRPPQCAAVALLPAVLLALLAP

SEQ ID NO: 909 - protein, 18 aa, Artificial Sequence
  Synthetic CPP Tat2-Nat
RKKRRQRRRRKKRRQRRR

SEQ ID NO: 910 - protein, 16 aa, Artificial Sequence
  Synthetic CPP DPV3
RKKRRRESRKKRRRES

SEQ ID NO: 911 - protein, 17 aa, Artificial Sequence
  Synthetic CPP DPV3
RKKRRRESRKKRRRESC

SEQ ID NO: 912 - protein, 18 aa, Artificial Sequence
  Synthetic CPP DPV3/10
RKKRRRESRRARRSPRHL

SEQ ID NO: 913 - protein, 29 aa, Artificial Sequence
  Synthetic CPP MMD49
RKKRRRESWVHLPPPVHLPPPGGHHHHHH

SEQ ID NO: 914 - protein, 6 aa, Artificial Sequence
  Synthetic CPP PAF26
RKKWFW

SEQ ID NO: 915 - protein, 20 aa, Artificial Sequence
  Synthetic CPP Camptide
RKLTTIFPLNWKYRKALSLG

SEQ ID NO: 916 - protein, 18 aa, Artificial Sequence
  Synthetic CPP C3
RLALRLALRALRAALRLA

SEQ ID NO: 917 - protein, 15 aa, Artificial Sequence
  Synthetic CPP No.14-13
RLAMRWYSPTTRRYG

SEQ ID NO: 918 - protein, 15 aa, Artificial Sequence
  Synthetic CPP No.14-25
RLFMRFYSPTTRRYG

SEQ ID NO: 919 - protein, 15 aa, Artificial Sequence
  Synthetic CPP D11
RLHHRLHRRLHRLHR

SEQ ID NO: 920 - protein, 29 aa, Artificial Sequence
  Synthetic CPP A2
RLHHRLHRRLHRLHRRLHRLHHRLHRRLH

SEQ ID NO: 921 - protein, 18 aa, Artificial Sequence
  Synthetic CPP C4
RLHLRLHLRHLRHHLRLH

SEQ ID NO: 922 - protein, 15 aa, Artificial Sequence
  Synthetic CPP E2
RLHRRLHRRLHRLHR

SEQ ID NO: 923 - protein, 29 aa, Artificial Sequence
  Synthetic CPP A5
RLHRRLHRRLHRLHRRLHRLHRRLHRRLH

SEQ ID NO: 924 - protein, 15 aa, Artificial Sequence
  Synthetic CPP 28
RLIMRIYAPTTRRYG

SEQ ID NO: 925 - protein, 15 aa, Artificial Sequence
  Synthetic CPP No.14-26
RLIMRIYSPTTRRYG

SEQ ID NO: 926 - protein, 15 aa, Artificial Sequence
  Synthetic CPP No.14-24
RLLMRLYSPTTRRYG

SEQ ID NO: 927 - protein, 18 aa, Artificial Sequence
  Synthetic CPP C6
RLLRLLLRLWRRLLRLLR

SEQ ID NO: 928 - protein, 9 aa, Artificial Sequence
  Synthetic CPP 1b
RLLRLLRLL

SEQ ID NO: 929 - protein, 19 aa, Artificial Sequence
  Synthetic CPP PL
RLLRLLRRLLRLLRRLLRC

SEQ ID NO: 930 - protein, 16 aa, Artificial Sequence
  Synthetic CPP Bac9-24
RLPRPRPRPLPFPRPG

SEQ ID NO: 931 - protein, 38 aa, Artificial Sequence
  Synthetic CPP D2
RLRLRLRLRLRLRLRLKLLKLLKLLKLLKKKKKKKGYK

SEQ ID NO: 932 - protein, 38 aa, Artificial Sequence
  Synthetic CPP D3
RLRLRLRLRLRLRLRLKNNKNNKNNKNNKKKKKKKGYK

SEQ ID NO: 933 - protein, 38 aa, Artificial Sequence
  Synthetic CPP D1
RLRLRLRLRLRLRLRLKRLKRLKRLKRLKKKKKKKGYK

SEQ ID NO: 934 - protein, 14 aa, Artificial Sequence
  Synthetic CPP SG3
RLSGMNEVLSFRWL

SEQ ID NO: 935 - protein, 15 aa, Artificial Sequence
  Synthetic CPP No.14-29
RLVMRVYSPTTRRYG

SEQ ID NO: 936 - protein, 15 aa, Artificial Sequence
  Synthetic CPP No.14-14
RLWARWYSPTTRRYG

SEQ ID NO: 937 - protein, 15 aa, Artificial Sequence
  Synthetic CPP No.14-15
RLWMAWYSPTTRRYG

SEQ ID NO: 938 - protein, 15 aa, Artificial Sequence
  Synthetic CPP No.14-16
RLWMRAYSPTTRRYG

SEQ ID NO: 939 - protein, 15 aa, Artificial Sequence
  Synthetic CPP No.14-17
RLWMRWASPTTRRYG

SEQ ID NO: 940 - protein, 15 aa, Artificial Sequence
  Synthetic CPP No.14-18
RLWMRWYAPTTRRYG

SEQ ID NO: 941 - protein, 15 aa, Artificial Sequence
  Synthetic CPP No.14-20
RLWMRWYSPATRRYG

SEQ ID NO: 942 - protein, 15 aa, Artificial Sequence
  Synthetic CPP RLW
RLWMRWYSPRTRAYG

SEQ ID NO: 943 - protein, 15 aa, Artificial Sequence
  Synthetic CPP No.14-21
RLWMRWYSPTARRYG

SEQ ID NO: 944 - protein, 15 aa, Artificial Sequence
  Synthetic CPP No.14-22
RLWMRWYSPTTARYG

SEQ ID NO: 945 - protein, 15 aa, Artificial Sequence
  Synthetic CPP No.14-3R
RLWMRWYSPTTRAYG

SEQ ID NO: 946 - protein, 15 aa, Artificial Sequence
  Synthetic CPP No.14-23
RLWMRWYSPTTRRAG

SEQ ID NO: 947 - protein, 15 aa, Artificial Sequence
  Synthetic CPP No.14-35
RLWMRWYSPTTRRYA

SEQ ID NO: 948 - protein, 15 aa, Artificial Sequence
  Synthetic CPP No.14-1
RLWMRWYSPTTRRYG

SEQ ID NO: 949 - protein, 15 aa, Artificial Sequence
  Synthetic CPP No.14-9
RLWMRWYSPWTRRWG

SEQ ID NO: 950 - protein, 15 aa, Artificial Sequence
  Synthetic CPP No.14-8
RLWMRWYSPWTRRYG

SEQ ID NO: 951 - protein, 16 aa, Artificial Sequence
  Synthetic CPP PN366
RLWRALPRVLRRLLRP

SEQ ID NO: 952 - protein, 15 aa, Artificial Sequence
  Synthetic CPP No.14-30
RLYMRYYSPTTRRYG

SEQ ID NO: 953 - protein, 6 aa, Artificial Sequence
  Synthetic CPP pAntp (53-58)
RMKWKK

SEQ ID NO: 954 - protein, 8 aa, Artificial Sequence
  Synthetic CPP Alpha Virus P130 (227-234)
RNRSRHRR

SEQ ID NO: 955 - protein, 7 aa, Artificial Sequence
  Synthetic CPP PA 1
RPARPAR

SEQ ID NO: 956 - protein, 16 aa, Artificial Sequence
  Synthetic CPP Ala45 substitution mutant of pAntp (43-58)
RQAKIWFQNRRMKWKK

SEQ ID NO: 957 - protein, 22 aa, Artificial Sequence
  Synthetic CPP RR-S4(13)
RQARRNRRRALWKTLLKKVLKA

SEQ ID NO: 958 - protein, 10 aa, Artificial Sequence
  Synthetic CPP Rev ARM
RQARRNRRRC

SEQ ID NO: 959 - protein, 27 aa, Artificial Sequence
  Synthetic CPP Erns1
RQGAARVTSWLGRQLRIAGKRLEGRSK

SEQ ID NO: 960 - protein, 16 aa, Artificial Sequence
  Synthetic CPP Ala46 substitution mutant of pAntp (43-58)
RQIAIWFQNRRMKWKK

SEQ ID NO: 961 - protein, 16 aa, Artificial Sequence
  Synthetic CPP Ala47 substitution mutant of pAntp (43-58)
RQIKAWFQNRRMKWKK

SEQ ID NO: 962 - protein, 16 aa, Artificial Sequence
  Synthetic CPP Ala48 substitution mutant of pAntp (43-58)
RQIKIAFQNRRMKWKK

SEQ ID NO: 963 - protein, 16 aa, Artificial Sequence
  Synthetic CPP Pen2W2F
RQIKIFFQNRRMKFKK

SEQ ID NO: 964 - protein, 16 aa, Artificial Sequence
  Synthetic CPP pAntp mutant
RQIKIFFQNRRMKWKK

SEQ ID NO: 965 - protein, 15 aa, Artificial Sequence
  Synthetic CPP Antennapedia
RQIKIQFQNRRKWKK

SEQ ID NO: 966 - protein, 6 aa, Artificial Sequence
  Synthetic CPP pAntp (43-48)
RQIKIW

SEQ ID NO: 967 - protein, 16 aa, Artificial Sequence
  Synthetic CPP Ala49 substitution mutant of pAntp (43-58)
RQIKIWAQNRRMKWKK

SEQ ID NO: 968 - protein, 16 aa, Artificial Sequence
  Synthetic CPP Ala50 substitution mutant of pAntp (43-58)
RQIKIWFANRRMKWKK

SEQ ID NO: 969 - protein, 16 aa, Artificial Sequence
  Synthetic CPP pAntpHD (Pro50)
RQIKIWFPNRRMKWKK

SEQ ID NO: 970 - protein, 8 aa, Artificial Sequence
  Synthetic CPP pAntp (43-50)
RQIKIWFQ

SEQ ID NO: 971 - protein, 16 aa, Artificial Sequence
  Synthetic CPP Ala51 substitution mutant of pAntp (43-58)
RQIKIWFQARRMKWKK

SEQ ID NO: 972 - protein, 9 aa, Artificial Sequence
  Synthetic CPP pAntp (43-51)
RQIKIWFQN

SEQ ID NO: 973 - protein, 16 aa, Artificial Sequence
  Synthetic CPP Ala52 substitution mutant of pAntp (43-58)
RQIKIWFQNARMKWKK

SEQ ID NO: 974 - protein, 16 aa, Artificial Sequence
  Synthetic CPP Met-Arg
RQIKIWFQNMRRKWKK

SEQ ID NO: 975 - protein, 10 aa, Artificial Sequence
  Synthetic CPP pAntp (43-52)
RQIKIWFQNR

SEQ ID NO: 976 - protein, 16 aa, Artificial Sequence
  Synthetic CPP Ala53 substitution mutant of pAntp (43-58)
RQIKIWFQNRAMKWKK

SEQ ID NO: 977 - protein, 11 aa, Artificial Sequence
  Synthetic CPP pAntp (43-53)
RQIKIWFQNRR

SEQ ID NO: 978 - protein, 16 aa, Artificial Sequence
  Synthetic CPP Ala54 substitution mutant of pAntp (43-58)
RQIKIWFQNRRAKWKK

SEQ ID NO: 979 - protein, 12 aa, Artificial Sequence
  Synthetic CPP pAntp (43-54)
RQIKIWFQNRRM

SEQ ID NO: 980 - protein, 16 aa, Artificial Sequence
  Synthetic CPP Ala55 substitution mutant of pAntp (43-58)
RQIKIWFQNRRMAWKK

SEQ ID NO: 981 - protein, 13 aa, Artificial Sequence
  Synthetic CPP pAntp (43-55)
RQIKIWFQNRRMK

SEQ ID NO: 982 - protein, 16 aa, Artificial Sequence
  Synthetic CPP Ala56 substitution mutant of pAntp (43-58)
RQIKIWFQNRRMKAKK

SEQ ID NO: 983 - protein, 14 aa, Artificial Sequence
  Synthetic CPP pAntp (43-56)
RQIKIWFQNRRMKW

SEQ ID NO: 984 - protein, 16 aa, Artificial Sequence
  Synthetic CPP Ala57 substitution mutant of pAntp (43-58)
RQIKIWFQNRRMKWAK

SEQ ID NO: 985 - protein, 15 aa, Artificial Sequence
  Synthetic CPP pAntp (43-57)
RQIKIWFQNRRMKWK

SEQ ID NO: 986 - protein, 16 aa, Artificial Sequence
  Synthetic CPP Ala58 substitution mutant of pAntp (43-58)
RQIKIWFQNRRMKWKA

SEQ ID NO: 987 - protein, 16 aa, Artificial Sequence
  Synthetic CPP Penetratin
RQIKIWFQNRRMKWKK

SEQ ID NO: 988 - protein, 17 aa, Artificial Sequence
  Synthetic CPP Pen-Cys
RQIKIWFQNRRMKWKKC

SEQ ID NO: 989 - protein, 35 aa, Artificial Sequence
  Synthetic CPP PN251
RQIKIWFQNRRMKWKKDIMGEWGNEIFGAIAGFLG

SEQ ID NO: 990 - protein, 18 aa, Artificial Sequence
  Synthetic CPP Pen
RQIKIWFQNRRMKWKKGC

SEQ ID NO: 991 - protein, 18 aa, Artificial Sequence
  Synthetic CPP CS-Lin-Pen
RQIKIWFQNRRMKWKKGG

SEQ ID NO: 992 - protein, 17 aa, Artificial Sequence
  Synthetic CPP Penetratin
RQIKIWFQNRRMKWKKK

SEQ ID NO: 993 - protein, 31 aa, Artificial Sequence
  Synthetic CPP Pen-GFP-Pen
RQIKIWFQNRRMKWKKRQIKIWFQNRRMKWK

SEQ ID NO: 994 - protein, 33 aa, Artificial Sequence
  Synthetic CPP pAntpPKI
RQIKIWFQNRRMKWKKTYADFIASGRTGRRNAI

SEQ ID NO: 995 - protein, 16 aa, Artificial Sequence
  Synthetic CPP PenArg
RQIRIWFQNRRMRWRR

SEQ ID NO: 996 - protein, 17 aa, Artificial Sequence
  Synthetic CPP PenArg-Cys
RQIRIWFQNRRMRWRRC

SEQ ID NO: 997 - protein, 15 aa, Artificial Sequence
  Synthetic CPP Erns11
RQLRIAGRRLRGRSR

SEQ ID NO: 998 - protein, 16 aa, Artificial Sequence
  Synthetic CPP pAntpHD (3Pro)
RQPKIWFPNRRKPWKK

SEQ ID NO: 999 - protein, 12 aa, Artificial Sequence
  Synthetic CPP Peptide 7
RQRSRRRPLNIR

SEQ ID NO: 1000 - protein, 15 aa, Artificial Sequence
  Synthetic CPP P5
RRARRPRRLRPAPGR

SEQ ID NO: 1001 - protein, 4 aa, Artificial Sequence
  Synthetic CPP R2
RRGC

SEQ ID NO: 1002 - protein, 6 aa, Artificial Sequence
  Synthetic CPP V1
RRGRRG

SEQ ID NO: 1003 - protein, 13 aa, Artificial Sequence
  Synthetic CPP hPER1-PTD
RRHHCRSKAKRSR

SEQ ID NO: 1004 - protein, 22 aa, Artificial Sequence
  Synthetic CPP B9
RRHLRRHLRHLRRHLRRHLRHL

SEQ ID NO: 1005 - protein, 10 aa, Artificial Sequence
  Synthetic CPP RSV-A9
RRIPNRRPRR

SEQ ID NO: 1006 - protein, 7 aa, Artificial Sequence
  Synthetic CPP Bac1-7
RRIRPRP

SEQ ID NO: 1007 - protein, 15 aa, Artificial Sequence
  Synthetic CPP Bac-1-15
RRIRPRPPRLPRPRP

SEQ ID NO: 1008 - protein, 17 aa, Artificial Sequence
  Synthetic CPP Bac1-17
RRIRPRPPRLPRPRPRP

SEQ ID NO: 1009 - protein, 24 aa, Artificial Sequence
  Synthetic CPP Bac-ELP43
RRIRPRPPRLPRPRPRPLPFPRPG

SEQ ID NO: 1010 - protein, 11 aa, Artificial Sequence
  Synthetic CPP M593
RRKLSQQKEKK

SEQ ID NO: 1011 - protein, 9 aa, Artificial Sequence
  Synthetic CPP R6L3
RRLLRRLRR

SEQ ID NO: 1012 - protein, 18 aa, Artificial Sequence
  Synthetic CPP Mgpe-3
RRLRHLRHHYRRRWHRFR

SEQ ID NO: 1013 - protein, 10 aa, Artificial Sequence
  Synthetic CPP SynB3
RRLSYSRRRF

SEQ ID NO: 1014 - protein, 7 aa, Artificial Sequence
  Synthetic CPP pAntp (52-58)
RRMKWKK

SEQ ID NO: 1015 - protein, 12 aa, Artificial Sequence
  Synthetic CPP Peptide 5
RRQRRTSKLMKR

SEQ ID NO: 1016 - protein, 9 aa, Artificial Sequence
  Synthetic CPP Lambda-N Truncated (50-58)
RRRERRAEK

SEQ ID NO: 1017 - protein, 15 aa, Artificial Sequence
  Synthetic CPP P3
RRRQKRIVVRRRLIR

SEQ ID NO: 1018 - protein, 9 aa, Artificial Sequence
  Synthetic CPP Retro - Tat (57-49)
RRRQRRKKR

SEQ ID NO: 1019 - protein, 26 aa, Artificial Sequence
  Synthetic CPP dfTAT
RRRQRRKKRGYCKCKYGRKKRRQRRR

SEQ ID NO: 1020 - protein, 29 aa, Artificial Sequence
  Synthetic CPP PN81
RRRQRRKRGGDIMGEWGNEIFGAIAGFLG

SEQ ID NO: 1021 - protein, 4 aa, Artificial Sequence
  Synthetic CPP R4
RRRR

SEQ ID NO: 1022 - protein, 17 aa, Artificial Sequence
  Synthetic CPP FHV coat (35-49)
RRRRNRTRRNRRRVRGC

SEQ ID NO: 1023 - protein, 5 aa, Artificial Sequence
  Synthetic CPP R5
RRRRR

SEQ ID NO: 1024 - protein, 8 aa, Artificial Sequence
  Synthetic CPP R5H3
RRRRRHHH

SEQ ID NO: 1025 - protein, 6 aa, Artificial Sequence
  Synthetic CPP R6
RRRRRR

SEQ ID NO: 1026 - protein, 9 aa, Artificial Sequence
  Synthetic CPP R6H3
RRRRRRHHH

SEQ ID NO: 1027 - protein, 7 aa, Artificial Sequence
  Synthetic CPP R7
RRRRRRR

SEQ ID NO: 1028 - protein, 23 aa, Artificial Sequence
  Synthetic CPP P7-6
RRRRRRRGGIYLATALAKWALKQ

SEQ ID NO: 1029 - protein, 25 aa, Artificial Sequence
  Synthetic CPP P7-4
RRRRRRRGGIYLATALAKWALKQGF

SEQ ID NO: 1030 - protein, 23 aa, Artificial Sequence
  Synthetic CPP R7-KLA
RRRRRRRGGKLAKLAKKLAKLAK

SEQ ID NO: 1031 - protein, 10 aa, Artificial Sequence
  Synthetic CPP R7H3
RRRRRRRHHH

SEQ ID NO: 1032 - protein, 25 aa, Artificial Sequence
  Synthetic CPP R6-Pen(W-L)
RRRRRRRQIKILFQNRRMKWKKGGC

SEQ ID NO: 1033 - protein, 8 aa, Artificial Sequence
  Synthetic CPP R8
RRRRRRRR

SEQ ID NO: 1034 - protein, 9 aa, Artificial Sequence
  Synthetic CPP R8
RRRRRRRRC

SEQ ID NO: 1035 - protein, 10 aa, Artificial Sequence
  Synthetic CPP R8 (Alexa)
RRRRRRRRGC

SEQ ID NO: 1036 - protein, 11 aa, Artificial Sequence
  Synthetic CPP R8H3
RRRRRRRRHHH

SEQ ID NO: 1037 - protein, 9 aa, Artificial Sequence
  Synthetic CPP R8
RRRRRRRRK

SEQ ID NO: 1038 - protein, 9 aa, Artificial Sequence
  Synthetic CPP R9
RRRRRRRRR

SEQ ID NO: 1039 - protein, 10 aa, Artificial Sequence
  Synthetic CPP PolyR-C-Cy5
RRRRRRRRRC

SEQ ID NO: 1040 - protein, 24 aa, Artificial Sequence
  Synthetic CPP RV24
RRRRRRRRRGPGVTWTPQAWFQWV

SEQ ID NO: 1041 - protein, 12 aa, Artificial Sequence
  Synthetic CPP R9H3
RRRRRRRRRHHH

SEQ ID NO: 1042 - protein, 10 aa, Artificial Sequence
  Synthetic CPP r9k
RRRRRRRRRK

SEQ ID NO: 1043 - protein, 10 aa, Artificial Sequence
  Synthetic CPP R12-alexa
RRRRRRRRRR

SEQ ID NO: 1044 - protein, 11 aa, Artificial Sequence
  Synthetic CPP R11
RRRRRRRRRRR

SEQ ID NO: 1045 - protein, 12 aa, Artificial Sequence
  Synthetic CPP R12
RRRRRRRRRRRR

SEQ ID NO: 1046 - protein, 14 aa, Artificial Sequence
  Synthetic CPP R12
RRRRRRRRRRRRGC

SEQ ID NO: 1047 - protein, 15 aa, Artificial Sequence
  Synthetic CPP R15
RRRRRRRRRRRRRRR

SEQ ID NO: 1048 - protein, 16 aa, Artificial Sequence
  Synthetic CPP R16
RRRRRRRRRRRRRRRR

SEQ ID NO: 1049 - protein, 18 aa, Artificial Sequence
  Synthetic CPP R16
RRRRRRRRRRRRRRRRGC

SEQ ID NO: 1050 - protein, 28 aa, Artificial Sequence
  Synthetic CPP R11-PKI
RRRRRRRRRRRTYADFIASGRTGRRNAI

SEQ ID NO: 1051 - protein, 8 aa, Artificial Sequence
  Synthetic CPP R7W
RRRRRRRW

SEQ ID NO: 1052 - protein, 8 aa, Artificial Sequence
  Synthetic CPP [R4W4]Cyclic
RRRRWWWW

SEQ ID NO: 1053 - protein, 12 aa, Artificial Sequence
  Synthetic CPP RWR
RRRRWWWWRRRR

SEQ ID NO: 1054 - protein, 23 aa, Artificial Sequence
  Synthetic CPP Erns4
RRVTSWLGRQLRIAGKRLEGRSK

SEQ ID NO: 1055 - protein, 15 aa, Artificial Sequence
  Synthetic CPP P4
RRVWRRYRRQRWCRR

SEQ ID NO: 1056 - protein, 15 aa, Artificial Sequence
  Synthetic CPP P8
RRWRRWNRFNRRRCR

SEQ ID NO: 1057 - protein, 16 aa, Artificial Sequence
  Synthetic CPP RW16
RRWRRWWRRWWRRWRR

SEQ ID NO: 1058 - protein, 9 aa, Artificial Sequence
  Synthetic CPP R6W3
RRWWRRWRR

SEQ ID NO: 1059 - protein, 16 aa, Artificial Sequence
  Synthetic CPP Erns12
RSRGRLRRGAIRLQRG

SEQ ID NO: 1060 - protein, 23 aa, Artificial Sequence
  Synthetic CPP Inv4
RSVTTEINTLFQTLTSIAEKVDP

SEQ ID NO: 1061 - protein, 15 aa, Artificial Sequence
  Synthetic CPP No.63
RTLVNEYKNTLKFSK

SEQ ID NO: 1062 - protein, 10 aa, Artificial Sequence
  Synthetic CPP FHV (40-49)
RTRRNRRRVR

SEQ ID NO: 1063 - protein, 16 aa, Artificial Sequence
  Synthetic CPP pISL
RVIRVWFQNKRCKDKK

SEQ ID NO: 1064 - protein, 15 aa, Artificial Sequence
  Synthetic CPP PN158
RVIRWFQNKRCKDKK

SEQ ID NO: 1065 - protein, 15 aa, Artificial Sequence
  Synthetic CPP PN316
RVIRWFQNKRSKDKK

SEQ ID NO: 1066 - protein, 15 aa, Artificial Sequence
  Synthetic CPP No. 2175
RVREWWYTITLKQES

SEQ ID NO: 1067 - protein, 13 aa, Artificial Sequence
  Synthetic CPP ARF(2-14) scr
RVRILARFLRTRV

SEQ ID NO: 1068 - protein, 22 aa, Artificial Sequence
  Synthetic CPP Erns5
RVRSWLGRQLRIAGKRLEGRSK

SEQ ID NO: 1069 - protein, 13 aa, Artificial Sequence
  Synthetic CPP ARF(19-31)
RVRVFVVHIPRLT

SEQ ID NO: 1070 - protein, 22 aa, Artificial Sequence
  Synthetic CPP Erns2
RVTSWLGRQLRIAGKRLEGRSK

SEQ ID NO: 1071 - protein, 8 aa, Artificial Sequence
  Synthetic CPP ECP(34-41)
RWRCKNQN

SEQ ID NO: 1072 - protein, 12 aa, Artificial Sequence
  Synthetic CPP RW MIX
RWRRWRRWRRWR

SEQ ID NO: 1073 - protein, 9 aa, Artificial Sequence
  Synthetic CPP RW9
RWRRWWRRW

SEQ ID NO: 1074 - protein, 9 aa, Artificial Sequence
  Synthetic CPP Crot (27-39) derevative
RWRWKCCKK

SEQ ID NO: 1075 - protein, 8 aa, Artificial Sequence
  Synthetic CPP (RW)4
RWRWRWRW

SEQ ID NO: 1076 - protein, 13 aa, Artificial Sequence
  Synthetic CPP Peptide 23
SAETVESCLAKSH

SEQ ID NO: 1077 - protein, 16 aa, Artificial Sequence
  Synthetic CPP hPER1-PTD alanine subsitution mutant
SARHHCRSKAKRSRHH

SEQ ID NO: 1078 - protein, 12 aa, Artificial Sequence
  Synthetic CPP Peptide 36
SATGAPWKMWVR

SEQ ID NO: 1079 - protein, 12 aa, Artificial Sequence
  Synthetic CPP Peptide 27
SFHQFARATLAS

SEQ ID NO: 1080 - protein, 37 aa, Artificial Sequence
  Synthetic CPP PN279
SGRGKQGGKARAKAKTRSSRAGLQFPVGRVHRLLRKG

SEQ ID NO: 1081 - protein, 38 aa, Artificial Sequence
  Synthetic CPP PN61
SGRGKQGGKARAKAKTRSSRAGLQFPVGRVHRLLRKGC

SEQ ID NO: 1082 - protein, 12 aa, Artificial Sequence
  Synthetic CPP Peptide 38
SHAFTWPTYLQL

SEQ ID NO: 1083 - protein, 12 aa, Artificial Sequence
  Synthetic CPP Peptide 39
SHNWLPLWPLRP

SEQ ID NO: 1084 - protein, 8 aa, Artificial Sequence
  Synthetic CPP TFIIE BETA
SKKKKTKV

SEQ ID NO: 1085 - protein, 60 aa, Artificial Sequence
  Synthetic CPP Fushi- tarazu (254-313)
SKRTRQTYTRYQTLELEKEFHFNRYITRRRRIDIANALSLSERQIKIWFQNRRMKSKKDR

SEQ ID NO: 1086 - protein, 12 aa, Artificial Sequence
  Synthetic CPP Peptide 37
SLGWMLPFSPPF

SEQ ID NO: 1087 - protein, 12 aa, Artificial Sequence
  Synthetic CPP Peptide 15
SMLKRNHSTSNR

SEQ ID NO: 1088 - protein, 12 aa, Artificial Sequence
  Synthetic CPP Peptide 63
SNPWDSLLSVST

SEQ ID NO: 1089 - protein, 12 aa, Artificial Sequence
  Synthetic CPP Peptide 17
SPMQKTMNLPPM

SEQ ID NO: 1090 - protein, 16 aa, Artificial Sequence
  Synthetic CPP hPER1-PTD alanine subsitution mutant
SRAHHCRSKAKRSRHH

SEQ ID NO: 1091 - protein, 16 aa, Artificial Sequence
  Synthetic CPP hPER1-PTD alanine subsitution mutant
SRRAHCRSKAKRSRHH

SEQ ID NO: 1092 - protein, 19 aa, Artificial Sequence
  Synthetic CPP DPV10/6
SRRARRSPRESGKKRKRKR

SEQ ID NO: 1093 - protein, 14 aa, Artificial Sequence
  Synthetic CPP DPV10
SRRARRSPRHLGSG

SEQ ID NO: 1094 - protein, 16 aa, Artificial Sequence
  Synthetic CPP hPER1-PTD alanine subsitution mutant
SRRHACRSKAKRSRHH

SEQ ID NO: 1095 - protein, 16 aa, Artificial Sequence
  Synthetic CPP hPER1-PTD alanine subsitution mutant
SRRHHARSKAKRSRHH

SEQ ID NO: 1096 - protein, 16 aa, Artificial Sequence
  Synthetic CPP hPER1-PTD alanine subsitution mutant
SRRHHCRAKAKRSRHH

SEQ ID NO: 1097 - protein, 16 aa, Artificial Sequence
  Synthetic CPP hPER1-PTD alanine subsitution mutant
SRRHHCRSAAKRSRHH

SEQ ID NO: 1098 - protein, 16 aa, Artificial Sequence
  Synthetic CPP hPER1-PTD alanine subsitution mutant
SRRHHCRSKAARSRHH

SEQ ID NO: 1099 - protein, 16 aa, Artificial Sequence
  Synthetic CPP hPER1-PTD alanine subsitution mutant
SRRHHCRSKAKASRHH

SEQ ID NO: 1100 - protein, 16 aa, Artificial Sequence
  Synthetic CPP hPER1-PTD alanine subsitution mutant
SRRHHCRSKAKRARHH

SEQ ID NO: 1101 - protein, 16 aa, Artificial Sequence
  Synthetic CPP hPER1-PTD alanine subsitution mutant
SRRHHCRSKAKRSAHH

SEQ ID NO: 1102 - protein, 12 aa, Artificial Sequence
  Synthetic CPP Peptide 9
SRRKRQRSNMRI

SEQ ID NO: 1103 - protein, 10 aa, Artificial Sequence
  Synthetic CPP SR9
SRRRRRRRRR

SEQ ID NO: 1104 - protein, 10 aa, Artificial Sequence
  Synthetic CPP Crot (27-39) derevative
SRWRWKCCKK

SEQ ID NO: 1105 - protein, 10 aa, Artificial Sequence
  Synthetic CPP Crot (27-39) derevative
SRWRWKCSKK

SEQ ID NO: 1106 - protein, 10 aa, Artificial Sequence
  Synthetic CPP Crot (27-39) derevative
SRWRWKSCKK

SEQ ID NO: 1107 - protein, 10 aa, Artificial Sequence
  Synthetic CPP Crot (27-39) derevative
SRWRWKSSKK

SEQ ID NO: 1108 - protein, 12 aa, Artificial Sequence
  Synthetic CPP Peptide 43
SSSIFPPWLSFF

SEQ ID NO: 1109 - protein, 12 aa, Artificial Sequence
  Synthetic CPP Peptide 42
SWAQHLSLPPVL

SEQ ID NO: 1110 - protein, 12 aa, Artificial Sequence
  Synthetic CPP Peptide 40
SWLPYPWHVPSS

SEQ ID NO: 1111 - protein, 12 aa, Artificial Sequence
  Synthetic CPP Peptide 41
SWWTPWHVHSES

SEQ ID NO: 1112 - protein, 12 aa, Artificial Sequence
  Synthetic CPP Peptide 25
SYIQRTPSTTLP

SEQ ID NO: 1113 - protein, 20 aa, Artificial Sequence
  Synthetic CPP PHI 21 N (12-29)
TAKTRYKARRAELIAERRGC

SEQ ID NO: 1114 - protein, 36 aa, Artificial Sequence
  Synthetic CPP IL-13p
TAMRAVDKLLLHLKKLFREGQFNRNFESIIICRDRT

SEQ ID NO: 1115 - protein, 22 aa, Artificial Sequence
  Synthetic CPP Inv3.8
TARRITPKDVIDVRSVTTEINT

SEQ ID NO: 1116 - protein, 8 aa, Artificial Sequence
  Synthetic CPP Peptide 1-NS-delta
TCTWLKYH

SEQ ID NO: 1117 - protein, 9 aa, Artificial Sequence
  Synthetic CPP Peptide 1-N-delta
TCTWLKYHS

SEQ ID NO: 1118 - protein, 12 aa, Artificial Sequence
  Synthetic CPP hCT (21-32)
TFPQTAIGVGAP

SEQ ID NO: 1119 - protein, 23 aa, Artificial Sequence
  Synthetic CPP Inv3.9
TKAARITPKDVIDVRSVTTEINT

SEQ ID NO: 1120 - protein, 22 aa, Artificial Sequence
  Synthetic CPP Inv3.3
TKRRITPDDVIDVRSVTTEINT

SEQ ID NO: 1121 - protein, 13 aa, Artificial Sequence
  Synthetic CPP Inv3.6
TKRRITPKDVIDV

SEQ ID NO: 1122 - protein, 22 aa, Artificial Sequence
  Synthetic CPP Inv3.7
TKRRITPKDVIDVESVTTEINT

SEQ ID NO: 1123 - protein, 22 aa, Artificial Sequence
  Synthetic CPP Inv3
TKRRITPKDVIDVRSVTTEINT

SEQ ID NO: 1124 - protein, 22 aa, Artificial Sequence
  Synthetic CPP Inv3.5
TKRRITPKDVIDVRSVTTKINT

SEQ ID NO: 1125 - protein, 22 aa, Artificial Sequence
  Synthetic CPP Inv3.4
TKRRITPKKVIDVRSVTTEINT

SEQ ID NO: 1126 - protein, 12 aa, Artificial Sequence
  Synthetic CPP Peptide 53
TLPSPLALLTVH

SEQ ID NO: 1127 - protein, 12 aa, Artificial Sequence
  Synthetic CPP Peptide 59
TPKTMTQTYDFS

SEQ ID NO: 1128 - protein, 24 aa, Artificial Sequence
  Synthetic CPP FITC-Rath
TPWWRLWTKWHHKRRDLPRKPEGC

SEQ ID NO: 1129 - protein, 17 aa, Artificial Sequence
  Synthetic CPP Rev (34-50)
TRQARRNRRRRWRERQR

SEQ ID NO: 1130 - protein, 19 aa, Artificial Sequence
  Synthetic CPP HIV-1 Rev (34-50)
TRQARRNRRRRWRERQRGC

SEQ ID NO: 1131 - protein, 15 aa, Artificial Sequence
  Synthetic CPP HTLV -II Rex(4-16)
TRRQRTRRARRNRGC

SEQ ID NO: 1132 - protein, 12 aa, Artificial Sequence
  Synthetic CPP Herpesvirus 8 k8 protein (124-135)
TRRSKRRSHRKF

SEQ ID NO: 1133 - protein, 24 aa, Artificial Sequence
  Synthetic CPP BF2d
TRSSRAGLQWPVGRVHRLLRKGGC

SEQ ID NO: 1134 - protein, 12 aa, Artificial Sequence
  Synthetic CPP Peptide 55
TSHTDAPPARSP

SEQ ID NO: 1135 - protein, 12 aa, Artificial Sequence
  Synthetic CPP HN-1
TSPLNIHNGQKL

SEQ ID NO: 1136 - protein, 19 aa, Artificial Sequence
  Synthetic CPP VP1 BC loop (V) peptides
TVDNPASTTNKDKLFAVRK

SEQ ID NO: 1137 - protein, 6 aa, Artificial Sequence
  Synthetic CPP Peptide 1-NTCS-delta
TWLKYH

SEQ ID NO: 1138 - protein, 4 aa, Artificial Sequence
  Synthetic CPP Xentry peptides
VCVR

SEQ ID NO: 1139 - protein, 18 aa, Artificial Sequence
  Synthetic CPP Sweet Arrow Protein (SAP) (E)
VELPPPVELPPPVELPPP

SEQ ID NO: 1140 - protein, 6 aa, Artificial Sequence
  Synthetic CPP PolyP 4
VHLPPP

SEQ ID NO: 1141 - protein, 12 aa, Artificial Sequence
  Synthetic CPP PolyP 5
VHLPPPVHLPPP

SEQ ID NO: 1142 - protein, 18 aa, Artificial Sequence
  Synthetic CPP PolyP 6
VHLPPPVHLPPPVHLPPP

SEQ ID NO: 1143 - protein, 13 aa, Artificial Sequence
  Synthetic CPP ARF(19-31) scr
VIRVHFRLPVRTV

SEQ ID NO: 1144 - protein, 6 aa, Artificial Sequence
  Synthetic CPP PolyP 7
VKLPPP

SEQ ID NO: 1145 - protein, 12 aa, Artificial Sequence
  Synthetic CPP PolyP 8
VKLPPPVKLPPP

SEQ ID NO: 1146 - protein, 18 aa, Artificial Sequence
  Synthetic CPP PolyP 9
VKLPPPVKLPPPVKLPPP

SEQ ID NO: 1147 - protein, 15 aa, Artificial Sequence
  Synthetic CPP B1-Lys
VKRFKKFFRKLKKKV

SEQ ID NO: 1148 - protein, 15 aa, Artificial Sequence
  Synthetic CPP B1-Leu
VKRFKKFFRKLKKLV

SEQ ID NO: 1149 - protein, 15 aa, Artificial Sequence
  Synthetic CPP B1
VKRFKKFFRKLKKSV

SEQ ID NO: 1150 - protein, 19 aa, Artificial Sequence
  Synthetic CPP DPV1047
VKRGLKLRHVRPRVTRMDV

SEQ ID NO: 1151 - protein, 20 aa, Artificial Sequence
  Synthetic CPP PV reverse-S4(13)
VKRKKKPALWKTLLKKVLKA

SEQ ID NO: 1152 - protein, 5 aa, Artificial Sequence
  Synthetic CPP Xentry peptides
VLCLR

SEQ ID NO: 1153 - protein, 12 aa, Artificial Sequence
  Synthetic CPP Peptide 57
VLGQSGYLMPMR

SEQ ID NO: 1154 - protein, 21 aa, Artificial Sequence
  Synthetic CPP Inv1
VNADIKATTVFGGKYVSLTTP

SEQ ID NO: 1155 - protein, 5 aa, Artificial Sequence
  Synthetic CPP Bip6
VPALK

SEQ ID NO: 1156 - protein, 5 aa, Artificial Sequence
  Synthetic CPP Bip3
VPALR

SEQ ID NO: 1157 - protein, 5 aa, Artificial Sequence
  Synthetic CPP Bip13
VPMIK

SEQ ID NO: 1158 - protein, 5 aa, Artificial Sequence
  Synthetic CPP Bip1
VPMLK

SEQ ID NO: 1159 - protein, 5 aa, Artificial Sequence
  Synthetic CPP Bip19
VPTLE

SEQ ID NO: 1160 - protein, 5 aa, Artificial Sequence
  Synthetic CPP Bip2
VPTLK

SEQ ID NO: 1161 - protein, 5 aa, Artificial Sequence
  Synthetic CPP Bip16
VPTLQ

SEQ ID NO: 1162 - protein, 15 aa, Artificial Sequence
  Synthetic CPP M630
VQAILRRNWNQYKIQ

SEQ ID NO: 1163 - protein, 8 aa, Artificial Sequence
  Synthetic CPP Peptide 10
VQLRRRWC

SEQ ID NO: 1164 - protein, 10 aa, Artificial Sequence
  Synthetic CPP NF-kB
VQRKRQKLMP

SEQ ID NO: 1165 - protein, 6 aa, Artificial Sequence
  Synthetic CPP PolyP 1
VRLPPP

SEQ ID NO: 1166 - protein, 12 aa, Artificial Sequence
  Synthetic CPP PolyP 2
VRLPPPVRLPPP

SEQ ID NO: 1167 - protein, 18 aa, Artificial Sequence
  Synthetic CPP PolyP 3 (SAP)
VRLPPPVRLPPPVRLPPP

SEQ ID NO: 1168 - protein, 13 aa, Artificial Sequence
  Synthetic CPP ARF(2-14)
VRRFLVTLRIRRA

SEQ ID NO: 1169 - protein, 5 aa, Artificial Sequence
  Synthetic CPP Bip4
VSALK

SEQ ID NO: 1170 - protein, 5 aa, Artificial Sequence
  Synthetic CPP Bip8
VSGKK

SEQ ID NO: 1171 - protein, 12 aa, Artificial Sequence
  Synthetic CPP Peptide 47
VSKQPYYMWNGN

SEQ ID NO: 1172 - protein, 5 aa, Artificial Sequence
  Synthetic CPP Bip7
VSLKK

SEQ ID NO: 1173 - protein, 14 aa, Artificial Sequence
  Synthetic CPP LMWP
VSRRRRRRGGRRRR

SEQ ID NO: 1174 - protein, 15 aa, Artificial Sequence
  Synthetic CPP Protamine
VSRRRRRRGGRRRRK

SEQ ID NO: 1175 - protein, 21 aa, Artificial Sequence
  Synthetic CPP VG-21
VTPHHVLVDEYTGEWVDSQFK

SEQ ID NO: 1176 - protein, 4 aa, Artificial Sequence
  Synthetic CPP Xentry peptides
VVVR

SEQ ID NO: 1177 - protein, 30 aa, Artificial Sequence
  Synthetic CPP GALA
WEAALAEALAEALAEHLAEALAEALEALAA

SEQ ID NO: 1178 - protein, 30 aa, Artificial Sequence
  Synthetic CPP KALA
WEAKLAKALAKALAKHLAKALAKALKACEA

SEQ ID NO: 1179 - protein, 24 aa, Artificial Sequence
  Synthetic CPP RALA peptide
WEARLARALARALARHLARALARA

SEQ ID NO: 1180 - protein, 30 aa, Artificial Sequence
  Synthetic CPP RALA
WEARLARALARALARHLARALARALRACEA

SEQ ID NO: 1181 - protein, 11 aa, Artificial Sequence
  Synthetic CPP pAntp (48-58)
WFQNRRMKWKK

SEQ ID NO: 1182 - protein, 12 aa, Artificial Sequence
  Synthetic CPP TCTP-CPP 25
WIIFKIAASHKK

SEQ ID NO: 1183 - protein, 12 aa, Artificial Sequence
  Synthetic CPP TCTP-CPP 18
WIIFRAAASHKK

SEQ ID NO: 1184 - protein, 12 aa, Artificial Sequence
  Synthetic CPP TCTP-CPP 19
WIIFRALISHKK

SEQ ID NO: 1185 - protein, 12 aa, Artificial Sequence
  Synthetic CPP TCTP-CPP 17
WIIFRIAASHKK

SEQ ID NO: 1186 - protein, 12 aa, Artificial Sequence
  Synthetic CPP TCTP-CPP 23
WIIFRIAATHKK

SEQ ID NO: 1187 - protein, 12 aa, Artificial Sequence
  Synthetic CPP TCTP-CPP 21
WIIFRIAAYHKK

SEQ ID NO: 1188 - protein, 15 aa, Artificial Sequence
  Synthetic CPP 48
WKARRQCFRVLHHWN

SEQ ID NO: 1189 - protein, 15 aa, Artificial Sequence
  Synthetic CPP 47
WKCRRQAFRVLHHWN

SEQ ID NO: 1190 - protein, 15 aa, Artificial Sequence
  Synthetic CPP 45
WKCRRQCFRVLHHWN

SEQ ID NO: 1191 - protein, 14 aa, Artificial Sequence
  Synthetic CPP NrTP8
WKQSHKKGGKKGSG

SEQ ID NO: 1192 - protein, 19 aa, Artificial Sequence
  Synthetic CPP PF21
WLKLLKKWLKLWKKLLKLW

SEQ ID NO: 1193 - protein, 23 aa, Artificial Sequence
  Synthetic CPP MK2i
WLRRIKAWLRRIKALNRQLGVAA

SEQ ID NO: 1194 - protein, 20 aa, Artificial Sequence
  Synthetic CPP PN291
WRFKAAVALLPAVLLALLAP

SEQ ID NO: 1195 - protein, 10 aa, Artificial Sequence
  Synthetic CPP PN290
WRFKKSKRKV

SEQ ID NO: 1196 - protein, 8 aa, Artificial Sequence
  Synthetic CPP PN287
WRFKWRFK

SEQ ID NO: 1197 - protein, 12 aa, Artificial Sequence
  Synthetic CPP PN288
WRFKWRFKWRFK

SEQ ID NO: 1198 - protein, 9 aa, Artificial Sequence
  Synthetic CPP WR8
WRRRRRRRR

SEQ ID NO: 1199 - protein, 8 aa, Artificial Sequence
  Synthetic CPP cyclic [W(RW)4]
WRWKKKKA

SEQ ID NO: 1200 - protein, 14 aa, Artificial Sequence
  Synthetic CPP Unknown
WRWRWRWRWRWRWR

SEQ ID NO: 1201 - protein, 10 aa, Artificial Sequence
  Synthetic CPP W2R8
WWRRRRRRRR

SEQ ID NO: 1202 - protein, 11 aa, Artificial Sequence
  Synthetic CPP W3R8
WWWRRRRRRRR

SEQ ID NO: 1203 - protein, 12 aa, Artificial Sequence
  Synthetic CPP W4R8
WWWWRRRRRRRR

SEQ ID NO: 1204 - protein, 11 aa, Artificial Sequence
  Synthetic CPP YARA
YARAAARQARA

SEQ ID NO: 1205 - protein, 22 aa, Artificial Sequence
  Synthetic CPP YARA
YARAAARQARAKALARQLGVAA

SEQ ID NO: 1206 - protein, 11 aa, Artificial Sequence
  Synthetic CPP CTP50
YARAARRAARR

SEQ ID NO: 1207 - protein, 11 aa, Artificial Sequence
  Synthetic CPP CTP505
YAREARRAARR

SEQ ID NO: 1208 - protein, 11 aa, Artificial Sequence
  Synthetic CPP CTP508
YARKARRAARR

SEQ ID NO: 1209 - protein, 11 aa, Artificial Sequence
  Synthetic CPP Hph-1
YARVRRRGPRR

SEQ ID NO: 1210 - protein, 11 aa, Artificial Sequence
  Synthetic CPP CTP506
YEREARRAARR

SEQ ID NO: 1211 - protein, 34 aa, Artificial Sequence
  Synthetic CPP I-TYR-L-Mca
YGDCLPHLKLCKENKDCCSKKCKRRGTNIEKRCR

SEQ ID NO: 1212 - protein, 11 aa, Artificial Sequence
  Synthetic CPP CTP504
YGRAARRAARR

SEQ ID NO: 1213 - protein, 11 aa, Artificial Sequence
  Synthetic CPP RTAT-ELPBC
YGRGGRRGRRR

SEQ ID NO: 1214 - protein, 12 aa, Artificial Sequence
  Synthetic CPP Tat
YGRKKKRRQRRR

SEQ ID NO: 1215 - protein, 11 aa, Artificial Sequence
  Synthetic CPP 1 (TAT)
YGRKKRPQRRR

SEQ ID NO: 1216 - protein, 11 aa, Artificial Sequence
  Synthetic CPP TAT(47-57)
YGRKKRRQRRR

SEQ ID NO: 1217 - protein, 29 aa, Artificial Sequence
  Synthetic CPP PEP-2
YGRKKRRQRRRAYFNGCSSPTAPLSPMSP

SEQ ID NO: 1218 - protein, 12 aa, Artificial Sequence
  Synthetic CPP Tat-C-Cy5
YGRKKRRQRRRC

SEQ ID NO: 1219 - protein, 48 aa, Artificial Sequence
  Synthetic CPP PEP-1
YGRKKRRQRRRDPYHATSGALSPAKDCGSQKYAYFNGCSSPTLSPMSP

SEQ ID NO: 1220 - protein, 13 aa, Artificial Sequence
  Synthetic CPP TAT
YGRKKRRQRRRGC

SEQ ID NO: 1221 - protein, 25 aa, Artificial Sequence
  Synthetic CPP PN204
YGRKKRRQRRRGCYGRKKRRQRRRG

SEQ ID NO: 1222 - protein, 34 aa, Artificial Sequence
  Synthetic CPP TAT-HA2
YGRKKRRQRRRGLFGAIAGFIENGWEGMIDGWYG

SEQ ID NO: 1223 - protein, 23 aa, Artificial Sequence
  Synthetic CPP TAT-NBD
YGRKKRRQRRRGTALDWSWLQTE

SEQ ID NO: 1224 - protein, 15 aa, Artificial Sequence
  Synthetic CPP TAT
YGRKKRRQRRRPPQG

SEQ ID NO: 1225 - protein, 25 aa, Artificial Sequence
  Synthetic CPP PEP-3
YGRKKRRQRRRQRRRPTAPLSPMSP

SEQ ID NO: 1226 - protein, 22 aa, Artificial Sequence
  Synthetic CPP Tat-GFP-Tat
YGRKKRRQRRRYGRKKRRQRRR

SEQ ID NO: 1227 - protein, 33 aa, Artificial Sequence
  Synthetic CPP SP-Tatm3xCherry
YGRKKRRQRRRYGRKKRRQRRRYGRKKRRQRRR

SEQ ID NO: 1228 - protein, 21 aa, Artificial Sequence
  Synthetic CPP Mutant tat-NBD
YGRKKRRQRRTALDASALQTE

SEQ ID NO: 1229 - protein, 21 aa, Artificial Sequence
  Synthetic CPP Biotin-labeled tat-NBD peptides
YGRKKRRQRRTALDWSWLQTE

SEQ ID NO: 1230 - protein, 11 aa, Artificial Sequence
  Synthetic CPP CTP510
YGRRARRAARR

SEQ ID NO: 1231 - protein, 11 aa, Artificial Sequence
  Synthetic CPP CTP511
YGRRARRRARR

SEQ ID NO: 1232 - protein, 11 aa, Artificial Sequence
  Synthetic CPP CTP512
YGRRARRRRRR

SEQ ID NO: 1233 - protein, 11 aa, Artificial Sequence
  Synthetic CPP CTP513
YGRRRRRRRRR

SEQ ID NO: 1234 - protein, 16 aa, Artificial Sequence
  Synthetic CPP M591
YIVLRRRRKRVNTKRS

SEQ ID NO: 1235 - protein, 12 aa, Artificial Sequence
  Synthetic CPP YKA peptide
YKALRISRKLAK

SEQ ID NO: 1236 - protein, 42 aa, Artificial Sequence
  Synthetic CPP Crotamine
YKQCHKKGGHCFPKEKICLPPSSDFGKMDCRWRWKCCKKGSG

SEQ ID NO: 1237 - protein, 14 aa, Artificial Sequence
  Synthetic CPP NrTP1
YKQCHKKGGKKGSG

SEQ ID NO: 1238 - protein, 11 aa, Artificial Sequence
  Synthetic CPP CTP507
YKRAARRAARR

SEQ ID NO: 1239 - protein, 11 aa, Artificial Sequence
  Synthetic CPP CTP509
YKRKARRAARR

SEQ ID NO: 1240 - protein, 10 aa, Artificial Sequence
  Synthetic CPP Peptide 3
YNNFAYSVFL

SEQ ID NO: 1241 - protein, 11 aa, Artificial Sequence
  Synthetic CPP CTP502
YPRAARRAARR

SEQ ID NO: 1242 - protein, 12 aa, Artificial Sequence
  Synthetic CPP Peptide 51
YPYDANHTRSPT

SEQ ID NO: 1243 - protein, 10 aa, Artificial Sequence
  Synthetic CPP Peptide 9
YQKQAKIMCS

SEQ ID NO: 1244 - protein, 10 aa, Artificial Sequence
  Synthetic CPP Peptide 7
YRDRFAFQPH

SEQ ID NO: 1245 - protein, 4 aa, Artificial Sequence
  Synthetic CPP PN267
YRFK

SEQ ID NO: 1246 - protein, 13 aa, Artificial Sequence
  Synthetic CPP PN282
YRFKYRFKYRLFK

SEQ ID NO: 1247 - protein, 14 aa, Artificial Sequence
  Synthetic CPP NrTP7
YRQSHRRGGRRGSG

SEQ ID NO: 1248 - protein, 11 aa, Artificial Sequence
  Synthetic CPP CTP503
YRRAARRAARA

SEQ ID NO: 1249 - protein, 11 aa, Artificial Sequence
  Synthetic CPP CTP514
YRRRRRRRRRR

SEQ ID NO: 1250 - protein, 8 aa, Artificial Sequence
  Synthetic CPP ECP(33-40)
YRWRCKNQ

SEQ ID NO: 1251 - protein, 9 aa, Artificial Sequence
  Synthetic CPP ECP(33-41)
YRWRCKNQN

SEQ ID NO: 1252 - protein, 12 aa, Artificial Sequence
  Synthetic CPP Peptide 24
YSHIATLPFTPT

SEQ ID NO: 1253 - protein, 24 aa, Artificial Sequence
  Synthetic CPP NFL-TBS.40-63
YSSYSAPVSSSLSVRRSYSSSSGS

SEQ ID NO: 1254 - protein, 16 aa, Artificial Sequence
  Synthetic CPP YTA2
YTAIAWVKAFIRKLRK

SEQ ID NO: 1255 - protein, 12 aa, Artificial Sequence
  Synthetic CPP Ypep-GFP
YTFGLKTSFNVQ

SEQ ID NO: 1256 - protein, 24 aa, Artificial Sequence
  Synthetic CPP Ypep-GFP-Ypep
YTFGLKTSFNVQYTFGLKTSFNVQ

SEQ ID NO: 1257 - protein, 21 aa, Artificial Sequence
  Synthetic CPP hCT(1232)
YTQDFNKFHTFPQTAIGVGAP

SEQ ID NO: 1258 - protein, 13 aa, Artificial Sequence
  Synthetic CPP Tyr-Oct-6
YYYAAGRKRKKRT

SEQ ID NO: 1259 - protein, 393 aa, Artificial Sequence
  Synthetic CPP mature CPG2
ALAQKRDNVLFQAATDEQPAVIKTLEKLVNIETGTGDAEGIAAAGNFLEAELKNLGFTVTRSKSAGLVVGDNIVGKIKGRGGKNLLLMSHMDTVYLKGILAKAPFRVEGDKAYGPGIADDKGGNAVILHTLKLLKEYGVRDYGTITVLFNTDEEKGSFGSRDLIQEEAKLADYVLSFEPTSAGDEKLSLGTSGIAYVQVNITGKASHAGAAPELGVNALVEASDLVLRTMNIDDKAKNLRFNWTIAKAGNVSNIIPASATLNADVRYARNEDFDAAMKTLEERAQQKKLPEADVKVIVTRGRPAFNAGEGGKKLVDKAVAYYKEAGGTLGVEERTGGGTDAAYAALSGKPVIESLGLPGFGYHSDKAEYVDISAIPRRLYMAARLIMDLGAGK

(19) United States
(12) Patent Application Publication
(10) Pub. No.: US 2022/0287968 A1
(43) Pub. Date: Sep. 15, 2022

(54) LIPID VESICLE-MEDIATED DELIVERY TO CELLS

(71) Applicant: FLORIDA STATE UNIVERSITY RESEARCH FOUNDATION, INC., TALLAHASSEE, FL (US)

(72) Inventors: ZUCAI SUO, TALLAHASSEE, FL (US); MANGESH D. HADE, TALLAHASSEE, FL (US)

(21) Appl. No.: 17/654,154

(22) Filed: Mar. 9, 2022

Related U.S. Application Data
(60) Provisional application No. 63/200,472, filed on Mar. 9, 2021.

(51) Int. Cl. A61K 9/127; A61K 47/60; A61K 47/54; A61K 47/69; A61K 9/16

(52) U.S. Cl. CPC A61K 9/1271; A61K 47/60; A61K 47/543; A61K 47/6911; A61K 9/167

(57) ABSTRACT

The invention concerns a lipid vesicle (LV), such as a liposome, that has been loaded with a cargo molecule covalently or non-covalently coupled to a cell penetrating polypeptide (resulting in a “binding complex”), and the binding complex or cargo molecule has been internalized by, or is associated with, the LV. Another aspect of the invention concerns a method for loading an LV with a cargo molecule, comprising contacting the LV with the binding complex, wherein the binding complex or cargo molecule becomes internalized by, or associated with, the LV. Another aspect of the invention concerns a method for delivering a cargo molecule into a cell in vitro or in vivo, comprising administering a loaded LV to the cell in vitro or in vivo, wherein the loaded LV is internalized into the cell, and wherein the loaded LV comprises the cargo molecule and a cell penetrating polypeptide.

### CROSS-REFERENCE TO RELATED APPLICATION

The present application claims the benefit of U.S. Provisional Application Ser. No. 63/200,472, filed Mar. 9, 2021, which is hereby incorporated by reference herein in its entirety, including any figures, tables, nucleic acid sequences, amino acid sequences, or drawings.

### SEQUENCE LISTING

The Sequence Listing for this application is labeled “2T18729.txt” which was created on Mar. 9, 2022 and is 348 KB. The entire contents of the sequence listing is incorporated herein by reference in its entirety.

### BACKGROUND OF THE INVENTION

Effective drug delivery usually proceeds through a succession of steps including a long circulation in the system, penetration of a biological barrier, uptake in recipient cells, and endosomal escape to the cytosolic space after endocytosis. Each of these steps has its own potential barriers and uncertainties. For example, since the plasma membrane normally acts as a biochemical barrier to prevent exogenous invasion, many bioactive molecules face hurdles in accessing and penetrating the target cell membrane in order to fulfill their therapeutic functions. Strategies commonly used for delivery of macromolecules may result in immunogenicity, degradation, chemical modification, poor specificity, high toxicity, and/or low delivery efficiency and efficacy. Therefore, a novel and innovative approach is urgently needed for the delivery of cargo molecules into target cells with high efficiency and efficacy.

### BRIEF SUMMARY OF THE INVENTION

Lipid vesicles (LVs) are vesicles that are enclosed by at least one lipid layer. The present invention relates to the utilization of LVs for delivery of loaded cargo molecules into cells. Any LVs may be utilized, such as liposomes, lipid nanoparticles, lipid droplets, micelles, reverse micelles, lipid-polymer hybrid nanoparticles, and artificial extracellular vesicles.

More particularly, the present invention relates to the use of cell-penetrating polypeptides (CPPs) in LV-mediated delivery of cargo molecules into cells in vitro or in vivo, e.g., for medical and biological applications. The present invention also relates to: (i) a method for efficient loading of cargo molecules into or onto LVs for delivery to cells, with the loading method comprising covalently or non-covalently coupling a CPP with the cargo molecule; (ii) the resulting loaded LVs themselves; and (iii) uses of the loaded LVs for biotech, diagnostics, medical imaging, cosmetic, therapeutic, and other purposes. The invention allows delivery of diverse cargo molecules such as drugs, nucleic acids, macromolecules, enzymes, proteins, and peptides, into eukaryotic cells without being degraded or modified by extracellular enzymes or neutralized by host immune responses. Moreover, this protection conferred by LV-mediated delivery can be achieved without the need for chemical modification of the cargo molecule as a countermeasure, though chemical modification remains an option.

One aspect of the invention concerns a method for loading an LV with a cargo molecule (one or more cargo molecules), comprising contacting the LV with the cargo molecule covalently or non-covalently coupled to a CPP. The construct comprising the CPP coupled to the cargo molecule is referred to herein as a “binding complex”. The binding complex becomes internalized by, or associated with, the LV. In some embodiments, the LV is a liposome, lipid nanoparticle, lipid droplet, micelle, reverse micelle, lipid-polymer hybrid nanoparticle, or artificial extracellular vesicle. Upon contacting a cell, the LV is internalized by the cell and the cargo is delivered into the cell.

The cargo molecule may belong to any class of substance or combination of classes. Examples of cargo molecules include, but are not limited to, a small molecule (e.g., a drug, a fluorophore, a luminophore), macromolecule, polypeptide of any length (natural or modified), nucleic acids (natural or modified, e.g., DNA, RNA, PNA, DNA-like or RNA-like molecule, small interfering RNA (siRNA), RNAi (e.g., small interfering RNA (siRNA), short hairpin RNA (shRNA), non-coding RNA (ncRNA) such as microRNA (miRNA), small nuclear RNA (snRNA), transfer RNA (tRNA), messenger RNA (mRNA)), antibody or antibody-fragment, lipoprotein, proteins (e.g., enzymes, membrane-bound proteins), carbohydrate, or glycoprotein. In some embodiments, the cargo molecule is a hormone, metabolite, signal molecule, vitamin, or anti-aging agent. In some embodiments, the cargo molecule is a medical imaging or detectable agent, or is attached to a medical imaging or detectable agent, such as a fluorescent compound (e.g., a fluorophore) to serve as a marker, dye, quantum dot, tag, or reporter. In some embodiments, the cargo molecule is a nucleic acid such as an antisense oligonucleotide, DNA, interfering RNA molecule (e.g., shRNA), miRNA, tRNA, mRNA, guide RNA (e.g., sgRNA) for gene editing by a gene editing enzyme (e.g., Clustered Regularly Interspaced Short Palindromic Repeats (CRISPR) associated protein 9 (Cas9)), catalytic RNA, RNAzyme, ribozyme, or a nucleic acid encoding a polypeptide of any length. In some embodiments, the cargo molecule is a labeled protein, such as a labeled protein useful in nuclear magnetic resonance (NMR) protein measurement.

Another aspect of the invention is the loaded LV itself, comprising a cargo molecule and a CPP. The cargo molecule may still be covalently or non-covalently coupled to a CPP (together referred to as a binding complex), wherein the binding complex has been internalized within the LV, or is associated with the LV membrane; or the cargo molecule may be uncoupled from the CPP once the cargo molecule has been internalized within the LV or is associated with the LV membrane (i.e., the components of the binding complex have become physically separated, no longer forming the complex).

Another aspect of the invention concerns a method for delivering a cargo molecule into a cell in vitro or in vivo by administering a loaded LV to a cell in vitro or in vivo, upon which the loaded LV is internalized into the cell, and wherein the loaded LV contains the cargo molecule and a CPP. The cargo molecule and CPP may still be coupled at the time of administration of the loaded LVs to cells or the cargo molecule and CPP may be in an uncoupled condition. In in vivo embodiments, the loaded LV is administered to a human or animal subject by any route suitable to reach the target cells.

In some embodiments of the delivery method, the cargo molecule is a growth factor or growth miRNA. The growth factor-loaded LV or growth miRNA-loaded LV may be administered to the cell of a wound in vivo. In some embodiments, the growth factor-loaded LV or growth miRNA-loaded LV is administered to a subject for treatment of an acute or chronic wound. For example, the growth factor-loaded LV or growth miRNA-loaded LV can be administered to a skin cell (e.g., a primary dermal fibroblast).

### BRIEF DESCRIPTION OF THE DRAWINGS

FIGS. 1A and 1B. TIRF image of liposomes loaded with the FAM-YARA peptide. (FIG. 1A) Through TIRF microscopy, bright fluorescence was observed under the 488 nm channel from the liposomes loaded with FAM-YARA. (FIG. 1B) A magnified TIRF image of a single liposome. Scale bars are 100 nm.

FIGS. 2A and 2B. TIRF image of liposomes encapsulated with Peptide H. (FIG. 2A) Through TIRF microscopy, bright fluorescence was observed under the 488 nm channel from the liposomes loaded with Peptide H. (FIG. 2B) A magnified TIRF image of a single liposome. Scale bars are 100 nm.

FIGS. 3A and 3B. TIRF image of liposomes encapsulated with a fusion protein YARA-FGF1-GFP. (FIG. 3A) Through TIRF microscopy, bright fluorescence was observed under the 488 nm channel from the liposomes loaded with YARA-FGF1-GFP. (FIG. 3B) A magnified TIRF image of a single liposome. Scale bars are 100 nm.

FIGS. 4A and 4B. (FIG. 4A) Standard curve of GFP fluorescence intensity versus the concentration of the recombinant GFP protein provided in the GFP Fluorometric Quantification Assay Kit (CELL BIOLABS, Inc., San Diego, Calif., USA). (FIG. 4B) Time-dependent loading of the purified recombinant YARA-FGF1-GFP into liposomes. The YARA-FGF1-GFP (50 μg) was incubated with liposomes (0.1 mg/mL, 5.8×10⁹ particles/mL) in PBS for various times. After washing and filtration to get rid of any unbound YARA-FGF1-GFP, the loaded liposome samples were subjected to fluorescence measurement.

FIGS. 5A and 5B. TIRF image of liposomes encapsulated with a nucleic acid cargo. (FIG. 5A) Through TIRF microscopy, bright fluorescence was observed under the 488 nm channel from the liposomes loaded with FAM-YARA-Cys-ssDNA. (FIG. 5B) A magnified TIRF image of a single liposome. Scale bars are 100 nm.

FIG. 6. Cellular uptake of the liposomes loaded with two cargos (the fluorescent dye FAM and a peptide) via a CPP was confirmed using confocal microscopy. Bright field, FAM, and superimposed images of human primary dermal fibroblast cells after four-hour incubation at 37° C. with the liposomes loaded with Peptide H. Scale bars are 50 μm.

FIG. 7. Cellular uptake of the liposomes loaded with a CPP fused with a protein cargo. Bright field, GFP, and superimposed images of human primary dermal fibroblast cells after four-hour incubation at 37° C. with the liposomes loaded with the fusion protein YARA-FGF1-GFP. Scale bars are 50 μm.

FIGS. 8A and 8B. Liposomes loaded with YARA-FGF1-GFP enhanced mouse embryonic fibroblast migration in the scratch assays. (FIG. 8A) Time-dependent scratch assays were performed and brightfield images of fibroblast migration were captured at various time points (t=0 to 24 h). (FIG. 8B) Closure of the scratched area in (FIG. 8A) was quantitatively analyzed by using ImageJ under four different conditions. Values are representative of mean±SD from four independent experiments. Statistical significance in comparison to the untreated control was derived by ANOVA and post-hoc Tukey HSD tests (*** denotes p<0.001; ** means p<0.01). Scale bars indicate 100 The scratch assays were used to assess the migration of mouse embryonic fibroblasts or human primary dermal fibroblasts treated with PBS, the liposomes, the liposomes loaded with YARA, or the liposomes loaded with YARA-FGF1-GFP. The liposome concentration in each case was 0.1 mg/mL (5.8×10⁹ particles/mL). The fibroblasts (1×10⁶ cells/well) were seeded onto 24-well plates containing scratch field inserts. After the formation of monolayer of cells, insertion parts were removed from wells to create a “wound” scratch (approximately 0.9 mm wide), as per supplier's instructions. The plates were then incubated at 37° C. under 5% CO₂ and the fibroblast migration was observed under microscope by bright field imaging. Scale bars indicate 100 μm.

FIGS. 9A and 9B. Liposomes loaded with YARA-FGF1-GFP enhance human primary dermal fibroblasts migration in the scratch assays. The scratch assays were performed as in FIG. 8A. (FIG. 9A) Time-dependent scratch assays were performed and brightfield images of fibroblast migration were captured at various time points (t=0 to 24 h). Scale bars indicate 100 (FIG. 9B) Closure of the scratched area in (FIG. 9A) was quantitatively analyzed by using ImageJ under four different conditions. Values are representative of mean±SD from four independent experiments. Statistical significance in comparison to the untreated control was derived by ANOVA and post-hoc Tukey HSD tests (*** denotes p<0.001; ** means p<0.01).

FIG. 10. Mouse embryonic fibroblasts treated with the liposomes loaded with YARA-FGF1-GFP showed significantly enhanced proliferation in MTS cell proliferation assays. Mouse embryonic fibroblasts were seeded at a density of 5×10⁴ cells/well into 96 well plates and exposed to indicated treatments. The liposome concentration in each case except the PBS-treated control was 0.1 mg/mL (5.8×10⁹ particles/mL). MTS assay was performed to assess cell proliferation after t=24, 48, and 72 h under normal growth conditions, as per manufacturer's instructions. Values are represented of mean±SD from four independent experiments. Statistical significance was derived by two-way ANOVA followed by Bonferroni's posttest (*** denotes p<0.001). Values are compared with the PBS-treated control.

FIG. 11. Human primary dermal fibroblasts treated with the liposomes loaded with YARA-FGF1-GFP show increased proliferation in MTS cell proliferation assays as performed in FIG. 10. The values are represented of mean±SD from four independent experiments. Statistical significance was derived by two-way ANOVA followed by Bonferroni's posttest (*** p<0.001). Values are compared with the PBS-treated control.

FIGS. 12A and 12B. Internalization of the liposomes loaded with YARA-FGF1-GFP enhanced the invasion of mouse embryonic fibroblasts in cell invasion assays. (FIG. 12A) Mouse embryonic fibroblasts were seeded at density 1×10⁶ cells/mL onto 24 well plates and then exposed to indicated treatments. The liposomes concentration in each treatment except the PBS-treated control was 0.1 mg/mL (5.8×10⁹ particles/mL). Cell invasion assays were performed after t=48 h under normal growth conditions, as per manufacturer's instructions. (FIG. 12B) Quantitation of the cell invasion assays in (FIG. 12A). Values are represented as mean±SD from four independent experiments. Statistical significance was derived by one-way ANOVA followed by Dunnett's test (*** p<0.001).

FIGS. 13A and 13B. Liposomes loaded with YARA-FGF1-GFP caused significantly increased invasion of human primary dermal fibroblasts in cell invasion assays. (FIG. 13A) Primary dermal fibroblasts were seeded at density 1×10⁶ cells/mL onto 24 well plates and exposed to indicated treatments. The liposome concentration in each treatment except the PBS-treated control was 0.1 mg/mL (5.8×10⁹ particles/mL). Cell invasion assays were performed after t=48 h under normal growth conditions, as per manufacturer's instructions. (FIG. 13B) Quantitation of the cell invasion assays in (FIG. 13A). Values are represented as mean±SD from four independent experiments. Statistical significance was derived by one-way ANOVA followed by Dunnett's test (*** p<0.001).

### BRIEF DESCRIPTION OF THE SEQUENCES

SEQ ID NO:1 is TAT peptide.
SEQ ID NO:2 is Antennapedia penetratin.
SEQ ID NO:55 is FAM-labeled YARA peptide.
SEQ ID NO:57 is YARA-Cys peptide.
SEQ ID Nos: 3-94 are cell penetrating polypeptides (CPPs) in Table 2.
SEQ ID NO:95 is Trans-activator protein from HIV.
SEQ ID NO:96 is Antennapedia homeobox peptide.
SEQ ID NO:97 is VP from HSV type 1.
SEQ ID NO:98 is CaP from brome mosaic virus.
SEQ ID NO:99 is YopM from Yersinia enterocolitica.
SEQ ID NO:100 is Artificial protein B1.
SEQ ID NO:101 is 30Kc19 from silkworm Bombyx mori.
SEQ ID NO:102 is engineered +36 GFP.
SEQ ID NO:103 is Naturally supercharged human protein.
SEQ ID NO:104 is fusion peptide H.
SEQ ID NO:105 is single-stranded oligomer S-1.
SEQ ID NO:106 is a peptide inhibitor.
SEQ ID NO:107 is a peptide cargo.
SEQ ID Nos: 108-1259 are cell penetrating polypeptides (CPPs) in Table 11.

### DETAILED DESCRIPTION OF THE INVENTION

One aspect of the invention concerns a method for loading a lipid vesicle (LV) such as a liposome, lipid nanoparticle, lipid droplet, micelle, reverse micelle, lipid-polymer hybrid nanoparticle, or artificial extracellular vesicle, with a cargo molecule, comprising contacting the LV with the cargo molecule covalently or non-covalently coupled to a cell penetrating polypeptide (CPP), upon which the cargo molecule and coupled CPP becomes internalized by, or associated with, the LV. The coupled cargo molecule and CPP is also referred to herein as a “binding complex”. Each LV has a core surrounded by one or more membranes comprising one or more lipid layers (e.g., at least one lipid monolayer or at least one lipid bilayer), and the cargo molecule or “binding complex” may be internalized and contained within the core of the LV, or be bound and/or embedded within the encapsulating membrane(s) of the LV.

Examples 1-5 herein demonstrate that CPPs can load different cargos into LVs. Examples 6 and 7 demonstrate cellular uptake of loaded LVs. Examples 8-10 describe functional studies of the cargos loaded into cells via LVs.

The cargo molecule selected for LV loading may be coupled with one or more CPPs by covalent or non-covalent binding. In some embodiments, non-covalent complexes between cargos and CPPs are formed. For example, a CPP called Pep-1 can non-covalently bind to a cargo and the resulting binding complex may be loaded into LVs (M. C. Morris, J. Depollier, J. Mery, F. Heitz, and G. Divita “A peptide carrier for the delivery of biologically active proteins into mammalian cells”, nature biotechnology, 2001, 19, 1173-1176). A CPP called Candy can non-covalently bind to a nucleic acid cargo and the resulting binding complex may be loaded into LVs (L. Crombez, et al., “A New Potent Secondary Amphipathic Cell-penetrating Peptide for siRNA Delivery Into Mammalian Cells”, Mol. Ther. 17, 95-103). An artificial protein called B1 can non-covalently bind to RNA or DNA and the resulting binding complex may be loaded into LVs (R. L. Simeon, A. M. Chamoun, T. McMillin, and Z. Chen, “Discovery and Characterization of a New Cell-Penetrating Protein”, ACS. Chem. Biol., 2013, 8, 2678-2687). An engineered superpositively charged GFP called +36 GFP can non-covalently bind to RNA or DNA and the resulting binding complex may be loaded into LVs (B. R. McNaughton, J. J. Cronican, D. B. Thompson, and D. R. Liu, “Mammalian cell penetration, siRNA transfection, and DNA transfection by supercharged proteins”, PNAS, 2009, 106, 6111-6116).

As used herein, the term “CPP” is intended to encompass one or more CPPs, and the term “cargo molecule” is intended to encompass one or more cargo molecules. For example, a single cargo molecule may be coupled with one or more CPPs, and multiple cargo molecules may be coupled with one or more CPPs.

The cargo molecule selected for LV loading may be chemically conjugated to a CPP by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage. “Click” chemistry reactions are a class of reactions commonly used in bio-conjugation, allowing the joining of selected substrates with specific biomolecules. Click chemistry is not a single specific reaction, but describes a method of generating products that follow examples in nature, which also generates substances by joining small modular units. Click chemistry is not limited to biological conditions: the concept of a “click” reaction has been used in pharmacological and various biomimetic applications; however, these reactions have proven useful in the detection, localization, and qualification of biomolecules (H. C. Kolb; M. G. Finn; K. B. Sharpless, “Click Chemistry: Diverse Chemical Function from a Few Good Reactions”, Angewandte Chemie International Edition, 2001, 40(11):2004-2021; and R. A. Evans, “The Rise of Azide—Alkyne 1,3-Dipolar ‘Click’ Cycloaddition and its Application to Polymer Science and Surface Modification”, Australian Journal of Chemistry, 2007, 60(6): 384-395).

Optionally, the cargo molecule is covalently coupled to the CPP by a cleavable domain or linker, which becomes cleaved upon exposure of the binding complex to the appropriate cleaving agent or condition, such as a chemical agent (e.g., dithiothreitol for reducing a disulfide bond linkage), environment (e.g., temperature or pH), or radiation. For example, the cleavable domain or linker may be photo-cleavable (Olejnik, J. et al., “Photocleavable peptide-DNA conjugates: synthesis and applications to DNA analysis using MALDI-MS”, Nucleic Acids Research, 1999, 27(23):4626-4631; Matsumoto R et al., “Effects of the properties of short peptides conjugated with cell-penetrating peptides on their internalization into cells,” Scientific Reports, 2015, 5:12884; and Usui, K. et al., “A novel array format for monitoring cellular uptake using a photo-cleavable linker for peptide release”, Chem Commun, 2013, 49:6394-6396; Kakiyama, T. et al., “A peptide release system using a photo-cleavable linker in a cell array format for cell-toxicity analysis”, Polymer J., 2013, 45:535-539; Wouters, S. F. A., Wijker, E., and Merkx, M., “Optical Control of Antibody Activity by Using Photocleavable Bivalent Peptide—DNA Locks”, ChemBioChem, 2019, 20:2463-2466). By linking the cargo molecule with a CPP via a photo-cleavable conjugation, once the binding complex is inside an LV, such as a liposome, the LV can be exposed to light of the proper wavelength, which will cleave the linker between the CPP and the cargo molecule, freeing the cargo inside the LV. Once the LV fuses with a cell, the free cargo will be delivered into the cell.

In embodiments in which the cargo molecule is a nucleic acid, fusion with the CPP may be achieved through a chemical bond.

Likewise, in embodiments in which the cargo molecule is a nucleic acid, tight association with the CPP may be achieved through non-covalent binding.

The loading method may include the step of covalently or non-covalently coupling the CPP to the cargo molecule, to produce the binding complex, before contacting the LV with the binding complex.

The loading method may also include the step of uncoupling the CPP and the cargo molecule once the cargo molecule has been internalized by, or associated with, the LV. Once the cargo is loaded into LVs, it is not necessary to have the binding complex stay intact as long as the cargo molecules are either inside the LVs or embedded onto the membrane of the LVs, depending on the intended use of the loaded LVs. If the CPP is non-covalently coupled to the cargo molecule, the complex can either associate or dissociate within the LVs. If the CPP is covalently coupled to the cargo molecule, the complex may be intact or be intentionally cleaved, for example by light, a reducing agent such as dithiothreitol (DTT) or other methods. The following factors should be taken into consideration:
1. It may be necessary for the CPP and cargo molecule to be uncoupled (physically separated) within the LVs if the CPP interferes with the in vivo function of the cargo, or the binding complex causes additional side effect(s) in vivo relative to the cargo itself (if there are such side effects).
2. It may not be necessary to uncouple the CPP and cargo molecule of the binding complex if the CPP does not interfere with the in vivo function of the cargo molecule and the binding complex has the same side effect profile as the cargo molecule alone (if there are such side effects).

Another aspect of the invention is the loaded LV itself, comprising a cargo molecule and a CPP, wherein the cargo molecule has been internalized by, or is associated with, the LV. The cargo molecule may remain coupled to the CPP covalently or non-covalently (together, the “binding complex”), wherein the binding complex has been internalized by, or is associated with, the LV. The loaded LV may be produced using any of the aforementioned embodiments of methods for loading the LV. Thus, the linkage between the CPP and cargo molecule may be covalent or non-covalent.

The cargo molecule of the loaded LV may be selected, for example, from among a small molecule, fluorescent dye, imaging agent, macromolecule, polypeptide (natural or modified), nucleic acid (e.g., DNA, RNA, PNA, DNA- or RNA-like molecule, snRNA, ncRNA (e.g., miRNA), RNAi (e.g., siRNA, shRNA), mRNA, tRNA), antibody or antibody-fragment, proteins (e.g., enzymes, membrane-bound proteins), growth factor, lipoprotein, protein, carbohydrate, or glycoprotein. The cargo molecule may be any class of substance or combination of classes. The cargo molecule may be in the form of an active pharmaceutical ingredient or a pharmaceutically acceptable salt, metabolite, derivative, or prodrug of an active pharmaceutical ingredient.

In some embodiments, the cargo molecule is a growth factor or growth miRNA. A growth factor-loaded and/or growth miRNA-loaded LVs may be administered to a subject for treatment of an acute or chronic wound, for example.

Another aspect of the invention concerns a method for delivering a cargo molecule into a cell in vitro or in vivo by administering loaded LVs to the cell in vitro or in vivo, upon which the loaded LVs are internalized into the cell, and wherein the loaded LV comprises the cargo molecule coupled to a CPP. In in vivo embodiments, the loaded LVs are administered to a human or animal subject by any suitable route to reach the target cells.

The cargo molecule may be covalently or non-covalently coupled to a CPP. In some embodiments of the delivery method, the cargo molecule is selected from among a small molecule, fluorescent dye, imaging agent, macromolecule, polypeptide (natural or modified), nucleic acid (e.g., DNA, RNA, PNA, DNA- or RNA-like molecule, RNAi (e.g., siRNA, shRNA) snRNA, ncRNA (e.g., miRNA), mRNA, tRNA), antibody or antibody-fragment, lipoprotein, proteins (e.g., enzymes, membrane-bound proteins), growth factor, lipoprotein, protein, carbohydrate, or glycoprotein.

In some embodiments of the delivery method, the cargo molecule is a growth factor or growth miRNA. The growth factor-loaded and/or growth miRNA-loaded LVs may be administered to the cell of a wound in vivo. In some embodiments, the growth factor-loaded and/or growth miRNA-loaded LVs are administered to a subject for treatment of an acute or chronic wound. For example, the growth factor-loaded and/or growth miRNA-loaded LVs can be administered to a skin cell (e.g., a primary dermal fibroblast).

The delivery method may further include, as a step in the method, loading the LVs with the cargo molecules prior to administering the loaded LVs to the cells in vitro or in vivo. The delivery method may further include, as a step in the method, covalently or non-covalently coupling the CPP to the cargo molecule prior to contacting the LV with the binding complex.

### Lipid Vesicles (LVs)

LVs used in the invention are particles having an interior core surrounded and enclosed by one or more membranes, with the membrane comprising one or more lipid layers. Each of the one or more lipid layers surrounding the core may be a lipid monolayer or a lipid bilayer. Any type of LV may be utilized, such as a liposome, lipid nanoparticle, lipid droplet, micelle, reverse micelle, lipid-polymer hybrid nanoparticle, artificial extracellular vesicle, or a mixture of two or more of the foregoing. The LV can be selected for a core that can carry a desired cargo. The LVs may be synthetic (artificially created or non-naturally occurring) or naturally occurring. Naturally occurring LVs may be in an isolated state (fully or partially isolated from their natural milieu) or in a non-isolated state. The LVs may be any shape but are typically spherical.

Although LVs have emerged as therapeutic carriers, the major limitation of using LVs has been the lack of a well-developed methodology for increasing cellular uptake of their intended content(s). The present invention facilitates loading of LVs with cargo using CPPs and delivery of the cargo to recipient cells in vitro or in vivo.

LVs may be unilamellar in structure (having a single lipid layer) or multilamellar in structure (a concentric arrangement of two or more lipid layers). LVs may be spherical or have a non-spherical or irregular, heterogeneous shape. Examples of LVs include liposomes, lipid nanoparticles, lipid droplets, micelles, reverse micelles, lipid-polymer hybrid nanoparticles, and artificial extracellular vesicles. The surrounding one or more lipid layers of LVs may be composed of synthetic lipids (e.g., a lipid manufactured by chemical synthesis from specified starting materials), semi-synthetic lipids (e.g., a lipid manufactured by modification of naturally occurring precursors such as dipalmitoylphosphatidylcholine (DPPC), distearoylphosphatidylcholine (DSPC), or dimyristoylphosphatidylcholine (DMPC)), naturally occurring lipids, or a combination of two or more of the foregoing, that are compatible with the lipid bilayer structure. In some embodiments, the lipid is a monoglyceride, diglyceride, or triglyceride, or a combination of two or more of the foregoing. Examples of lipids include phospholipids (such as phosphatidylcholine) and egg phosphatidylethanolamine.

Lipid nanoparticles or LNPs have a solid lipid core matrix surrounded by a lipid monolayer (Puri A et al., “Lipid-Based Nanoparticles as Pharmaceutical Drug Carriers: From Concepts to Clinic”, Crit Rev Ther Drug Carrier Syst, 2009; 26(6): 523-580; Saupe A and T Rades, “Solid Lipid Nanoparticles”, Nanocarrier Technologies, In: Mozafari M. R. (eds) Nanocarrier Technologies, 2006, p. 4; and Jenning, V et al., “Characterisation of a novel solid lipid nanoparticle carrier system based on binary mixtures of liquid and solid lipids”, International Journal of Pharmaceutics, 2000, 199(2):167-77). The LNP core is stabilized by surfactants and can solubilize lipophilic molecules. The core lipids can be fatty acids, acylglycerols, waxes, and mixtures of these surfactants. By “solid,” it is meant that at least a portion of the LNP are solid at room or body temperature and atmospheric pressure. However, an LNP can include portions of liquid lipid and/or entrapped solvent. Formulation methods for LNPs include high shear homogenization and ultrasound, solvent emulsification/evaporation, or microemulsion. Obtaining size distributions in the range of 30-180 nm is possible using ultrasonification at the cost of long sonication time. Solvent-emulsification is suitable in preparing small, homogeneously-sized lipid nanoparticles dispersions with the advantage of avoiding heat (Mehnert W, and K. Mäder, “Solid lipid nanoparticles: Production, characterization and applications,” Advanced Drug Delivery Reviews, 2012, Volume 64, Pages 83-101).

A liposome is a vesicle having an interior aqueous core surrounded by, and enclosed by, at least one lipid bilayer (Akbarzadeh A et al., “Liposome: classification, preparation, and applications”, Nanoscale Res Lett. 2013; 8(1): 102; Wagner A and K Vorauer-Uhl, “Liposome Technology for Industrial Purposes”, Journal of Drug Delivery, 2011, Volume 2011, Article ID 591325, 9 pages).

Liposomes are typically spherical in shape but their shape and size may be controlled by their components, cargo, and preparation methods (Kawamura J et al., “Size-Controllable and Scalable Production of Liposomes Using a V-Shaped Mixer Micro-Flow Reactor”, Org. Process Res. Dev., 2020, 24, 10, 2122-2127; Miyata H and Hotani, “Morphological changes in liposomes caused by polymerization of encapsulated actin and spontaneous formation of actin bundles (cytoskeleton)”, Proc. Natl. Acad. Sci. USA, December 1992, Vol. 89, pp. 11547-11551; Yager P et al., “Changes in size and shape of liposomes undergoing chain melting transitions as studied by optical microscopy”, Biochimica et Biophysica Acta (BBA)—Biomembranes, 22 Dec. 1982, Volume 693, Issue 2, Pages 485-491).

In a liposome delivery product, the cargo (e.g., a drug substance) is generally “contained” in liposomes. The word “contained” in this context includes both encapsulated and intercalated cargo. The term “encapsulated” refers to cargo within an aqueous space and “intercalated” refers to incorporation of the cargo within a bilayer. Typically, water soluble cargos are contained in the aqueous compartment(s) and hydrophobic cargos are contained in the lipid bilayer(s) of the liposomes.

A liposome drug formulation is different from (1) an emulsion, which is a dispersed system of oil-in-water, or water-in-oil phases containing one or more surfactants, (2) a microemulsion, which is a thermodynamically stable two phase system containing oil or lipid, water, and surfactants, and (3) a drug-lipid complex.

Liposome structural components typically include phospholipids or synthetic amphiphiles incorporated with sterols, such as cholesterol, to influence membrane permeability. Thin-film hydration is a widely used preparation method for liposomes, in which lipid components with or without cargo are dissolved in an organic solvent. The solvent will be evaporated by rotary evaporation followed by rehydration of the film in an aqueous solvent. Other preparation methods include, for example, reverse-phase evaporation, freeze-drying and ethanol injection (Torchilin, V and V Weissig, “Liposomes: A Practical Approach”, Oxford University Press: Kettering, UK, 2003, pp. 77-101). Techniques such as membrane extrusion, sonication, homogenization and/or freeze-thawing are being employed to control the size and size distribution. Liposomes can be formulated and processed to differ in size, composition, charge, and lamellarity.

The major types of liposomes are the multilamellar vesicle (MLV, with multiple lamellar phase lipid bilayers), the small unilamellar liposome vesicle (SUV, with one lipid bilayer), the large unilamellar vesicle (LUV), and the cochleate vesicle. Some liposomes are multivesicular, in which one vesicle contains one or more smaller vesicles.

Liposome technology has been successfully translated into clinical applications. Delivery of therapeutics by liposomes alters their biodistribution profile, which can enhance the therapeutic index of drugs. Therapeutic areas in which lipid-based products have been used include, but are not limited to, cancer therapy (Doxil®, DaunoXome®, Depocyte®, Marqibo®, Myocet®, and Onivyde™), fungal diseases (Abelcet®, Ambisome®, and Amphotec®), analgesics (DepoDur™ and Exparel®), viral vaccines (Epaxal® and Inflexal® V), and photodynamic therapy (Visudyne®) (Bulbake U et al., “Liposomal Formulations in Clinical Use: An Updated Review”, Pharmaceutics, 2017, 9(2):12; and Puri A et al. (2009). The invention may be used to load these agents into their respective liposomes, as well as a variety of other cargo-liposome combinations. Examples of lipid components used clinically in liposome-based products and in clinical trials can be found, for example, in Tables 1 and 2 of Bulbake U et al. (2017), which are incorporated herein by reference in their entirety.

The invention may be used with a variety of liposomal platforms, such as “stealth liposomes” (e.g., PEGylated liposomes), non-PEGylated liposomes, multivesicular liposomes (e.g., DepoFoam™ extended-release technology), and thermosensitive liposomes. In the case of DepoFoam™ extended-release technology, each particle contains numerous non-concentric aqueous chambers bounded by a single bilayer lipid membrane. Each chamber is partitioned from the adjacent chambers by bilayer lipid membranes composed of synthetic analogs of naturally existing lipids (DOPC, DPPG, cholesterol, triolein, etc.) (Murry D J and SM Blaney, “Clinical pharmacology of encapsulated sustained-release cytarabine”, Ann. Pharmacother., 2000, 34:1173-1178). Upon administration, DepoFoam™ particles release the drug over a period of time (hours to days) following erosion and/or reorganization of the lipid membranes.

Whereas liposomes are composed of a lipid bilayer separating an aqueous internal compartment from the bulk aqueous phase, micelles are closed lipid monolayers with a fatty acid core and polar surface, or polar core with fatty acids on the surface (reverse micelle).

The LV may be a lipid-polymer hybrid nanoparticle or “LPHNP”, which refers to a lipid vesicle having a polymer core that can contain cargo, with the polymer core encapsulated by a lipid monolayer (Mukherjee et al., “Lipid-polymer hybrid nanoparticles as a next-generation drug delivery platform: state of the art, emerging technologies, and perspectives”, Int J Nanomedicine, 2019, 14:1937-1952).

The LVs used in the invention are not “extracellular vesicles” or “EVs” per se. “Extracellular vesicle” is a collective term encompassing various subtypes of cell-released or cell-secreted, membranous structures, often referred to as exosomes, microvesicles, mitovesicles, apoptotic bodies, etc., and have been defined variously in the literature by their size, biogenesis pathway, cellular source, and function; however, the LV used in the invention may be an “artificial extracellular vesicle” (also known as a “synthetic extracellular vesicle”), as described in Garcia-Manrique P et al., “Therapeutic biomaterials based on extracellular vesicles: classification of bio-engineering and mimetic preparation routes”, Journal of Extracellular Vesicles, 2018, vol. 7, 1422676, which is incorporated herein by reference in its entirety. Artificial extracellular vesicles (artificial EVs) are vesicles that are modified or manufactured from (from natural or synthetic sources), with the objective to mimic or recapitulate the functions of EVs, for therapeutic or other uses. Artificial EVs may be semi-synthetic or fully synthetic. Artificial EVs are also described in Staufer O et al., “Bottom-up assembly of biomedical relevant fully synthetic extracellular vesicles”, Science Advances, 2021, 7:eabg6666; Li Y-J et al., “Artificial exosomes for translational medicine”, Journal of Nanobiotechnology, 2021, 19:242; Man K et al., “Engineered Extracellular Vesicles: Tailor-Made Nanomaterials for Medical Applications”, Nanomaterials, 2020, 10:1838; and Ramasubramanian L et al., “Engineering Extracellular Vesicles as Nanotherapeutics for Regenerative Medicine”, Biomolecules, 2020, 10:48, which are each incorporated herein by reference in their entireties.

The LV may be a lipid droplet, which is a cellular organelle containing a neutral-lipid core enclosed by a phospholipid monolayer (and associated proteins), and may be isolated from cells.

### Cellular Delivery

LVs loaded with cargo may be administered to cells in vitro by contacting the cells with the loaded LVs, and LVs loaded with cargo may be administered to cells in vivo by administering the loaded LVs to organisms having the recipient cells, such as human or non-human animals, and plants. For delivery to cells in vivo, the LVs are administered by any route appropriate to reach the desired cells. Examples of routes include but are not limited to, oral, rectal, nasal, topical (including buccal and sublingual), vaginal and parenteral (including subcutaneous, intramuscular, intravenous, intradermal, intrathecal and epidural), and the like. For therapy or prophylaxis of a condition in a subject (e.g., human or animal diseases such as cancer, infectious diseases, genetic diseases, central nervous system disorders, etc.), it will be appreciated that the preferred route may vary with, for example, the condition in question and the health of the subject. In some embodiments, the LVs are administered locally at an anatomic site where the recipient cells are found, such as on the skin, topically, or at the site of a wound or tumor. In other embodiments, the LVs are administered systemically for delivery to cells that may be anatomically remote from the site of administration. In some embodiments, LVs are administered orally, sublingually, nasally, rectally, parenterally, subcutaneously, intramuscularly, or intravascularly (e.g., intravenously).

In addition to LV-mediated delivery of cargo to mature or specialized cells, LVs may be used to deliver cargo to immature progenitor cells or stem cells. Recipient cells can range in plasticity from totipotent or pluripotent stem cells (e.g., adult or embryonic), precursor or progenitor cells, to highly specialized cells, such as those of the central nervous system (e.g., neurons and glia). Stem cells and progenitor cells can be found in a variety of tissues, including embryonic tissue, fetal tissue, adult tissue, adipose tissue, umbilical cord blood, peripheral blood, bone marrow, and brain, for example.

As will be understood by one of skill in the art, there are over 200 cell types in the human body. LVs can be delivered to any of these cell types. For example, any cell arising from the ectoderm, mesoderm, or endoderm germ cell layers can be a recipient of LVs and their loaded cargo molecules. Recipient cells may be natural or wild-type cells, or cells of a cell line, for example.

Table 1 is a non-limiting list of examples of cells to which cargo molecules can be delivered using the invention.

TABLE 1
Examples of Cells

Keratinizing Epithelial Cells
keratinocyte of epidermis
basal cell of epidermis
keratinocyte of fingernails and toenails
basal cell of nail bed
hair shaft cells
medullary
cortical
cuticular
hair-root sheath cells
cuticular
of Huxley's layer
of Henle's layer
external
hair matrix cell
Cells of Wet Stratified Barrier Epithelia
surface epithelial cell of stratified squamous
epithelium of cornea tongue, oral cavity,
esophagus, anal canal, distal urethra, vagina
basal cell of these epithelia
cell of urinary epithelium
Epithelial Cells Specialized for Exocrine Secretion
cells of salivary gland
mucous cell
serous cell
cell of von Ebner's gland in tongue
cell of mammary gland, secreting milk
cell of lacrimal gland, secreting tears
cell of ceruminous gland of ear, secreting wax
cell of eccrine sweat gland, secreting glycoproteins
cell of eccrine sweat gland, secreting small molecules
cell of apocrine sweat gland
cell of gland of Moll in eyelid
cell of sebaceous gland, secreting lipid-rich sebum
cell of Bowman's gland in nose
cell of Brunner's gland in duodenum, secreting alkaline
solution of mucus and enzymes
cell of seminal vesicle, secreting components of seminal
fluid, including fructose
cell of prostate gland, secreting other components of
seminal fluid
cell of bulbourethral gland, secreting mucus
cell of Bartholin's gland, secreting vaginal lubricant
cell of gland of Littre, secreting mucus
cell of endometrium of uterus, secreting mainly carbohydrates
isolated goblet cell of respiratory and digestive tracts,
secreting mucus
mucous cell of lining of stomach
zymogenic cell of gastric gland, secreting pepsinogen
oxyntic cell of gastric gland, secreting HCl
acinar cell of pancreas, secreting digestive enzymes and
bicarbonate
Paneth cell of small intestine, secreting lysozyme
type II pneumocyte of lung, secreting surfactant
Clara cell of lung
Cells Specialized for Secretion of Hormones
cells of anterior pituitary, secreting
growth hormone
follicle-stimulating hormone
luteinizing hormone
prolactin
adrenocorticotropic hormone
thyroid-stimulating hormone
cell of intermediate pituitary, secreting melanocyte-
stimulating hormone
cells of posterior pituitary, secreting
oxytocin
vasopressin
cells of gut and respiratory tract, secreting
serotonin
endorphin
somatostatin
gastrin
secretin
cholecystokinin
insulin
glucagons
bombesin
cells of thyroid gland, secreting
thyroid hormone
calcitonin
cells of parathyroid gland, secreting
parathyroid hormone
oxyphil cell
cells of adrenal gland, secreting
epinephrine
norepinephrine
steroid hormones
mineralocorticoids
glucocorticoids
cells of gonads, secreting
testosterone
estrogen
progesterone
cells of juxtaglomerular apparatus of kidney
juxtaglomerular cell
macula densa cell
peripolar cell
mesangial cell
Epithelial Absorptive Cells in Gut, Exocrine Glands,
and Urogenital Tract
brush border cell of intestine
striated duct cell of exocrine glands
gall bladder epithelial cell
brush border cell of proximal tubule of kidney
distal tubule cell of kidney
nonciliated cell of ductulus efferens
epididymal principal cell
epididymal basal cell
Cells Specialized for Metabolism and Storage
Hepatocyte
fat cells (e.g., adipocyte)
white fat
brown fat
lipocyte of liver
Epithelial Cells Serving Primarily a Barrier Function,
Lining the Lung, Gut, Exocrine Glands, and Urogenital Tract
type I pneumocyte
pancreatic duct cell
nonstriated duct cell of sweat gland, salivary gland,
mammary gland, etc.
parietal cell of kidney glomerulus
podocyte of kidney glomerulus
cell of thin segment of loop of Henle
collecting duct cell
duct cell of seminal vesicle, prostate gland, etc.
Epithelial Cells Lining Closed Internal Body Cavities
vascular endothelial cells of blood vessels and
lymphatics (e.g., microvascular cell)
fenestrated
continuous
splenic
synovial cell
serosal cell
squamous cell lining perilymphatic space of ear
cells lining endolymphatic space of ear
squamous cell
columnar cells of endolymphatic sac
with microvilli
without microvilli
“dark” cell
vestibular membrane cell
stria vascularis basal cell
stria vascularis marginal cell
cell of Claudius
cell of Boettcher
choroid plexus cell
squamous cell of pia-arachnoid
cells of ciliary epithelium of eye
pigmented
nonpigmented
corneal “endothelial” cell
Ciliated Cells with Propulsive Function
of respiratory tract
of oviduct and of endometrium of uterus
of rete testis and ductulus efferens
of central nervous system
Cells Specialized for Secretion of Extracellular Matrix
epithelial:
ameloblast
planum semilunatum cell of vestibular apparatus of ear
interdental cell of organ of Corti
nonepithelial:
fibroblasts
pericyte of blood capillary (Rouget cell)
nucleus pulposus cell of intervertebral disc
cementoblast/cementocyte
odontoblast/odontocyte
chondrocytes
of hyaline cartilage
of fibrocartilage
of elastic cartilage
osteoblast/osteocyte
osteoprogenitor cell
hyalocyte of vitreous body of eye
stellate cell of perilymphatic space of ear
Contractile Cells
skeletal muscle cells
red
white
intermediate
muscle spindle-nuclear bag
muscle spindle-nuclear chain
satellite cell
heart muscle cells
ordinary
nodal
Purkinje fiber
Cardiac valve tissue
smooth muscle cells
myoepithelial cells:
of iris
of exocrine glands
Cells of Blood and Immune System
red blood cell (erythrocyte)
Megakaryocyte
Macrophages
monocyte
connective tissue macrophage
Langerhan's cell
osteoclast
dendritic cell
microglial cell
Neutrophil
Eosinophil
Basophil
mast cell
plasma cell
T lymphocyte
helper T cell
suppressor T cell
killer T cell
B lymphocyte
IgM
IgG
IgA
IgE
killer cell
stem cells and committed progenitors for the blood
and immune system
Sensory Transducers
Photoreceptors
rod
cones
blue sensitive
green sensitive
red sensitive
Hearing
inner hair cell of organ of Corti
outer hair cell of organ of Corti
acceleration and gravity
type I hair cell of vestibular apparatus of ear
type II hair cell of vestibular apparatus of ear
Taste
type II taste bud cell
Smell
olfactory neuron
basal cell of olfactory epithelium
blood pH
carotid body cell
type I
type II
touch
Merkel cell of epidermis
primary sensory neurons specialized for touch
temperature
primary sensory neurons specialized for temperature
cold sensitive
heat sensitive
pain
primary sensory neurons specialized for pain
configurations and forces in musculoskeletal system
proprioceptive primary sensory neurons
Autonomic Neurons
Cholinergic
Adrenergic
Peptidergic
Supporting Cells of Sense Organs and of Peripheral Neurons
supporting cells of organ of Corti
inner pillar cell
outer pillar cell
inner phalangeal cell
outer phalangeal cell
border cell
Hensen cell
supporting cell of vestibular apparatus
supporting cell of taste bud
supporting cell of olfactory epithelium
Schwann cell
satellite cell
enteric glial cell
Neurons and Glial Cells of Central Nervous System
Neurons
glial cells
astrocyte
oligodendrocyte
Lens Cells
anterior lens epithelial cell
lens fiber
Pigment Cells
Melanocyte
retinal pigmented epithelial cell
iris pigment epithelial cell
Germ Cells
oogonium/oocyte
Spermatocyte
Spermatogonium
blast cells
fertilized ovum
Nurse Cells
ovarian follicle cell
Sertoli cell
thymus epithelial cell (e.g, reticular cell)
placental cell

Optionally, LVs such as liposomes may include a targeting agent (also referred to as a targeting ligand) that targets the LV to a cellular compartment, cell type, organ, or tissue. A ligand such as an antibody, antibody fragment, and/or peptide may be bound to the surface of the LV (to the outer lipid layer). The ligand has a binding partner that is more abundant in or on the target cellular compartment, cell type, tissue, or organ, allowing the LV to target a cellular compartment or bind to and fuse with a specific cell type, tissue, or organ and deliver the cargo into the target cellular compartment, cells, tissue, or organ.

For example, if the targeting agent is an antibody or antibody fragment, the binding partner may be the antibody's/fragment's corresponding target antigen. If the target agent is a polypeptide that serves as a ligand for a receptor, the binding partner may be the ligand's corresponding target receptor. In some embodiments, the target for the targeting agent is a protein that is over-expressed on one or more cancer cell types (e.g., a tumor-associated antigen). Strategies for targeting LVs using targeting ligands are described in Puri et al. (2009), which are incorporated herein by reference. For example, a galactosylated conjugated DOPE lipid carrying an anti-cancer agent as cargo may be used to specifically target the asialo-glycoprotein receptor on hepatocellular carcinoma. Folate-targeted LVs carrying anti-cancer agent as cargo may be used to target cells with folate receptors, such as tumor cells. For liver targeting, an LV with galactosylated or mannosylated lipids may be used.

A CPP may be covalently or non-covalently coupled to the outer lipid layer of the LV to target a cell type, cellular compartment, tissue, or organ. The CPP selected as a targeting agent may be the same or different from the CPP selected for loading cargo into the LV. The BR2 and TAT peptides are examples of CPPs that may be used to target LVs in this way. For example, the CPP BR2 may be used to form cancer cell-targeting liposomes (BR2-liposomes) to deliver anti-cancer agents (Zhang X et al., “Liposomes equipped with cell penetrating peptide BR2 enhances chemotherapeutic effects of cantharadin against hepatocellular carcinoma”, Drug Delivery, 2017, 24(1):986-998). A CPP such as TAT may be conjugated to lipids to form TAT-liposomes which exhibit enhanced cellular internalization for delivery of therapeutic agents (Torchilin V P et al., “TAT peptide on the surface of liposomes affords their efficient intracellular delivery even at low temperature and in the presence of metabolic inhibitors”, PNAS, Jul. 17, 2001, 98(15):8786-8791). A different CPP may be used to load cargo into the TAT-liposome.

In addition to the medical field, the invention may be used in other industries in which LVs may be loaded with cargo for delivery to cells. For example, LVs may be used in agriculture to deliver cargo such as nutrients to plant cells (Karny A et al., “Therapeutic nanoparticles penetrate leaves and deliver nutrients to agricultural crops” Scientific Reports, 2018, 8(1):7589; and Temming M, “Nanoparticles could help rescue malnourished crops” Science News).

### Cell-Penetrating Polypeptides (CPPs)

In the past several decades, there have been many basic and preclinical research reports focused on the abilities of CPPs to carry and translocate various types of cargo molecules across the cellular plasma membrane. The inventors have determined that CPPs may be used to load LVs such as liposomes with a cargo molecule, and the loaded LVs may then be used to deliver the cargo molecules to desired cells. The loaded cargo molecule may be carried by the LV in or on the vesicle's one or more membranes (“membrane cargo”) or within the core of the vesicle (“luminal cargo”). CPPs disclosed herein may be coupled to cargo for loading LVs, and/or the CPPs may be coupled to the lipid surface of the LVs to target cells, cellular compartments, tissues, or organs.

Structurally, CPPs tend to be small natural or artificial peptides composed of about 5 to 30 amino acids; however, they may be longer. As used herein, the terms “cell penetrating polypeptide” and “CPP” refer to amino acid sequences of any length that have the membrane-traversing carrier function, and are inclusive of short peptides and full-length proteins. CPPs may be any configuration, such as linear or cyclic (Park S E et al., “Cyclic Cell-Penetrating Peptides as Efficient Drug Delivery Tools”, Mol. Pharmaceutics, 2019, 16, 9, 3727-3743; Dougherty P G et al. “Understanding Cell Penetration of Cyclic Peptides”, Chem. Rev., 2019, 119(17):10241-10287; Song J et al., “Cyclic Cell-Penetrating Peptides with Single Hydrophobic Groups”, Chembiochem. 2019 Aug. 16; 20(16):2085-2088).

The CPP may be linear or cyclic. The CPP may be composed of L-amino acids, D-amino acids, or a mixture of both. The CPP may be protein derived, synthetic, or chimeric.

Cargo molecules may be associated with the CPPs through chemical linkage via covalent bonds or through non-covalent binding interactions, for example. CPPs typically have an amino acid composition that either contains a high relative abundance of positively charged amino acids such as lysine or arginine or have sequences that contain an alternating pattern of polar, charged amino acids and non-polar, hydrophobic amino acids. These two types of structures are referred to as polycationic or amphipathic, respectively. In some embodiments, the CPP is an arginine-rich peptide, lysine-rich peptide, or both. Another class of CPPs is the hydrophobic peptide, containing only apolar residues with low net charge or hydrophobic amino acid groups that are crucial for cellular uptake.

In some embodiments, the CPP is cationic, amphipathic, both cationic and amphipathic, or anionic.

Transactivating transcriptional activator (TAT), GRKKRRQRRRPPQ (SEQ ID NO:1), from human immunodeficiency virus 1 (HIV-1), and Antennapedia penetratin, RQIKIWFQNRRMKWKK (SEQ ID NO:2), were among the first CPPs to be discovered. Since then, the number of known CPPs has expanded considerably, and small molecule synthetic analogues and cyclized peptides with more effective protein transduction properties have been generated (Habault J et al., “Recent Advances in Cell Penetrating Peptide-Based Anticancer Therapies”, Molecules, 2019 March; 24(5):927; Derakhshankhah H et al., “Cell penetrating peptides: A concise review with emphasis on biomedical applications,” Biomedicine & Pharmacotherapy, 2018, 108:1090-1096; Borrelli A et al., “Cell Penetrating Peptides as Molecular Carriers for Anti-Cancer Agents”, Molecules, 2018, 23:295; and Okuyama M et al., “Small-molecule mimics of an alpha-helix for efficient transport of proteins into cells”, Nature Methods., 2007, 4(2):153-9, which are each incorporated herein by reference in their entireties).

In some embodiments, the CPP is 3 to 5 amino acids in length. In some embodiments, the CPP is 6 to 10 amino acids in length. In some embodiments, the CPP is 11 to 15 amino acids in length. In some embodiments, the CPP is 16 to 20 amino acids in length. In some embodiments, the CPP is 21 to 30 amino acids in length. In some embodiments, the CPP is over 30 amino acids in length.

In some embodiments, the CPP is cationic. In some embodiments, the CPP is amphipathic. In some embodiments, the CPP is anionic.

The CPPs may have chemical modifications in-sequence (e.g., beta-alanine, linkers (e.g., Ahx), amino isobutyric acid (Aib), L-2-naphthyalalnine, or ornithine), N-terminal modifications (e.g., free, biotinylation, acetylation, or stearylation), and/or C-terminal modifications (e.g., free or amidated).

In some embodiments, two or more CPPs (which may be identical or different CPPs) are fused to the same cargo molecule in order to enhance their LV penetration power or capability.

The N-terminus or C-terminus of a protein cargo are usually intended for covalent linkage with a CPP. Alternatively, a CPP can be inserted within a loop region of the protein cargo and the loop preferably does not have any secondary structure and cannot interact with other parts of the protein cargo.

The website CPPsite 2.0 is the updated version of the cell penetrating peptides database (CPPsite): webs.iiitd.edu.in/raghava/cppsite/information.php. It is a manually curated database holding many entries on CPPs that may be utilized in the invention. The website includes fields on (i) diverse chemical modifications, (ii) in vitro/in vivo model systems, and (iii) different cargoes delivered by CPPs. The CCPsite 2.0 covers different types of CPPs, including linear and cyclic CPPs, and CPPs with non-natural amino acid residues. The CPPsite 2.0 includes detailed structural information on CPPs, such as predicted secondary and tertiary structures of CPPs, including the structure of CPPs having D-amino acids and modified residues such as ornithine and beta-alanine. The CPPsite 2.0 includes information on diverse chemical modifications of CPPs that may be employed, including endo modifications (e.g., acylation, amidation, stearylation, biotinylation), non-natural residues (e.g., ornithine, beta-alanine), side chain modifications, peptide backbone modifications, and linkers (e.g., amino hexanoic acid). All CPPs on the CPPsite 2.0 database have been assigned a unique id number, which is constant throughout the database. CPPs are organized and can be browsed by length (up to 5 amino acids, 6-10 amino acids, 11-15 amino acids, 16-20 amino acids, 21-30 amino acids, and over 30 amino acids), and by category, including peptide type (linear or cyclic), peptide class (cationic or amphipathic), peptide nature (protein derived, synthetic, or chimeric), and peptide chirality (L, D, or mixed).

Examples of CPPs that may be used in the invention are provided in Behzadipour Y and S Hemmati “Considerations on the Rational Design of Covalently Conjugated Cell Penetrating Peptides (CPPs) for Intracellular Delivery of Proteins: A Guide to CPP Selection Using Glucarpidase as the Model Cargo Molecule”, Molecules, 2019, 24:4318, which is incorporated herein by reference in its entirety, including but not limited to the supplementary tables, and particularly the 1,155 peptides of Table Si (provided in Table 11 herein).

A class of peptidomimetics known as gamma-AApeptides (γ-AApeptides) can penetrate cell membranes and, therefore, may be used as CPPs in the invention. Examples of gamma-AApeptides and provided in Nimmagadda A et al., “γ-AApeptides as a new strategy for therapeutic development”, Curr Med Chem., 2019, 26(13): 2313-2329, and Li Y et al., “Helical Antimicrobial Sulfono-γ-AApeptides”, J. Med. Chem. 2015, 58, 11, 4802-4811, which are each incorporated herein by reference in their entireties, including but not limited to all gamma-AApeptides disclosed therein.

Examples of CPPs that may be used in the invention are also provided in Table 2 and Table 11 herein. In some embodiments, the CPP is one listed in Table 2, Table 11, or specifically identified elsewhere herein (e.g., by amino acid sequence).

TABLE 2
Examples of Natural and Artificial Cell-Penetrating Polypeptides

| | |
|---|---|
| Polyarginine: R(nR)R (n > 2) | LCLRPVG (SEQ ID NO: 48) |
| Poly D-arginine: n(D-R) (n > 5; D-R, D- | RKKRRQRRR (SEQ ID NO: 49) |
| arginine) | |
| KRRRGRKKRR (SEQ ID NO: 3) | RRRKKRRRRR (SEQ ID NO: 50) |
| RQIKIWFQNRRMKWKK (SEQ ID NO: 2) | KETWWETWWTEWSQPKKKRKV (SEQ ID NO: 51) |
| GWTLNSAGYLLGKINLKALAALAKKIL (SEQ ID NO: 4) | VQRKRQKLMP (SEQ ID NO: 52) |
| RRGRKKRRKR (SEQ ID NO: 5) | RRKKRRRRRG (SEQ ID NO: 53) |
| RGRKKRRKRR (SEQ ID NO: 6) | RKKRRRRRGG (SEQ ID NO: 54) |
| GRKKRRKRRR (SEQ ID NO: 7) | YARAAARQARA (used here) (SEQ ID NO: 55) |
| KRRRGRKKRR (SEQ ID NO: 8) | YARAAARQARAC (SEQ ID NO: 56) |
| YGRKKRRQRRR (SEQ ID NO: 9) | YARAAARQARAGC (used here) (SEQ ID NO: 57) |
| RKKRRKRRRR (SEQ ID NO: 10) | KKIFKKILKFL (SEQ ID NO: 58) |
| KKRRKRRRRK (SEQ ID NO: 11) | KKLFKKIVKY (SEQ ID NO: 59) |
| KRRKRRRRKK (SEQ ID NO: 12) | KLFFKKILKYL (SEQ ID NO: 60) |
| RRRGRKKRRK (SEQ ID NO: 13) | CYARAAARQARAC (SEQ ID NO: 61) |
| RRKRRRRKKR (SEQ ID NO: 14) | KLIFKKILKYLKVFTISGKIILVGK (SEQ ID NO: 62) |
| RKRRRRKKRR (SEQ ID NO: 15) | KRKRKKLFKKILK (SEQ ID NO: 63) |
| KRRRRKKRRR (SEQ ID NO: 16) | SFATRFIPSP (SEQ ID NO: 64) |
| RRRRKKRRRR (SEQ lD NO: 17) | YRQERRARRRRRRERER (SEQ ID NO: 65) |
| ALKFGLKLAL (SEQ ID NO: 18) | ALKLALKLCL (SEQ ID NO: 66) |
| ALKLCLKLGL (SEQ ID NO: 19) | ASISQLKRSF (SEQ ID NO: 67) |
| CLKLALKLAL (SEQ ID NO: 20) | CLKLGLKLGL (SEQ ID NO: 68) |
| GLKLALKFGL (SEQ ID NO: 21) | KLALKFGLKL (SEQ ID NO: 69) |
| KLALKLALKL (SEQ ID NO: 22) | KLCLKLALKL (SEQ ID NO: 70) |
| KLALKLGLKL (SEQ ID NO: 23) | LALKLALKLA (SEQ ID NO: 71) |
| LGLKLALKLC (SEQ ID NO: 24) | LKLALKLALK (SEQ ID NO: 72) |
| GQAGRARAAC (SEQ ID NO: 25) | AGRARAACKL (SEQ ID NO: 73) |
| KLALKLGLKLALKLCLKLGLKLGLKLALKFGLK (SEQ ID | GRARAACKLA (SEQ ID NO: 74) |
| NO: 26) | |
| RARAACKLAL (SEQ ID NO: 27) | ARAACKLALR (SEQ ID NO: 75) |
| RAACKLALRL (SEQ ID NO: 28) | RLNPGALRPA (SEQ ID NO: 76) |
| QGARLRSARK (SEQ ID NO: 29) | GARLRSARKV (SEQ ID NO: 77) |
| RLRSARKVLR (SEQ ID NO: 30) | LRSARKVLRA (SEQ ID NO: 78) |
| RKVLRATLKR (SEQ ID NO: 31) | RKVLRAKLKR (SEQ ID NO: 79) |
| GDIMGEWGNEIFGAIAGFLGYGRKKRRQRRR | GRKKRWFRRRRMKWKK (SEQ ID NO: 80) |
| (SEQ ID NO: 32) | |
| RKKRWFRRRRPKWKK (SEQ ID NO: 33) | RIKRRFRRLRPKWKK (SEQ ID NO: 81) |
| Ac-GLWRALWRLLRSLWRLLWRA-cysteamide | RRKKIWFRRLRMK (SEQ ID NO: 82) |
| (SEQ ID NO: 34) | |
| FₓrFₓKFₓrFₓK (Fₓ: cyclohexylalanine; | FrFKFrFK (SEQ ID NO: 83) |
| r: D-Arginine) (SEQ ID NO: 35) | |
| PLILLRLLRGQF (SEQ ID NO: 36) | PLIYLRLLRGQF (SEQ ID NO: 84) |
| RRILLQLLRGQF (SEQ ID NO: 37) | pliylrllrgqf (all residues: D-form) (SEQ ID |
| | NO: 85) |
| cyclo(FNₐRRRRQ) (Nₐ: L-2-naphthylalanine) | cyclo(fNₐRrRrQ) (f: D-phenylalanine) (SEQ ID |
| (SEQ ID NO: 38) | NO: 86) |
| cyclo(FfNₐRrRrQ) (SEQ ID NO: 39) | cyclo(ZRRRRQ) (Z: L-Aspartic acid decylamine |
| | amide) (SEQ ID NO: 87) |
| cyclo(CRRRRRRRRC) (Cyclization via a disulfide | cyclo(CYGRKKRRQRRRC) (Cyclization via a disulfide |
| bond) (SEQ ID NO: 40) | bond) (SEQ ID NO: 88) |
| cyclo(RRRRR) (SEQ ID NO: 41) | cyclo(RRRRRR) (SEQ ID NO: 89) |
| Dodecanoyl-cyclo(RRRRR) (SEQ ID NO: 42) | Dodecanoyl-cyclo(RRRRRR) (SEQ ID NO: 90) |
| LSTAADMQGVVTDGMASGLDKDYLKPDD (SEQ ID NO: 43) | SPANLDQIVSAKKPKIVQERLEKVIASA (SEQ ID NO: 91) |
| LSTAADMQGVVTDGMASG (SEQ ID NO: 44) | SFEVHDKKNPTLEIPAGATVDVTFIN (SEQ ID NO: 92) |
| VKKKKIKAEIKI (SEQ ID NO: 45) | GLFDIIKKIAESF (SEQ ID NO: 93) |
| KGEGAAVLLPVLLAAPG (SEQ ID NO: 46) | GFWFG (SEQ ID NO: 94) |
| ACTGSTQHQCG (SEQ ID NO: 47) | |

Examples of cell-penetrating proteins that have the membrane-traversing carrier function, and thus considered CPPs, are listed below:
Tat from human immunodeficiency virus type 1 (M. Green and P. M. Loewenstein, “Autonomous functional domains of chemically synthesized human immunodeficiency virus tat trans-activator protein”, Cell, 1988 Dec. 23, 55(6), 1179-1188. doi: 10.1016/0092-8674(88)90262-0) (A. D. Frankel and C. O. Pabo, “Cellular uptake of the tat protein from human immunodeficiency virus”, Cell, 1988 Dec. 23, 55(6), 1189-1193. doi: 10.1016/0092-8674(88)90263-2):

(SEQ ID NO: 95)
MEPVDPRLEPWKHPGSQPKTACTNCYCKKCCFHCQVCFITKALGISYGR
KKRRQRRRAHQNSQTHQASLSKQPTSQPRGDPTGPKE

Antennapedia from Drosophila melanogaster (A. Joliot, C. Pernelle, H. Deagostini-Bazin, and A. Prochiantz, “Antennapedia homeobox peptide regulates neural morphogenesis”, Proc. Natl. Acad. Sci. U.S.A 1991, 88, 1864-1868) (P. E. G. Thorén, D. Persson, M. Karlsson, and B. Nordén, “The Antennapedia peptide penetratin translocates across lipid bilayers—the first direct observation”, FEBS Lett. 2000, 482, 265-268):

(SEQ ID NO: 96)
MTMSTNNCESMTSYFTNSYMGADMHHGHYPGNGVTDLDAQQMHHYSQNA
NHQGNMPYPRFPPYDRMPYYNGQGMDQQQQHQVYSRPDSPSSQVGGVMP
QAQTNGQLGVPQQQQQQQQQPSQNQQQQQAQQAPQQLQQQLPQVTQQVT
HPQQQQQQPVVYASCKLQAAVGGLGMVPEGGSPPLVDQMSGHHMNAQMT
LPHHMGHPQAQLGYTDVGVPDVTEVHQNHHNMGMYQQQSGVPPVGAPPQ
GMMHQGQGPPQMHQGHPGQHTPPSQNPNSQSSGMPSPLYPWMRSQFGKC
QERKRGRQTYTRYQTLELEKEFEIFNRYLTRRRRIEIAHALCLTERQIK
IWFQNRRMKWKKENKTKGEPGSGGEGDEITPPNSPQ

VP22 from herpes simplex virus type 1 (G. Elliott and P. O'Hare, “Intercellular Trafficking and Protein Delivery by a Herpesvirus Structural Protein”, Cell, 1997, 88, 223-233) (L. A. Kueltzo, N. Normand, P. O'Hare, and C. R. Middaugh, “Conformational lability of herpesvirus protein VP22”, J. Biol. Chem. 2000, 275, 33213-33221):

(SEQ ID NO: 97)
MTSRRSVKSGPREVPRDEYEDLYYTPSSGMASPDSPPDTSRRGALQTRS
RQRGEVRFVQYDESDYALYGGSSSEDDEHPEVPRTRRPVSGAVLSGPGP
ARAPPPPAGSGGAGRTPTTAPRAPRTQRVATKAPAAPAAETTRGRKSAQ
PESAALPDAPASTAPTRSKTPAQGLARKLHFSTAPPNPDAPWTPRVAGF
NKRVFCAAVGRLAAMHARMAAVQLWDMSRPRTDEDLNELLGITTIRVTV
CEGKNLLQRANELVNPDVVQDVDAATATRGRSAASRPTERPRAPARSAS
RPRRPVE

CaP from brome mosaic virus (X. Qi, T. Droste, and C. C. Kao, “Cell-penetrating peptides derived from viral capsid proteins”, Mol. Plant-Microbe Interact. 2010, 24, 25-36. doi: 10.1094/MPMI-07-10-0147):

(SEQ ID NO: 98)
MSTSGTGKMTRAQRRAAARRNRRTARVQPVIVEPLAAGQGKAIKAIAGY
SISKWEASSDAITAKATNAMSITLPHELSSEKNKELKVGRVLLWLGLLP
SVAGRIKACVAEKQAQAEAAFQVALAVADSSKEVVAAMYTDAFRGATLG
DLLNLQIYLYASEAVPAKAVVVHLEVEHVRPTFDDFFTPVYR

YopM from Yersinia enterocolitica (C. Rüter, C. Buss, J. Scharnert, G. Heusipp, and M. A. Schmidt, “A newly identified bacterial cell-penetrating peptide that reduces the transcription of pro-inflammatory cytokines”. J. Cell Sci., 2010 July; 123, 2190-2198. doi: 10.1242/jcs.063016):

(SEQ ID NO: 99)
MFINPRNVSNTFLQEPLRHSSDLTEMPVEAENVKSKAEYYNAWSEWERN
APPGNGEQRGMAVSRLRDCLDRQAHELELNNLGLSSLPELPPHLESLVA
SCNSLTELPELPQSLKSLQVDNNNLKALSDLPPLLEYLGAANNQLEELP
ELQNSSFLTSIDVDNNSLKTLPDLPPSLEFLAAGNNQLEELSELQNLPF
LTAIYADNNSLKTLPDLPPSLKTLNVRENYLTDLPELPQSLTFLDVSDN
IFSGLSELPPNLYNLNASSNEIRSLCDLPPSLVELDVRDNQLIELPALP
PRLERLIASENHLAEVPELPQNLKLLHVEYNALREFPDIPESVEDLRMD
SERVIDPYEFAHETIDKLEDDVFE

Artificial protein B1 (R. L. Simeon, A. M. Chamoun, T. McMillin, and Z. Chen, “Discovery and Characterization of a New Cell-Penetrating Protein”, ACS. Chem. Biol., 2013; 8, 2678-2687. doi: 10.1021/cb4004089):

(SEQ ID NO: 100)
MWFKREQGRGAVHRGGAHPGRAGRRRKRPQVQRVRRGRGRCHLRQADPE
VHLHHRQAARALAHPRDHPDLRRAVLQPLPRPHEAARLLQVRHARRLRP
GAHHLLQGRRQLQDPRRGEVRGRHPGEPHRAEGHRLQGGRQHPGAQAGV
QLQQPQRLYHGRQAEERHQGELQDPPQHRGRQRAAHRPLPAEHPHRRRP
RAAARQPLPEHPVRPEQRPQREARSHGPAGVRDRRRDHSRHGRGLNLE

30Kc19 from silkworm Bombyx mori. (J. H. Park, J. H. Lee, H. H. Park, W. J. Rhee, S. S. Choi, and T. H. Park, “A protein delivery system using 30Kc19 cell-penetrating protein originating from silkworm”, Biomaterials, 2012, 33, 9127-9134. doi: 10.1016/j.biomaterials.2012.08.063):

(SEQ ID NO: 101)
MKPAIVILCLFVASLYAADSDVPNDILEEQLYNSVVVADYDSAVEKSKH
LYEEKKSEVITNVVNKLIRNNKMNCMEYAYQLWLQGSKDIVRDCFPVEF
RLIFAENAIKLMYKRDGLALTLSNDVQGDDGRPAYGKDKTSPRVSWKLI
ALWENNKVYFKILNTERNQYLVLGVGTNWNGDHMAFGVNSVDSFRAQWY
LQPAKYDNDVLFYIYNREYSKALTLSRTVEPSGHRMAWGYNGRVIGSPE
HYAWGIKAF

Engineered +36 GFP (Cronican J. J. et al., “Potent Delivery of Functional Proteins into Mammalian Cells in Vitro and in Vivo Using a Supercharged Protein”, ACS Chem. Biol. 2010, 5, 8, 747-752; doi: 10.1021/cb1001153):

(SEQ ID NO: 102)
MGHHEIHREIGGASKGERLFRGKVPILVELKGDVNGHKFSVRGKGKGDA
TRGKLTLKFICTTGKLPVPWPTLVTTLTYGVQCFSRYPKHMKRHDFFKS
AMPKGYVQERTISFKKDGKYKTRAEVKFEGRTLVNRIKLKGRDFKEKGN
ILGHKLRYNFNSHKVYITADKRKNGIKAKFKIRHNVKDGSVQLADHYQQ
NTPIGRGPVLLPRNHYLSTRSKLSKDPKEKRDHMVLLEFVTAAGIKHGR
DERYK

Naturally supercharged human proteins, e.g. N-DEK (primary sequence shown below) (Cronican J. J. et al., “A Class of Human Proteins That Deliver Functional Proteins Into Mammalian Cells In Vitro and In Vivo”, Chem. Biol., 2011, 18(7): 833-838; doi: 10.1016/j.chembiol.2011.07.003):

(SEQ ID NO: 103)
MFTIAQGKGQKLCEIERIHFFLSKKKTDELRNLHKLLYNRPGTVSSLKK
NVGQFSGFPFEKGSVQYKKKEEMLKKFRNAMLKSICEVLDLERSGVNSE
LVKRILNFLMHPKPSGKPLPKSKKTCSKGSKKER

Optionally, a CPP may be utilized that carries cargo molecules to a particular intracellular compartment, such as the cytosol or particular organelle. For example, an organelle-specific CPP may be used, capable of carrying cargo molecules to an organelle, such as the nucleus, mitochondria, Golgi apparatus, endoplasmic reticulum, lysosome/endosome, etc. (Cerrato C P et al., “Cell-penetrating peptides with intracellular organelle targeting”, Review Expert Opin Drug Deliv., 2017 February; 14(2):245-255; Sakhrani N M and H Padh, “Organelle targeting: third level of drug targeting,” Drug Des Devel Ther. 2013, 7: 585-599, which are each incorporated herein by reference in their entireties).

### Cargo Molecules

The payload to be delivered to cells in vitro or in vivo is referred to herein as the “cargo” or a “cargo molecule” and may belong to any class of substance or combination of classes. Examples of cargo molecules include, but are not limited to, a small molecule (e.g., a drug), macromolecule such as polyimides, proteins (e.g., enzymes, membrane-bound proteins), polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, catalytic RNA, RNAzyme, ribozyme, non-coding RNA (ncRNA) such as miRNA, snRNA, interfering RNA such siRNA or shRNA, single guide RNA for Cas9, and mRNA, tRNA, and ribosomal RNA (rRNA)), antibody or antibody-fragment, lipoprotein, carbohydrate, or glycoprotein. In some embodiments, the cargo molecule is a hormone, metabolite, signal molecule, vitamin, or anti-aging agent.

First, the intended cargo molecule can be covalently or non-covalently coupled with a natural, modified, or artificial CPP at its N- or C-terminus. In the case of covalent coupling, the cargo molecule can be coupled to a CPP via either a disulfide bond, an amide bond, a chemical bond formed between a sulfhydryl group and a maleimide group, a chemical bond formed between a primary amine group and an N-Hydroxysuccinimide (NETS) ester, a chemical bond formed via Click chemistry, or other covalent linkages. The coupled cargo is denoted as “the binding complex”. Following are several scenarios: i) if the cargo is a polypeptide with a small to medium size, the binding complex can be chemically synthesized; ii) if the binding complex is a CPP fused to either the N-terminus or C-terminus of a large sized polypeptide such as a protein (or inserted into any chosen site of the protein), the encoding DNA sequence of the fusion protein can be inserted into an expression vector for expression in bacteria, yeast, plants, or insect or mammalian cells for expression and purification; iii) if the cargo is a nucleic acid, the cargo can be chemically synthesized, made by polymerase chain reaction (PCR), made by ligation from smaller pieces of nucleic acids, or by other means. The nucleic acid will then be purified by high performance liquid chromatography (HPLC) or other means. The purified nucleic acid can then be covalently or non-covalently coupled to a CPP to form the binding complex; and iv) if the cargo is a lipid, a metabolite, a small or large chemical molecule, a dye, a sugar, a medical imaging agent, or a small molecule drug, the cargo can be chemically synthesized and HPLC purified. The purified cargo can then be coupled to a CPP via either disulfide, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkages to form the binding complex.

Second, the binding complex can be purified via column chromatography, HPLC, or other means. Third, the purified binding complex can be incubated with and then enter the LVs. These are referred to as a “loaded LV”. Fourth, the linkages of certain covalent conjugation, e.g. the disulfide linkage, can be broken by incubating the loaded vesicles with small lipid layer-penetrating molecules, e.g. dithiothreitol (DTT) for reducing the disulfide linkage, leading to the formation of cargos free of the CPP inside the loaded LVs. Alternatively, once the loaded LV fuse with host cells and the CPP-cargo conjugated via a disulfide linkage enter the cells, the disulfide linkage will be broken by a cellular reducing environment, freeing the cargo inside the cells. If the cargo molecule is covalently linked with a CPP via photo-cleavable conjugation, the binding complex inside an LV can be cleaved into the CPP and the cargo molecule once the LV is exposed to light of the proper wavelength. This will free the cargo inside the LV. Finally, the loaded LVs will be administered to cells in vitro or an organism in vivo, e.g. a human or non-human animal subject, and then fuse with various organism's cells for cargo delivery. Once inside the organism's cells, the cargo molecules can play various biological roles and affect the function and behavior of the organism's cells, relevant tissues, organs, and/or even the entire organism.

In some embodiments, the CPP can be inserted in a position of any loop regions which do not have secondary structure and do not interact with other parts of the polypeptide cargo.

In some embodiments, the cargo molecule is DNA, which may be inhibitory, such as an antisense oligonucleotide, or the DNA may encode a polypeptide and can optionally include a promoter operably linked to the encoding DNA. In some embodiments, the cargo molecule is an RNA molecule such as snRNA, ncRNA (e.g. miRNA), mRNA, tRNA, catalytic RNA, RNAzyme, ribozyme, interfering RNA (e.g., shRNA, siRNA), or guide RNA (e.g., sgRNA) for gene editing by a gene editing enzyme (e.g., Cas9).

Optionally, small RNAs (tRNAs, Y RNAs, sn/sno RNAs) can be glycosylated (called “glycoRNAs”) and anchored to the membrane or outer lipid layer of the LVs. Small noncoding RNAs bearing sialylated glycans have been found on the cell surface of multiple cell types and mammalian species, in cultured cells, and in vivo, and were determined to interact with anti-dsRNA antibodies and members of the Siglec receptor family (Flynn R A et al., “Small RNAs are modified with N-glycans and displayed on the surface of living cells”, Cell 2021, 184:3109-3124). GlycoRNAs can be included as part of the cargo molecule, which is coupled to the CPP to form a binding complex and loaded onto the LV. Alternatively, glycoRNA may itself be a cargo molecule, coupled to a CPP to form another binding complex, which is loaded onto the LV. In either case, the glycoRNA can be loaded onto the LV for display on the outer lipid layer of the LV.

In some embodiments, the cargo molecule is a monoclonal or polyclonal antibody, or antigen-binding fragment thereof. The antibody or antibody fragment may be a human antibody or fragment, animal antibody fragment, chimeric antibody or fragment, or humanized antibody or fragment.

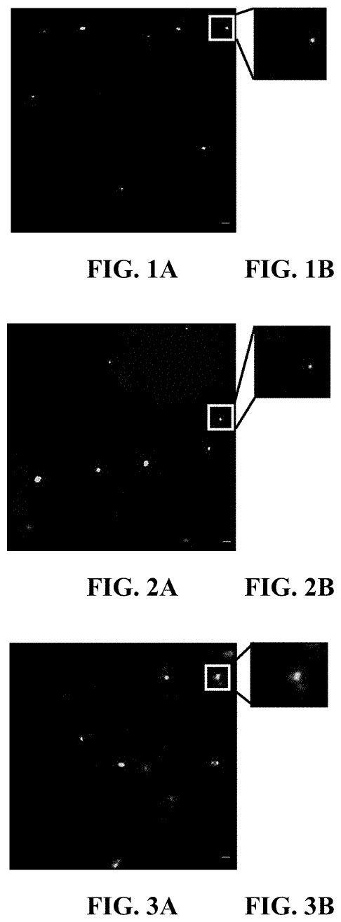

For the fusion between the CPP and an antibody or antibody fragment, the CPP may be coupled at the C-termini of the heavy chains of the antibody, as opposed to the N-termini of the heavy or light chains (as shown by FIG. 2B of Zhang J-F et al., “A cell-penetrating whole molecule antibody targeting intracellular HBx suppresses hepatitis B virus via TRIM21-dependent pathway”, Theranostics, 2018, 8(2):549-562). Fusion of the CPP may also be done at a position before or after the hinge (as described in the Abstract and FIG. 1 of Gaston J et al., “Intracellular delivery of therapeutic antibodies into specific cells using antibody-peptide fusions”, Scientific Reports, 2019, 9:18688). Preferably, the CPP is fused at the C-termini of the heavy chains or around the hinges although other fusions sites may be used. For other polypeptide cargos (i.e., polypeptides other than antibodies or antibody fragments), fusion may be done at the N-terminus or C-terminus, or internal loop areas of the polypeptide cargo molecule. Interference with the cargo molecule's function(s) should be avoided.

In some embodiments, the cargo molecule is, or has coupled to it, a detectable agent such as a fluorescent (e.g., a fluorophore), luminescent (e.g. a luminophore, Quantum dots), radioactive (e.g. ¹³¹I-Sodium iodide, ¹⁸F-Sodium fluoride) compound to serve as a marker, dye, tag, reporter, medical imaging agent, or contrast agent. Examples of fluorescent proteins include green fluorescent protein (GFP) and GFP-like proteins (Stepanenko O V et al., “Fluorescent Proteins as Biomarkers and Biosensors: Throwing Color Lights on Molecular and Cellular Processes”, Curr Protein Pept Sci, 2008, 9(4):338-369, which is incorporated herein by reference in its entirety”). In some embodiments, the detectable agent is a quantum dot or other fluorescent probe that may be used, for example, as a contrast agent with an imaging modality such as magnetic resonance imaging (MM). The detectable agent may be coupled to a cargo molecule, such as a polypeptide or nucleic acid (e.g., DNA or RNA), to detect, track the location of, and/or quantify the cargo molecule to which it is coupled.

In some embodiments, the cargo molecule is a labeled protein, such as an isotope-labeled protein. Such labeled proteins may be used in nuclear magnetic resonance imaging (NMR) protein analysis (Hu Y et al., “NMR-Based Methods for Protein Analysis”, Anal. Chem., 2021, 93:1866-1878; Lee K R et al., “Stable Isotope Labeling of Proteins in Mammalian Cells”, Journal of the Korean Magnetic Resonance Society, 2020, 24:77-85; and Verardi R et al., “Isotope Labeling for Solution and Solid-State NMR Spectroscopy of Proteins”, Adv Exp Med Biol., 2012, 992: 35-62, which are each incorporated herein by reference in their entireties). One ore more CPPs may be used to load a stable isotope-labeled protein into LVs for protein NMR measurements. Various isotopes are available for labeling (e.g., ¹H, ¹⁵N, ¹³C, ²H). The CPPs can potentially load several millimolar of a protein into each LV and the local protein concentration would be ideal for protein NMR studies.

The cargo molecule may be covalently conjugated to the CPP by a disulfide bond, Click chemistry, other covalent linkage, or be non-covalently bound to the CPP.

Optionally, the binding complex includes two or more cargo molecules, which may be the same class of molecule (e.g., two or more polypeptides) or molecules of a different class (e.g., a polypeptide and a small molecule).

In some embodiments, the cargo molecule comprises a growth factor or growth miRNA, and the loaded LV may be administered to an acute or chronic wound of a subject to promote wound healing. For example, growth factors and/or miRNAs may be delivered into skin cells via LVs for wound healing purposes.

Growth factors have previously been applied to wounds for wound healing; however, their positive effects on wound healing are limited. For example, growth factors and growth miRNAs are prone to be degraded by extracellular enzymes or bound and neutralized by a subject's extracellular proteins and immune responses in the wound environment. Advantageously, the invention may be used to deliver growth factors and/or growth miRNAs, or combinations thereof, into skin cells, e.g. human primary dermal fibroblasts, via LVs which protect these growth factors from being degraded by extracellular enzymes of a subject, bound by extracellular proteins of the subject, and/or neutralized by the subject's immune responses.

First, the intended cargos such as growth factors and/or miRNAs will be covalently or non-covalently coupled with a CPP to make a binding complex. For example, in the case of covalent coupling, this can be achieved via either a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkages. Both CPPs and growth miRNAs can be chemically synthesized and purified by HPLC. A CPP can be genetically fused with a growth factor and the fusion protein can be expressed in bacteria, yeast cells, plants, insect cells, or mammalian cells. Second, each binding complex can be purified via either HPLC or column chromatography. Third, the purified binding complex can be incubated with and then enter LVs (forming loaded LVs). Certain bioconjugation linkages can be utilized that can be broken to free the cargo inside LVs. For example, the disulfide bond linkage can be reduced by DTT which enters LVs after the incubation of DTT and LVs. Finally, the loaded LVs can be directly administered to wounds in order to accelerate wound healing.

The invention will allow any combinations of growth factors and/or growth miRNAs to be first loaded into LVs, which protect the loaded growth factors and/or growth miRNAs from degradation by extracellular enzymes, binding by host extracellular proteins, or neutralization by host immune responses. Such growth factors-loaded and/or growth miRNAs-loaded LVs will be applied to wounds, leading to the delivery of the intended growth factors and/or growth miRNAs into skin cells. Once inside the skin cells, the growth factors and/or growth miRNAs will play biological roles and accelerate wound healing.

Skin is the outer covering of the human body which protects the body from heat, light, injury, and numerous forms of infections. However, it is prone to undergo frequent damage by the occurrence of acute and chronic non-healing wounds. The latter wounds are often caused by diabetic foot ulcers, pressure ulcers, arterial insufficiency ulcers, and venous ulcers. Research in the field of wound healing has focused on expediting wound healing processes. There have been advancements on developing stem cell transplantation therapy, exploiting the use of microRNAs in tissue regeneration and engineering, and examining the role of the exosome in wound healing. Various preclinical and early clinical studies have shown the propitious results of the application of mesenchymal stem cells (MSC), embryonic stem cells, or pluripotent stem cells, especially adipose stem cells having an MSC origin, considered as most promising in the treatment of skin wounds. Notably, human umbilical cords are rich source of MSCs and hematopoietic stem cells (HSC) and such MSCs have been used to treat different types of disorders like wound healing, bone repair, neurological diseases, cancer, and cardiac and liver diseases.

The growth factors secreted by various cells have gained more clinical attention for wound management. Growth factors such as those in the table below are important signaling molecules which are known to regulate cellular processes responsible for wound healing. These molecules are upregulated in response to tissue injury and mainly secreted by fibroblasts, leukocytes, platelets, and epithelial cells. Even at very low concentrations, these proteins can have remarkable impact on the injury area, leading to rapid enhancement in cell migration, differentiation, and proliferation. Various recombinant growth factors have been tested in order to identify their roles in wound healing processes including cell migration, differentiation, and proliferation. In vitro and in vivo studies of chronic wounds have revealed that various growth factors have been down regulated. If these down-regulated growth factors are made recombinantly and delivered into cells at injury sites, they may stimulate wound healing, resulting in new therapies.

Examples of growth factors that may be used in the invention are provided in Table 3 below.

TABLE 3
Examples of Growth Factors

| Growth | | Molecular |
|---|---|---|
| factor | Source | Function |
| VEGF | Keratinocytes, | Inflammation, |
| | Fibroblasts, | Angiogenesis |
| | Macrophages, | |
| | Endothelial cells | |
| | Smooth muscle cells | |
| CX3CL1 | Macrophages, | Inflammation, |
| | Endothelial cells | Angiogenesis, |
| | | Collagen deposition |
| TGF-β | Fibroblasts, | Inflammation, |
| | keratinocytes, | Angiogenesis, |
| | macrophages, | Granulation tissue |
| | platelets | formation, Collagen |
| | | synthesis, Tissue |
| | | remodelling, Leukocyte |
| | | chemotactic function |
| IL-6 | Fibroblasts, | Inflammation, |
| | Endothelial | Angiogenesis, |
| | cells, Macrophages, | re-epithelialization, |
| | Keratinocytes | Collagen |
| | | deposition, tissue |
| | | remodeling |
| IL-1 | Macrophages, | Inflammation, |
| | Leukocytes, | Angiogenesis, |
| | Keratinocytes, | Re-epithelialization, |
| | Fibroblasts | Tissue remodeling |
| PDGF | Platelets | Inflammation, |
| | | Re-epithelialization, |
| | | Collagen |
| | | deposition, Tissue |
| | | remodeling |
| IL-27 | Macrophages | Suppression of |
| | | inflammation, |
| | | collagen synthesis |
| HGF | Fibroblasts | Suppression of |
| | | inflammation, |
| | | Granulation |
| | | tissue formation, |
| | | Angiogenesis, |
| | | Re-epithelialization |
| Activin | Keratinocytes, | Granulation tissue |
| | Fibroblasts | formation, |
| | | Keratinocyte |
| | | Differentiation, |
| | | Re-epithelialization, |
| FGF-2 | Keratinocytes, | Angiogenesis, |
| | Fibroblasts, | Granulation |
| | Endothelial cells | tissue formation |
| Angiopoietin- | Fibroblasts | Angiogenesis |
| 1/−2 | | |
| EGF, HB-EGF, | Keratinocytes, | Re-epithelialization |
| TGF-α | Macrophages | |
| FGF-7, | Fibroblasts, | Re-epithelialization, |
| FGF-10 | Keratinocytes | Detoxification of ROS |
| CXCL10, | Keratinocytes, | Re-epithelialization, |
| CXCL11 | Endothelial cells | Tissue remodelling |
| IL-4 | Leukocytes | Collagen synthesis |
| GM-CSF | Macrophages, T cells, | Recruit Langerhans |
| | Mast cells, Natural | cells, Stimulate |
| | killer cells, Fibroblast, | proliferation |
| | Endothelial cells | and differentiation |
| TNF-α | Neutrophils | Inflammation |
| | Macrophages | Reepithelialization |

Besides growth factors, quite a few miRNAs, one type of small noncoding RNAs, have also been found to play important roles in wound healing. The growth miRNAs are known to regulate cellular expression of various genes involved in numerous aspects and phases of wound healing. Table 4 below is a list of examples of miRNAs that are known to accelerate chronic wound healing processes, and may be used with the invention.

TABLE 4
Examples of Growth Micro RNAs

| | Proliferation phase | | | | | |
|---|---|---|---|---|---|---|
| Inflammatory | Re- | Angiogenesis | Granulation Tissue | Remodeling | | |
| phase | epithelialization | Process | Formation | phase | Migration | Invasion |
| miR-221/222 | miR-21 | miR-1 | miR-29 | miR-29a | miR-196a | miR-200b |
| miR-17-5p | miR-31 | miR-21 | miR-98 | miR-29b | | miR-200c |
| miR-18a | miR-203 | miR-23a | miR-141-3p | miR-29c | | miR-141 |
| miR-106b | miR-204 | miR-29b | miR-185 | miR-192 | | |
| miR-193b | miR-205 | miR-126 | miR-15a | | | |
| miR-210 | miR-210 | miR-133a/b | miR-15b | | | |
| miR-34a | | miR-146a | miR-16 | | | |
| miR-181a/b | | miR-210 | miR-17 | | | |
| | | miR-218 | miR-17-92 | | | |
| | | miR-377 | miR-20a | | | |
| | | miR-939 | miR-20b | | | |
| | | miR-4530 | miR-21 | | | |
| | | | miR-92a | | | |
| | | | miR-101 | | | |
| | | | miR-126 | | | |
| | | | miR-130a | | | |
| | | | miR-184 | | | |
| | | | miR-200b | | | |
| | | | miR-203 | | | |
| | | | miR-205 | | | |
| | | | miR-206 | | | |
| | | | miR-210 | | | |
| | | | miR-221 | | | |
| | | | miR-222 | | | |
| | | | miR-296 | | | |
| | | | miR-320 | | | |
| | | | miR-378 | | | |

According to the Global Wound Dressings Market 2018-2022 report, it is estimated that more than 305 million patients globally are affected by traumatic, acute and chronic non-healing wounds each year. It is more than nine times higher than the total number of individuals affected by cancer around the world. In developed countries, nearly 1 to 2% population suffers from non-healing chronic wounds and the population is expected to rise at the rate of 2% each year over the next decade. The diabetic foot ulcers and surgical wounds account a significant portion of wound care costs.

Based on chronic wound epidemic cited in the United States, the rise in the incidence of chronic wounds is due to changing lifestyle, aging population, and rapid increase in conditions like obesity and diabetes. It is estimated that more than 50% of patients who undergo limb amputation will die within a year. In the United States, medical healthcare spends more than $32 billion each year while approximately $96.8 billion per year are spent on non-healing chronic wound treatment. To make it worse, more than 8.2 million individuals have suffered from chronic non-healing wound disorders.

Eukaryotic cell membrane is a tough barrier that protects the cells from external bioactive molecules. During the last decade, numerous studies demonstrated the use of CPPs as a promising carrier for delivering several therapeutic agents to their targets. Many CPPs are cost effective, short peptide sequences that facilitate the entry of cargo molecules across biological membranes, without using specific receptors or transporters. In accordance with the invention, CPPs can be used to transport cargo molecules into LVs which can fuse with cells for eventual cargo delivery into cells.

The present invention may be used for efficient wound healing and based on the inventors' surprising discovery that human fibroblast growth factor-1 (FGF-1) conjugated with a CPP can be loaded into LVs such as liposomes, and the loaded LVs will enhance processes that are beneficial in wound healing, such as cell migration, cell proliferation, and cell invasion. It is likely that FGF1-loaded LVs can significantly enhance wound healing through one or more of its phases (hemostasis, inflammation, proliferation, and maturation/remodeling). The present invention can employ CPPs as delivery agents that carry and load growth factors and growth miRNAs into LVs, and use these loaded LVs as wound healing therapies.

### Exemplified Embodiments

Embodiment 1. A method for loading a lipid vesicle (LV) with a cargo molecule, comprising contacting the LV with a binding complex, wherein the binding complex comprises the cargo molecule and a cell penetrating polypeptide (CPP) covalently or non-covalently coupled to the cargo molecule, and wherein the binding complex becomes internalized by, or associated with, the LV.

Embodiment 2. The method of embodiment 1, wherein the CPP is non-covalently coupled to the cargo molecule.

Embodiment 3. The method of embodiment 1, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage.

Embodiment 4. The method of embodiment 3, wherein the CPP is covalently coupled to the cargo molecule by a cleavable linker.

Embodiment 5. The method of embodiment 4, wherein the cleavable linker is a photo-cleavable linker.

Embodiment 6. The method of embodiment 4, further comprising uncoupling the cargo molecule and CPP of the binding complex by cleaving the cleavable linker after the binding complex becomes internalized by, or associated with, the LV.

Embodiment 7. The method of any one of embodiments 1 to 6, wherein the cargo molecule is selected from among a small molecule (e.g., a drug, a fluorophore, a luminophore), macromolecule such as polyimide, proteins (e.g., enzymes, membrane-bound proteins), polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, glycoRNA, enzymatic catalytic RNA, RNAzyme, ribozyme, non-coding RNA (ncRNA) such as microRNA (miRNA), small nuclear RNA (snRNA), interfering RNA such siRNA or shRNA, single guide RNA for a gene editing enzyme (e.g., Cas9), messenger RNA (mRNA), transfer RNA (tRNA), and ribosomal RNA (rRNA)), antibody or antibody-fragment, lipoprotein, carbohydrate, or glycoprotein.

Embodiment 8. The method of any one of embodiments 1 to 7, wherein the LV is a liposome.

Embodiment 9. The method of any one of embodiments 1 to 7, wherein the LV is a lipid nanoparticle, lipid droplet, micelle, reverse micelle, lipid-polymer hybrid nanoparticle, or artificial extracellular vesicle.

Embodiment 10. The method of any one of embodiments 1 to 9, wherein the cargo molecule comprises a growth factor or growth miRNA.

Embodiment 11. The method of any one of embodiments 1 to 10, wherein the cargo molecule is a detectable agent or medical imaging agent, or is attached to a detectable or medical imaging agent, such as a fluorescent compound (e.g., a fluorophore) to serve as a marker, dye, tag, or reporter.

Embodiment 12. The method of any one of embodiments 1 to 11, wherein the cargo molecule is a labeled protein (e.g., an isotope-labeled protein).

Embodiment 13. The method of any preceding embodiment, wherein the LV further comprises a targeting agent that targets the LV to a cell type, organ, or tissue (e.g., cancer cells, neural cells of the central nervous system or peripheral nervous system, or muscle cells).

Embodiment 14. The method of any preceding embodiment, wherein the CPP is one listed in Table 2 or Table 11.

Embodiment 15. The method of any one of embodiments 1 to 13, wherein the CPP is selected from among the following: Tat, Antennapedia, VP22, CaP, YopM, Artificial protein B1, 30Kc19, engineered +36 GFP, naturally supercharged human protein, and gamma-AA peptide.

Embodiment 16. The method of any preceding embodiment, wherein the method further comprises the step of coupling CPP to the cargo molecule prior to contacting the LV with the binding complex.

Embodiment 17. The loaded LV produced by the method of any one of embodiments 1 to 16.

Embodiment 18. A loaded lipid vesicle (LV), comprising a cargo molecule and a cell penetrating peptide (CPP), wherein the cargo molecule has been internalized by, or associated with, the LV.

Embodiment 19. The loaded LV of embodiment 18, where the loaded LV comprises a binding complex, wherein the binding complex comprises the cargo molecule and a CPP covalently or non-covalently coupled to the cargo molecule, and wherein the binding complex has been internalized by, or associated with, the LV.

Embodiment 20. The loaded LV of embodiment 19, wherein two or more CPP are covalently or non-covalently coupled to the cargo molecule.

Embodiment 21. The loaded LV of embodiment 20, wherein the CPP is non-covalently coupled to the cargo molecule.

Embodiment 22. The loaded LV of embodiment 19, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage.

Embodiment 23. The loaded LV of embodiment 22, wherein the CPP is coupled to the cargo molecule by a cleavable linker.

Embodiment 24. The loaded LV of embodiment 23, wherein the cleavable linker is a photo-cleavable linker.

Embodiment 25. The loaded LV of any one of embodiments 18 to 24, wherein the cargo molecule is selected from among a small molecule (e.g., a drug, a fluorophore, a luminophore), macromolecule such as polyimide, proteins such as enzymes or membrane bound proteins, polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, glycoRNA, enzymatic catalytic RNA, RNAzyme, ribozyme, ncRNA (e.g., miRNA), small nuclear RNA (snRNA), interfering RNA such siRNA or shRNA, single guide RNA for a gene editing enzyme (e.g., Cas9), messenger RNA (mRNA), transfer RNA (tRNA), and ribosomal RNA (rRNA)), antibody or antibody-fragment, lipoprotein, carbohydrate, or glycoprotein.

Embodiment 26. The loaded LV of any one of embodiments 18 to 25, wherein the LV is a liposome.

Embodiment 27. The loaded LV of any one of embodiments 18 to 25, wherein the LV is a lipid nanoparticle, lipid droplet, micelle, reverse micelle, lipid-polymer hybrid nanoparticle, or artificial extracellular vesicle.

Embodiment 28. The loaded LV of any one of embodiments 18 to 27, wherein the cargo molecule comprises a growth factor or growth miRNA.

Embodiment 29. The loaded LV of any one of embodiments 18 to 28, wherein the cargo molecule is a detectable agent or medical imaging agent, or is attached to a detectable agent or medical imaging agent, such as a fluorescent compound (e.g., a fluorophore) to serve as a marker, dye, tag, or reporter.

Embodiment 30. The loaded LV of any one of embodiments 18 to 29, wherein the cargo molecule is a labeled protein (e.g., an isotope-labeled protein).

Embodiment 31. The loaded LV of any one of embodiments 18 to 30, wherein the LV further comprises a targeting agent that targets the LV to a cell type, organ, or tissue (e.g., cancer cells, neural cells of the central nervous system or peripheral nervous system, or muscle cells).

Embodiment 32. The loaded LV of any one of embodiments 18 to 30, wherein the CPP is one listed in Table 2 or Table 11.

Embodiment 33. The loaded LV of any one of embodiments 18 to 31, wherein the CPP is selected from among the following: Tat, Antennapedia, VP22, CaP, YopM, Artificial protein B1, 30Kc19, engineered +36 GFP, naturally supercharged human protein, and gamma-AA peptide.

Embodiment 34. A method for delivering a cargo molecule into a cell in vitro or in vivo, comprising administering a loaded lipid vesicle (LV) to the cell in vitro or in vivo, wherein the loaded LV comprises a binding complex, wherein the binding complex comprises the cargo molecule and a cell penetrating polypeptide (CPP) covalently or non-covalently coupled to the cargo molecule, and wherein the loaded LV is internalized into the cell.

Embodiment 35. The method of embodiment 34, wherein the loaded LV comprises a binding complex, wherein the binding complex comprises the cargo molecule and a CPP covalently or non-covalently coupled to the cargo molecule, and wherein the binding complex has been internalized by, or associated with, the LV.

Embodiment 36. The method of embodiment 35, wherein the CPP is non-covalently coupled to the cargo molecule.

Embodiment 37. The method of embodiment 35, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage.

Embodiment 38. The method of embodiment 35, wherein the CPP is coupled to the cargo molecule by a cleavable linker.

Embodiment 39. The method of embodiment 38, wherein the cleavable linker is a photo-cleavable linker.

Embodiment 40. The method of any one of embodiments 34 to 39, wherein the cargo molecule is selected from among a small molecule (e.g., a drug, a fluorophore, a luminophore), macromolecule such as polyimide, proteins such as enzymes or membrane bound proteins, polypeptide (natural or modified), nucleic acid (e.g., natural, damaged or chemically modified DNA, DNA plasmid or vector, telomere, DNA quadruplex, DNAzyme, DNA-like molecule, antisense oligonucleotide, locked nucleic acid, threose nucleic acid, peptide nucleic acid (PNA), single or double-stranded nucleic acid, natural, damaged or chemically modified RNA, glycoRNA, enzymatic catalytic RNA, RNAzyme, ribozyme, non-coding RNA (ncRNA) such as microRNA (miRNA), small nuclear RNA (snRNA), interfering RNA such siRNA or shRNA, single guide RNA for a gene editing enzyme (e.g., Cas9), and mRNA, transfer RNA (tRNA), and ribosomal RNA (rRNA)), antibody or antibody-fragment, lipoprotein, carbohydrate, or glycoprotein.

Embodiment 41. The method of any one of embodiments 34 to 40, wherein the loaded LV is administered to the cell in vitro by contacting the cell with the loaded vesicle in vitro.

Embodiment 42. The method of any one of embodiments 34 to 40, wherein the loaded LV is administered to the cell in vivo by administering the loaded vesicle to a subject having the cell.

Embodiment 43. The method of any one of embodiments 34 to 42, wherein the LV is a liposome.

Embodiment 44. The method of any one of embodiments 34 to 42, wherein the LV is a lipid nanoparticle, lipid droplet, micelle, reverse micelle, lipid-polymer hybrid nanoparticle, or artificial extracellular vesicle.

Embodiment 45. The method of any one of embodiments 34 to 44, wherein the cargo molecule comprises a growth factor or growth miRNA.

Embodiment 46. The method of any one of embodiments 34 to 45, wherein the cell to which the loaded LV is administered is a skin cell (e.g., a primary dermal fibroblast).

Embodiment 47. The method of embodiment 45 or 46, wherein the cell to which the loaded LV is administered is a cell of a wound of a human or non-human animal subject, and wherein the loaded vesicle is administered to the wound in vivo.

Embodiment 48. The method of any one of embodiments 34 to 47, wherein the cargo molecule is a detectable agent or medical imaging agent, or is attached to a detectable agent or medical imaging agent, such as a fluorescent compound (e.g., a fluorophore) to serve as a marker, dye, tag, or reporter.

Embodiment 49. The method of any one of embodiments 34 to 48, wherein the cargo molecule is a labeled protein (e.g., an isotope-labeled protein).

Embodiment 50. The method of embodiment 49, further comprising carrying out NMR measurement on the labeled protein in vitro or in vivo.

Embodiment 51. The method of any preceding embodiment, wherein the LV further comprises a targeting agent that targets the LV to a cell type, organ, or tissue (e.g., cancer cells, neural cells of the central nervous system or peripheral nervous system, or muscle cells).

Embodiment 52. The method of any preceding embodiment, wherein the CPP is one listed in Table 2 or Table 11.

Embodiment 53. The method of any one of embodiments 34 to 51, wherein the CPP is selected from among the following: Tat, Antennapedia, VP22, CaP, YopM, Artificial protein B1, 30Kc19, engineered +36 GFP, naturally supercharged human protein, and gamma-AA peptide.

Embodiment 54. The method of any one of embodiments 34 to 53, wherein the method further comprises the step of loading the LV with the cargo molecule prior to administering the loaded LV to the cell.

Embodiment 55. The method of any one of embodiments 34 to 54, wherein the method further comprises the step of coupling the CPP to the cargo molecule prior to contacting the LV with the binding complex.

### Further Definitions

As used herein, the terms “a,” “an,” “the” and similar terms used in the context of the present invention (especially in the context of the claims) are to be construed to cover both the singular and plural unless otherwise indicated herein or clearly contradicted by the context. Thus, for example, reference to “a cell”, or “a cargo molecule”, or “a CPP” should be construed to encompass or cover a singular cell, singular cargo molecule, or singular CPP, respectively, as well as a plurality of cells, a plurality of cargo molecules, and a plurality of CPPs, unless indicated otherwise or clearly contradicted by the context.

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. Administration can be local at a particular anatomical site, or systemic.

As used herein, the term “antibody” refers to whole antibodies and any antigen binding fragment (i.e., “antigen-binding portion”) or single chains thereof. A whole antibody is a glycoprotein comprising at least two heavy (H) chains and two light (L) chains inter-connected by disulfide bonds. Each heavy chain comprises a heavy chain variable region (VH) and a heavy chain constant region comprising three domains, CH1, CH2 and CH3. Each light chain comprises a light chain variable region (VL or Vk) and a light chain constant region comprising one single domain, CL. The VH and VL regions can be further subdivided into regions of hyper-variability, termed complementarity determining regions (CDRs), interspersed with more conserved framework regions (FRs). Each VH or VL comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (C1q) of the classical complement system. An antibody is said to “specifically bind” to an antigen X if the antibody binds to antigen X with a K_{D} of 5×10⁻⁸ M or less, more preferably 1×10⁻⁸ M or less, more preferably 6×10⁻⁹ M or less, more preferably 3×10⁻⁹ M or less, even more preferably 2×10⁻⁹ M or less. The antibody can be chimeric, humanized, or, preferably, human. The heavy chain constant region can be engineered to affect glycosylation type or extent, to extend antibody half-life, to enhance or reduce interactions with effector cells or the complement system, or to modulate some other property. The engineering can be accomplished by replacement, addition, or deletion of one or more amino acids or by replacement of a domain with a domain from another immunoglobulin type, or a combination of the foregoing. The antibody may be any isotype, such as IgM or IgG.

As used herein, the terms “antibody fragment”, “antigen-binding fragment”, and “antigen-binding portion” of an antibody (or simply “antibody portion”) refer to one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, for example, Abbas et al., Cellular and Molecular Immunology, 6th Ed., Saunders Elsevier 2007); (iv) an Fd fragment consisting of the VH and CH1 domains; (v) an Fv fragment consisting of the VL and VH domains of a single arm of an antibody, (vi) a dAb fragment (Ward et al., Nature, 1989, 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” or “antigen-binding fragment” of an antibody.

As used herein, the term “cell penetrating polypeptide” or “CPP” refers to a polypeptide of any length having the ability to cross cellular membranes with a cargo molecule. These polypeptides are sometimes referred to as cell penetrating peptides, cell penetrating proteins, transport peptides, carrier peptides, and peptide transduction domains. The CPPs used in the invention have the capability, when coupled to a cargo molecule, of facilitating entrapment of a cargo molecule by an LV. The loaded cargo molecule may be carried by the LV in or on the vesicle's one or more membranes (“membrane cargo”) or within the core of the vesicle (“luminal cargo”). Structurally, CPPs tend to be small peptides, typically about 5 to 30 amino acids in length, though they may be longer. As used herein, the terms “cell penetrating polypeptide” and “CPP” are inclusive of short peptides and full-length proteins having the membrane-traversing carrier function. CPPs may be any configuration, such as linear or cyclic, may be artificial or naturally occurring, may be synthesized or recombinantly produced, and may be composed of traditional amino acids or may include one or more non-traditional amino acids. A non-exhaustive list of examples of CPPs is provided in Table 2 and Table 11.

As used herein, the term “contacting” in the context of contacting a cell with a loaded LV of the invention in vitro or in vivo means bringing at least one loaded LV into contact with the cell, or vice-versa, or any other manner of causing the loaded LV and the cell to come into contact.

As used herein, the term “gene editing enzyme” refers to an enzyme having gene editing function, such as nuclease function. The gene editing enzyme may be, for example, a Zinc finger nuclease (ZFN), transcription-activator like effector nuclease (TALEN), meganuclease, or component of the Clustered Regularly Interspaced Short Palindromic Repeats (CRISPR) system. CRISPRs are genetic elements that bacteria and archaea use as an acquired immunity to protect against bacteriophages. They consist of short sequences that originate from bacteriophage genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. CRISPR associated protein 9 (Cas9) is one example of a CRISPR gene editing enzyme that may be used with the invention. A small piece of RNA is created with a short guide sequence that binds to a specific target sequence of DNA in a genome. The RNA also binds to the Cas9 enzyme. As in bacteria, the modified RNA is used to recognize the DNA sequence, and the Cas9 enzyme cuts the DNA at the targeted location. As described below, although Cas9 is the enzyme that is used most often, other enzymes (for example, Cas12a (also known as Cpf1)) can also be used. Once the DNA is cut, the cell's own DNA repair machinery is used to add or delete pieces of genetic material, or to make changes to the DNA by replacing an existing segment with a customized DNA sequence.

Cas9 is the most well characterized Cas endonuclease and most often used in CRISPR laboratories; however, its use is often limited by its large size, its protospacer adjacent motif (PAM) sequence stringency, and its propensity to cut off-target DNA sequences. Many have addressed these limitations of Cas9 by engineering derivatives with more desirable properties, in particular increased specificity and reduced PAM stringency. Alternative Cas endonucleases with overlapping as well as unique properties may be used, such as Cas3, Cas12 (e.g., Cas12a, Cas12d, Cas12e), Cas13 (Cas13a, Cas13b), and Cas14. Depending upon the particular intended application, potentially any class, type, or subtype of CRISPR-Cas system may be used in the invention (Meaker G A and EV Koonen, “Advances in engineering CRISPR-Cas9 as a molecular Swiss Army knife”, Synth Biol (Oxf)., 2020; 5(1): ysaa021; Jamehdor S et al., “An overview of applications of CRISPR-Cas technologies in biomedical engineering”, Folia Histochemica et Cytobiologica, 2020, 58(3): 163-173; Zhu Y. and Zhiwei Huang, “Recent advances in structural studies of the CRISPR-Cas-mediated genome editing tools”, National Science Review, 2019, 6: 438-451; Murugan K et al., “The revolution continues: Newly discovered systems expand the CRISPR-Cas toolkit”, Mol Cell. 2017 Oct. 5; 68(1): 15-25; and Makarova K S et al., “Annotation and Classification of CRISPR-Cas Systems”, Methods Mol Blot, 2015; 1311: 47-75, which are each incorporated herein by reference in their entireties).

As used herein, the term “human antibody” means an antibody having variable regions in which both the framework and CDR regions (and the constant region, if present) are derived from human germline immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human antibody” does not include antibodies in which CDR sequences derived from the germline of another mammalian species, such as a mouse, have been grafted onto human framework sequences.

As used herein, the term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

As used herein, the term “human monoclonal antibody” refers to an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

As used herein, the term “isolated antibody” means an antibody or antibody fragment that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

As used herein, the term “monoclonal antibody” or “monoclonal antibody composition” means a preparation of antibody molecules of single molecular composition, which displays a single binding specificity and affinity for a particular epitope.

As used herein, the term “nucleic acid” means any DNA-based or RNA-based molecule, and may be a cargo molecule of the invention. The term is inclusive of polynucleotides and oligonucleotides. The term is inclusive of synthetic or semi-synthetic, recombinant molecules which are optionally amplified or cloned in vectors, and chemically modified, comprising unnatural bases or modified nucleotides comprising, for example, a modified bond, a modified purine or pyrimidine base, or a modified sugar. The nucleic acid may be in the form of single-stranded or double-stranded DNA and/or RNA. The nucleic acid may be a synthesized molecule, or isolated using recombinant techniques well-known to those skilled in the art. The nucleic acid may encode a polypeptide of any length, or the nucleic acid may be a non-coding nucleic acid. The nucleic acid may be a messenger RNA (mRNA). The nucleic acid may be a morpholino oligomer. For nucleic acids encoding polypeptides, the nucleic acid sequence may be deduced from the sequence of the polypeptide and the codon usage may be adjusted according to the host cell in which the nucleic acid is to be transcribed. DNA encoding a polypeptide optionally includes a promoter operably linked to the encoding DNA for expression.

In some embodiments, the nucleic acid is a DNA or RNA having an enzymatic activity (e.g., a DNAzyme or RNAzyme). In some embodiments, the nucleic acid is a ribonucleic acid (RNA) enzyme that catalyzes chemical reactions. RNAzyme is usually an artificial enzyme derived from in vitro RNA evolution method such as SELEX. A ribozyme, also called catalytic RNA, is usually an RNA enzyme which forms a complex with protein(s) or exists in the RNA/protein complex, e.g. ribosome. In some embodiments, the nucleic acid is a catalytic RNA, RNAzyme, or ribozyme.

In some embodiments, the nucleic acid is an antisense oligonucleotide, DNA, interfering RNA molecule (e.g., shRNA), microRNA, tRNA, mRNA, guide RNA (e.g., sgRNA) for gene editing by a gene editing enzyme such as CRISPR Cas9, catalytic RNA, RNAzyme, or ribozyme.

In some embodiments, the nucleic acid is inhibitory, such as an antisense oligonucleotide. In some embodiments, the nucleic acid is an RNA molecule such as snRNA, ncRNA (e.g. miRNA), mRNA, tRNA, catalytic RNA, RNAzyme, ribozyme, interfering RNA (e.g., shRNA, siRNA), or guide RNA (e.g., sgRNA) for a gene editing enzyme such as CRISPR Cas9. In some embodiments, the nucleic acid is a peptide nucleic acid (PNA).

As used herein, the terms “patient”, “subject”, and “individual” are used interchangeably and are intended to include human and non-human animal species. For example, the subject may be a human or non-human mammal. In some embodiments, the subject is a non-human animal model or veterinary patient. For example, the non-human animal patient may be a mammal, reptile, fish, or amphibian. In some embodiments, the non-human animal is a dog, cat, mouse, rat, guinea pig. In some embodiments, the non-human animal is a primate.

As used herein, the terms “protein”, “polypeptide”, and “peptide” are used interchangeably to refer to a polymeric form of amino acids of any length, which can include coded and non-coded amino acids, natural amino acids, chemically or biochemically modified or derivatized amino acids, and polypeptides having modified peptide backbones. The term “polypeptide” includes full-length proteins and fragments or subunits of proteins. For example, in the case of enzymes, the polypeptide may be the full-length enzyme or an enzymatically active subunit or portion of the enzyme. The term “polypeptide” includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes polypeptides comprising one or more of a fatty acid moiety, a lipid moiety, a sugar moiety, and a carbohydrate moiety. The term “polypeptides” includes post-translationally modified polypeptides. The polypeptide may be a cargo molecule of the invention. The polypeptide may be a cell penetrating polypeptide (CPP) of the invention.

As used herein, the phrase “therapeutically effective amount” or “efficacious amount” means the amount of an agent, such as a cargo molecule, that, when administered to a human or animal subject for treating a disease, is sufficient, in combination with another agent, or alone in one or more doses, to effect such treatment for the disease. The “therapeutically effective amount” will vary depending on the agent, the disease and its severity and the age, weight, etc., of the subject to be treated.

As used herein, the term “treat”, “treating” or “treatment” of any disease, disorder, or condition refers in one embodiment, to ameliorating the disease, disorder, or condition (i.e., slowing or arresting or reducing the development of the disease, disorder, or condition, or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease, disorder, or condition, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset or development or progression of the disease, disorder, or condition).

As used herein, the terms “lipid vesicle” or “LV” refer to a naturally occurring or an artificially created (non-naturally occurring) particle having an interior compartment or cavity (core) surrounded and enclosed by at least one lipid layer (e.g., a lipid monolayer or a lipid bilayer). LVs may be unilamellar in structure (having a single lipid layer) or multilamellar in structure (a concentric arrangement of two or more lipid layers). LVs may be spherical or have a non-spherical or irregular, heterogeneous shape. Examples of LVs include liposomes, lipid nanoparticles, lipid droplets, micelles, reverse micelles, lipid-polymer hybrid nanoparticles, and artificial extracellular vesicles; thus, the term LV is inclusive of liposomes, lipid nanoparticles, lipid droplets, micelles, reverse micelles, lipid-polymer hybrid nanoparticles, and artificial extracellular vesicles. The surrounding lipid layer may be composed of synthetic lipids, semi-synthetic lipids, naturally occurring lipids, or a combination of two or more of the foregoing, that are compatible with the lipid layer structure. The term lipid is used in a broader sense and includes, for example, triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate).

As used herein, the term “liposome” refers to a vesicle having an interior aqueous core surrounded by, and enclosed by, at least one lipid bilayer. Liposomes are typically spherical in shape but their shape and size may be controlled by their components, cargo, and preparation methods. In a liposome delivery product, the cargo (e.g., a drug substance) is generally “contained” in liposomes. The word “contained” in this context includes both encapsulated and intercalated cargo. The term “encapsulated” refers to cargo within an aqueous space and “intercalated” refers to incorporation of the cargo within a bilayer. Typically, water soluble cargos are contained in the aqueous compartment(s) and hydrophobic cargos are contained in the lipid bilayer(s) of the liposomes.

The major types of liposomes are the multilamellar vesicle (MLV, with multiple lamellar phase lipid bilayers), the small unilamellar liposome vesicle (SUV, with one lipid bilayer), the large unilamellar vesicle (LUV), and the cochleate vesicle. Some liposomes are multivesicular, in which one vesicle contains one or more smaller vesicles.

As used herein, the terms “lipid nanoparticle” or “LNP” and “solid lipid nanoparticle” or “SLNP” are interchangeable and refer to nanoparticles composed of lipids. LNPs have a solid lipid core matrix surrounded by a lipid monolayer. The LNP core is stabilized by surfactants and can solubilize lipophilic molecules. The core lipids can be fatty acids, acylglycerols, waxes, and mixtures of these surfactants. By “solid,” it is meant that at least a portion of the LNP is solid at room temperature or body temperature and atmospheric pressure. However, the LNP can include portions of liquid lipid and/or entrapped solvent.

As used herein, a “lipid droplet” refers to a cellular organelle containing a neutral-lipid core enclosed by a phospholipid monolayer (and associated proteins). Lipid droplets may be isolated from cells.

As used herein, the term “micelle” refers to an LV with a closed lipid monolayer and a fatty acid core and polar surface, whereas a “reverse micelle” or “inverted micelle” has a polar core with fatty acids on its surface.

Liposomes are composed of a lipid bilayer separating an aqueous internal compartment from the bulk aqueous phase. Micelles are closed lipid monolayers with a fatty acid core and polar surface, or polar core with fatty acids on the surface (inverted micelle).

As used herein, the term “lipid-polymer hybrid nanoparticles” or “LPHNP” refers to a lipid vesicle having a polymer core that can contain cargo, with the polymer core encapsulated by a lipid monolayer.

As used herein, the terms “artificial extracellular vesicle” or “synthetic extracellular vesicle” are interchangeable and refer to vesicles that are modified or manufactured (from natural or synthetic sources), with the aim to mimic EVs (such as exosomes) for therapeutic or other uses, as described in Garcia-Manrique P et al., Journal of Extracellular Vesicles, 2018, vol. 7, 1422676, which is incorporated by reference herein in its entirety. Artificial EVs may be semi-synthetic (e.g., starting from a natural substrate and subsequently modified before or after their isolation) or fully synthetic (e.g., manufactured top-down from cultured cells or bottom-up from individual molecules), as depicted in FIG. 1 of Garcia-Manrique P et al.

As used herein, a “lipid bilayer” refers to a structure composed of two layers of lipid molecules organized in two sheets, functioning as a barrier. A lipid bilayer surrounds cells as a biological membrane, providing the cell membrane structure. Liposomes have a lipid bilayer that creates an inner aqueous compartment due to the hydrophilic heads and the hydrophobic tails of the lipids.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Materials and Methods

Cell culture. Mouse embryonic fibroblasts and human primary dermal fibroblasts were purchased from ATTC (Cell Biology Collection), cultured in Dulbecco's modified Eagle's medium (DMEM) (Life Technologies, Carlsbad, Calif., USA) or fibroblast complete medium (PromoCell—C-23010). Fibroblasts were grown at 37° C. and under 5% CO₂ in cell culture flasks (BD falcon) as per manufacturer's instructions.

Peptide synthesis and purification. The N-terminal 5(6)-carboxyfluorescein (FAM)-labeled peptide FAM-YARA (FAM-YARAAARQARA-NH₂) (SEQ ID NO:55) and Peptide H (FAM-YARAAARQARAGGGGSVVIVGQIILSGR-NH₂) (SEQ ID NO:104) were chemically synthesized by Peptide International (Louisville, Ky., USA). The N-terminal 5(6)-carboxyfluorescein-labeled peptide FAM-YARA-Cys (FAM-YARAAARQARAGC-NH₂) (SEQ ID NO:57) was chemically synthesized by LifeTein, LLC (Somerset, N.J., USA). The C-termini of these peptides contain an amide. Each of the peptides was purified by high performance liquid chromatography (HPLC).

Construction and purification of chimera YARA-FGF1-GFP. The full-length DNA fragment, consisting of the coding sequence of YARA-FGF1-GFP, was cloned onto a pET expression vector by using restriction sites EcoRI and HindIII to generate a plasmid (pET28c-YARA-FGF1-GFP). The fusion protein YARA-FGF1-GFP was then expressed in E. coli Rosetta cells under a T7 RNA polymerase promoter in the plasmid. The YARA-FGF1-GFP protein was purified by column chromatography and its purity was evaluated through SDS PAGE.

Liposomes. Pre-formed pegylated remote loadable liposomes (3002025-1EA) were purchased from AVANTI POLAR LIPIDS INC MS (Alabaster, Ala., USA). These pegylated liposomes have a mean particle size of ˜90 nm and are composed of N-(carbonyl-ethoxypolyethylene glycol 2000)-1,2-di stearoyl-sn-glycero-3-phosphoethanolamine sodium salt (MPEG-DSPE).

Loading of peptides into liposomes. Purified FAM-YARA or Peptide H in water was added to a solution of the liposomes (0.1 mg/mL, 5.8×10⁹ particles/mL) in phosphate buffered saline (PBS) and the mixture was incubated for nearly 6 hours at room temperature. Internalization of each of the peptides into the liposomes was confirmed using Total Internal Reflection Fluorescence (TIRF) microscopy after removal of unattached peptides by washing the liposomes with PBS for three times and then filtration using Amicon Ultra-centrifugal filters (100 K device, Merck Millipore, Billerica, Mass., USA).

Loading of the fusion protein YARA-FGF1-GFP into liposomes. Purified recombinant protein YARA-FGF1-GFP (50 μg) in PBS was added to the solution of liposomes (0.1 mg/mL, 5.8×10⁹ particles/mL) and the mixture was incubated for overnight at room temperature. The internalization of the fusion protein YARA-FGF1-GFP into the liposomes was confirmed using TIRF microscopy after removal of unattached YARA-FGF1-GFP by washing the liposomes with PBS for three times and then filtration using Amicon Ultra-centrifugal filters (100 K device, Merck Millipore, Billerica, Mass., USA).

Thiol conjugation of a peptide with a DNA oligomer and loading into liposomes. A thiol-modified DNA oligomer S-1 (5′-/5ThioMC6-D/TCAACATCAGTCTGATAAGCTA-3′) (SEQ ID NO:105) was synthesized by IDT integrated DNA technologies (Redwood City, Calif., USA). S-1 was reduced by TCEP and purified by 17% polyacrylamide gel electrophoresis. The purified FAM-YARA-Cys, containing a thiol group at its C-terminal cysteine residue, was reacted overnight with the reduced and purified S-1 in a 1:1 molar ratio in the presence of 0.2 mM CuCl₂ (an oxidant) at room temperature in order to form the covalent conjugate FAM-YARA-Cys-ssDNA via a disulfide bond. Analysis of the formed covalent conjugate was examined by running the reaction mixture on a 2% agarose gel. The ethidium bromide-stained agarose gel was first photographed and then scanned under the Cy2 channel (Typhoon GE) to confirm the FAM-YARA-Cys-ssDNA conjugate formation. The desired product band was then cut and the product FAM-YARA-Cys-ssDNA was subsequently eluted by using the gel extraction kit QIAEXII (Qiagen, Hilden, Germany) as per manufacturer's instructions.

The purified FAM-YARA-Cys-ssDNA was added to a solution of the liposomes and the mixture was incubated for nearly 6 hours at room temperature. After the removal of unattached FAM-YARA-Cys-ssDNA by washing the liposomes with PBS for three times (Spin Columns MW 3000, Invitrogen), the internalization of FAM-YARA-Cys-ssDNA into the liposomes was confirmed using TIRF microscopy.

TIRF microscopy and image analysis. The liposomes in a 35 mm μ-dish glass bottom culture dish were initially incubated with either a peptide (FAM-YARA, or Peptide H), a peptide-DNA covalent conjugate (FAM-YARA-Cys-ssDNA), or a recombinant fusion protein (YARA-FGF1-GFP, 50 μg/mL) for 6 hours at room temperature. The liposomes were then washed for three times with PBS to remove any unattached peptides, peptide-DNA covalent conjugates, or proteins. After washing, the liposomes were subjected to TIRF imaging measurements using Nikon Eclipse Ti microscope and the images were processed and analyzed by using ImageJ.

Internalization of the liposomes loaded with either a peptide or a fusion protein into human primary dermal fibroblast cells monitored by confocal microscopy. Human primary dermal fibroblast cells in a 35 mm μ-dish glass bottom culture dish were initially incubated with a culture medium containing the liposomes loaded with either Peptide H or the fusion protein YARA-FGF1-GFP for 4 hours at 37° C. under 5% CO₂. The medium was then removed and the fibroblasts were washed for three times with PBS. The fibroblast cells were fixed with image-iT fixative solution (Invitrogen) as per manufactures protocol. The fibroblasts were then subjected to confocal microscopy measurements.

Cell migration assay. The migration capacity of fibroblasts was assessed with commercially available Cytoselect 24-well wound healing assay (Cell Biolabs, San Diego, Calif., USA) using wound field inserts that create a consistent gap of 0.9 mm between the cells. The assay was performed by following manufacturer's instructions. Specifically, fibroblasts were seeded into a 24-well plate with the cell density of 1×10⁶ cells/well with complete growth medium. Once achieving 100% confluency, the wells were washed twice with culture media to remove any detached cells. Next, the fibroblast culture medium containing PBS (the control), liposomes, liposomes loaded with YARA, or liposomes loaded with YARA-FGF1-GFP was added to respective wells. The liposomes concentration in each case except the control was 0.1 mg/mL (5.8×10⁹ particles/mL). The fibroblasts were then incubated at 37° C. under 5% CO₂ for different time periods (0, 6, 12, 24 hours). Cell migration was observed and images were taken under bright field microscope with 4× magnification at various time points (0, 6, 12, 24 hours). Cells were stained with the staining solution provided with the kit 24 h after inserts were removed. The scratch width at four different positions was measured at each time point in each treatment group. The rate of cell migration to close the wounded area was analyzed by using ImageJ software.

Cell proliferation assay. Prior to the MTS assay, the fibroblasts were cultured onto a 96-well culture plate at a cell density of 5×10⁴ cells/well. After 24 hr of incubation at 37° C. under 5% CO₂, the individual fibroblasts were supplemented with PBS (the control), liposomes, liposomes loaded with YARA, or liposomes loaded with YARA-FGF1-GFP. The liposomes concentration in each case except the control was 0.1 mg/mL (5.8×10⁹ particles/mL). At different time points (24, 48, and 72 hours), cell proliferation was measured by following the manufacturer's protocol. In brief, 20 μL of MTS labelling reagent was added to each well and the plate was incubated at 37° C. for 1 hour. After incubation, the absorbance was read at 490 nm.

Invasion assay. The effects of loaded or unloaded liposomes on fibroblast invasion were investigated using a CYTOSELECT™ 24-Well Cell Invasion Assay (Cell Biolabs, San Diego, Calif., USA) by following the manufacturer's instructions. Specifically, the fibroblasts were seeded in serum-free medium containing PBS (the control), the liposomes, the liposomes loaded with YARA, or the liposomes loaded with YARA-FGF1-GFP. The treated fibroblasts were added into the upper chambers of the assay system (1×10⁶ cells/well), whereas the bottom wells were filled with the complete medium. Incubation was carried out for 48 hours at 37° C. and under 5% CO₂. The liposomes concentration in each case except the control was 0.1 mg/mL (5.8×10⁹ particles/mL). Subsequently, non-invasive fibroblasts in the upper chamber were removed from the upper inserts, and the cells that had invaded through the basement membrane were stained with cell stain solution provided in the kit for 10 min at room temperature. Subsequently, the stained cells were photographed under a brightfield microscope. Finally, the photographed inserts were transferred to an empty well filled with 200 μl extraction solution. After 10 minutes incubation on an orbital shaker, 100 μl of the samples were transferred to a 96-well microtiter plate for absorbance measurement at 560 nm by using a microplate reader (Spectramax iD5).

Statistical analysis. All the experiments were independently performed at least four times. All data are means±SD. All statistical analysis and graphical representation were performed using GraphPad Prism or SigmaStat. The statistically significant differences were assessed by one-way and two-way ANOVA, and Tukey post hoc HSD tests. p values <0.05 were considered as statistically significant (*<0.05; **<0.01; ***<0.001).

### Example 1—Cell Penetrating Peptide YARA can Carry a Fluorescent Dye Cargo into Liposomes

For peptide loading, the pre-formed pegylated remote loadable liposomes were incubated with FAM-YARA (FAM-YARAAARQARA-NH₂) (SEQ ID NO:55) at room temperature for 6 hours. After washing for three times with PBS, the liposomes were analysed via TIRF microscopy. As shown in FIGS. 1A and 1B, bright fluorescence was observed from the liposomes under the 488 nm channel, indicating that multiple copies of FAM-YARA were encapsulated into individual liposomes. Thus, a CPP (YARA) can load a small molecule dye FAM into liposomes.

### Example 2—Cell Penetrating Peptide YARA can Simultaneously Load a Dye and a Peptide into Liposomes

Peptide H (FAM-YARAAARQARAGGGGSVVIVGQIILSGR-NH₂) (SEQ ID NO:104) contains the FAM-labeled YARA peptide, a three amino acid residue linker (GGG), and a peptide inhibitor (GSVVIVGQIILSGR) (SEQ ID NO:106) which disrupts and inhibits the formation of hepatitis C (HCV) NS3/NS4A protease complex. For the peptide cargo loading, the pre-formed pegylated liposomes were mixed with Peptide H and incubated at room temperature for nearly 6 hours (Material and Methods). After washing, the liposomes loaded with Peptide H were subjected to TIRF microscopy analysis. As shown in FIGS. 2A and 2B, bright fluorescence was observed from the liposomes under the 488 nm channel, indicating that multiple copies of Peptide H were encapsulated into individual liposomes. Thus, a CPP (YARA) can simultaneously load a small molecule dye and a peptide inhibitor into liposomes.

### Example 3—Cell-Penetrating Peptide YARA is Able to Carry and Load a Protein Cargo into Liposomes

For loading, the pre-formed pegylated liposomes were mixed with the purified fusion protein YARA-FGF1-GFP and incubated overnight at room temperature (Material and Methods). The internalization of YARA-FGF1-GFP into the liposomes was evaluated using TIRF microscopy. As shown in FIGS. 3A and 3B, bright fluorescence was observed from the liposomes under the 488 nm channel, indicating that multiple copies of YARA-FGF1-GFP were encapsulated into each liposome. Thus, a CPP (YARA) can load a protein cargo into liposomes.

### Example 4—Time-Dependent Loading of a CPP-Conjugated Protein into Liposomes

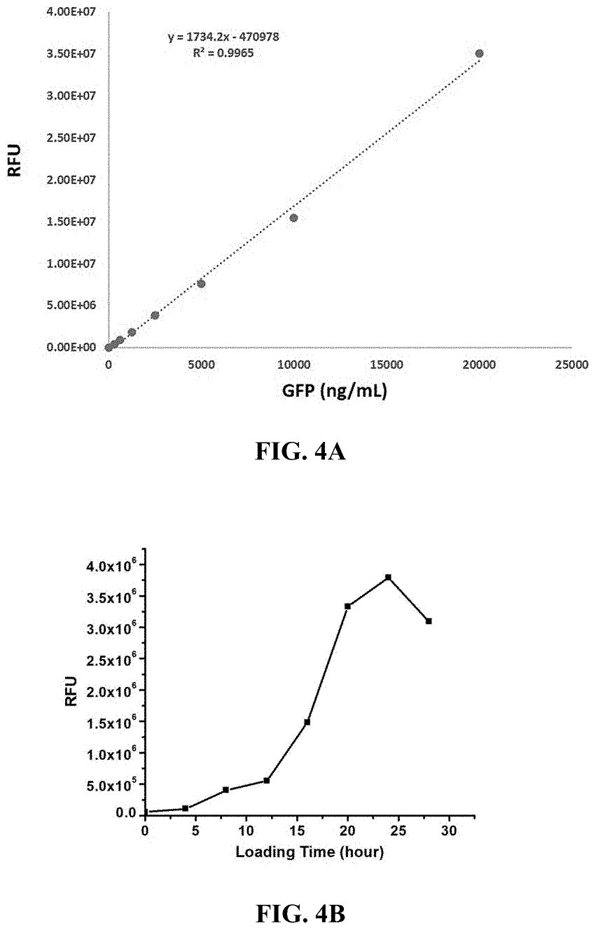

The quantity of the encapsulated fusion protein YARA-FGF1-GFP in loaded liposomes was determined by comparing the fluorescence intensity of the liposomes with that of the standard curve built with the recombinant GFP protein provided in the GFP Fluorometric Quantification Assay Kit (CELL BIOLABS, Inc., San Diego, Calif., USA). The recombinant and purified YARA-FGF1-GFP (50 μg) in PBS was added to a solution of the liposomes (0.1 mg/mL, 5.8×10⁹ particles/mL) in PBS and the mixture was incubated for 0, 4, 8, 12, 16, 20, 24, 28 hours at room temperature. The unattached YARA-FGF1-GFP was removed by washing the liposomes with PBS for three times and then filtration using Amicon Ultra-centrifugal filters (100 K device, Merck Millipore, Billerica, Mass., USA). The filtered liposomes were then resuspended in 100 μl of 1× Assay buffer/Lysis buffer. The GFP fluorescence of 100 μl samples at room temperature was measured by using a SpectraMax iD5 Multimode Microplate Reader with 485/538 nm filters. The YARA-FGF1-GFP concentration was determined from the standard curve (FIG. 4A) using the GFP Fluorometric Quantification Assay Kit. The loading of the CPP-conjugated protein (YARA-FGF1-GFP) into the liposomes was time-dependent and the maximum loading capacity was achieved after 20 hours of incubation of YARA-FGF1-GFP with the liposomes at room temperature (FIG. 4B). Interestingly, the maximum concentration of the loaded protein YARA-FGF1-GFP into the liposomes at 20 hours was calculated to be 2.2 μg/mL, corresponding to an average of 5,000 molecules of YARA-FGF1-GFP per liposome.

### Example 5—Cell-Penetrating Peptide YARA can Load a Single-Stranded DNA Cargo into Liposomes

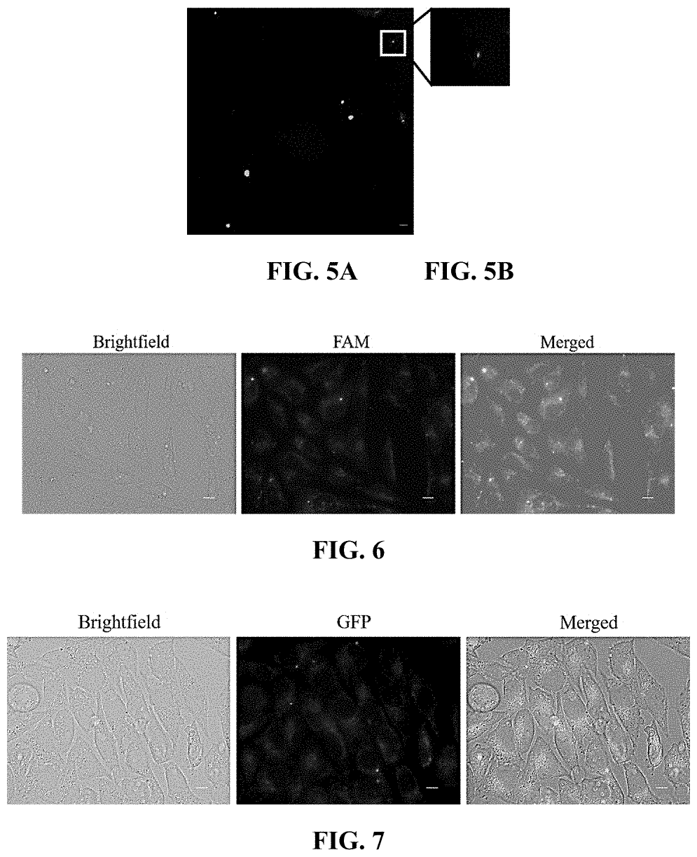

For cargo loading, the pre-formed pegylated liposomes were mixed with the purified conjugate conjugated FAM-YARA-Cys-ssDNA and incubated at room temperature for nearly 6 hours (Material and Methods). The internalization of FAM-YARA-Cys-ssDNA into the liposomes was evaluated using TIRF microscopy. As shown in FIGS. 5A and 5B, bright fluorescence was observed from the liposomes under the 488 nm channel, indicating that multiple copies of FAM-YARA-Cys-ssDNA were encapsulated into individual liposomes. Thus, a CPP (YARA) can load a single-stranded DNA cargo into liposomes.

### Example 6—Cellular Uptake of Liposomes Loaded with a Cell-Penetrating Peptide Covalently Conjugated with Both a Small Molecule Dye Cargo and a Peptide Cargo

Confocal microscopy was used to assess the internalization of the loaded liposomes by human primary dermal fibroblast cells. Briefly, fibroblast cells in a 35 mm μ-dish glass bottom culture dish were first incubated with a culture medium containing the liposomes loaded with Peptide H (FAM-YARAAARQARAGGGGSVVIVGQIILSGR-NH₂) (SEQ ID NO:104) for 4 hours at 37° C. and under 5% CO₂. The medium was then discarded and the fibroblasts were washed for three times with PBS. Image-iT fixative solution was used to fix the fibroblast cells which were then subjected to confocal microscopy measurements. The strong fluorescence signals and quite a few intense spots were observed in the cytoplasm, around and inside the nuclei of each fibroblast cell (FIG. 6), indicating that the loaded liposomes were fused with human fibroblast cells and multiple copies of Peptide H containing the CPP (YARA), the dye FAM, and the peptide (GGGGSVVIVGQIILSGR) (SEQ ID NO:107) were loaded into the fibroblast cells. Thus, employing the liposomes loaded with a fusion peptide coupled with a CPP is an efficient way to simultaneously deliver both a peptide cargo and a dye cargo into mammalian cells.

### Example 7—Cellular Uptake of the Liposomes Loaded with a Cell-Penetrating Peptide Fused with a Protein Cargo

In order to evaluate whether the liposomes loaded with a fusion protein could be taken up by human primary dermal fibroblast cells, confocal microscopy was used to assess cellular internalization of the loaded liposomes. Briefly, the fibroblast cells in a 35 mm μ-dish glass bottom culture dish were first incubated with a culture medium containing the liposomes loaded with the fusion protein YARA-FGF1-GFP for 4 hours at 37° C. and under 5% CO₂. The medium was then discarded and the fibroblasts were washed for three times with PBS. Image-iT fixative solution was used to fix the fibroblast cells which were then subjected to confocal microscopy measurements. The strong fluorescence signals and quite a few intense spots were observed in the cytoplasm, around and inside the nuclei of each fibroblast cell (FIG. 7), indicating that the loaded liposomes were fused with human fibroblast cells and multiple copies of the fusion protein cargo YARA-FGF1-GFP were loaded into individual cells. Thus, using the liposomes loaded with a CPP fused with a protein cargo is an efficient way to deliver the protein cargo into mammalian cells.

### Example 8—Liposomes Loaded with YARA-FGF1-GFP Enhance Cell Migration In Vitro

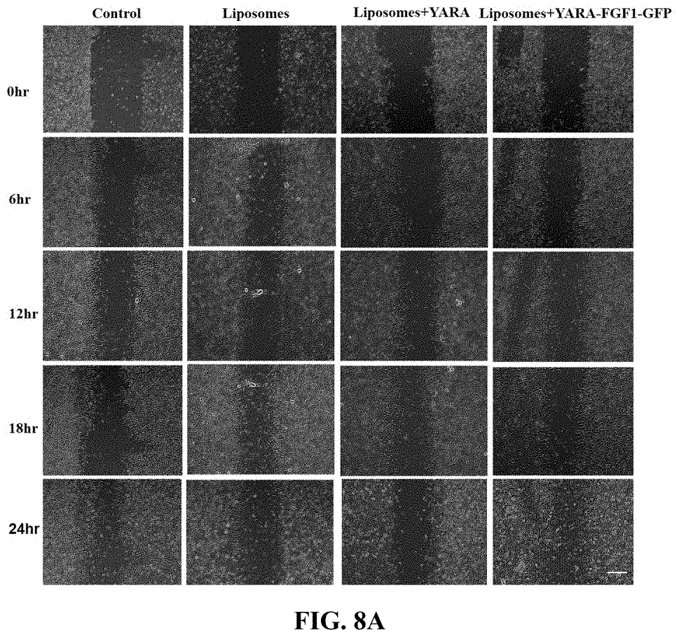

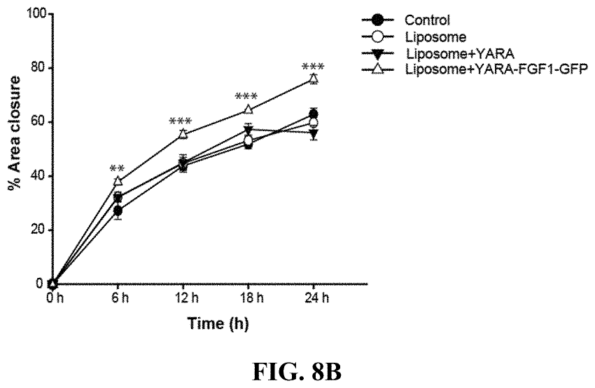

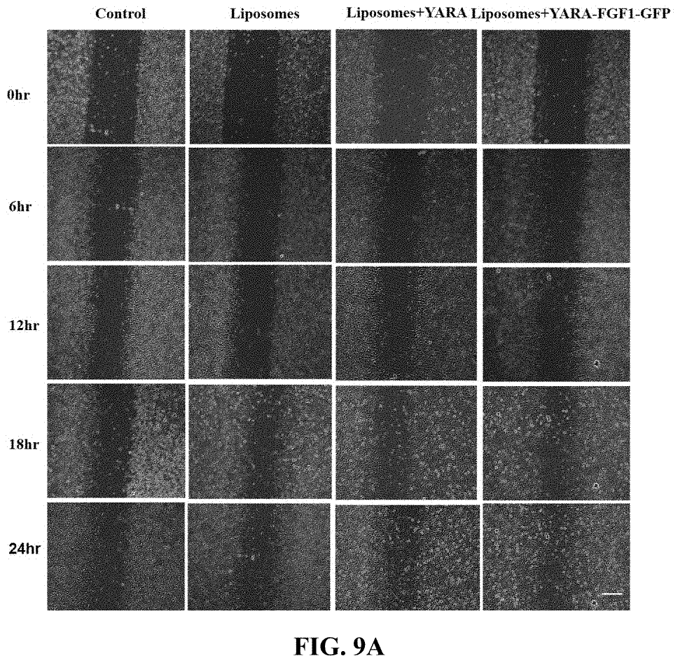

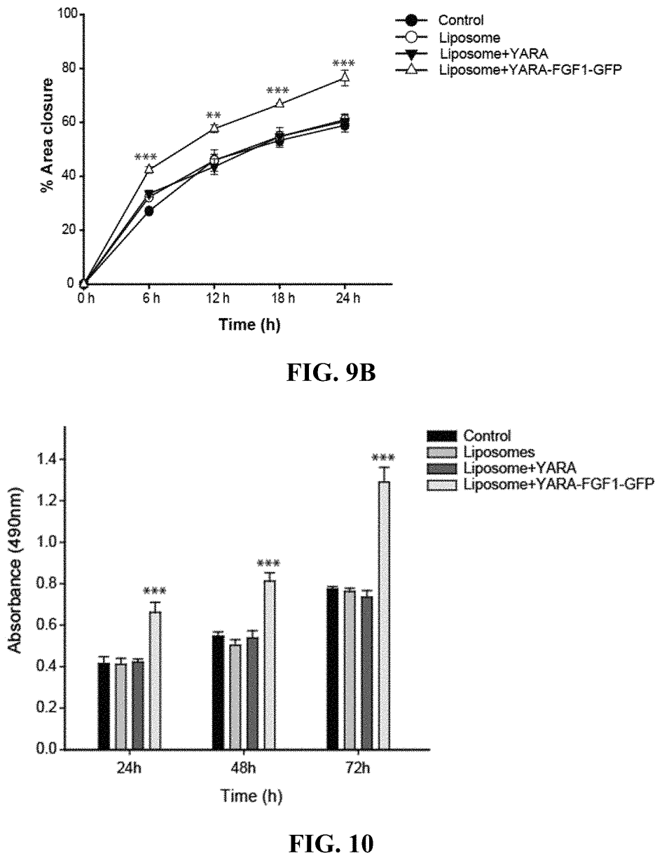

The effect of liposomes loaded with YARA-FGF1-GFP on wound healing was assessed. Two different sets of wound healing scratch assay experiments were performed using mouse embryonic fibroblasts and human primary dermal fibroblasts. In each of the experiments, the cultured fibroblasts were treated with the liposomes, the liposomes containing YARA, and the liposomes loaded with YARA-FGF1-GFP, whereas the PBS treated cells were kept as the control groups. The fibroblast migration towards the scratched (“wounded”) area was observed microscopically at 0, 6, 12, 18, and 24 hour time points. Our data showed enhanced migration rates of both cultured mouse embryonic fibroblasts and human primary dermal fibroblasts liposomes treated with the liposomes loaded with YARA-FGF1-GFP at the site of the wound in comparison with the control groups at 6, 12, 18, and 24 h time points (FIGS. 8 and 9). Moreover, in the case of mouse embryonic fibroblasts, our data showed the significant differences in the migration rates between the cells treated with the liposomes loaded with YARA-FGF1-GFP and the cells treated with either the liposomes or the liposomes containing YARA at only 12 and 24 h (FIGS. 8A and 8B). With human primary dermal fibroblasts, we also observed the significant differences in the migration rates of the cells treated with liposomes loaded with YARA-FGF1-GFP when compared to the cells treated with either the liposomes or the liposomes loaded with YARA at 6, 12, 18, and 24 h time points (FIGS. 9A and 9B). Finally, no notable differences were observed in the migration rates of both mouse embryonic fibroblasts and human primary dermal fibroblasts treated with either the liposomes, the liposomes loaded with YARA, or PBS (the control) (FIGS. 8A, 8B, 9A and 9B). The migration rate increases observed with mouse embryonic fibroblasts and human primary dermal fibroblasts treated with the liposomes loaded with YARA-FGF1-GFP relative to the treatments with the liposomes loaded with YARA, the liposomes, and PBS (control) after 24 hours are listed in Tables 5 and 6, respectively. Taken together, the internalization of the liposomes loaded with YARA-FGF1-GFP into mouse and human fibroblasts enhanced fibroblast migration. In contrast, there were no significant effects on the migration of the cells treated with either PBS (the control), the liposomes, or the liposomes loaded with YARA. These results further suggest that the positive influence on fibroblast migration were most likely attributed to the internalized fusion protein YARA-FGF1-GFP. Considering that GFP is a fluorescent marker and has no known cellular effect, and that the internalized YARA had no effect on cell migration, we conclude that the enhanced cell migration effect by the internalized YARA-FGF1-GFP via liposomes was caused by the portion of FGF1, a growth factor.

TABLE 5
Migration rate enhancement of mouse embryonic fibroblasts
treated with the liposomes loaded with YARA-FGF1-GFP
(Liposome + YARA-FGF1-GFP) relative to other treatments.

| | 24 hours |
|---|---|
| “Liposome + YARA-FGF1-GFP” | 1.094-fold |
| “the control” | |
| “Liposome + YARA-FGF1-GFP” | 1.057-fold |
| “Liposome” | |
| “Liposome + YARA-FGF1-GFP” | 1.099-fold |
| “Liposome + YARA” | |

TABLE 6
Migration rate enhancement of human primary dermal fibroblasts
treated with “Liposome + YARA-FGF1-GFP”
relative to other treatments.

| | 24 hours |
|---|---|
| “Liposome + YARA-FGF1-GFP” | 1.085-fold |
| “the control” | |
| “Liposome + YARA-FGF1-GFP” | 1.042-fold |
| “Liposome” | |
| “Liposome + YARA-FGF1-GFP” | 1.139-fold |
| “Liposome + YARA” | |

### Example 9—Liposomes Loaded with YARA-FGF1-GFP Enhanced Cell Proliferation

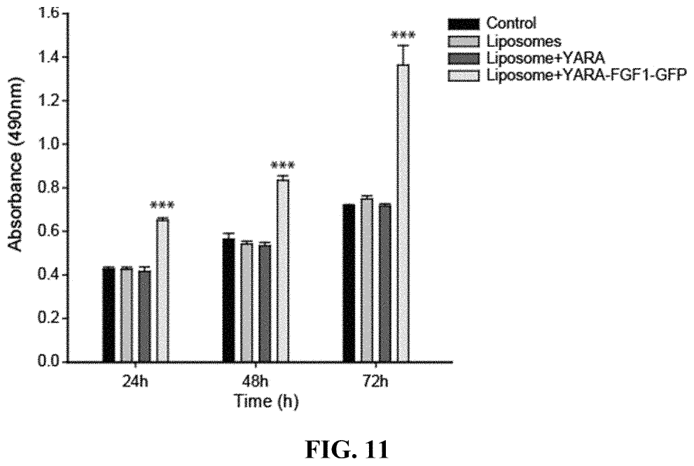

Increasing evidence demonstrates the importance of fibroblast proliferation during wound healing from the late inflammatory stage until the healing process of the injured tissue. Therefore, we analyzed fibroblast proliferation by colorimetric MTS proliferation assay using either mouse embryonic fibroblasts or human primary dermal fibroblasts. In each of the experiments, both mouse embryonic fibroblasts and human primary dermal fibroblast cells were treated with PBS (the control), the liposomes, the liposomes loaded with YARA, or the liposomes loaded with YARA-FGF1-GFP and the effect of these external factors on fibroblast proliferation was measured at various time points (24, 48, and 72 h). Interestingly, the proliferation of both mouse embryonic fibroblasts and human primary dermal fibroblasts treated with the liposomes loaded with YARA-FGF1-GFP increased significantly when compared to their respective control groups at 24, 48, and 72 h (FIGS. 10 and 11). In comparison, no significant differences in fibroblast proliferation were observed among the treatments with PBS (the control), the liposomes, and the liposomes loaded with YARA (FIGS. 10 and 11). The proliferation enhancement of the fibroblasts treated with the liposomes loaded with YARA-FGF1-GFP relative to the treatments with PBS (the control), the liposomes, and the liposomes loaded with YARA is given in Tables 7 and 8. Collectively, our experiments demonstrated that the internalization of the liposomes loaded with YARA-FGF1-GFP into the fibroblasts had a positive effect on fibroblast proliferation. Considering that the liposomes alone and the liposomes loaded with YARA had little effect on fibroblast proliferation, and that GFP is a fluorescent marker and has no known cellular effect, we conclude that the fibroblast proliferation enhancement effect of the internalized YARA-FGF1-GFP was most likely due to FGF1 (a known growth factor) in the fusion protein.

TABLE 7
Proliferation rate enhancement of mouse embryonic fibroblasts
treated with “Liposome + YARA-FGF1-GFP” relative
to other treatments.

| | 24 hours | 48 hours | 72 hours |
|---|---|---|---|
| “Liposome + YARA-FGF1-GFP” | 1.5-fold | 1.4-fold | 1.6-fold |
| “the control” | | | |
| “Liposome + YARA-FGF1-GFP” | 1.6-fold | 1.6-fold | 1.6-fold |
| “Liposome” | | | |
| “Liposome + YARA-FGF1-GFP” | 1.5-fold | 1.5-fold | 1.7-fold |
| “Liposome + YARA” | | | |

TABLE 8
Proliferation rate enhancement of human primary dermal
fibroblasts treated with “Liposome + YARA-FGF1-
GFP” relative to other treatments.

| | 24 hours | 48 hours | 72 hours |
|---|---|---|---|
| “Liposome + YARA-FGF1-GFP” | 1.5-fold | 1.4-fold | 1.9-fold |
| “the control” | | | |
| “Liposome + YARA-FGF1-GFP” | 1.5-fold | 1.5-fold | 1.8-fold |
| “Liposome” | | | |
| “Liposome + YARA-FGF1-GFP” | 1.5-fold | 1.5-fold | 1.9-fold |
| “Liposome + YARA” | | | |

### Example 10—Liposomes Loaded with YARA-FGF1-GFP Promote Cell Invasion

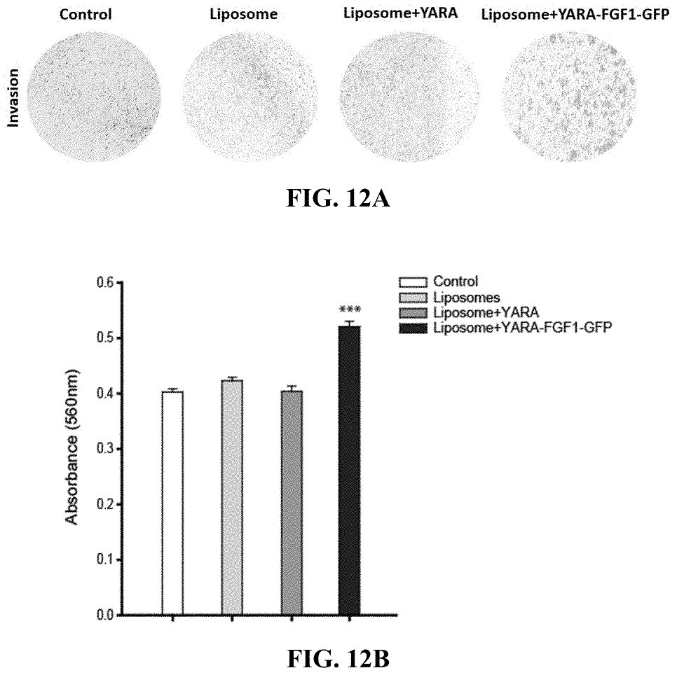

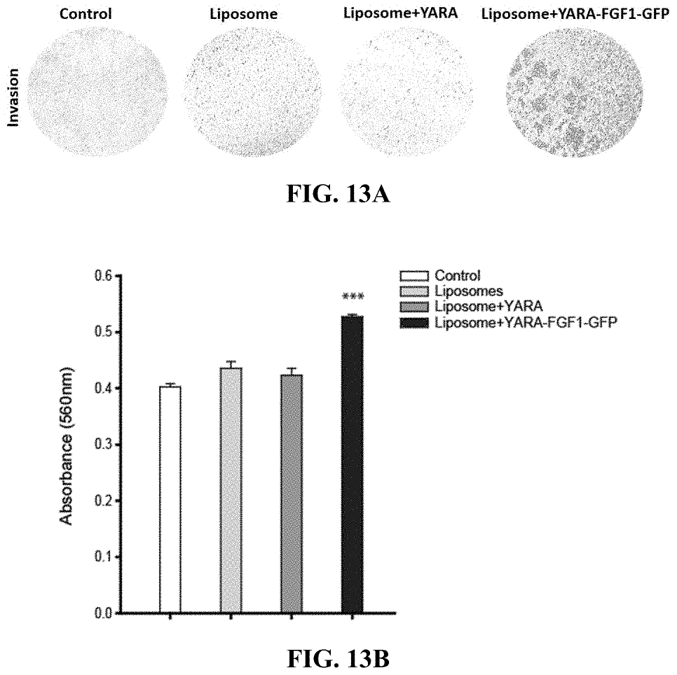

Cell invasion assays were performed to check the effect of the liposomes loaded with YARA-FGF1-GFP on the invasion of mouse embryonic and human primary dermal fibroblasts using colorimetric transwell invasion assay. Treatment of mouse embryonic fibroblasts with the liposomes loaded with YARA-FGF1-GFP for 48 h increased cell invasion relative to the treatment with the liposomes, the liposomes loaded with YARA, or PBS (the control) (FIGS. 12A and 12B). Similarly, the treatment with the liposomes loaded with YARA-FGF1-GFP for 48 h enhanced the invasion of human primary dermal fibroblasts compared to the treatment with the liposomes, the liposomes loaded with YARA, or PBS (FIGS. 13A and 13B). The fibroblast invasion enhancement with the treatment of the liposomes loaded with YARA-FGF1-GFP relative to other treatments is given in Tables 9 and 10. Together, these experiments indicate that the internalization of the liposomes loaded with YARA-FGF1-GFP had major impact on the invasion of the fibroblasts while the internalization of the liposomes alone or the liposomes loaded with YARA had no effect. Since GFP, a fluorescent marker, is not known to cause any cellular effect, and since the internalized YARA in the fibroblasts did not cause any cell invasion impact, the observed favorable effect on fibroblast invasion was most likely due to the FGF1, a growth factor, within the internalized fusion protein YARA-FGF1-GFP.

TABLE 9
Invasion rate of mouse embryonic fibroblasts
treated with “Liposome + YARA-FGF1-GFP”
relative to other treatments.

| | 48 hours |
|---|---|
| “Liposome + YARA-FGF1-GFP” | 1.3-fold |
| “the control” | |
| “Liposome + YARA-FGF1-GFP” | 1.2-fold |
| “Liposome” | |
| “Liposome + YARA-FGF1-GFP” | 1.3-fold |
| “Liposome + YARA” | |

TABLE 10
Invasion rate of human primary dermal fibroblasts
treated with “Liposome + YARA-FGF1-
GFP” relative to other treatments.

| | 48 hours |
|---|---|
| “Liposome + YARA-FGF1-GFP” | 1.3-fold |
| “the control” | |
| “Liposome + YARA-FGF1-GFP” | 1.2-fold |
| “Liposome” | |
| “Liposome + YARA-FGF1-GFP” | 1.2-fold |
| “Liposome + YARA” | |

TABLE 11
Examples of Cell-Penetrating Polypeptides (from Table S1 of Behzadipour Y and S Hemmati
Molecules, 2019, 24:4318)

| | SEQ ID | | | Prediction | Uptake | Prediction |
|---|---|---|---|---|---|---|
| CPPs' name | NO | Amino acid sequence | Cell-Penetrating or not | Confidence* | Efficiency | Confidence** |
| PAF95 | 108 | AAAWFW | Cell-penetrating | 0.69 | Low | 0.68 |
| PN225 | 109 | AAVACRICMRNFSTRQARRNHRRRHRR | Cell-penetrating | 0.89 | High | 0.6 |
| MPS | 110 | AAVALLPAVLLALLAK | Cell-penetrating | 0.84 | High | 0.55 |
| MPS-Galphai2 | 111 | AAVALLPAVLLALLAKKNNLKDCGLF | Cell-penetrating | 0.91 | High | 0.55 |
| MPS-Galphai3 | 112 | AAVALLPAVLLALLAKKNNLKECGLY | Cell-penetrating | 0.85 | Low | 0.54 |
| MTS | 113 | AAVALLPAVLLALLAP | Cell-penetrating | 0.85 | Low | 0.843 |
| SKP | 114 | AAVALLPAVLLALLAPEILLPNNYNAYESYK | Cell-penetrating | 0.85 | Low | 0.59 |
| | | YPGMFIALSK | | | | |
| PN227 | 115 | AAVALLPAVLLALLAPRKKRRQRRRPPQ | Cell-penetrating | 0.99 | Low | 0.503 |
| PN27 | 116 | AAVALLPAVLLALLAPRKKRRQRRRPPQC | Cell-penetrating | 0.99 | High | 0.508 |
| PN365 | 117 | AAVALLPAVLLALLAPRRRRRR | Cell-penetrating | 0.96 | High | 0.57 |
| PN29 | 118 | AAVALLPAVLLALLAPSGASGLDKRDYV | Cell-penetrating | 0.91 | Low | 0.68 |
| SN50 | 119 | AAVALLPAVLLALLAPVQRKRQKLMP | Cell-penetrating | 0.98 | High | 0.53 |
| Anti- | 120 | AAVALLPAVLLALLAVTDQLGEDFFAVDLEA | Cell-penetrating | 0.83 | Low | 0.55 |
| BetaGamma | | FLQEFGLLPEKE | | | | |
| IA6d | 121 | ACGRGRGRCGRGRGRCG | Cell-penetrating | 1 | Low | 0.602 |
| IA6b | 122 | ACGRGRGRCRGRGRGCG | Cell-penetrating | 1 | Low | 0.652 |
| IA5_2H1W | 123 | ACHGRRWGCGRHRGRCG | Cell-penetrating | 0.98 | Low | 0.52 |
| kCA3 | 124 | ACRDRFRNCPADEALCG | Non-cell-penetrating | 0.53 | — | — |
| kCA4 | 125 | ACRDRFRNCPADERLCG | Cell-penetrating | 0.66 | Low | 0.675 |
| kCA5 | 126 | ACRDRFRRCPADERLCG | Cell-penetrating | 0.87 | Low | 0.62 |
| kCA6 | 127 | ACRDRFRRCPADRRLCG | Cell-penetrating | 0.88 | Low | 0.613 |
| IA6a | 128 | ACRGRGRGCGRGRGRCG | Cell-penetrating | 1 | Low | 0.61 |
| CA3 | 129 | ACRGRGRGCGSGSGSCG | Cell-penetrating | 0.86 | Low | 0.73 |
| CA4 | 130 | ACRGRGRGCGSGSRSCG | Cell-penetrating | 0.99 | Low | 0.7 |
| IA6c | 131 | ACRGRGRGCRGRGRGCG | Cell-penetrating | 1 | Low | 0.68 |
| CA6 | 132 | ACRGRGRRCGSGRRSCG | Cell-penetrating | 0.99 | Low | 0.66 |
| CA5 | 133 | ACRGRGRRCGSGSRSCG | Cell-penetrating | 1 | Low | 0.69 |
| IA8a | 134 | ACRGRRRGCGRRRGRCG | Cell-penetrating | 0.99 | Low | 0.508 |
| IA4a | 135 | ACRGSGRGCGRGSGRCG | Cell-penetrating | 0.99 | Low | 0.685 |
| IA8b L (Linear | 136 | ACRRSRRGCGRRSRRCG | Cell-penetrating | 0.99 | Low | 0.57 |
| variants) | | | | | | |
| kCA2 | 137 | ACSDRFRNCPADEALCG | Non-cell-penetrating | 0.63 | — | — |
| (Kallikrein | | | | | | |
| inhibitor with | | | | | | |
| internal | | | | | | |
| arginines) | | | | | | |
| kEA1 8 | 138 | ACSDRFRNCPADEALCGRRRRRRRR | Cell-penetrating | 0.86 | Low | 0.6 |
| IA4b | 139 | ACSGRGRGCGRGRGSCG | Cell-penetrating | 0.97 | Low | 0.695 |
| CA2 (Control | 140 | ACSGRGRGCGSGSGSCG | Cell-penetrating | 0.9 | Low | 0.79 |
| internal | | | | | | |
| arginine) | | | | | | |
| IA2 | 141 | ACSGRGSGCGSGRGSCG | Cell-penetrating | 0.95 | Low | 0.785 |
| IA0 (Bicyclic) | 142 | ACSGSGSGCGSGSGSCG | Cell-penetrating | 0.84 | Low | 0.68 |
| (integral | | | | | | |
| arginine | | | | | | |
| peptides) | | | | | | |
| EA1x8 L | 143 | ACSGSGSGCGSGSGSCGRRRRRRRR | Cell-penetrating | 0.96 | Low | 0.66 |
| EA8_4H | 144 | ACSHSGHGCGHGSHSCGRRRRRRRR | Cell-penetrating | 0.98 | Low | 0.7 |
| (Histidine/ | | | | | | |
| tryptophan | | | | | | |
| peptides) | | | | | | |
| EA8_2H2W | 145 | ACSHSGWGCGHGSWSCGRRRRRRRR | Cell-penetrating | 0.94 | Low | 0.7 |
| F4 | 146 | ACSSSPSKHCG | Cell-penetrating | 0.7 | Low | 0.705 |
| B1 | 147 | ACSSSPSKHCGGGGRRRRRRRRR | Cell-penetrating | 0.98 | Low | 0.59 |
| Inv9 | 148 | ADVFDRGGPYLQRGVADLVPTATLLDTYSP | Cell-penetrating | 0.79 | Low | 0.93 |
| C11 | 149 | AEAEAEAEAKAKAKAK | Cell-penetrating | 0.92 | Low | 0.71 |
| A9 | 150 | AEAEAEAEAKAKAKAKAGGGHRRRRRRR | Cell-penetrating | 0.99 | Low | 0.6 |
| Inv5 | 151 | AEKVDPVKLNLTLSAAAEALTGLGDK | Cell-penetrating | 0.87 | High | 0.72 |
| TH peptide | 152 | AGYLLGHINLHHLAHLHHIL | Cell-penetrating | 0.84 | Low | 0.59 |
| TH peptide | 153 | AGYLLGHINLHHLAHLHHILC | Cell-penetrating | 0.89 | Low | 0.54 |
| Transportan 10 | 154 | AGYLLGKINLKALAALAKKIL | Cell-penetrating | 0.98 | High | 1 |
| (TP10) | | | | | | |
| Transportan 10 | 155 | AGYLLGKINLKALAALAKKILGGC | Cell-penetrating | 0.93 | High | 0.6 |
| Transportan- | 156 | AGYLLGKINLKALAALAKKILTYADFIASGRT | Cell-penetrating | 0.94 | High | 0.76 |
| PKI | | GRRNAI | | | | |
| TK peptide | 157 | AGYLLGKINLKKLAKLLLIL | Cell-penetrating | 0.95 | Low | 0.54 |
| TP14 | 158 | AGYLLGKLKALAALAKKIL | Cell-penetrating | 0.98 | Low | 0.74 |
| NF1 | 159 | AGYLLGKTNLKALAALAKKIL | Cell-penetrating | 0.97 | High | 0.63 |
| pAntpHD | 160 | AHALCLTERQIKIWFQNRRMKWKKEN | Cell-penetrating | 0.82 | High | 0.527 |
| pAntpHD 40P2 | 161 | AHALCPPERQIKIWFQNRRMKWKKEN | Cell-penetrating | 0.72 | High | 0.5 |
| TCTP(1-9) | 162 | AIIYRDLIS | Non-cell-penetrating | 0.66 | — | — |
| M1A | | | | | | |
| subsetution | | | | | | |
| mutant | | | | | | |
| Peptide 49 | 163 | AIPNNQLGFPFK | Cell-penetrating | 0.82 | Low | 0.59 |
| 30 A-K | 164 | AKKAKAAKKAKAAKKAKAAKKAKAAKKA | Cell-penetrating | 1 | Low | 0.662 |
| | | KA | | | | |
| 24 A-K | 165 | AKKKAAKAAKKKAAKAAKKKAAKA | Cell-penetrating | 1 | Low | 0.7 |
| 32 A-K | 166 | AKKKAAKAAKKKAAKAAKKKAAKAAKKK | Cell-penetrating | 1 | Low | 0.71 |
| | | AAKA | | | | |
| Ala49 | 167 | AKKRRQRRR | Cell-penetrating | 1 | Low | 0.83 |
| substitution | | | | | | |
| mutant of Tat | | | | | | |
| (49-57) | | | | | | |
| MTat2-Nat | 168 | AKKRRQRRRAKKRRQRRR | Cell-penetrating | 1 | Low | 0.55 |
| F3 | 169 | AKVKDEPQRRSARLSAKPAPPKPEPKPKKAP | Cell-penetrating | 0.94 | Low | 0.69 |
| | | AKK | | | | |
| D5 | 170 | ALALALALALALALALKIKKIKKIKKIKKLAK | Cell-penetrating | 1 | High | 0.57 |
| | | LAKKIK | | | | |
| pVEC mutant | 171 | ALIILRRRIRKQAHAHSK | Cell-penetrating | 0.99 | Low | 0.96 |
| S4(13) | 172 | ALWKTLLKKVLKA | Cell-penetrating | 0.98 | High | 0.51 |
| S4(13)-PV | 173 | ALWKTLLKKVLKAPKKKRKV | Cell-penetrating | 0.98 | High | 0.52 |
| No.14-11 | 174 | ALWMRWYSPTTRRYG | Cell-penetrating | 0.8 | Low | 0.78 |
| Dermaseptin | 175 | ALWMTLLKKVLKAAAKAALNAVLVGANA | Cell-penetrating | 0.93 | Low | 0.62 |
| S4 | | | | | | |
| CTP (cardiac | 176 | APWHLSSQYSRT | Cell-penetrating | 0.84 | Low | 0.75 |
| targetting | | | | | | |
| peptide) | | | | | | |
| Ala43 | 177 | AQIKIWFQNRRMKWKK | Cell-penetrating | 0.95 | High | 0.962 |
| substitution | | | | | | |
| mutant of | | | | | | |
| pAntp (43-58) | | | | | | |
| kEA2x1 | 178 | ARCSDRFRNCPADEALCGR | Cell-penetrating | 0.57 | Low | 0.655 |
| (Kallikrein | | | | | | |
| inhibitor with | | | | | | |
| external | | | | | | |
| arginines) | | | | | | |
| EA2x1 | 179 | ARCSGSGSGCGSGSGSCGR | Cell-penetrating | 0.9 | Low | 0.66 |
| (External | | | | | | |
| arginines) | | | | | | |
| 30 A-R | 180 | ARRARAARRARAARRARAARRARAARRAR | Cell-penetrating | 1 | Low | 0.651 |
| | | A | | | | |
| kEA2x2 | 181 | ARRCSDRFRNCPADEALCGRR | Cell-penetrating | 0.69 | Low | 0.595 |
| EA2x2 | 182 | ARRCSGSGSGCGSGSGSCGRR | Cell-penetrating | 0.89 | Low | 0.69 |
| 24 A-R | 183 | ARRRAARAARRRAARAARRRAARA | Cell-penetrating | 1 | Low | 0.689 |
| 32 A-R | 184 | ARRRAARAARRRAARAARRRAARAARRRA | Cell-penetrating | 1 | Low | 0.699 |
| | | ARA | | | | |
| kEA2x3 | 185 | ARRRCSDRFRNCPADEALCGRRR | Cell-penetrating | 0.84 | High | 0.56 |
| EA2x3 | 186 | ARRRCSGSGSGCGSGSGSCGRRR | Cell-penetrating | 0.96 | Low | 0.63 |
| kEA2x4 | 187 | ARRRRCSDRFRNCPADEALCGRRRR | Cell-penetrating | 0.91 | High | 0.53 |
| EA2x4 | 188 | ARRRRCSGSGSGCGSGSGSCGRRRR | Cell-penetrating | 0.98 | Low | 0.66 |
| Inv8 | 189 | ARTINAQQAELDSALLAAAGFGNTTADVFDR | Cell-penetrating | 0.89 | Low | 0.86 |
| | | G | | | | |
| FHV gamma | 190 | ASMWERVKSIIKSSLAAASNI | Cell-penetrating | 0.74 | Low | 0.64 |
| peptide | | | | | | |
| Peptide 26 | 191 | AVPAENALNNPF | Cell-penetrating | 0.85 | Low | 0.695 |
| pAntpHD 50A | 192 | AYALCLTERQIKIWFANRRMKWKKEN | Cell-penetrating | 0.67 | High | 0.51 |
| TAT-cysteine | 193 | AYGRKKRRQRRR | Cell-penetrating | 1 | Low | 0.525 |
| peptide | | | | | | |
| TP10 | 194 | AYLLGKINLKALAALAKKIL | Cell-penetrating | 0.97 | High | 0.7 |
| L1 (Ala32 | 195 | AYRIKPTFRRLKWKYKGKFW | Cell-penetrating | 0.98 | High | 0.567 |
| substitution | | | | | | |
| mutant of | | | | | | |
| LALF (32-51)) | | | | | | |
| CAR | 196 | CARSKNKDC | Cell-penetrating | 0.6 | Low | 0.662 |
| Peptide 2 | 197 | CASGQQGLLKLC | Cell-penetrating | 0.96 | Low | 0.69 |
| S-TAT | 198 | CAYGGQQGGQGGG | Cell-penetrating | 0.89 | Low | 0.69 |
| PTX-TAT-LP | 199 | CAYGRKKRRQRRR | Cell-penetrating | 1 | Low | 0.533 |
| TAT | 200 | CCTGRKKRRQRRR | Cell-penetrating | 0.98 | High | 0.64 |
| Alexa488- | 201 | CELAGIGILTVKKKKKQKKK | Cell-penetrating | 0.96 | Low | 0.753 |
| Melan-A- | | | | | | |
| polyLys | | | | | | |
| (control | | | | | | |
| peptide) | | | | | | |
| Alexa488- | 202 | CELAGIGILTVRKKRRQRRR | Cell-penetrating | 0.96 | Low | 0.603 |
| Melan-A-TAT | | | | | | |
| DPV15b | 203 | CGAYDLRRRERQSRLRRRERQSR | Cell-penetrating | 0.81 | Low | 0.727 |
| POD | 204 | CGGGARKKAAKAARKKAAKAARKKAAKA | Cell-penetrating | 1 | Low | 0.665 |
| | | ARKKAAKA | | | | |
| TAT | 205 | CGGGGYGRKKRRQRRR | Cell-penetrating | 0.98 | High | 0.537 |
| sgRNA-CPP | 206 | CGGGRRRRRRRRRLLLL | Cell-penetrating | 1 | High | 0.514 |
| AgNP-TAT | 207 | CGGGYGRKKRRQRRR | Cell-penetrating | 0.99 | High | 0.604 |
| b-WT1-pTj | 208 | CGGKDCERRFSRSDQLKRHQRRHTGVKPFQ | Cell-penetrating | 0.88 | Low | 0.515 |
| M918(C-S) | 209 | CGGMVTVLFRRLRIRRASGPPRVRV | Cell-penetrating | 0.95 | High | 0.72 |
| tLyp-1 | 210 | CGNKRTR | Cell-penetrating | 0.86 | Low | 0.52 |
| Lyp-1 | 211 | CGNKRTRGC | Cell-penetrating | 0.82 | Low | 0.523 |
| IX | 212 | CGRKKRAARQRAARAARPPQ | Cell-penetrating | 1 | Low | 0.696 |
| VI | 213 | CGRKKRAARQRRRPPQ | Cell-penetrating | 0.97 | High | 0.595 |
| XIII | 214 | CGRKKRLLRQRLLRLLRPPQ | Cell-penetrating | 0.99 | Low | 0.592 |
| X | 215 | CGRKKRLLRQRRRPPQ | Cell-penetrating | 0.99 | High | 0.623 |
| VIII | 216 | CGRKKRRQRAARRPPQ | Cell-penetrating | 0.96 | High | 0.61 |
| XII | 217 | CGRKKRRQRLLRRPPQ | Cell-penetrating | 0.98 | High | 0.593 |
| VII | 218 | CGRKKRRQRRAARPPQ | Cell-penetrating | 0.96 | High | 0.61 |
| XI | 219 | CGRKKRRQRRLLRPPQ | Cell-penetrating | 0.98 | High | 0.593 |
| C16NTD | 220 | CGRKKRRQRRRPPQ | Cell-penetrating | 0.97 | High | 0.797 |
| III | 221 | CGRKKRRQRRWWRPPQ | Cell-penetrating | 0.98 | High | 0.725 |
| IV | 222 | CGRKKRRQRWWRRPPQ | Cell-penetrating | 0.98 | High | 0.705 |
| II | 223 | CGRKKRWWRQRRRPPQ | Cell-penetrating | 0.99 | High | 0.745 |
| V | 224 | CGRKKRWWRQRWWRWWRPPQ | Cell-penetrating | 0.99 | High | 0.677 |
| TAT | 225 | CGYGRKKRRQRRRGC | Cell-penetrating | 0.98 | High | 0.532 |
| T7-LP | 226 | CHAIYPRH | Cell-penetrating | 0.57 | Low | 0.55 |
| HR9 | 227 | CHHHHHRRRRRRRRRHHHHHC | Cell-penetrating | 0.99 | High | 0.579 |
| CH2 R4 H2 C | 228 | CHHRRRRHHC | Cell-penetrating | 0.93 | High | 0.583 |
| Melittin | 229 | CIGAVLKVLTTGLPALISWIKRKRQQ | Cell-penetrating | 0.85 | High | 0.555 |
| TCTP-CPP 6 | 230 | CIISRDLISH | Non-cell-penetrating | 0.65 | — | — |
| F3 Peptide | 231 | CKDEPQRRSARLSAKPAPPKPEPKPKKAPAK | Cell-penetrating | 0.85 | Low | 0.68 |
| | | K | | | | |
| ck9 | 232 | ckkkkkkkkk | Cell-penetrating | 0.97 | Low | 0.64 |
| acFTAT | 233 | CKYGRKKRRQRRR | Cell-penetrating | 0.99 | High | 0.543 |
| Dox-pVEC- | 234 | CLLIILRRRIRKQAHAHSKNHQQQNPHQPPM | Cell-penetrating | 0.88 | Low | 0.53 |
| gHo (Dox- | | | | | | |
| gHoPe2) | | | | | | |
| Mgpe-10 | 235 | CLLYWFRRRHRFIHRRRHRRC | Cell-penetrating | 0.99 | High | 0.575 |
| NGR | 236 | CNGRC | Cell-penetrating | 0.54 | Low | 0.59 |
| Crot (27-39) | 237 | CRFRFKCCKK | Cell-penetrating | 0.96 | High | 0.98 |
| derevative | | | | | | |
| Crot (27-39) | 238 | CRFRWKCCKK | Cell-penetrating | 0.96 | High | 0.99 |
| derevative | | | | | | |
| RGD | 239 | CRGDC | Non-cell-penetrating | 0.54 | — | — |
| CRGDK | 240 | CRGDK | Cell-penetrating | 0.71 | Low | 0.69 |
| iRGD | 241 | CRGDKGDPC | Cell-penetrating | 0.54 | Low | 0.73 |
| iRGD-CDD | 242 | CRGDKGPDC | Cell-penetrating | 0.51 | Low | 0.71 |
| D-TAT | 243 | CRKARYRGRKRQR | Cell-penetrating | 1 | Low | 0.553 |
| iNGR | 244 | CRNGRGPDC | Cell-penetrating | 0.59 | Low | 0.71 |
| Reduced linear | 245 | CRQIKIWFPNRRMKWKKC | Cell-penetrating | 0.87 | High | 0.718 |
| penetratin | | | | | | |
| Penetratin | 246 | CRQIKIWFQNRRMKWKK | Cell-penetrating | 0.97 | High | 0.589 |
| KLA-Pen | 247 | CRQIKIWFQNRRMKWKKKLAKLAKKLAKLA | Cell-penetrating | 0.97 | High | 0.56 |
| | | K | | | | |
| Mgpe-9 | 248 | CRRLRHLRHHYRRRWHRFRC | Cell-penetrating | 0.99 | High | 0.562 |
| R8 | 249 | CRRRRRRRR | Cell-penetrating | 1 | High | 0.565 |
| Crot (27-39) | 250 | CRWRFKCCKK | Cell-penetrating | 0.96 | High | 1 |
| derevative | | | | | | |
| CyLoP-1 | 251 | CRWRWKCCKK | Cell-penetrating | 0.95 | High | 1 |
| Crot (27-39) | 252 | CRWRWKCG | Cell-penetrating | 0.8 | High | 0.88 |
| derevative | | | | | | |
| Crot (27-39) | 253 | CRWRWKCGCKK | Cell-penetrating | 0.92 | High | 0.99 |
| derevative | | | | | | |
| Crot (27-39) | 254 | CRWRWKCSKK | Cell-penetrating | 0.94 | High | 0.86 |
| derevative | | | | | | |
| Crot (27-39) | 255 | CRWRWKSSKK | Cell-penetrating | 0.95 | Low | 0.89 |
| derevative | | | | | | |
| C105Y | 256 | CSIPPEVKFNKPFVYLI | Cell-penetrating | 0.65 | Low | 0.605 |
| C105Y | 257 | CSIPPEVKFNPFVYLI | Non-cell-penetrating | 0.61 | — | — |
| CSK | 258 | CSKSSDYQC | Non-cell-penetrating | 0.63 | — | — |
| 1A | 259 | CSSLDEPGRGGFSSESKV | Cell-penetrating | 0.81 | Low | 0.827 |
| LI | 260 | CTSTTAKRKKRKLK | Cell-penetrating | 0.97 | Low | 0.665 |
| Peptide 1- | 261 | CTWLKY | Cell-penetrating | 0.6 | High | 0.55 |
| NTHSΔ | | | | | | |
| Peptide 1- | 262 | CTWLKYH | Cell-penetrating | 0.54 | Low | 0.51 |
| NTSΔ | | | | | | |
| DPV1048 | 263 | CVKRGLKLRHVRPRVTRDV | Cell-penetrating | 0.83 | Low | 0.615 |
| S41 | 264 | CVQWSLLRGYQPC | Cell-penetrating | 0.76 | Low | 0.627 |
| LMWP | 265 | CVSRRRRRRGGRRRR | Cell-penetrating | 0.98 | High | 0.55 |
| AlkCWK3 | 266 | CWKKK | Cell-penetrating | 0.83 | High | 0.565 |
| AlkCWK8 | 267 | CWKKKKKKKK | Cell-penetrating | 0.97 | Low | 0.61 |
| AlkCWK13 | 268 | CWKKKKKKKKKKKKK | Cell-penetrating | 0.98 | Low | 0.58 |
| AlkCWK18 | 269 | CWKKKKKKKKKKKKKKKKKK | Cell-penetrating | 0.98 | Low | 0.64 |
| PTX-N-TAT- | 270 | CYGRKKRRQRRR | Cell-penetrating | 1 | High | 0.561 |
| LP | | | | | | |
| EGFP-VP_22 | 271 | DAATARGRGRSAASRPTERPRAPARSASRPR | Cell-penetrating | 0.96 | Low | 0.785 |
| | | RPVD | | | | |
| VP22 | 272 | DAATATRGRSAASRPTQRPRAPARSASRPRR | Cell-penetrating | 0.95 | Low | 0.76 |
| | | PVE | | | | |
| Crot (27-39) | 273 | DCRWRWKCCKK | Cell-penetrating | 0.82 | High | 0.99 |
| derivative | | | | | | |
| hCT(15â€“32) | 274 | DFNKFHTFPQTAIGVGAP | Non-cell-penetrating | 0.63 | — | — |
| rV1aR (102- | 275 | DITYRFRGPDWL | Cell-penetrating | 0.79 | Low | 0.72 |
| 113a) | | | | | | |
| Peptide 52 | 276 | DPATNPGPHFPR | Cell-penetrating | 0.82 | Low | 0.69 |
| VT5 | 277 | DPKGDPKGVTVTVTVTVTGKGDPKPD | Cell-penetrating | 0.86 | Low | 0.765 |
| Secretory | 278 | DPVDTPNPTRRKPGK | Cell-penetrating | 0.88 | Low | 0.61 |
| leukoprotease | | | | | | |
| inhibitor | | | | | | |
| derived PTD | | | | | | |
| Unknown | 279 | DRDDRDDRDDRDDRDDR | Cell-penetrating | 0.9 | Low | 0.615 |
| Unknown | 280 | DRDRDRDRDR | Cell-penetrating | 0.91 | Low | 0.705 |
| RSG 1.2 | 281 | DRRRRGSRPSGAERRRR | Cell-penetrating | 0.93 | Low | 0.615 |
| truncated | | | | | | |
| RSG 1.2 | 282 | DRRRRGSRPSGAERRRRRAAAA | Cell-penetrating | 0.98 | Low | 0.642 |
| 2 | 283 | DSLKSYWYLQKFSWR | Cell-penetrating | 0.79 | High | 0.78 |
| C45D18 | 284 | DTWAGVEAIIRILQQLLFIHFR | Cell-penetrating | 0.74 | Low | 0.57 |
| GV1001 | 285 | EARPALLTSRLRFIPK | Cell-penetrating | 0.89 | Low | 0.68 |
| Peptide 4 | 286 | ECYPKKGQDP | Non-cell-penetrating | 0.69 | — | — |
| Glu | | EEE | Cell-penetrating | 0.71 | Low | 0.63 |
| Glu-Ala | 287 | EEEAA | Cell-penetrating | 0.69 | Low | 0.88 |
| Glu-Oct-6 | 288 | EEEAAGRKRKKRT | Cell-penetrating | 0.97 | High | 0.66 |
| Glu-Lys | 289 | EEEAAKKK | Cell-penetrating | 0.78 | Low | 0.92 |
| ACPP | 290 | EEEEEEEEPLGLAGRRRRRRRRN | Cell-penetrating | 0.97 | Low | 0.52 |
| Cyt 4-13 | 291 | EKGKKIFIMK | Cell-penetrating | 0.58 | Low | 0.828 |
| Engrailed (454- | 292 | EKRPRTAFSSEQLARLKREFNENRYLTTERRR | Cell-penetrating | 0.9 | High | 0.785 |
| 513) | | QQLSSELGLNEAQIKIWFQNKRAKIKKST | | | | |
| X | 293 | ELALELALEALEAALELA | Cell-penetrating | 0.95 | Low | 0.71 |
| Bip18 | 294 | ELPVM | Non-cell-penetrating | 0.61 | — | — |
| Peptide 65 | 295 | EPDNWSLDFPRR | Cell-penetrating | 0.76 | Low | 0.75 |
| Unknown | 296 | ERERERERERERER | Cell-penetrating | 0.96 | Low | 0.61 |
| HATF3 | 297 | ERKKRRRE | Cell-penetrating | 0.97 | Low | 0.744 |
| c-Myc-R11 | 298 | ESGGGGSPGRRRRRRRRRRR | Cell-penetrating | 1 | Low | 0.55 |
| Peptide 34 | 299 | FAPWDTASFMLG | Cell-penetrating | 0.73 | Low | 0.835 |
| Peptide 33 | 300 | FDPFFWKYSPRD | Cell-penetrating | 0.8 | Low | 0.6 |
| Phe-Oct-6 | 301 | FFFAAGRKRKKRT | Cell-penetrating | 0.99 | Low | 0.91 |
| F6R8 (Alexa) | 302 | FFFFFFGRRRRRRRRGC | Cell-penetrating | 0.99 | Low | 0.531 |
| F4R8 (Alexa) | 303 | FFFFGRRRRRRRRGC | Cell-penetrating | 0.99 | High | 0.549 |
| F2R8 (Alexa) | 304 | FFGRRRRRRRGC | Cell-penetrating | 0.98 | High | 0.538 |
| LAH4-X1F2 | 305 | FFKKLALHALHLLALLWLHLAHLALKK | Cell-penetrating | 0.97 | High | 0.6 |
| PEG- | 306 | FFLIGRRRRRRRRGC | Cell-penetrating | 0.99 | High | 0.549 |
| PasΔPKR8 | | | | | | |
| (Alexa) | | | | | | |
| PasR8 (Alexa) | 307 | FFLIPKGRRRRRRRRGC | Cell-penetrating | 0.98 | High | 0.556 |
| PR9 | 308 | FFLIPKGRRRRRRRRR | Cell-penetrating | 0.99 | High | 0.52 |
| F10 | 309 | FHFHFRFR | Cell-penetrating | 0.87 | High | 0.534 |
| TCTP-CPP 15 | 310 | FIIFRIAASHKK | Cell-penetrating | 0.93 | Low | 0.55 |
| LR8DRIHF | 311 | FIRIGC | Non-cell-penetrating | 0.57 | — | — |
| Tat (37-53) | 312 | FITKALGISYGRKKRR | Cell-penetrating | 0.93 | Low | 0.87 |
| Tat (37-60) | 313 | FITKALGISYGRKKRRQRRRPPQ | Cell-penetrating | 0.98 | High | 0.81 |
| C.e SDC3 | 314 | FKKFRKF | Cell-penetrating | 0.94 | Low | 0.85 |
| LAH4-X1F1 | 315 | FKKLALHALHLLALLWLHLAHLALKK | Cell-penetrating | 0.96 | High | 0.56 |
| PN285 | 316 | FKQqQqQqQqQq | Cell-penetrating | 0.72 | Low | 0.67 |
| M 511 | 317 | FLGKKFKKYFLQLLK | Cell-penetrating | 0.97 | High | 0.89 |
| G53-4 | 318 | FLIFIRVICIVIAKLKANLMCKT | Cell-penetrating | 0.86 | High | 0.8 |
| PF22 | 319 | FLKLLKKFLKLFKKLLKLF | Cell-penetrating | 1 | Low | 0.513 |
| C1 | 320 | FQFNFQFNGGGHRRRRRRR | Cell-penetrating | 0.98 | High | 0.546 |
| pAntp (49-58) | 321 | FQNRRMKWKK | Cell-penetrating | 0.84 | High | 0.91 |
| Peptide 32 | 322 | FQPYDHPAEVSY | Cell-penetrating | 0.78 | Low | 0.777 |
| M4 | 323 | FQWQRNMRKVRGPPVS | Cell-penetrating | 0.77 | Low | 0.828 |
| Single | 324 | FrFKFrFK | Cell-penetrating | 0.99 | High | 0.569 |
| mitochondrial | | | | | | |
| penetrating | | | | | | |
| peptide | | | | | | |
| ARF(1-37) scr | 325 | FRVPLRIRPCVVAPRLVMVRHTFGRIARWVA | Cell-penetrating | 0.87 | High | 0.602 |
| | | GPLETR | | | | |
| F8 | 326 | FTFHFTFHF | Cell-penetrating | 0.6 | Low | 0.54 |
| Peptide 35 | 327 | FTYKNFFWLPEL | Cell-penetrating | 0.76 | Low | 0.57 |
| ARF(1-22) scr | 328 | FVTRGCPRRLVARLIRVMVPRR | Cell-penetrating | 0.95 | High | 0.805 |
| SFTI-M1 | 329 | GACTKSIPPICFPD | Cell-penetrating | 0.62 | Low | 0.73 |
| MPGα | 330 | GALFLAFLAAALSLMGLWSQPKKKRKV | Cell-penetrating | 1 | Low | 0.577 |
| P(alpha) | 331 | GALFLAFLAAALSLMGLWSQPKKKRRV | Cell-penetrating | 0.99 | Low | 0.547 |
| MPGβ | 332 | GALFLGFLGAAGSTMGAWSQPKKKRKV | Cell-penetrating | 0.93 | Low | 0.86 |
| EGFP-MPG | 333 | GALFLGWLGAAGSTMGAPKKKRKV | Cell-penetrating | 0.9 | Low | 0.77 |
| MPG-NLS | 334 | GALFLGWLGAAGSTMGAPKSKRKVGGC | Cell-penetrating | 0.88 | Low | 0.8 |
| DPV15b | 335 | GAYDLRRRERQSRLRRRERQSR | Cell-penetrating | 0.99 | High | 0.542 |
| Tat | 336 | GCGGGYGRKKRRQRRR | Cell-penetrating | 0.99 | High | 0.547 |
| Inv7 | 337 | GDVYADAAPDLFDFLDSSVTTARTINA | Cell-penetrating | 0.79 | Low | 0.95 |
| | 338 | GEQIAQLIAGYIDIILKKKKSK | Cell-penetrating | 0.79 | Low | 0.63 |
| CF-Vim- | 339 | GGAYVTRSSAVRLRSSVPGVRLLQ | Cell-penetrating | 0.92 | Low | 0.76 |
| TBS.58-81 | | | | | | |
| POD | 340 | GGGARKKAAKAARKKAAKAARKKAAKAA | Cell-penetrating | 0.99 | Low | 0.675 |
| | | RKKAAKA | | | | |
| m9R | 341 | GGGGRRRRRRRRRLLLL | Cell-penetrating | 1 | Low | 0.502 |
| G3R6TAT | 342 | GGGRRRRRRYGRKKRRQRR | Cell-penetrating | 0.99 | High | 0.568 |
| CTP | 343 | GGRRARRRRRR | Cell-penetrating | 1 | Low | 0.53 |
| MCoK6A | 344 | GGVCPAILKKCRRDSDCPGACICRGNGYCGS | Cell-penetrating | 0.69 | Low | 0.76 |
| mutant | | GSD | | | | |
| MCoKKAA | 345 | GGVCPKILAACRRDSDCPGACICRGNGYCGS | Cell-penetrating | 0.66 | Low | 0.79 |
| double mutant | | GSD | | | | |
| MCoK9A | 346 | GGVCPKILAKCRRDSDCPGACICRGNGYCGS | Cell-penetrating | 0.65 | Low | 0.77 |
| mutant | | GSD | | | | |
| MCoK10A | 347 | GGVCPKILKACRRDSDCPGACICRGNGYCGS | Cell-penetrating | 0.66 | Low | 0.77 |
| mutant | | GSD | | | | |
| MCoTI-M1 | 348 | GGVCPKILKKCRRDSDCPGACICRGNGWCGS | Cell-penetrating | 0.68 | Low | 0.71 |
| | | GSD | | | | |
| MCoTI-II | 349 | GGVCPKILKKCRRDSDCPGACICRGNGYCGS | Cell-penetrating | 0.74 | Low | 0.73 |
| | | GSD | | | | |
| MCoTI-M3 | 350 | GGVCPKILRRCRRDSDCPGACICRGNGWCGS | Cell-penetrating | 0.62 | Low | 0.675 |
| | | GSD | | | | |
| MCoTI-M2 | 351 | GGVCPKILRRCRRDSDCPGACICRGNGYCGS | Cell-penetrating | 0.67 | Low | 0.705 |
| | | GSD | | | | |
| MCoTI-M4 | 352 | GGVCPKILRRCRRDSDCPGACICRGNGYCGS | Cell-penetrating | 0.69 | Low | 0.61 |
| | | GSR | | | | |
| MCoTI-M5 | 353 | GGVCPRILRRCRRDSDCPGACICRGNGYCGS | Cell-penetrating | 0.69 | Low | 0.617 |
| | | GSK | | | | |
| MG2A | 354 | GIGKFLHSAKKFGKAFVGEIMNSGGKKWKM | Cell-penetrating | 0.92 | Low | 0.508 |
| | | RRNQFWVKVQRG | | | | |
| MG2d | 355 | GIGKFLHSAKKWGKAFVGQIMNC | Non-cell-penetrating | 0.59 | — | — |
| Cyclin L ania- | 356 | GKHRHERGHHRDRRER | Cell-penetrating | 0.98 | Low | 0.588 |
| 6a | | | | | | |
| | 357 | GKINLKALAALAKKIL | Cell-penetrating | 0.95 | High | 0.5 |
| GKK peptide | 358 | GKKALKLAAKLLKKC | Cell-penetrating | 1 | Low | 0.52 |
| Lys9 | 359 | GKKKKKKKKK | Cell-penetrating | 0.97 | Low | 0.61 |
| TCF1-ALPHA | 360 | GKKKKRKREKL | Cell-penetrating | 1 | High | 0.88 |
| beta Zip TF | 361 | GKKKRKLSNRESAKRSR | Cell-penetrating | 0.98 | Low | 0.552 |
| ABL-1 | 362 | GKKTNLFSALIKKKKTA | Cell-penetrating | 0.96 | Low | 0.707 |
| GCN-4 | 363 | GKRARNTEAARRSRARKL | Cell-penetrating | 0.98 | Low | 0.706 |
| HB-EGF | 364 | GKRKKKGKGLGKKRDPCLRKYK | Cell-penetrating | 0.93 | Low | 0.507 |
| DPV7 | 365 | GKRKKKGKLGKKRDP | Cell-penetrating | 0.96 | Low | 0.655 |
| DPV7b | 366 | GKRKKKGKLGKKRPRSR | Cell-penetrating | 1 | Low | 0.647 |
| HEN2/NSLC2 | 367 | GKRRRRATAKYRSAH | Cell-penetrating | 0.99 | Low | 0.672 |
| Thyroid A-1 | 368 | GKRVAKRKLIEQNRERRR | Cell-penetrating | 0.98 | High | 0.523 |
| Inv2 | 369 | GKYVSLTTPKNPTKRRITPKDV | Cell-penetrating | 0.89 | Low | 0.785 |
| Peptide 599 | 370 | GLFEAIEGFIENGWEGMIDGWYGGGGrrrrrrrrr | Cell-penetrating | 0.78 | Low | 0.684 |
| | | K | | | | |
| JST-1 | 371 | GLFEALLELLESLWELLLEA | Cell-penetrating | 0.8 | Low | 0.57 |
| ppTG1 | 372 | GLFKALLKLLKSLWKLLLKA | Cell-penetrating | 0.99 | High | 0.6 |
| ppTG | 373 | GLFKALLKLLKSLWKLLLKAGGC | Cell-penetrating | 0.99 | Low | 0.545 |
| EGFP-ppTG20 | 374 | GLFRALLRLLRSLWRLLLRA | Cell-penetrating | 1 | Low | 0.53 |
| Inv6 | 375 | GLGDKFGESIVNANTVLDDLNSRMPQSRHDI | Cell-penetrating | 0.62 | Low | 0.91 |
| | | QQL | | | | |
| PN283 | 376 | GLGSLLKKAGKKLKQPKSKRKV | Cell-penetrating | 0.98 | Low | 0.72 |
| Peptide 2C- | 377 | GLKKLAELAHKLLKLG | Cell-penetrating | 0.89 | Low | 0.59 |
| GNS | | | | | | |
| EA | 378 | GLKKLAELAHKLLKLGC | Cell-penetrating | 0.85 | Low | 0.52 |
| TAMARA- | 379 | GLKKLAELFHKLLKLG | Cell-penetrating | 0.84 | Low | 0.575 |
| peptide 1 | | | | | | |
| EF | 380 | GLKKLAELFHKLLKLGC | Cell-penetrating | 0.83 | High | 0.51 |
| RA | 381 | GLKKLARLAHKLLKLGC | Cell-penetrating | 0.98 | Low | 0.527 |
| RF | 382 | GLKKLARLFHKLLKLGC | Cell-penetrating | 0.99 | High | 0.515 |
| N-E5L-Sc18 | 383 | GLLEALAELLEGLRKRLRKFRNKIKEK | Cell-penetrating | 0.98 | Low | 0.57 |
| DSPE-PEG- | 384 | GLPRRRRRRRRR | Cell-penetrating | 0.98 | High | 0.567 |
| CPP (CPP-Lp) | | | | | | |
| kT20K mutant | 385 | GLPVCGETCVGGTCNTPGCKCSWPVCTRN | Cell-penetrating | 0.69 | Low | 0.65 |
| kV25K mutant | 386 | GLPVCGETCVGGTCNTPGCTCSWPKCTRN | Cell-penetrating | 0.57 | Low | 0.68 |
| CF-sC18 | 387 | GLRKRLRKFRNKIKEK | Cell-penetrating | 0.99 | High | 0.856 |
| CADY-1c | 388 | GLWRALWRALRSLWKLKRKV | Cell-penetrating | 0.99 | High | 0.51 |
| CADY-2c | 389 | GLWRALWRALWRSLWKKKRKV | Cell-penetrating | 0.99 | High | 0.598 |
| CADY-1b | 390 | GLWRALWRALWRSLWKLKRKV | Cell-penetrating | 1 | High | 0.54 |
| CADY-2 | 391 | GLWRALWRALWRSLWKLKWKV | Cell-penetrating | 0.98 | High | 0.52 |
| CADY-2b | 392 | GLWRALWRALWRSLWKSKRKV | Cell-penetrating | 0.98 | Low | 0.53 |
| CADY-1e | 393 | GLWRALWRGLRSLWKKKRKV | Cell-penetrating | 0.99 | Low | 0.518 |
| CADY-1d | 394 | GLWRALWRGLRSLWKLKRKV | Cell-penetrating | 0.99 | Low | 0.52 |
| CAD-2 (des- | 395 | GLWRALWRLLRSLWRLLWKA | Non-cell-penetrating | 0 | — | — |
| acetyl, Lys19- | | | | | | |
| CADY) | | | | | | |
| CADY-2e | 396 | GLWRALWRLLRSLWRLLWSQPKKKRKV | Cell-penetrating | 1 | High | 0.52 |
| CADY-1 | 397 | GLWWKAWWKAWWKSLWWRKRKRKA | Cell-penetrating | 0.97 | High | 0.51 |
| CADY2 | 398 | GLWWRLWWRLRSWFRLWFRA | Cell-penetrating | 0.99 | High | 0.565 |
| Hip C | 399 | GNYAHRVGAGAPVWL | Cell-penetrating | 0.8 | Low | 0.767 |
| 435B peptide | 400 | GPFHFYQFLFPPV | Cell-penetrating | 0.82 | High | 0.75 |
| SFTI-M2 | 401 | GRCTKSIPPICFPA | Cell-penetrating | 0.63 | Low | 0.72 |
| SFTI-1 | 402 | GRCTKSIPPICFPD | Cell-penetrating | 0.77 | Low | 0.73 |
| SFTI-M3 | 403 | GRCTKSIPPICWPD | Cell-penetrating | 0.69 | Low | 0.69 |
| SFTI-M4 | 404 | GRCTKSIPPICWPK | Cell-penetrating | 0.66 | Low | 0.6 |
| SFTI-M5 | 405 | GRCTRSIPPKCWPD | Cell-penetrating | 0.86 | Low | 0.713 |
| Pep3(Mutant) | 406 | GRGDGPRRKKKKGPRRKKKKGPRR | Cell-penetrating | 0.99 | Low | 0.56 |
| Pep1 | 407 | GRGDSPRR | Cell-penetrating | 0.88 | Low | 0.82 |
| Pep3 | 408 | GRGDSPRRKKKKSPRRKKKKSPRR | Cell-penetrating | 0.99 | Low | 0.612 |
| Pep2 | 409 | GRGDSPRRSPRR | Cell-penetrating | 0.96 | Low | 0.785 |
| hPER3 NLS | 410 | GRKGKHKRKKLP | Cell-penetrating | 0.99 | Low | 0.623 |
| Ala substitution | 411 | GRKKRRQARAPPQC | Cell-penetrating | 0.94 | Low | 0.84 |
| mutant of Tat | | | | | | |
| (48-60) | | | | | | |
| Arg deletion | 412 | GRKKRRQPPQC | Cell-penetrating | 0.94 | Low | 0.92 |
| mutant of Tat | | | | | | |
| (48-60) | | | | | | |
| Ala substitution | 413 | GRKKRRQRARPPQC | Cell-penetrating | 0.96 | High | 0.68 |
| mutant of Tat | | | | | | |
| (48-60) | | | | | | |
| Arg deletion | 414 | GRKKRRQRPPQC | Cell-penetrating | 0.96 | Low | 0.78 |
| mutant of Tat | | | | | | |
| (48-60) | | | | | | |
| Arg deletion | 415 | GRKKRRQRRPPQC | Cell-penetrating | 0.97 | High | 0.78 |
| mutant of Tat | | | | | | |
| (48-60) | | | | | | |
| Tat (48-57) | 416 | GRKKRRQRRR | Cell-penetrating | 0.99 | High | 0.795 |
| Pro deletion | 417 | GRKKRRQRRRC | Cell-penetrating | 0.99 | High | 0.83 |
| mutant of Tat | | | | | | |
| (48-60) | | | | | | |
| Tat-CG | 418 | GRKKRRQRRRCG | Cell-penetrating | 1 | High | 0.695 |
| TAT | 419 | GRKKRRQRRRG | Cell-penetrating | 1 | High | 0.659 |
| TatsMTS | 420 | GRKKRRQRRRMVSAL | Cell-penetrating | 0.96 | Low | 0.528 |
| (TMG) | | | | | | |
| TAT (47-57) | 421 | GRKKRRQRRRP | Cell-penetrating | 0.99 | High | 0.815 |
| Tat (48-59) | 422 | GRKKRRQRRRPP | Cell-penetrating | 1 | High | 0.71 |
| Tat (48-60) | 423 | GRKKRRQRRRPPQ | Cell-penetrating | 0.97 | High | 0.94 |
| HIV-1 Tat (48- | 424 | GRKKRRQRRRPPQC | Cell-penetrating | 0.96 | High | 0.81 |
| 60) | | | | | | |
| | 425 | GRKKRRQRRRPPQGRKKRRQRRRPPQGRKK | Cell-penetrating | 0.99 | High | 0.72 |
| | | RRQRRRPPQ | | | | |
| TAT | 426 | GRKKRRQRRRPPQK | Cell-penetrating | 0.98 | High | 0.69 |
| Tat | 427 | GRKKRRQRRRPPQRKC | Cell-penetrating | 0.99 | High | 0.658 |
| Tat-PKI | 428 | GRKKRRQRRRPPQTYADFIASGRTGRRNAI | Cell-penetrating | 0.99 | High | 0.82 |
| Tat-Dex | 429 | GRKKRRQRRRPPQY | Cell-penetrating | 0.93 | High | 0.685 |
| HIV-1 TAT | 430 | GRKKRRQRRRPQ | Cell-penetrating | 0.99 | High | 0.7 |
| peptide-- | | | | | | |
| Crystallins | | | | | | |
| TatP59W | 431 | GRKKRRQRRRPWQ | Cell-penetrating | 0.98 | High | 0.87 |
| HME-1 | 432 | GRKLKKKKNEKEDKRPRT | Cell-penetrating | 0.97 | Low | 0.53 |
| 06-Oct | 433 | GRKRKKRT | Cell-penetrating | 0.99 | Low | 0.514 |
| DPV6 | 434 | GRPRESGKKRKRKRLKP | Cell-penetrating | 0.99 | High | 0.553 |
| Erns3 | 435 | GRQLRIAGKRLEGRSK | Cell-penetrating | 0.97 | Low | 0.715 |
| Erns6 | 436 | GRQLRIAGKRLRGRSK | Cell-penetrating | 0.99 | Low | 0.695 |
| Erns7 | 437 | GRQLRIAGRRLRGRSR | Cell-penetrating | 1 | Low | 0.67 |
| Erns9 | 438 | GRQLRIAGRRLRRRSR | Cell-penetrating | 1 | Low | 0.61 |
| Erns8 | 439 | GRQLRRAGRRLRGRSR | Cell-penetrating | 1 | Low | 0.573 |
| Erns10 | 440 | GRQLRRAGRRLRRRSR | Cell-penetrating | 0.99 | Low | 0.583 |
| Nucleoplasmin | 441 | GRRERNKMAAAKCRNRRR | Cell-penetrating | 0.91 | High | 0.51 |
| X | | | | | | |
| hPER1-PTD | 442 | GRRHHCRSKAKRSRHH | Cell-penetrating | 1 | Low | 0.724 |
| (830-846) NLS | | | | | | |
| HEN1/NSLC1 | 443 | GRRRRATAKYRTAH | Cell-penetrating | 0.96 | Low | 0.715 |
| HNF3 | 444 | GRRRRKRLSHRT | Cell-penetrating | 1 | Low | 0.69 |
| cAMP | 445 | GRRRRRERNK | Cell-penetrating | 0.97 | High | 0.67 |
| dependent TF | | | | | | |
| R9 | 446 | GRRRRRRRRR | Cell-penetrating | 1 | High | 0.73 |
| R9-TAT | 447 | GRRRRRRRRRPPQ | Cell-penetrating | 0.99 | High | 0.885 |
| (42-38)-(9-1) | 448 | GSGKKGGKKHCQKY | Cell-penetrating | 0.95 | Low | 0.727 |
| Crot | | | | | | |
| D form of (1- | 449 | GSGKKGGKKICQKY | Cell-penetrating | 0.92 | Low | 0.843 |
| 9)-(38-42) Crot | | | | | | |
| 439A peptide | 450 | GSPWGLQHHPPRT | Cell-penetrating | 0.88 | High | 0.7 |
| Peptide 16 | 451 | GSRHPSLIIPRQ | Cell-penetrating | 0.92 | Low | 0.643 |
| HSV-1 | 452 | GSRVQIRCRFRNSTR | Cell-penetrating | 0.96 | Low | 0.505 |
| glycoprotein C | | | | | | |
| gene (gC)-- | | | | | | |
| Crystallins | | | | | | |
| LMWP-EGFP | 453 | GSVSRRRRRRGGRRRR | Cell-penetrating | 0.97 | Low | 0.52 |
| Cyt C 71-101 | 454 | GTKMIFVGIKKKEERADLIAYLKKA | Cell-penetrating | 0.84 | High | 0.725 |
| TP5 | 455 | GWTLNPAGYLLGKINLKALAALAKKIL | Cell-penetrating | 0.96 | High | 0.815 |
| TP6 | 456 | GWTLNPPGYLLGKINLKALAALAKKIL | Cell-penetrating | 0.94 | High | 0.755 |
| TP4 | 457 | GWTLNSAGYLLGKFLPLILRKIVTAL | Cell-penetrating | 0.87 | Low | 0.82 |
| Transportan | 458 | GWTLNSAGYLLGKINLKALAALAKKIL | Cell-penetrating | 0.96 | High | 0.83 |
| TP2 | 459 | GWTLNSAGYLLGKINLKALAALAKKLL | Cell-penetrating | 0.97 | High | 0.79 |
| TP16 | 460 | GWTLNSAGYLLGKINLKAPAALAKKIL | Cell-penetrating | 0.94 | Low | 0.74 |
| TP9 | 461 | GWTLNSAGYLLGKLKALAALAKKIL | Cell-penetrating | 0.95 | High | 0.8 |
| Galanin | 462 | GWTLNSAGYLLGPHAVGNHRSFSDKNGLTS | Cell-penetrating | 0.84 | Low | 0.94 |
| TP11 | 463 | GWTLNSKINLKALAALAKKIL | Cell-penetrating | 0.88 | Low | 0.74 |
| No. 440 | 464 | GYGNCRHFKQKPRRD | Cell-penetrating | 0.89 | High | 0.8 |
| YM-3 | 465 | GYGRKKRRGRRRTHRLPRRRRRR | Cell-penetrating | 1 | High | 0.558 |
| Tat (47-57) | 466 | GYGRKKRRQRRRG | Cell-penetrating | 1 | High | 0.531 |
| D4 | 467 | GYGYGYGYGYGYGYGYKKRKKRKKRKKR | Cell-penetrating | 0.97 | High | 0.513 |
| | | KQQKQQKRRK | | | | |
| A8 | 468 | HALAHKLKHLLHRLRHLLHRHLRHALAH | Cell-penetrating | 0.97 | Low | 0.53 |
| L2 (Ala33 | 469 | HARIKPTFRRLKWKYKGKFW | Cell-penetrating | 0.95 | Low | 0.548 |
| substitution | | | | | | |
| mutant of | | | | | | |
| LALF (32-51)) | | | | | | |
| Peptide 6 | 470 | HATKSQNINF | Non-cell-penetrating | 0.76 | — | — |
| GST- | 471 | HEHEHEHEHEHEHEHEEFGGGGGYGRGRGR | Cell-penetrating | 0.85 | Low | 0.635 |
| (HE)8EFG5YG | | GRGRGRG | | | | |
| (RG)6 | | | | | | |
| GST- | 472 | HEHEHEHEHEHEHEHEEFGGGGGYGRRRRR | Cell-penetrating | 0.79 | Low | 0.59 |
| (HE)8EFG5YG | | RGGGGGG | | | | |
| R6G6 | | | | | | |
| GST- | 473 | HEHEHEHEHEHEHEHEHEHEEFGGGGGYGR | Cell-penetrating | 0.89 | Low | 0.645 |
| (HE)10EFG5Y | | GRGRGRGRGRG | | | | |
| G(RG)6 | | | | | | |
| GST- | 474 | HEHEHEHEHEHEHEHEHEHEEFGGGGGYGR | Cell-penetrating | 0.84 | Low | 0.59 |
| (HE)10EFG5Y | | RRRRRGGGGGG | | | | |
| GR6G6 | | | | | | |
| GST-HE-MAP | 475 | HEHEHEHEHEHEHEHEHEHEGGGGGKLALK | Cell-penetrating | 0.92 | Low | 0.65 |
| | | LALKALKAALKLA | | | | |
| GST- | 476 | HEHEHEHEHEHEHEHEHEHEHEHEEFGGGG | Cell-penetrating | 0.89 | Low | 0.625 |
| (HE)12EFG5Y | | GYGRGRGRGRGRGRG | | | | |
| G(RG)6 | | | | | | |
| GST- | 477 | HEHEHEHEHEHEHEHEHEHEHEHEEFGGGG | Cell-penetrating | 0.85 | Low | 0.526 |
| (HE)12EFG5- | | GYGRKKRRQRRR | | | | |
| TAT | | | | | | |
| GST- | 478 | HEHEHEHEHEHEHEHEHEHEHEHEEFGGGG | Cell-penetrating | 0.85 | Low | 0.61 |
| (HE)12EFG5Y | | GYGRRRRRRGGGGGG | | | | |
| GR6G6 | | | | | | |
| Peptide 29 | 479 | HFAAWGGWSLVH | Cell-penetrating | 0.83 | Low | 0.73 |
| Foxp3-11R | 480 | HHHHHHESGGGGSPGRRRRRRRRRRR | Cell-penetrating | 1 | Low | 0.6 |
| STR-H20R8 | 481 | HHHHHHHHHHHHHHHHHHHHRRRRRRRRR | Cell-penetrating | 1 | Low | 0.59 |
| | | RRRRRR | | | | |
| H16R8 | 482 | HHHHHHHHHHHHHHHHRRRRRRRRRRRRR | Cell-penetrating | 1 | Low | 0.57 |
| | | RR | | | | |
| STR-H12R8 | 483 | HHHHHHHHHHHHRRRRRRRRRRRRRRR | Cell-penetrating | 1 | Low | 0.56 |
| STR-H8R8 | 484 | HHHHHHHHRRRRRRRR | Cell-penetrating | 1 | Low | 0.6 |
| H8R15 | 485 | HHHHHHHHRRRRRRRRRRRRRRR | Cell-penetrating | 1 | Low | 0.555 |
| D9 | 486 | HHHHHHRRRRRRRRR | Cell-penetrating | 1 | Low | 0.525 |
| Inv3.10 | 487 | HHHHHHTKRRITPKDVIDVRSVTTEINT | Cell-penetrating | 0.76 | High | 0.72 |
| 5-FAM-H3R8 | 488 | HHHRRRRRRRR | Cell-penetrating | 1 | High | 0.575 |
| D8 | 489 | HHHRRRRRRRRRHHH | Cell-penetrating | 1 | High | 0.517 |
| DNA-IL-PEI | 490 | HILPWKWPWWPWRR | Cell-penetrating | 0.93 | High | 0.55 |
| Peptide 30 | 491 | HIQLSPFSQSWR | Cell-penetrating | 0.83 | Low | 0.647 |
| Peptide 54 | 492 | HPGSPFPPEHRP | Cell-penetrating | 0.93 | Low | 0.68 |
| Peptide 62 | 493 | HQHKPPPLTNNW | Cell-penetrating | 0.85 | Low | 0.735 |
| Peptide 12 | 494 | HRHIRRQSLIML | Cell-penetrating | 0.93 | Low | 0.79 |
| A7 | 495 | HRLRHALAHLLHKLKHLLHALAHRLRH | Cell-penetrating | 0.99 | Low | 0.53 |
| VIP-TAT | 496 | HSDAVFTDNYTALRKQMAVKKYLNSILNYG | Cell-penetrating | 0.91 | High | 0.508 |
| | | RKKRRQRRR | | | | |
| PACAP | 497 | HSDGIFTDSYSRYRKQMAVKKYLAAVLGKR | Cell-penetrating | 0.81 | High | 0.543 |
| | | YKQRVKNK | | | | |
| L8 (Ala39 | 498 | HYRIKPTARRLKWKYKGKFW | Cell-penetrating | 0.96 | Low | 0.543 |
| substitution | | | | | | |
| mutant of | | | | | | |
| LALF (32-51)) | | | | | | |
| L12 (Ala43 | 499 | HYRIKPTFRRLAWKYKGKFW | Cell-penetrating | 0.9 | Low | 0.543 |
| substitution | | | | | | |
| mutant of | | | | | | |
| LALF (32-51)) | | | | | | |
| L20 (Ala51 | 500 | HYRIKPTFRRLKWKYKGKFA | Cell-penetrating | 0.94 | High | 0.527 |
| substitution | | | | | | |
| mutant of | | | | | | |
| LALF (32-51)) | | | | | | |
| YTA4 | 501 | IAWVKAFIRKLRKGPLG | Cell-penetrating | 0.93 | Low | 0.575 |
| Penetration | 502 | IGCRH | Cell-penetrating | 0.57 | High | 0.57 |
| Xentry peptides | 503 | IIIR | Cell-penetrating | 0.7 | High | 0.594 |
| TCTP (2-10) | 504 | HYRDLISH | Non-cell-penetrating | 0.7 | — | — |
| deletion mutant | | | | | | |
| D7 | 505 | IKIKIKIKIKIKIKIKKLAKLAKLAKLAKLAKL | Cell-penetrating | 0.99 | Low | 0.52 |
| | | AKKIK | | | | |
| pAntp (45-58) | 506 | IKIWFQNRRMKWKK | Cell-penetrating | 0.93 | High | 0.912 |
| TAM-MP | 507 | INLKALAALAKKIL | Cell-penetrating | 0.9 | Low | 0.63 |
| Bip14 | 508 | IPALK | Cell-penetrating | 0.72 | High | 0.827 |
| IPL | 509 | IPLVVPLC | Cell-penetrating | 0.67 | High | 0.56 |
| RIPL peptide | 510 | IPLVVPLRRRRRRRRC | Cell-penetrating | 0.98 | High | 0.595 |
| Bip10 | 511 | IPMIK | Non-cell-penetrating | 0.58 | — | — |
| Bip15 | 512 | IPMLK | Cell-penetrating | 0.56 | High | 0.92 |
| No.143 | 513 | IPSRWKDQFWKRWHY | Cell-penetrating | 0.85 | High | 0.807 |
| IRQ | 514 | IRQRRRR | Cell-penetrating | 0.98 | Low | 0.566 |
| NYAD-41 | 515 | ISFDELLDYYGESGS | Cell-penetrating | 0.85 | Low | 0.82 |
| pAntp (47-58) | 516 | IWFQNRRMKWKK | Cell-penetrating | 0.89 | High | 0.97 |
| Peptide 8 | 517 | IWRYSLASQQ | Cell-penetrating | 0.59 | Low | 0.58 |
| P7-5 | 518 | IYLATALAKWALKQGFGGRRRRRRR | Cell-penetrating | 1 | Low | 0.596 |
| P7-7 | 519 | IYLATALAKWALKQGGRRRRRRR | Cell-penetrating | 0.99 | Low | 0.542 |
| TCTP (3-10) | 520 | IYRDLISH | Non-cell-penetrating | 0.67 | — | — |
| deletion mutant | | | | | | |
| KAFAK | 521 | KAFAKLAARLYRKALARQLGVAA | Cell-penetrating | 1 | Low | 0.53 |
| II | 522 | KALAALLKKLAKLLAALK | Cell-penetrating | 1 | High | 0.93 |
| KLA8 | 523 | KALAALLKKWAKLLAALK | Cell-penetrating | 1 | High | 0.89 |
| KLA12 | 524 | KALAKALAKLWKALAKAA | Cell-penetrating | 0.99 | High | 0.72 |
| KLA10 | 525 | KALKKLLAKWLAAAKALL | Cell-penetrating | 0.99 | High | 0.84 |
| NAP | 526 | KALKLKLALALLAKLKLA | Cell-penetrating | 1 | High | 0.64 |
| Crot (27-39) | 527 | KCCKWRWRCK | Cell-penetrating | 0.95 | High | 0.94 |
| derevative | | | | | | |
| rLF | 528 | KCFMWQEMLNKAGVPKLRCARK | Cell-penetrating | 0.83 | Low | 0.8 |
| M3 | 529 | KCFQWQRNMRKVR | Cell-penetrating | 0.94 | Low | 0.83 |
| M1 | 530 | KCFQWQRNMRKVRGPPVSC | Cell-penetrating | 0.68 | High | 0.805 |
| hLF WT | 531 | KCFQWQRNMRKVRGPPVSCIKR | Cell-penetrating | 0.92 | High | 0.72 |
| M2 | 532 | KCFQWQRNMRKVRGPPVSSIKR | Cell-penetrating | 0.87 | Low | 0.71 |
| Crot (27-39) | 533 | KCGCRWRWKCGCKK | Cell-penetrating | 0.95 | High | 0.907 |
| derevative | | | | | | |
| ALPHA Virus | 534 | KCPSRRPKR | Cell-penetrating | 0.97 | Low | 0.62 |
| nucelocapsid | | | | | | |
| (311-320) | | | | | | |
| Crot (27-39) | 535 | KCRWRWKCCKK | Cell-penetrating | 0.95 | High | 0.98 |
| derevative | | | | | | |
| FITC-WT1-pTj | 536 | KDCERRFSRSDQLKRHQRRHTGVKPFQK | Cell-penetrating | 0.85 | High | 0.605 |
| Crot (27-39) | 537 | KDCRWRWKCCKK | Cell-penetrating | 0.78 | High | 0.99 |
| derevative | | | | | | |
| Pep-2 | 538 | KETWFETWFTEWSQPKKKRKV | Cell-penetrating | 0.81 | Low | 0.68 |
| PN183 | 539 | KETWWETWWTEWSQPGRKKRRQRRRPPQ | Cell-penetrating | 0.93 | High | 0.568 |
| EGFP-Pep-1 | 540 | KETWWETWWTEWSQPKKKRKV | Cell-penetrating | 0.88 | Low | 0.67 |
| FP-lipo | 541 | KETWWETWWTEWSQPKKKRKVC | Cell-penetrating | 0.81 | Low | 0.61 |
| CPP-PNA | 542 | KFFKFFKFFK | Cell-penetrating | 0.94 | Low | 0.55 |
| hCT (18â€“32) | 543 | KFHTFPQTAIGVGAP | Cell-penetrating | 0.66 | Low | 0.67 |
| IP-1 | 544 | KFLNRFWHWLQLKPGQPMY | Cell-penetrating | 0.87 | Low | 0.58 |
| Cyt c (5-13) | 545 | KGKKIFIMK | Cell-penetrating | 0.66 | High | 0.74 |
| q-NTD | 546 | KGRKKRRQRRRPPQ | Cell-penetrating | 0.96 | High | 0.7 |
| Res4 | 547 | KGRTPIKFGKADCDRPPKHSQNGMGK | Cell-penetrating | 0.66 | Low | 0.575 |
| PN509 | 548 | KGSKKAVTKAQKKDGKKRKRSRKESYSVYV | Cell-penetrating | 0.98 | Low | 0.66 |
| | | YKVLKQ | | | | |
| MMD45 | 549 | KHHWHHVRLPPPVRLPPPGNHHHHHH | Cell-penetrating | 0.86 | Low | 0.55 |
| LAH6-X1 | 550 | KHKALHALHLLALLWLHLAHLAKHK | Cell-penetrating | 0.96 | High | 0.56 |
| (KH)9-Bp100 | 551 | KHKHKHKHKHKHKHKHKHKKLFKKILKYL | Cell-penetrating | 0.96 | Low | 0.59 |
| LAH6-X1L-W | 552 | KHKLLHLLHLLALLWLHLLHLLKHK | Cell-penetrating | 0.96 | Low | 0.51 |
| KLA5 | 553 | KIAAKSIAKIWKSILKIA | Cell-penetrating | 0.97 | Low | 0.92 |
| fGeT | 554 | KIAKLKAKIQKLKQKIAKLK | Cell-penetrating | 0.99 | Low | 0.595 |
| KLA11 | 555 | KITLKLAIKAWKLALKAA | Cell-penetrating | 0.98 | Low | 0.78 |
| pAntp (46-58) | 556 | KIWFQNRRMKWKK | Cell-penetrating | 0.93 | High | 0.96 |
| APP521 | 557 | KKAAQIRSQVMTHLRVI | Cell-penetrating | 0.78 | Low | 0.86 |
| LAH4-L1 | 558 | KKALLAHALHLLALLALHLAHALKKA | Cell-penetrating | 0.99 | High | 0.56 |
| PN361 | 559 | KKDGKKRKRSRKESYSVYVYKVLKQ | Cell-penetrating | 0.8 | Low | 0.63 |
| M867 | 560 | KKICTRKPRFMSAWAQ | Cell-penetrating | 0.94 | High | 0.71 |
| Cyt C 86-101 | 561 | KKKEERADLIAYLKKA | Cell-penetrating | 0.78 | Low | 0.79 |
| CL22 | 562 | KKKKKKGGFLGFWRGENGRKTRSAYERMCI | Cell-penetrating | 0.96 | Low | 0.58 |
| | | LKGK | | | | |
| K8-lip | 563 | KKKKKKKK | Cell-penetrating | 0.98 | Low | 0.62 |
| K9 | 564 | KKKKKKKKK | Cell-penetrating | 0.98 | Low | 0.55 |
| Polylysine19 | 565 | KKKKKKKKKKKKKKKKKKK | Cell-penetrating | 0.98 | Low | 0.69 |
| P1 | 566 | KKKKKKNKKLQQRGD | Cell-penetrating | 0.94 | Low | 0.617 |
| LAH4-X1 | 567 | KKLALHALHLLALLWLHLAHLALKK | Cell-penetrating | 0.98 | High | 0.57 |
| CF-BP16 | 568 | KKLFKKILKKL | Cell-penetrating | 0.97 | Low | 0.55 |
| RSV-A11 | 569 | KKPGKKTTTKPTKK | Cell-penetrating | 0.89 | Low | 0.735 |
| RSV-A10 | 570 | KKPGKKTTTKPTKKPTIKTTKK | Cell-penetrating | 0.93 | Low | 0.61 |
| RSV-A12 | 571 | KKPTIKTTKK | Cell-penetrating | 0.83 | Low | 0.678 |
| Tat (50-57) | 572 | KKRRQRRR | Cell-penetrating | 1 | Low | 0.77 |
| RSV-A13 | 573 | KKTTTKPTKK | Cell-penetrating | 0.87 | Low | 0.645 |
| MMD47 | 574 | KKWALLALALHHLAHLALHLALALKKAHH | Cell-penetrating | 0.95 | Low | 0.54 |
| | | HHHH | | | | |
| Pen7-9Ã-Arg | 575 | kkwkmrrGaGrrrrrrrrr | Cell-penetrating | 0.97 | High | 0.51 |
| pAntpHD (58- | 576 | KKWKMRRNQFWIKIQR | Cell-penetrating | 0.91 | High | 0.85 |
| 43) | | | | | | |
| KLA15 | 577 | KLAAALLKKWKKLAAALL | Cell-penetrating | 1 | High | 0.83 |
| KLA | 578 | KLAKLAKKLAKLAK | Cell-penetrating | 0.99 | Low | 0.59 |
| KLA-R7 | 579 | KLAKLAKKLAKLAKGGRRRRRRR | Cell-penetrating | 1 | High | 0.535 |
| KLA-TAT(47- | 580 | KLAKLAKKLAKLAKGRKKRRQRRRP | Cell-penetrating | 1 | High | 0.66 |
| 57) | | | | | | |
| KLA-ECP(32- | 581 | KLAKLAKKLAKLAKNYRWRCKNQN | Cell-penetrating | 0.97 | High | 0.548 |
| 41) | | | | | | |
| KLA3 | 582 | KLALKAAAKAWKAAAKAA | Cell-penetrating | 0.99 | Low | 0.87 |
| KLA2 | 583 | KLALKAALKAWKAAAKLA | Cell-penetrating | 1 | Low | 0.84 |
| IV | 584 | KLALKALKAALKLA | Cell-penetrating | 0.99 | Low | 0.87 |
| V | 585 | KLALKLALKALKAA | Cell-penetrating | 0.99 | Low | 0.87 |
| III | 586 | KLALKLALKALKAALK | Cell-penetrating | 1 | High | 0.77 |
| I | 587 | KLALKLALKALKAALKLA | Cell-penetrating | 1 | High | 0.72 |
| MAP | 588 | KLALKLALKALKAALKLAGC | Cell-penetrating | 1 | High | 0.825 |
| VII | 589 | KLALKLALKALQAALQLA | Cell-penetrating | 0.9 | Low | 0.72 |
| KLA1 | 590 | KLALKLALKAWKAALKLA | Cell-penetrating | 1 | High | 0.67 |
| KLA13 | 591 | KLALKLALKWAKLALKAA | Cell-penetrating | 1 | Low | 0.86 |
| VIII | 592 | KLALQLALQALQAALQLA | Cell-penetrating | 0.93 | High | 0.85 |
| PePM | 593 | KLFMALVAFLRFLTIPPTAGILKRWGTI | Cell-penetrating | 0.88 | Low | 0.58 |
| VI | 594 | KLGLKLGLKGLKGGLKLG | Cell-penetrating | 0.99 | Low | 0.79 |
| Bip11 | 595 | KLGVM | Non-cell-penetrating | 0.55 | — | — |
| Res7 | 596 | KLIKGRTPIKFGK | Cell-penetrating | 0.86 | Low | 0.595 |
| Res5 | 597 | KLIKGRTPIKFGKADCDRPPKHSGK | Cell-penetrating | 0.77 | Low | 0.628 |
| Res3 | 598 | KLIKGRTPIKFGKADCDRPPKHSQNGK | Cell-penetrating | 0.73 | Low | 0.61 |
| Res2 | 599 | KLIKGRTPIKFGKADCDRPPKHSQNGM | Cell-penetrating | 0.52 | Low | 0.573 |
| Res1 | 600 | KLIKGRTPIKFGKADCDRPPKHSQNGMGK | Cell-penetrating | 0.85 | Low | 0.57 |
| Res6 | 601 | KLIKGRTPIKFGKARCRRPPKHSGK | Cell-penetrating | 0.94 | Low | 0.58 |
| KLA14 | 602 | KLLAKAAKKWLLLALKAA | Cell-penetrating | 0.99 | Low | 0.84 |
| KLA9 | 603 | KLLAKAALKWLLKALKAA | Cell-penetrating | 1 | Low | 0.91 |
| C5 | 604 | KLLKLLLKLWKKLLKLLK | Cell-penetrating | 0.99 | High | 0.5 |
| A6 | 605 | KLLKLLLKLWKKLLKLLKGGGRRRRRRR | Cell-penetrating | 1 | High | 0.635 |
| G55-9 | 606 | KLPCRSNTFLNIFRRKKPG | Cell-penetrating | 0.91 | Low | 0.535 |
| Bip9 | 607 | KLPVM | Cell-penetrating | 0.55 | High | 0.8 |
| Bip12 | 608 | KLPVT | Cell-penetrating | 0.67 | High | 0.54 |
| CCMV GAG | 609 | KLTRAQRRAAARKNKRNTRGC | Cell-penetrating | 0.99 | High | 0.78 |
| 7 | 610 | KLWMRWWSPTTRRYG | Cell-penetrating | 0.98 | High | 0.93 |
| No.14-2 | 611 | KLWMRWYSATTRRYG | Cell-penetrating | 0.98 | High | 0.97 |
| No.14 | 612 | KLWMRWYSPTTRRYG | Cell-penetrating | 0.98 | High | 0.96 |
| No.14-7 | 613 | KLWMRWYSPWTRRYG | Cell-penetrating | 0.96 | High | 0.92 |
| PN228 | 614 | KLWSAWPSLWSSLWKP | Cell-penetrating | 0.89 | Low | 0.68 |
| Crot (27-39) | 615 | KMDCRPRPKCCKK | Cell-penetrating | 0.91 | Low | 0.73 |
| derevative | | | | | | |
| Crot (27-39) | 616 | KMDCRWRPKCCKK | Cell-penetrating | 0.81 | High | 0.84 |
| derevative | | | | | | |
| Crot (27-39) | 617 | KMDCRWRWKCCKK | Cell-penetrating | 0.8 | High | 0.94 |
| Crot (27-39) | 618 | KMDCRWRWKCKK | Cell-penetrating | 0.78 | High | 0.95 |
| derevative | | | | | | |
| Crot (27-39) | 619 | KMDCRWRWKCSKK | Cell-penetrating | 0.82 | High | 0.95 |
| derevative | | | | | | |
| Crot (27-39) | 620 | KMDCRWRWKKK | Cell-penetrating | 0.77 | High | 0.86 |
| derevative | | | | | | |
| Crot (27-39) | 621 | KMDCRWRWKSCKK | Cell-penetrating | 0.83 | High | 0.95 |
| derevative | | | | | | |
| Crot (27-39) | 622 | KMDCRWRWKSSKK | Cell-penetrating | 0.88 | Low | 0.76 |
| derevative | | | | | | |
| Crot (27-39) | 623 | KMDRWRWKKK | Cell-penetrating | 0.78 | Low | 0.81 |
| derevative | | | | | | |
| Crot (27-39) | 624 | KMDSRWRWKCCKK | Cell-penetrating | 0.81 | Low | 0.68 |
| derevative | | | | | | |
| Crot (27-39) | 625 | KMDSRWRWKCSKK | Cell-penetrating | 0.88 | High | 0.6 |
| derevative | | | | | | |
| Crot (27-39) | 626 | KMDSRWRWKSCKK | Cell-penetrating | 0.89 | Low | 0.84 |
| derevative | | | | | | |
| Crot (27-39) | 627 | KMDSRWRWKSSKK | Cell-penetrating | 0.93 | Low | 0.87 |
| derevative | | | | | | |
| Cyt 79-88 | 628 | KMIFVGIKKK | Cell-penetrating | 0.62 | Low | 0.793 |
| Cyt 79-92 | 629 | KMIFVGIKKKEERA | Cell-penetrating | 0.67 | Low | 0.92 |
| BMV GAG | 630 | KMTRAQRRAAARRNRWTARGC | Cell-penetrating | 0.99 | Low | 0.561 |
| No. 2028 | 631 | KNAWKHSSCEIHRHQI | Cell-penetrating | 0.72 | High | 0.787 |
| RSV-B3 | 632 | KPRSKNPPKKPK | Cell-penetrating | 0.95 | Low | 0.67 |
| Yeast GCN 4 | 633 | KRARNTEAARRSRARKLQRMKQGC | Cell-penetrating | 0.96 | Low | 0.821 |
| (231-252) | | | | | | |
| Peptide 2 | 634 | KRIHPRLTRSIR | Cell-penetrating | 0.99 | Low | 0.633 |
| Peptide 1 | 635 | KRIIQRILSRNS | Cell-penetrating | 0.97 | Low | 0.665 |
| RSV-A7 | 636 | KRIPNKKPGKK | Cell-penetrating | 0.86 | Low | 0.59 |
| RSV-A6 | 637 | KRIPNKKPGKKT | Cell-penetrating | 0.85 | Low | 0.55 |
| RSV-A5 | 638 | KRIPNKKPGKKTTTKPTKK | Cell-penetrating | 0.9 | Low | 0.588 |
| RSV-A4 | 639 | KRIPNKKPGKKTTTKPTKKPTIK | Cell-penetrating | 0.91 | Low | 0.54 |
| RSV-A3 | 640 | KRIPNKKPGKKTTTKPTKKPTIKTTKK | Cell-penetrating | 0.89 | Low | 0.587 |
| RSV-A2 | 641 | KRIPNKKPGKKTTTKPTKKPTIKTTKKDLK | Cell-penetrating | 0.84 | Low | 0.55 |
| RSV-A1 | 642 | KRIPNKKPGKKTTTKPTKKPTIKTTKKDLKPQ | Cell-penetrating | 0.97 | Low | 0.595 |
| | | TTKPK | | | | |
| RSV-A8 | 643 | KRIPNKKPKK | Cell-penetrating | 0.87 | Low | 0.59 |
| KW | 644 | KRKRWHW | Cell-penetrating | 0.89 | Low | 0.551 |
| Bipartite | 645 | KRPAAIKKAGQAKKKK | Cell-penetrating | 0.98 | Low | 0.693 |
| nucleoplasmin | | | | | | |
| NLS (155-170) | | | | | | |
| 44 | 646 | KRPTMRFRYTWNPMK | Cell-penetrating | 0.81 | High | 0.517 |
| Human c Fos | 647 | KRRIRRERNKMAAAKSRNRRRELTDTGC | Cell-penetrating | 0.93 | Low | 0.77 |
| (139-164) | | | | | | |
| Tat (51-57) | 648 | KRRQRRR | Cell-penetrating | 1 | Low | 0.88 |
| hClock-(35-47) | 649 | KRVSRNKSEKKRR | Cell-penetrating | 0.97 | High | 0.84 |
| Crot (27-39) | 650 | KRWRWKCCKK | Cell-penetrating | 0.93 | High | 0.89 |
| derevative | | | | | | |
| Retro-pVEC | 651 | KSHAHAQKRIRRRLIILL | Cell-penetrating | 0.99 | Low | 0.9 |
| RSV-B 1 | 652 | KSICKTIPSNKPKKK | Cell-penetrating | 0.94 | Low | 0.65 |
| KST | 653 | KSTGKANKITITNDKGRLSK | Cell-penetrating | 0.92 | Low | 0.672 |
| Peptide 64 | 654 | KTIEAHPPYYAS | Cell-penetrating | 0.88 | Low | 0.725 |
| RSV-B2 | 655 | KTIPSNKPKKK | Cell-penetrating | 0.89 | Low | 0.63 |
| E162 | 656 | KTVLLRKLLKLLVRKI | Cell-penetrating | 0.99 | High | 0.81 |
| MTp1-3 | 657 | KWCFAVCYAGICYAACAGK | Cell-penetrating | 0.84 | Low | 0.54 |
| Tpl | 658 | KWCFRVCYRGICYRRCRGK | Cell-penetrating | 0.98 | High | 0.62 |
| Pep-3 | 659 | KWFETWFTEWPKKRK | Cell-penetrating | 0.73 | Low | 0.545 |
| Pep-3 | 660 | KWFETWFTEWPKKRKGGC | Cell-penetrating | 0.89 | Low | 0.548 |
| PenetraMax | 661 | KWFKIQMQIRRWKNKR | Cell-penetrating | 0.99 | High | 0.606 |
| MTp1-2 | 662 | KWFRVYRGIYRRRGK | Cell-penetrating | 0.98 | High | 0.685 |
| MTp1-1 | 663 | KWSFRVSYRGISYRRSRGK | Cell-penetrating | 0.96 | Low | 0.69 |
| A11 | 664 | LAELLAELLAELGGGGRRRRRRRRR | Cell-penetrating | 0.99 | Low | 0.605 |
| pVEC mutant | 665 | LAIILRRRIRKQAHAHSK | Cell-penetrating | 0.99 | Low | 0.91 |
| D9 | 666 | LALALALALALALAKLAKLAKLAKLAKIKKI | Cell-penetrating | 1 | High | 0.58 |
| | | KKKIK | | | | |
| D8 | 667 | LALALALALALALALAKIKKIKKIKKIKKLAK | Cell-penetrating | 1 | High | 0.59 |
| | | LAKKIK | | | | |
| D6 | 668 | LALALALALALALALAKKLKKLKKLKKLKK | Cell-penetrating | 1 | High | 0.53 |
| | | LKKLKYAK | | | | |
| D10 | 669 | LALALALALALALALAKLAKLAKLAKLAKL | Cell-penetrating | 1 | High | 0.5 |
| | | AKKIK | | | | |
| A12 | 670 | LAQLLAQLLAQLGGGGRRRRRRRRR | Cell-penetrating | 0.99 | Low | 0.55 |
| Xentry peptides | | lcl | Cell-penetrating | 0.67 | High | 0.57 |
| Xentry peptides | 671 | LCLE | Cell-penetrating | 0.56 | High | 0.628 |
| Xentry peptides | 672 | LCLH | Cell-penetrating | 0.69 | Low | 0.507 |
| Xentry peptides | 673 | LCLK | Cell-penetrating | 0.75 | High | 0.68 |
| Xentry peptides | 674 | LCLN | Cell-penetrating | 0.6 | Low | 0.51 |
| Xentry peptides | 675 | LCLQ | Cell-penetrating | 0.68 | High | 0.61 |
| Xentry peptides | 676 | LCLR | Cell-penetrating | 0.78 | High | 0.72 |
| Peptide 45 | 677 | LDITPFLSLTLP | Cell-penetrating | 0.86 | Low | 0.725 |
| Inv10 | 678 | LDTYSPELFCTIRNFYDADRPDRGAAA | Cell-penetrating | 0.78 | Low | 0.98 |
| Tat (43-60) | 679 | LGISYGRKKRRQRRRPPQ | Cell-penetrating | 0.96 | High | 0.84 |
| PN86 | 680 | LGLLLRHLRFIHSNLLANI | Cell-penetrating | 0.91 | Low | 0.58 |
| EGFP-hcT(9- | 681 | LGTYTQDFNKFHTFPQTAIGVGAP | Cell-penetrating | 0.82 | Low | 0.805 |
| 32) | | | | | | |
| B8 | 682 | LHHLLHHLLHLLHHLLHHLHHL | Cell-penetrating | 0.9 | Low | 0.513 |
| TCTP-CPP 34 | 683 | LIIFAIAASHKK | Cell-penetrating | 0.86 | Low | 0.53 |
| TCTP-CPP 35 | 684 | LIIFAILISHKK | Cell-penetrating | 0.82 | Low | 0.53 |
| TCTP-CPP 16 | 685 | LIIFRIAASHKK | Cell-penetrating | 0.94 | Low | 0.57 |
| TCTP-CPP 33 | 686 | LIIFRILISH | Cell-penetrating | 0.65 | Low | 0.59 |
| TCTP-CPP 30 | 687 | LIIFRILISHHH | Cell-penetrating | 0.72 | Low | 0.55 |
| TCTP-CPP 31 | 688 | LIIFRILISHK | Cell-penetrating | 0.72 | Low | 0.51 |
| TCTP-CPP 27 | 689 | LIIFRILISHKK | Cell-penetrating | 0.9 | Low | 0.54 |
| TCTP-CPP 32 | 690 | LIIFRILISHR | Cell-penetrating | 0.77 | Low | 0.51 |
| TCTP-CPP 29 | 691 | LIIFRILISHRR | Cell-penetrating | 0.91 | Low | 0.59 |
| TAM-rMP | 692 | LIKKALAALAKLNI | Cell-penetrating | 0.95 | Low | 0.59 |
| LILIR8 (Alexa) | 693 | LILIGRRRRRRRRGC | Cell-penetrating | 0.99 | High | 0.547 |
| D11 | 694 | LILILILILILILILIKRKKRKKRKKRKKRAKRA | Cell-penetrating | 0.98 | Low | 0.51 |
| | | KHSK | | | | |
| EB1 | 695 | LIRLWSHLIHIWFQNRRLKWKKK | Cell-penetrating | 0.92 | High | 0.668 |
| EB1-Cys | 696 | LIRLWSHLIHIWFQNRRLKWKKKC | Cell-penetrating | 0.89 | High | 0.71 |
| EB-1 | 697 | LIRLWSHLIHIWFQNRRLKWKKKGGC | Cell-penetrating | 0.87 | High | 0.622 |
| TAMARA- | 698 | LKKLAELAHKLLKLG | Cell-penetrating | 0.85 | Low | 0.52 |
| peptide 2 | | | | | | |
| LK-2 | 699 | LKKLCKLLKKLCKLAG | Cell-penetrating | 0.98 | Low | 0.52 |
| LK-1 | 700 | LKKLLKLLKKLLKLAG | Cell-penetrating | 0.99 | Low | 0.51 |
| IDI-K6L9 | 701 | LK1LKkL1kKLLkLL | Cell-penetrating | 0.98 | Low | 0.53 |
| pepR | 702 | LKRWGTIKKSKAINVLRGFRKEIGRMLNILNR | Cell-penetrating | 0.99 | High | 0.655 |
| | | RRR | | | | |
| XI | 703 | LKTLATALTKLAKTLTTL | Cell-penetrating | 0.96 | High | 0.74 |
| XIII | 704 | LKTLTETLKELTKTLTEL | Cell-penetrating | 0.88 | Low | 0.85 |
| pVEC mutant | 705 | LLAILRRRIRKQAHAHSK | Cell-penetrating | 0.99 | Low | 0.96 |
| PN202 | 706 | LLETLLKPFQCRICMRNFSTRQARRNHRRRH | Cell-penetrating | 0.97 | High | 0.523 |
| | | RR | | | | |
| LL-37 | 707 | LLGDFFRKSKEKIGKEFKRIVQRIKDFLRNLV | Cell-penetrating | 0.84 | High | 0.525 |
| | | PRTESC | | | | |
| TP8 | 708 | LLGKINLKALAALAKKIL | Cell-penetrating | 0.97 | Low | 0.78 |
| S6KR | 709 | LLHILRRSIRKQAHAIRK | Cell-penetrating | 0.98 | High | 0.53 |
| S6R | 710 | LLHILRRSIRRQAHAIRR | Cell-penetrating | 0.99 | High | 0.541 |
| pVEC mutant | 711 | LLIALRRRIRKQAHAHSK | Cell-penetrating | 1 | Low | 0.94 |
| pVEC mutant | 712 | LLIIARRRIRKQAHAHSK | Cell-penetrating | 0.99 | Low | 0.92 |
| pVEC mutant | 713 | LLIILARRIRKQAHAHSK | Cell-penetrating | 0.97 | High | 0.89 |
| pVEC mutant | 714 | LLIILRARIRKQAHAHSK | Cell-penetrating | 0.98 | High | 0.9 |
| pVEC mutant | 715 | LLIILRRAIRKQAHAHSK | Cell-penetrating | 0.98 | High | 0.95 |
| pVEC mutant | 716 | LLIILRRRARKQAHAHSK | Cell-penetrating | 1 | Low | 0.89 |
| pVEC mutant | 717 | LLIILRRRIARKQAHAHSK | Cell-penetrating | 0.99 | High | 0.77 |
| pVEC mutant | 718 | LLIILRRRIRAQAHAHSK | Cell-penetrating | 0.98 | High | 0.94 |
| pVEC mutant | 719 | LLIILRRRIRKAAHAHSK | Cell-penetrating | 1 | High | 0.86 |
| pVEC mutant | 720 | LLIILRRRIRKQAAAHSK | Cell-penetrating | 1 | Low | 0.72 |
| pVEC mutant | 721 | LLIILRRRIRKQAHAASK | Cell-penetrating | 1 | High | 0.72 |
| pVEC mutant | 722 | LLIILRRRIRKQAHAHAK | Cell-penetrating | 1 | High | 0.84 |
| pVEC mutant | 723 | LLIILRRRIRKQAHAHSA | Cell-penetrating | 0.97 | High | 0.87 |
| pVEC | 724 | LLIILRRRIRKQAHAHSK | Cell-penetrating | 1 | High | 0.53 |
| FAM-pVEC- | 725 | LLIILRRRIRKQAHAHSKNHQQQNPHQPPM | Cell-penetrating | 0.91 | Low | 0.54 |
| gHo (FAM- | | | | | | |
| gHoPe2) | | | | | | |
| P9R | 726 | LLIILRRRIRRRARARSR | Cell-penetrating | 0.99 | High | 0.582 |
| E165 | 727 | LLKKRKVVRLIKFLLK | Cell-penetrating | 1 | High | 0.87 |
| PF20 | 728 | LLKLLKKLLKLLKKLLKLL | Cell-penetrating | 1 | Low | 0.513 |
| XII | 729 | LLKTTALLKTTALLKTTA | Cell-penetrating | 0.96 | Low | 0.793 |
| XIV | 730 | LLKTTELLKTTELLKTTE | Cell-penetrating | 0.88 | Low | 0.86 |
| Xentry peptides | 731 | LLLLR | Cell-penetrating | 0.82 | High | 0.63 |
| Xentry peptides | 732 | LLLR | Cell-penetrating | 0.84 | High | 0.55 |
| Xentry peptides | 733 | LLLRR | Cell-penetrating | 0.88 | High | 0.51 |
| Xentry peptides | | LLR | Cell-penetrating | 0.8 | High | 0.56 |
| P6 | 734 | LLRARWRRRRSRRFR | Cell-penetrating | 1 | Low | 0.558 |
| S9RH | 735 | LLRHLRRHIRRARRHIRR | Cell-penetrating | 0.99 | High | 0.503 |
| S9R | 736 | LLRILRRSIRRARRAIRR | Cell-penetrating | 1 | Low | 0.561 |
| Mgpe-4 | 737 | LLYWFRRRHRHHRRRHRR | Cell-penetrating | 0.98 | High | 0.6 |
| TP13 | 738 | LNSAGYLLGKALAALAKKIL | Cell-penetrating | 0.92 | Low | 0.81 |
| TP7 | 739 | LNSAGYLLGKINLKALAALAKKIL | Cell-penetrating | 0.92 | High | 0.86 |
| TP15 | 740 | LNSAGYLLGKLKALAALAK | Cell-penetrating | 0.92 | Low | 0.9 |
| TP12 | 741 | LNSAGYLLGKLKALAALAKIL | Cell-penetrating | 0.91 | Low | 0.54 |
| Peptide 44 | 742 | LNVPPSWFLSQR | Cell-penetrating | 0.86 | Low | 0.6 |
| Peptide 46 | 743 | LPHPVLHMGPLR | Cell-penetrating | 0.92 | High | 0.5 |
| A4 | 744 | LRHHLRHLLRHLRHLLRHLRHHLRHLLRH | Cell-penetrating | 0.99 | High | 0.508 |
| D12 | 745 | LRHLLRHLLRHLRHL | Cell-penetrating | 0.97 | Low | 0.543 |
| A3 | 746 | LRHLLRHLLRHLRHLLRHLRHLLRHLLRH | Cell-penetrating | 0.99 | Low | 0.503 |
| DPV15 | 747 | LRRERQSRLRRERQSR | Cell-penetrating | 0.98 | Low | 0.52 |
| p28 | 748 | LSTAADMQGVVTDGMASGLDKDYLKPDD | Cell-penetrating | 0.58 | High | 0.77 |
| Peptide 31 | 749 | LTMPSDLQPVLW | Cell-penetrating | 0.7 | Low | 0.79 |
| Peptide 22 | 750 | LTRNYEAWVPTP | Cell-penetrating | 0.72 | Low | 0.758 |
| X-Pep | 751 | MAARL | Cell-penetrating | 0.6 | Low | 0.623 |
| derivative | | | | | | |
| X-Pep | 752 | MAARLCCQ | Cell-penetrating | 0.5 | Low | 0.54 |
| N-terminus of | 753 | MAARLCCQLDPARDV | Non-cell-penetrating | 0.52 | — | — |
| X-Pep | | | | | | |
| N-terminus of | 754 | MAARLCCQLDPARDVLCLRP | Cell-penetrating | 0.83 | Low | 0.63 |
| X-Pep | | | | | | |
| TCTP(I-9) I2A | 755 | MAIYRDLIS | Non-cell-penetrating | 0.69 | — | — |
| subsetution | | | | | | |
| mutant | | | | | | |
| CPPK | 756 | MAMPGEPRRANVMAHKLEPASLQLR NSCA | Cell-penetrating | 0.86 | Low | 0.715 |
| Human Prp (1- | 757 | MANLGCWMLVLFVATWSDLGLCKKRPKP | Cell-penetrating | 0.94 | Low | 0.58 |
| 28) | | | | | | |
| Mouse Prp (1- | 758 | MANLGYWLLALFVTMWTDVGLCKKRPKP | Cell-penetrating | 0.9 | Low | 0.61 |
| 28) | | | | | | |
| CPPL | 759 | MAPQRDTVGGRTTPPSWGPAKAQLRNSCA | Cell-penetrating | 0.82 | Low | 0.775 |
| LAMBDA N | 760 | MDAQTRRRERRAEKQAQWKAANGC | Cell-penetrating | 0.92 | Low | 0.875 |
| (1-22) | | | | | | |
| Crot (27-39) | 761 | MDCRWRWKCCKK | Cell-penetrating | 0.79 | High | 0.93 |
| derevative | | | | | | |
| Peptide 2 | 762 | MGLGLHLLVLAAALQGAKKKRKV | Cell-penetrating | 0.94 | High | 0.53 |
| Peptide 1 | 763 | MGLGLHLLVLAAALQGAWSQPKKKRKV | Cell-penetrating | 0.98 | Low | 0.607 |
| Peptide 6 | 764 | MHKRPTTPSRKM | Cell-penetrating | 0.88 | Low | 0.58 |
| TCTP(1-9 I3A | 765 | MIAYRDLIS | Non-cell-penetrating | 0.74 | — | — |
| subsetution | | | | | | |
| mutant | | | | | | |
| TCTP(1-9) | 766 | MIIARDLIS | Non-cell-penetrating | 0.71 | — | — |
| Y4A | | | | | | |
| subsetution | | | | | | |
| mutant | | | | | | |
| TCTP-CPP 26 | 767 | MIIFAIAASHKK | Cell-penetrating | 0.76 | Low | 0.53 |
| TCTP-CPP 24 | 768 | MIIFKIAASHKK | Cell-penetrating | 0.8 | Low | 0.545 |
| TCTP-CPP 14 | 769 | MIIFRAAASHKK | Cell-penetrating | 0.97 | Low | 0.59 |
| TCTP-CPP 13 | 770 | MIIFRALISHKK | Cell-penetrating | 0.86 | Low | 0.57 |
| TCTP-CPP 3 | 771 | MIIFRDLISH | Non-cell-penetrating | 0.71 | - | - |
| TCTP-CPP 12 | 772 | MIIFRIAASHKK | Cell-penetrating | 0.91 | Low | 0.57 |
| TCTP-CPP 22 | 773 | MIIFRIAATHKK | Cell-penetrating | 0.87 | Low | 0.55 |
| TCTP-CPP 20 | 774 | MIIFRIAAYHKK | Cell-penetrating | 0.88 | Low | 0.55 |
| TCTP-CPP 28 | 775 | MIIFRILISHKK | Cell-penetrating | 0.82 | Low | 0.57 |
| TCTP-CPP 9 | 776 | MIIRRDLISE | Non-cell-penetrating | 0.59 | — | — |
| TCTP-CPP 4 | 777 | MIISRDLISH | Non-cell-penetrating | 0.7 | — | — |
| TCTP(1-9) | 778 | MIIYADLIS | Non-cell-penetrating | 0.76 | — | — |
| RSA | | | | | | |
| subsetution | | | | | | |
| mutant | | | | | | |
| TCTP-CPP 11 | 779 | MIIYARRAEE | Non-cell-penetrating | 0.53 | — | — |
| TCTP-CPP 10 | 780 | MITYRAEISH | Non-cell-penetrating | 0.87 | — | — |
| TCTP(1-9) | 781 | MITYRALIS | Non-cell-penetrating | 0.58 | — | — |
| D6A | | | | | | |
| subsetution | | | | | | |
| mutant | | | | | | |
| TCTP-CPP 7 | 782 | MIIYRALISHKK | Cell-penetrating | 0.92 | Low | 0.55 |
| TCTP (1-6) | 783 | MIIYRD | Non-cell-penetrating | 0.71 | — | — |
| deletion mutant | | | | | | |
| TCTP(1-9) | 784 | MIIYRDAIS | Non-cell-penetrating | 0.8 | — | — |
| L7A | | | | | | |
| subsetution | | | | | | |
| mutant | | | | | | |
| TCTP-CPP 2 | 785 | MIIYRDKKSH | Cell-penetrating | 0.58 | Low | 0.66 |
| TCTP (1-7) | 786 | MIIYRDL | Non-cell-penetrating | 0.68 | — | — |
| deletion mutant | | | | | | |
| TCTP(1-9) I8A | 787 | MIIYRDLAS | Non-cell-penetrating | 0.75 | — | — |
| subsetution | | | | | | |
| mutant | | | | | | |
| TCTP (1-8) | 788 | MIIYRDLI | Non-cell-penetrating | 0.71 | — | — |
| deletion mutant | | | | | | |
| TCTP(1-9) | 789 | MIIYRDLIA | Non-cell-penetrating | 0.73 | — | — |
| S9A | | | | | | |
| subsetution | | | | | | |
| mutant | | | | | | |
| TCTP (1-9) | 790 | MIIYRDLIS | Non-cell-penetrating | 0.74 | — | — |
| deletion mutant | | | | | | |
| TCTPPTD | 791 | MITYRDLISH | Non-cell-penetrating | 0.76 | — | — |
| TCTP-CPP 1 | 792 | MIIYRDLISKK | Cell-penetrating | 0.79 | Low | 0.615 |
| TCTP-CPP 8 | 793 | MIIYRIAASHKK | Cell-penetrating | 0.94 | Low | 0.56 |
| BagP | 794 | MLLLTRRRST | Cell-penetrating | 0.7 | Low | 0.554 |
| Bac-ELP-H1 | 795 | MRRIRPRPPRLPRPRPRPLPFPRPGGCYPG | Cell-penetrating | 0.92 | Low | 0.76 |
| Peptide 56 | 796 | MTPSSLSTLPWP | Cell-penetrating | 0.96 | Low | 0.79 |
| Bovine Prp (1- | 797 | MVKSKIGSWILVLFVAMWSDVGLCKKRPKP | Cell-penetrating | 0.83 | Low | 0.675 |
| 30) | | | | | | |
| ARF(1-22) | 798 | MVRRFLVTLRIRRACGPPRVRV | Cell-penetrating | 0.88 | High | 0.935 |
| ARF(1-37) | 799 | MVRRFLVTLRIRRACGPPRVRVFVVHIPRLTG | Cell-penetrating | 0.86 | High | 0.582 |
| | | EWAAP | | | | |
| M918(R-K) | 800 | MVTVLFKRLRIRRACGPPRVKV | Cell-penetrating | 0.89 | High | 0.84 |
| M918 | 801 | MVTVLFRRLRIRRACGPPRVRV | Cell-penetrating | 0.9 | High | 0.94 |
| P22 N | 802 | NAKTRRHERRRKLAIERGC | Cell-penetrating | 0.95 | High | 0.76 |
| FAM-gHo | 803 | NHQQQNPHQPPM | Cell-penetrating | 0.53 | Low | 0.76 |
| FAM-gHo- | 804 | NHQQQNPHQPPMLLIILRRRIRKQAHAHSK | Cell-penetrating | 0.91 | Low | 0.54 |
| pVEC (FAM- | | | | | | |
| gHoPe3) | | | | | | |
| Peptide 50 | 805 | NIENSTLATPLS | Cell-penetrating | 0.9 | Low | 0.79 |
| SRAM C105Y | 806 | NKPILVFY | Non-cell-penetrating | 0.56 | — | — |
| Peptide 18 | 807 | NKRILIRIMTRP | Cell-penetrating | 0.94 | Low | 0.655 |
| Asn-Oct-6 | 808 | NNNAAGRKRKKRT | Cell-penetrating | 0.98 | Low | 0.855 |
| FHV-TA (39- | 809 | NRARRNRRRVR | Cell-penetrating | 0.97 | High | 0.588 |
| 49) | | | | | | |
| E8 | 810 | NRHFRFFFNFTNR | Cell-penetrating | 0.71 | High | 0.55 |
| pAntp (51-58) | 811 | NRRMKWKK | Cell-penetrating | 0.9 | High | 0.91 |
| Peptide 60 | 812 | NSGTMQSASRAT | Cell-penetrating | 0.87 | Low | 0.77 |
| Peptide 1-SΔ | 813 | NTCTWLKYH | Non-cell-penetrating | 0.61 | — | — |
| Peptide 1 | 814 | NTCTWLKYHS | Non-cell-penetrating | 0.63 | — | — |
| Peptide 1-C3G | 815 | NTGTWLKYHS | Cell-penetrating | 0.51 | Low | 0.82 |
| EDN(32-41) | 816 | NYQRRCKNQN | Cell-penetrating | 0.75 | Low | 0.71 |
| ECP(32- | 817 | NYQWRCKNQN | Cell-penetrating | 0.51 | Low | 0.703 |
| 41) R3Q | | | | | | |
| ECP(32- | 818 | NYRRRCKNQN | Cell-penetrating | 0.87 | Low | 0.63 |
| 41) W4R | | | | | | |
| ECP(32-38) | 819 | NYRWRCK | Cell-penetrating | 0.85 | High | 0.77 |
| ECP(32-39) | 820 | NYRWRCKN | Cell-penetrating | 0.8 | High | 0.63 |
| ECP(32-40) | 821 | NYRWRCKNQ | Cell-penetrating | 0.76 | High | 0.54 |
| ECP(32-41) | 822 | NYRWRCKNQN | Cell-penetrating | 0.69 | Low | 0.58 |
| Peptide 48 | 823 | NYTTYKSHFQDR | Cell-penetrating | 0.74 | Low | 0.675 |
| CTP501 | 824 | PARAARRAARR | Cell-penetrating | 0.99 | Low | 0.692 |
| C105Y | 825 | PFVYLI | Cell-penetrating | 0.69 | Low | 0.54 |
| derivative | | | | | | |
| Peptide 4 | 826 | PIRRRKKLRRLK | Cell-penetrating | 1 | High | 0.619 |
| SV40 | 827 | PKKKRKV | Cell-penetrating | 0.95 | Low | 0.868 |
| PV-S4(13) | 828 | PKKKRKVALWKTLLKKVLKA | Cell-penetrating | 0.99 | High | 0.52 |
| NS | 829 | PKKKRKVWKLLQQFFGLM | Cell-penetrating | 0.96 | Low | 0.61 |
| PreS2 (41-52) | 830 | PLSSIFSRIGDP | Cell-penetrating | 0.9 | Low | 0.72 |
| Bip5 | 831 | PMLKE | Non-cell-penetrating | 0.64 | — | — |
| Peptide 21 | 832 | PNTRVRPDVSF | Cell-penetrating | 0.84 | Low | 0.76 |
| Peptide 14 | 833 | PPHNRIQRRLNM | Cell-penetrating | 0.94 | Low | 0.65 |
| Secretory | 834 | PPKKSAQCLRYKKPE | Cell-penetrating | 0.91 | Low | 0.607 |
| leukoprotease | | | | | | |
| inhibitor | | | | | | |
| derived PTD | | | | | | |
| Bac7-24 | 835 | PPRLPRPRPRPLPFPRPG | Cell-penetrating | 0.95 | Low | 0.96 |
| Peptide 3 | 836 | PPRLRKRRQLNM | Cell-penetrating | 1 | Low | 0.53 |
| Peptide 13 | 837 | PQNRLQIRRHSK | Cell-penetrating | 1 | Low | 0.611 |
| Bac15-24 | 838 | PRPLPFPRPG | Cell-penetrating | 0.84 | High | 0.71 |
| Bac5-24 | 839 | PRPPRLPRPRPRPLPFPRPG | Cell-penetrating | 0.97 | Low | 0.95 |
| Bac13-24 | 840 | PRPRPLPFPRPG | Cell-penetrating | 0.87 | Low | 0.87 |
| Bac11-24 | 841 | PRPRPRPLPFPRPG | Cell-penetrating | 0.92 | Low | 0.94 |
| Peptide 11 | 842 | PSKRLLHNNLRR | Cell-penetrating | 0.96 | Low | 0.53 |
| PreS2 3S | 843 | PSSSSSSRIGDP | Cell-penetrating | 0.9 | Low | 0.76 |
| Mutant | | | | | | |
| Peptide 61 | 844 | QAASRVENYMHR | Cell-penetrating | 0.77 | Low | 0.59 |
| TCTP-CPP 5 | 845 | QIISRDLISH | Non-cell-penetrating | 0.67 | — | — |
| pAntp (44-58) | 846 | QIKIWFQNRRMKWKK | Cell-penetrating | 0.96 | High | 0.929 |
| IX | 847 | QLALQLALQALQAALQLA | Cell-penetrating | 0.89 | High | 0.88 |
| Bip17 | 848 | QLPVM | Cell-penetrating | 0.51 | High | 0.6 |
| pAntp (50-58) | 849 | QNRRMKWKK | Cell-penetrating | 0.88 | High | 0.96 |
| Peptide 58 | 850 | QPIIITSPYLPS | Cell-penetrating | 0.94 | Low | 0.72 |
| No. 2510 | 851 | QQHLLIAINGYPRYN | Cell-penetrating | 0.85 | High | 0.695 |
| Peptide 10 | 852 | QRIRKSKISRTL | Cell-penetrating | 0.92 | Low | 0.682 |
| Peptide 28 | 853 | QSPTDFTFPNPL | Cell-penetrating | 0.84 | Low | 0.755 |
| Lambda-N (48- | 854 | QTRRRERRAEKQAQW | Cell-penetrating | 0.89 | Low | 0.58 |
| 62) | | | | | | |
| M6 | 855 | QWQRNMRKVR | Cell-penetrating | 0.87 | Low | 0.89 |
| M5 | 856 | QWQRNMRKVRGPPVSCIKR | Cell-penetrating | 0.82 | Low | 0.67 |
| Buforin-II | 857 | RAGLQFPVGRVHRLLRK | Cell-penetrating | 0.94 | Low | 0.54 |
| Ala44 | 858 | RAIKIWFQNRRMKWKK | Cell-penetrating | 1 | High | 0.99 |
| substitution | | | | | | |
| mutant of | | | | | | |
| pAntp (43-58) | | | | | | |
| Ala50 | 859 | RAKRRQRRR | Cell-penetrating | 1 | Low | 0.96 |
| substitution | | | | | | |
| mutant of Tat | | | | | | |
| (49-57) | | | | | | |
| 32 RA | 860 | RARARARARARARARARARARARARARAR | Cell-penetrating | 1 | Low | 0.674 |
| | | ARA | | | | |
| No.14-12 | 861 | RAWMRWYSPTTRRYG | Cell-penetrating | 0.97 | High | 0.89 |
| E3 | 862 | RFTFHFRFEFTFHFE | Non-cell-penetrating | 0.71 | — | — |
| A10 | 863 | RFTFHFRFEFTFHFEGGGRRRRRRR | Cell-penetrating | 0.96 | High | 0.59 |
| cRGD | 864 | RGDfK | Cell-penetrating | 0.66 | Low | 0.745 |
| P2 | 865 | RGDGPRRRPRKRRGR | Cell-penetrating | 0.99 | Low | 0.555 |
| PD1 | 866 | RGDRGDRRDLRLDRGDLRC | Cell-penetrating | 0.93 | Low | 0.805 |
| PD2 | 867 | RGDRLDRRDLRLDRRDLRC | Cell-penetrating | 0.89 | Low | 0.627 |
| PE1 | 868 | RGERGERRELRLERGELRC | Cell-penetrating | 0.96 | Low | 0.697 |
| PE2 | 869 | RGERLERRELRLERRELRC | Cell-penetrating | 0.92 | High | 0.5 |
| SynB5 | 870 | RGGRLAYLRRRWAVLGR | Cell-penetrating | 1 | Low | 0.81 |
| SynB1 | 871 | RGGRLSYSRRRFSTSTGR | Cell-penetrating | 0.95 | Low | 0.925 |
| SynB1-ELP- | 872 | RGGRLSYSRRRFSTSTGRA | Cell-penetrating | 0.97 | Low | 0.828 |
| H1 | | | | | | |
| P7 | 873 | RGPRRQPRRHRRPRR | Cell-penetrating | 1 | High | 0.578 |
| PN404 | 874 | RGSRRAVTRAQRRDGRRRRRSRRESYSVYV | Cell-penetrating | 0.97 | Low | 0.652 |
| | | YRVLRQ | | | | |
| F3 | 875 | RHHLRHLRRHL | Cell-penetrating | 1 | Low | 0.545 |
| B5 | 876 | RHHLRHLRRHLRHLLRHLRHHL | Cell-penetrating | 1 | High | 0.528 |
| A1 | 877 | RHHLRHLRRHLRHLLRHLRHHLRHLRRHLR | Cell-penetrating | 0.99 | Low | 0.533 |
| | | HLL | | | | |
| B6 | 878 | RHHRRHHRRHRRHHRRHHRHHR | Cell-penetrating | 1 | Low | 0.51 |
| PDX-1-PTD | 879 | RHIKIWFQNRRMKWKK | Cell-penetrating | 0.99 | High | 0.927 |
| E7 | 880 | RHNFRFFFNFRTNR | Cell-penetrating | 0.96 | High | 0.56 |
| Peptide 5 | 881 | RHVYHVLLSQ | Cell-penetrating | 0.59 | Low | 0.603 |
| LR8DHFRI | 882 | RIFIGC | Non-cell-penetrating | 0.59 | — | — |
| LR15DL | 883 | RIFIHFRIGC | Cell-penetrating | 0.5 | Low | 0.58 |
| LR8DHF | 884 | RIFIRIGC | Cell-penetrating | 0.57 | Low | 0.665 |
| Human c Jun | 885 | RIKAERKRMRNRIAASKSRKRKLERIARGC | Cell-penetrating | 0.98 | High | 0.845 |
| (252-279) | | | | | | |
| LR11 | 886 | RILQQLLFIHF | Cell-penetrating | 0.73 | Low | 0.64 |
| LR15 | 887 | RILQQLLFIHFRIGC | Cell-penetrating | 0.65 | Low | 0.58 |
| LR17 | 888 | RILQQLLFIHFRIGCRH | Cell-penetrating | 0.73 | High | 0.537 |
| LR20 | 889 | RILQQLLFIHFRIGCRHSRI | Cell-penetrating | 0.93 | High | 0.51 |
| DS4.3 | 890 | RIMRILRILKLAR | Cell-penetrating | 0.98 | Low | 0.66 |
| Peptide 8 | 891 | RIRMIQNLIKKT | Cell-penetrating | 0.96 | Low | 0.605 |
| Ala51 | 892 | RKARRQRRR | Cell-penetrating | 1 | Low | 0.942 |
| substitution | | | | | | |
| mutant of Tat | | | | | | |
| (49-57) | | | | | | |
| PAF96 | 893 | RKKAAA | Cell-penetrating | 0.84 | Low | 0.705 |
| A1a52 | 894 | RKKARQRRR | Cell-penetrating | 1 | Low | 0.96 |
| substitution | | | | | | |
| mutant of Tat | | | | | | |
| (49-57) | | | | | | |
| hBCPP | 895 | RKKNPNCRRH | Cell-penetrating | 0.87 | Low | 0.548 |
| Ala53 | 896 | RKKRAQRRR | Cell-penetrating | 0.98 | Low | 0.91 |
| substitution | | | | | | |
| mutant of Tat | | | | | | |
| (49-57) | | | | | | |
| Ala54 | 897 | RKKRRARRR | Cell-penetrating | 0.99 | High | 0.74 |
| substitution | | | | | | |
| mutant of Tat | | | | | | |
| (49-57) | | | | | | |
| Ala55 | 898 | RKKRRQARR | Cell-penetrating | 0.98 | Low | 0.9 |
| substitution | | | | | | |
| mutant of Tat | | | | | | |
| (49-57) | | | | | | |
| Tat (49-55) | 899 | RKKRRQR | Cell-penetrating | 1 | Low | 0.803 |
| Ala56 | 900 | RKKRRQRAR | Cell-penetrating | 0.99 | Low | 0.94 |
| substitution | | | | | | |
| mutant of Tat | | | | | | |
| (49-57) | | | | | | |
| Tat (49-56) | 901 | RKKRRQRR | Cell-penetrating | 1 | High | 0.68 |
| Ala57 | 902 | RKKRRQRRA | Cell-penetrating | 0.99 | Low | 0.865 |
| substitution | | | | | | |
| mutant of Tat | | | | | | |
| (49-57) | | | | | | |
| Tat (49-57) | 903 | RKKRRQRRR | Cell-penetrating | 1 | High | 0.88 |
| Tat-Cys | 904 | RKKRRQRRRGC | Cell-penetrating | 0.98 | High | 0.548 |
| Tat | 905 | RKKRRQRRRGGG | Cell-penetrating | 0.96 | Low | 0.535 |
| TatLK15 | 906 | RKKRRQRRRGGGKLLKLLLKLLLKLLK | Cell-penetrating | 0.99 | Low | 0.56 |
| dTAT | 907 | RKKRRQRRRHRRKKR | Cell-penetrating | 1 | High | 0.527 |
| PN28 | 908 | RKKRRQRRRPPQCAAVALLPAVLLALLAP | Cell-penetrating | 0.98 | Low | 0.577 |
| Tat2-Nat | 909 | RKKRRQRRRRKKRRQRRR | Cell-penetrating | 1 | High | 0.546 |
| DPV3 | 910 | RKKRRRESRKKRRRES | Cell-penetrating | 0.98 | High | 0.83 |
| DPV3 | 911 | RKKRRRESRKKRRRESC | Cell-penetrating | 0.85 | Low | 0.843 |
| DPV3/10 | 912 | RKKRRRESRRARRSPRHL | Cell-penetrating | 0.98 | Low | 0.554 |
| MMD49 | 913 | RKKRRRESWVHLPPPVHLPPPGGHHHHHH | Cell-penetrating | 0.96 | Low | 0.65 |
| PAF26 | 914 | RKKWFW | Cell-penetrating | 0.75 | Low | 0.633 |
| Camptide | 915 | RKLTTIFPLNWKYRKALSLG | Cell-penetrating | 0.93 | Low | 0.63 |
| C3 | 916 | RLALRLALRALRAALRLA | Cell-penetrating | 1 | High | 0.512 |
| No.14-13 | 917 | RLAMRWYSPTTRRYG | Cell-penetrating | 0.97 | High | 0.87 |
| No.14-25 | 918 | RLFMRFYSPTTRRYG | Cell-penetrating | 0.95 | High | 0.93 |
| D11 | 919 | RLHHRLHRRLHRLHR | Cell-penetrating | 0.99 | Low | 0.56 |
| A2 | 920 | RLHHRLHRRLHRLHRRLHRLHHRLHRRLH | Cell-penetrating | 1 | High | 0.54 |
| C4 | 921 | RLHLRLHLRHLRHHLRLH | Cell-penetrating | 0.99 | Low | 0.59 |
| E2 | 922 | RLHRRLHRRLHRLHR | Cell-penetrating | 1 | Low | 0.51 |
| AS | 923 | RLHRRLHRRLHRLHRRLHRLHRRLHRRLH | Cell-penetrating | 1 | High | 0.51 |
| 28 | 924 | RLIMRIYAPTTRRYG | Cell-penetrating | 0.97 | High | 0.79 |
| No.14-26 | 925 | RLIMRIYSPTTRRYG | Cell-penetrating | 0.98 | High | 0.89 |
| No.14-24 | 926 | RLLMRLYSPTTRRYG | Cell-penetrating | 0.97 | Low | 0.73 |
| C6 | 927 | RLLRLLLRLWRRLLRLLR | Cell-penetrating | 0.99 | Low | 0.58 |
| 1b | 928 | RLLRLLRLL | Cell-penetrating | 0.84 | Low | 0.55 |
| PL | 929 | RLLRLLRRLLRLLRRLLRC | Cell-penetrating | 0.99 | Low | 0.55 |
| Bac9-24 | 930 | RLPRPRPRPLPFPRPG | Cell-penetrating | 0.95 | Low | 0.95 |
| D2 | 931 | RLRLRLRLRLRLRLRLKLLKLLKLLKLLKKK | Cell-penetrating | 1 | High | 0.537 |
| | | KKKKGYK | | | | |
| D3 | 932 | RLRLRLRLRLRLRLRLKNNKNNKNNKNNKK | Cell-penetrating | 0.99 | High | 0.598 |
| | | KKKKKGYK | | | | |
| D1 | 933 | RLRLRLRLRLRLRLRLKRLKRLKRLKRLKKK | Cell-penetrating | 1 | High | 0.591 |
| | | KKKKGYK | | | | |
| SG3 | 934 | RLSGMNEVLSFRWL | Cell-penetrating | 0.74 | Low | 0.64 |
| No.14-29 | 935 | RLVMRVYSPTTRRYG | Cell-penetrating | 0.97 | High | 0.78 |
| No.14-14 | 936 | RLWARWYSPTTRRYG | Cell-penetrating | 0.99 | High | 0.88 |
| No.14-15 | 937 | RLWMAWYSPTTRRYG | Cell-penetrating | 0.83 | Low | 0.82 |
| No.14-16 | 938 | RLWMRAYSPTTRRYG | Cell-penetrating | 1 | Low | 0.68 |
| No.14-17 | 939 | RLWMRWASPTTRRYG | Cell-penetrating | 0.99 | High | 0.96 |
| No.14-18 | 940 | RLWMRWYAPTTRRYG | Cell-penetrating | 0.98 | High | 0.98 |
| No.14-20 | 941 | RLWMRWYSPATRRYG | Cell-penetrating | 0.99 | High | 1 |
| RLW | 942 | RLWMRWYSPRTRAYG | Cell-penetrating | 0.96 | High | 0.655 |
| No.14-21 | 943 | RLWMRWYSPTARRYG | Cell-penetrating | 0.99 | High | 1 |
| No.14-22 | 944 | RLWMRWYSPTTARYG | Cell-penetrating | 0.91 | Low | 0.85 |
| No .14-3R | 945 | RLWMRWYSPTTRAYG | Cell-penetrating | 0.91 | Low | 0.92 |
| No.14-23 | 946 | RLWMRWYSPTTRRAG | Cell-penetrating | 0.98 | High | 0.89 |
| No.14-35 | 947 | RLWMRWYSPTTRRYA | Cell-penetrating | 0.98 | High | 0.98 |
| No.14-1 | 948 | RLWMRWYSPTTRRYG | Cell-penetrating | 0.99 | High | 0.98 |
| No.14-9 | 949 | RLWMRWYSPWTRRWG | Cell-penetrating | 0.97 | Low | 0.65 |
| No.14-8 | 950 | RLWMRWYSPWTRRYG | Cell-penetrating | 0.98 | High | 0.87 |
| PN366 | 951 | RLWRALPRVLRRLLRP | Cell-penetrating | 0.99 | Low | 0.52 |
| No.14-30 | 952 | RLYMRYYSPTTRRYG | Cell-penetrating | 0.97 | High | 0.93 |
| pAntp (53-58) | 953 | RMKWKK | Cell-penetrating | 0.89 | Low | 0.77 |
| Alpha Virus | 954 | RNRSRHRR | Cell-penetrating | 0.99 | Low | 0.562 |
| P130 (227-234) | | | | | | |
| PA 1 | 955 | RPARPAR | Cell-penetrating | 0.86 | Low | 0.69 |
| Ala45 | 956 | RQAKIWFQNRRMKWKK | Cell-penetrating | 0.98 | High | 0.98 |
| substitution | | | | | | |
| mutant of | | | | | | |
| pAntp (43-58) | | | | | | |
| RR-S4(13) | 957 | RQARRNRRRALWKTLLKKVLKA | Cell-penetrating | 0.99 | High | 0.522 |
| Rev ARM | 958 | RQARRNRRRC | Cell-penetrating | 0.97 | Low | 0.508 |
| Ems1 | 959 | RQGAARVTSWLGRQLRIAGKRLEGRSK | Cell-penetrating | 0.96 | Low | 0.575 |
| Ala46 | 960 | RQIAIWFQNRRMKWKK | Cell-penetrating | 0.98 | High | 0.914 |
| substitution | | | | | | |
| mutant of | | | | | | |
| pAntp (43-58) | | | | | | |
| Ala47 | 961 | RQIKAWFQNRRMKWKK | Cell-penetrating | 0.99 | High | 0.99 |
| substitution | | | | | | |
| mutant of | | | | | | |
| pAntp (43-58) | | | | | | |
| Ala48 | 962 | RQIKIAFQNRRMKWKK | Cell-penetrating | 1 | High | 0.945 |
| substitution | | | | | | |
| mutant of | | | | | | |
| pAntp (43-58) | | | | | | |
| Pen2W2F | 963 | RQIKIFFQNRRMKFKK | Cell-penetrating | 0.96 | High | 0.623 |
| pAntp mutant | 964 | RQIKIFFQNRRMKWKK | Cell-penetrating | 0.99 | High | 0.844 |
| Antennapedia | 965 | RQIKIQFQNRRKWKK | Cell-penetrating | 1 | High | 0.615 |
| pAntp (43-48) | 966 | RQIKIW | Cell-penetrating | 0.64 | Low | 0.94 |
| Ala49 | 967 | RQIKIWAQNRRMKWKK | Cell-penetrating | 1 | High | 0.98 |
| substitution | | | | | | |
| mutant of | | | | | | |
| pAntp (43-58) | | | | | | |
| Ala50 | 968 | RQIKIWFANRRMKWKK | Cell-penetrating | 0.99 | High | 0.99 |
| substitution | | | | | | |
| mutant of | | | | | | |
| pAntp (43-58) | | | | | | |
| pAntpHD | 969 | RQIKIWFPNRRMKWKK | Cell-penetrating | 0.99 | High | 0.968 |
| (Pro 50) | | | | | | |
| pAntp (43-50) | 970 | RQIKIWFQ | Cell-penetrating | 0.61 | Low | 0.93 |
| Ala51 | 971 | RQIKIWFQARRMKWKK | Cell-penetrating | 0.99 | High | 0.94 |
| substitution | | | | | | |
| mutant of | | | | | | |
| pAntp (43-58) | | | | | | |
| pAntp (43-51) | 972 | RQIKIWFQN | Cell-penetrating | 0.53 | Low | 0.96 |
| Ala52 | 973 | RQIKIWFQNARMKWKK | Cell-penetrating | 0.95 | High | 0.927 |
| substitution | | | | | | |
| mutant of | | | | | | |
| pAntp (43-58) | | | | | | |
| Met-Arg | 974 | RQIKIWFQNMRRKWKK | Cell-penetrating | 1 | High | 0.932 |
| pAntp (43-52) | 975 | RQIKIWFQNR | Cell-penetrating | 0.79 | Low | 0.95 |
| A1a53 | 976 | RQIKIWFQNRAMKWKK | Cell-penetrating | 0.94 | High | 0.89 |
| substitution | | | | | | |
| mutant of | | | | | | |
| pAntp (43-58) | | | | | | |
| pAntp (43-53) | 977 | RQIKIWFQNRR | Cell-penetrating | 0.98 | Low | 0.97 |
| Ala54 | 978 | RQIKIWFQNRRAKWKK | Cell-penetrating | 0.99 | High | 0.97 |
| substitution | | | | | | |
| mutant of | | | | | | |
| pAntp (43-58) | | | | | | |
| pAntp (43-54) | 979 | RQIKIWFQNRRM | Cell-penetrating | 0.96 | Low | 0.83 |
| Ala55 | 980 | RQIKIWFQNRRMAWKK | Cell-penetrating | 0.96 | Low | 0.82 |
| substitution | | | | | | |
| mutant of | | | | | | |
| pAntp (43-58) | | | | | | |
| pAntp (43-55) | 981 | RQIKIWFQNRRMK | Cell-penetrating | 0.96 | High | 0.735 |
| Ala56 | 982 | RQIKIWFQNRRMKAKK | Cell-penetrating | 0.99 | High | 0.883 |
| substitution | | | | | | |
| mutant of | | | | | | |
| pAntp (43-58) | | | | | | |
| pAntp (43-56) | 983 | RQIKIWFQNRRMKW | Cell-penetrating | 0.98 | High | 0.794 |
| Ala57 | 984 | RQIKIWFQNRRMKWAK | Cell-penetrating | 0.99 | Low | 0.91 |
| substitution | | | | | | |
| mutant of | | | | | | |
| pAntp (43-58) | | | | | | |
| pAntp (43-57) | 985 | RQIKIWFQNRRMKWK | Cell-penetrating | 1 | Low | 0.533 |
| Ala58 | 986 | RQIKIWFQNRRMKWKA | Cell-penetrating | 0.99 | Low | 0.868 |
| substitution | | | | | | |
| mutant of | | | | | | |
| pAntp (43-58) | | | | | | |
| Penetratin | 987 | RQIKIWFQNRRMKWKK | Cell-penetrating | 1 | High | 0.973 |
| Pen-Cys | 988 | RQIKIWFQNRRMKWKKC | Cell-penetrating | 0.96 | High | 0.742 |
| PN251 | 989 | RQIKIWFQNRRMKWKKDIMGEWGNEIFGAI | Cell-penetrating | 0.67 | Low | 0.54 |
| | | AGFLG | | | | |
| Pen | 990 | RQIKIWFQNRRMKWKKGC | Cell-penetrating | 0.95 | High | 0.623 |
| CS-Lin-Pen | 991 | RQIKIWFQNRRMKWKKGG | Cell-penetrating | 0.94 | High | 0.599 |
| Penetratin | 992 | RQIKIWFQNRRMKWKKK | Cell-penetrating | 0.98 | High | 0.878 |
| Pen-GFP-Pen | 993 | RQIKIWFQNRRMKWKKRQIKIWFQNRRMKW | Cell-penetrating | 0.91 | Low | 0.6 |
| | | K | | | | |
| pAntpâ€“PKI | 994 | RQIKIWFQNRRMKWKKTYADFIASGRTGRR | Cell-penetrating | 0.97 | High | 0.845 |
| | | NAI | | | | |
| PenArg | 995 | RQIRIWFQNRRMRWRR | Cell-penetrating | 0.99 | High | 0.875 |
| PenArg-Cys | 996 | RQIRIWFQNRRMRWRRC | Cell-penetrating | 0.99 | High | 0.667 |
| Erns11 | 997 | RQLRIAGRRLRGRSR | Cell-penetrating | 1 | Low | 0.637 |
| pAntpHD | 998 | RQPKIWFPNRRKPWKK | Cell-penetrating | 0.96 | High | 0.84 |
| (3 Pro) | | | | | | |
| Peptide 7 | 999 | RQRSRRRPLNIR | Cell-penetrating | 0.99 | Low | 0.645 |
| P5 | 1000 | RRARRPRRLRPAPGR | Cell-penetrating | 1 | Low | 0.58 |
| R2 | 1001 | RRGC | Cell-penetrating | 0.74 | Low | 0.637 |
| V1 | 1002 | RRGRRG | Cell-penetrating | 1 | Low | 0.582 |
| hPER1-PTD | 1003 | RRHHCRSKAKRSR | Cell-penetrating | 0.99 | Low | 0.623 |
| B9 | 1004 | RRHLRRHLRHLRRHLRRHLRHL | Cell-penetrating | 1 | Low | 0.51 |
| RSV-A9 | 1005 | RRIPNRRPRR | Cell-penetrating | 0.94 | Low | 0.55 |
| Bac1-7 | 1006 | RRIRPRP | Cell-penetrating | 0.94 | Low | 0.917 |
| Bac-1-15 | 1007 | RRIRPRPPRLPRPRP | Cell-penetrating | 0.97 | High | 0.68 |
| Bac1-17 | 1008 | RRIRPRPPRLPRPRPRP | Cell-penetrating | 0.97 | Low | 0.82 |
| Bac-ELP43 | 1009 | RRIRPRPPRLPRPRPRPLPFPRPG | Cell-penetrating | 0.93 | Low | 0.94 |
| M593 | 1010 | RRKLSQQKEKK | Cell-penetrating | 0.98 | Low | 0.83 |
| R6L3 | 1011 | RRLLRRLRR | Cell-penetrating | 1 | High | 0.53 |
| Mgpe-3 | 1012 | RRLRHLRHHYRRRWHRFR | Cell-penetrating | 0.97 | Low | 0.523 |
| SynB3 | 1013 | RRLSYSRRRF | Cell-penetrating | 0.93 | Low | 0.763 |
| pAntp (52-58) | 1014 | RRMKWKK | Cell-penetrating | 0.91 | High | 0.8 |
| Peptide 5 | 1015 | RRQRRTSKLMKR | Cell-penetrating | 0.97 | Low | 0.625 |
| TMR-R3 | | RRR | Cell-penetrating | 0.96 | High | 0.58 |
| Lambda-N | 1016 | RRRERRAEK | Cell-penetrating | 0.93 | Low | 0.58 |
| Truncated (50- | | | | | | |
| 58) | | | | | | |
| P3 | 1017 | RRRQKRIVVRRRLIR | Cell-penetrating | 1 | Low | 0.52 |
| Retro-Tat (57- | 1018 | RRRQRRKKR | Cell-penetrating | 1 | High | 0.9 |
| 49) | | | | | | |
| dfTAT | 1019 | RRRQRRKKRGYCKCKYGRKKRRQRRR | Cell-penetrating | 0.99 | High | 0.627 |
| PN81 | 1020 | RRRQRRKRGGDIMGEWGNEIFGAIAGFLG | Cell-penetrating | 0.85 | Low | 0.71 |
| R4 | 1021 | RRRR | Cell-penetrating | 1 | High | 0.59 |
| FHV coat (35- | 1022 | RRRRNRTRRNRRRVRGC | Cell-penetrating | 0.99 | High | 0.86 |
| 49) | | | | | | |
| R5 | 1023 | RRRRR | Cell-penetrating | 0.99 | Low | 0.71 |
| R5H3 | 1024 | RRRRRHHH | Cell-penetrating | 0.95 | High | 0.547 |
| R6 | 1025 | RRRRRR | Cell-penetrating | 1 | High | 0.915 |
| R6H3 | 1026 | RRRRRRHHH | Cell-penetrating | 0.96 | High | 0.583 |
| R7 | 1027 | RRRRRRR | Cell-penetrating | 1 | High | 0.89 |
| P7-6 | 1028 | RRRRRRRGGIYLATALAKWALKQ | Cell-penetrating | 0.99 | High | 0.513 |
| P7-4 | 1029 | RRRRRRRGGIYLATALAKWALKQGF | Cell-penetrating | 0.99 | High | 0.57 |
| R7-KLA | 1030 | RRRRRRRGGKLAKLAKKLAKLAK | Cell-penetrating | 1 | Low | 0.502 |
| R7H3 | 1031 | RRRRRRRHHH | Cell-penetrating | 0.98 | High | 0.573 |
| R6-Pen(W-L) | 1032 | RRRRRRRQIKILFQNRRMKWKKGGC | Cell-penetrating | 0.97 | High | 0.555 |
| R8 | 1033 | RRRRRRRR | Cell-penetrating | 1 | High | 0.73 |
| R8 | 1034 | RRRRRRRRC | Cell-penetrating | 1 | High | 0.648 |
| R8 (Alexa) | 1035 | RRRRRRRRGC | Cell-penetrating | 0.98 | High | 0.56 |
| R8H3 | 1036 | RRRRRRRRHHH | Cell-penetrating | 0.99 | High | 0.563 |
| R8 | 1037 | RRRRRRRRK | Cell-penetrating | 1 | High | 0.815 |
| R9 | 1038 | RRRRRRRRR | Cell-penetrating | 1 | High | 0.91 |
| PolyR-C-Cy5 | 1039 | RRRRRRRRRC | Cell-penetrating | 1 | High | 0.522 |
| RV24 | 1040 | RRRRRRRRRGPGVTWTPQAWFQWV | Cell-penetrating | 0.97 | Low | 0.61 |
| R9H3 | 1041 | RRRRRRRRRHHH | Cell-penetrating | 1 | Low | 0.593 |
| r9k | 1042 | rrrrrrrrrk | Cell-penetrating | 1 | High | 0.66 |
| R12-alexa | 1043 | RRRRRRRRRR | Cell-penetrating | 1 | High | 0.76 |
| R11 | 1044 | RRRRRRRRRRR | Cell-penetrating | 1 | High | 0.83 |
| R12 | 1045 | RRRRRRRRRRRR | Cell-penetrating | 1 | Low | 0.82 |
| R12 | 1046 | RRRRRRRRRRRRGC | Cell-penetrating | 0.98 | High | 0.598 |
| R15 | 1047 | RRRRRRRRRRRRRRR | Cell-penetrating | 1 | High | 0.53 |
| R16 | 1048 | RRRRRRRRRRRRRRRR | Cell-penetrating | 1 | Low | 0.82 |
| R16 | 1049 | RRRRRRRRRRRRRRRRGC | Cell-penetrating | 0.99 | High | 0.592 |
| R11-PKI | 1050 | RRRRRRRRRRRTYADFIASGRTGRRNAI | Cell-penetrating | 0.99 | High | 0.866 |
| R7W | 1051 | RRRRRRRW | Cell-penetrating | 0.99 | High | 0.583 |
| [R4W4]Cyclic | 1052 | RRRRWWWW | Cell-penetrating | 0.88 | Low | 0.59 |
| RWR | 1053 | RRRRWWWWRRRR | Cell-penetrating | 0.99 | High | 0.535 |
| Erns4 | 1054 | RRVTSWLGRQLRIAGKRLEGRSK | Cell-penetrating | 0.92 | Low | 0.605 |
| P4 | 1055 | RRVWRRYRRQRWCRR | Cell-penetrating | 0.99 | High | 0.667 |
| P8 | 1056 | RRWRRWNRFNRRRCR | Cell-penetrating | 0.99 | High | 0.699 |
| RW16 | 1057 | RRWRRWWRRWWRRWRR | Cell-penetrating | 1 | High | 0.598 |
| R6W3 | 1058 | RRWWRRWRR | Cell-penetrating | 0.99 | High | 0.676 |
| Erns12 | 1059 | rsrgrlrrgairlqrg | Cell-penetrating | 0.95 | Low | 0.572 |
| Inv4 | 1060 | RSVTTEINTLFQTLTSIAEKVDP | Cell-penetrating | 0.71 | Low | 0.882 |
| No.63 | 1061 | RTLVNEYKNTLKFSK | Cell-penetrating | 0.82 | High | 0.675 |
| FHV (40-49) | 1062 | RTRRNRRRVR | Cell-penetrating | 0.98 | High | 0.515 |
| pISL | 1063 | RVIRVWFQNKRCKDKK | Cell-penetrating | 0.96 | High | 0.88 |
| PN158 | 1064 | RVIRWFQNKRCKDKK | Cell-penetrating | 0.97 | High | 0.814 |
| PN316 | 1065 | RVIRWFQNKRSKDKK | Cell-penetrating | 0.97 | High | 0.677 |
| No. 2175 | 1066 | RVREWWYTITLKQES | Cell-penetrating | 0.71 | High | 0.8 |
| ARF(2-14) scr | 1067 | RVRILARFLRTRV | Cell-penetrating | 0.98 | Low | 0.84 |
| Erns5 | 1068 | RVRSWLGRQLRIAGKRLEGRSK | Cell-penetrating | 0.94 | Low | 0.642 |
| ARF(19-31) | 1069 | RVRVFVVHIPRLT | Cell-penetrating | 0.57 | High | 0.76 |
| Erns2 | 1070 | RVTSWLGRQLRIAGKRLEGRSK | Cell-penetrating | 0.89 | Low | 0.545 |
| ECP(34-41) | 1071 | RWRCKNQN | Cell-penetrating | 0.75 | Low | 0.6 |
| RW MIX | 1072 | RWRRWRRWRRWR | Cell-penetrating | 1 | High | 0.648 |
| RW9 | 1073 | RWRRWWRRW | Cell-penetrating | 0.95 | Low | 0.54 |
| Crot (27-39) | 1074 | RWRWKCCKK | Cell-penetrating | 0.91 | High | 0.97 |
| derevative | | | | | | |
| (RW)4 | 1075 | RWRWRWRW | Cell-penetrating | 0.98 | High | 0.537 |
| Peptide 23 | 1076 | SAETVESCLAKSH | Cell-penetrating | 0.83 | Low | 0.74 |
| hPER1-PTD | 1077 | SARHHCRSKAKRSRHH | Cell-penetrating | 0.99 | Low | 0.79 |
| alanine | | | | | | |
| subsitution | | | | | | |
| mutant | | | | | | |
| Peptide 36 | 1078 | SATGAPWKMWVR | Cell-penetrating | 0.83 | Low | 0.59 |
| Peptide 27 | 1079 | SFHQFARATLAS | Cell-penetrating | 0.89 | Low | 0.72 |
| PN279 | 1080 | SGRGKQGGKARAKAKTRSSRAGLQFPVGRV | Cell-penetrating | 0.97 | Low | 0.72 |
| | | HRLLRKG | | | | |
| PN61 | 1081 | SGRGKQGGKARAKAKTRSSRAGLQFPVGRV | Cell-penetrating | 0.98 | Low | 0.69 |
| | | HRLLRKGC | | | | |
| Peptide 38 | 1082 | SHAFTWPTYLQL | Cell-penetrating | 0.86 | Low | 0.613 |
| Peptide 39 | 1083 | SHNWLPLWPLRP | Cell-penetrating | 0.87 | Low | 0.53 |
| TFIIE BETA | 1084 | SKKKKTKV | Cell-penetrating | 0.9 | Low | 0.867 |
| Fushi-tarazu | 1085 | SKRTRQTYTRYQTLELEKEFHFNRYITRRRRI | Cell-penetrating | 0.9 | High | 0.79 |
| (254-313) | | DIANALSLSERQIKIWFQNRRMKSKKDR | | | | |
| Peptide 37 | 1086 | SLGWMLPFSPPF | Cell-penetrating | 0.87 | Low | 0.72 |
| Peptide 15 | 1087 | SMLKRNHSTSNR | Cell-penetrating | 0.95 | Low | 0.595 |
| Peptide 63 | 1088 | SNPWDSLLSVST | Cell-penetrating | 0.87 | Low | 0.79 |
| Peptide 17 | 1089 | SPMQKTMNLPPM | Cell-penetrating | 0.81 | Low | 0.68 |
| hPER1-PTD | 1090 | SRAHHCRSKAKRSRHH | Cell-penetrating | 0.99 | Low | 0.81 |
| alanine | | | | | | |
| subsitution | | | | | | |
| mutant | | | | | | |
| hPER1-PTD | 1091 | SRRAHCRSKAKRSRHH | Cell-penetrating | 1 | Low | 0.79 |
| alanine | | | | | | |
| subsitution | | | | | | |
| mutant | | | | | | |
| DPV10/6 | 1092 | SRRARRSPRESGKKRKRKR | Cell-penetrating | 0.99 | Low | 0.553 |
| DPV10 | 1093 | SRRARRSPRHLGSG | Cell-penetrating | 0.96 | Low | 0.73 |
| hPER1-PTD | 1094 | SRRHACRSKAKRSRHH | Cell-penetrating | 0.99 | Low | 0.82 |
| alanine | | | | | | |
| subsitution | | | | | | |
| mutant | | | | | | |
| hPER1-PTD | 1095 | SRRHHARSKAKRSRHH | Cell-penetrating | 0.99 | Low | 0.761 |
| alanine | | | | | | |
| subsitution | | | | | | |
| mutant | | | | | | |
| hPER1-PTD | 1096 | SRRHHCRAKAKRSRHH | Cell-penetrating | 1 | Low | 0.714 |
| alanine | | | | | | |
| subsitution | | | | | | |
| mutant | | | | | | |
| hPER1-PTD | 1097 | SRRHHCRSAAKRSRHH | Cell-penetrating | 1 | Low | 0.818 |
| alanine | | | | | | |
| subsitution | | | | | | |
| mutant | | | | | | |
| hPER1-PTD | 1098 | SRRHHCRSKAARSRHH | Cell-penetrating | 1 | Low | 0.811 |
| alanine | | | | | | |
| subsitution | | | | | | |
| mutant | | | | | | |
| hPER1-PTD | 1099 | SRRHHCRSKAKASRHH | Cell-penetrating | 1 | Low | 0.814 |
| alanine | | | | | | |
| subsitution | | | | | | |
| mutant | | | | | | |
| hPER1-PTD | 1100 | SRRHHCRSKAKRARHH | Cell-penetrating | 1 | Low | 0.734 |
| alanine | | | | | | |
| subsitution | | | | | | |
| mutant | | | | | | |
| hPER1-PTD | 1101 | SRRHHCRSKAKRSAHH | Cell-penetrating | 0.97 | Low | 0.784 |
| alanine | | | | | | |
| subsitution | | | | | | |
| mutant | | | | | | |
| Peptide 9 | 1102 | SRRKRQRSNMRI | Cell-penetrating | 0.99 | Low | 0.572 |
| SR9 | 1103 | SRRRRRRRRR | Cell-penetrating | 1 | High | 0.665 |
| Crot (27-39) | 1104 | SRWRWKCCKK | Cell-penetrating | 0.94 | High | 0.93 |
| derevative | | | | | | |
| Crot (27-39) | 1105 | SRWRWKCSKK | Cell-penetrating | 0.97 | Low | 0.89 |
| derevative | | | | | | |
| Crot (27-39) | 1106 | SRWRWKSCKK | Cell-penetrating | 0.97 | Low | 0.86 |
| derevative | | | | | | |
| Crot (27-39) | 1107 | SRWRWKSSKK | Cell-penetrating | 0.96 | Low | 0.96 |
| derevative | | | | | | |
| Peptide 43 | 1108 | SSSIFPPWLSFF | Cell-penetrating | 0.88 | Low | 0.62 |
| Peptide 42 | 1109 | SWAQHLSLPPVL | Cell-penetrating | 0.92 | Low | 0.67 |
| Peptide 40 | 1110 | SWLPYPWHVPSS | Cell-penetrating | 0.95 | Low | 0.75 |
| Peptide 41 | 1111 | SWWTPWHVHSES | Cell-penetrating | 0.76 | Low | 0.695 |
| Peptide 25 | 1112 | SYIQRTPSTTLP | Cell-penetrating | 0.91 | Low | 0.78 |
| PHI 21 N (12- | 1113 | TAKTRYKARRAELIAERRGC | Cell-penetrating | 0.95 | Low | 0.805 |
| 29) | | | | | | |
| IL-13p | 1114 | TAMRAVDKLLLHLKKLFREGQFNRNFESIIIC | Cell-penetrating | 0.82 | High | 0.659 |
| | | RDRT | | | | |
| Inv3.8 | 1115 | TARRITPKDVIDVRSVTTEINT | Non-cell-penetrating | 0.57 | — | — |
| Peptide 1-NSΔ | 1116 | TCTWLKYH | Cell-penetrating | 0.6 | Low | 0.52 |
| Peptide 1-NΔ | 1117 | TCTWLKYHS | Cell-penetrating | 0.55 | Low | 0.66 |
| hCT (21â€“32) | 1118 | TFPQTAIGVGAP | Cell-penetrating | 0.8 | Low | 0.86 |
| Inv3.9 | 1119 | TKAARITPKDVIDVRSVTTEINT | Non-cell-penetrating | 0.6 | — | — |
| Inv3.3 | 1120 | TKRRITPDDVIDVRSVTTEINT | Non-cell-penetrating | 0.57 | — | — |
| Inv3.6 | 1121 | TKRRITPKDVIDV | Cell-penetrating | 0.6 | Low | 0.89 |
| Inv3.7 | 1122 | TKRRITPKDVIDVESVTTEINT | Non-cell-penetrating | 0.64 | — | — |
| Inv3 | 1123 | TKRRITPKDVIDVRSVTTEINT | Non-cell-penetrating | 0.54 | — | — |
| Inv3.5 | 1124 | TKRRITPKDVIDVRSVTTKINT | Cell-penetrating | 0.63 | High | 0.816 |
| Inv3.4 | 1125 | TKRRITPKKVIDVRSVTTEINT | Cell-penetrating | 0.68 | High | 0.848 |
| Peptide 53 | 1126 | TLPSPLALLTVH | Cell-penetrating | 0.96 | Low | 0.69 |
| Peptide 59 | 1127 | TPKTMTQTYDFS | Cell-penetrating | 0.75 | Low | 0.76 |
| FITC-Rath | 1128 | TPWWRLWTKWHHKRRDLPRKPEGC | Cell-penetrating | 0.87 | High | 0.57 |
| Rev (34-50) | 1129 | TRQARRNRRRRWRERQR | Cell-penetrating | 0.98 | High | 0.9 |
| HIV-1 Rev | 1130 | TRQARRNRRRRWRERQRGC | Cell-penetrating | 0.96 | High | 0.9 |
| (34-50) | | | | | | |
| HTLV-II | 1131 | TRRQRTRRARRNRGC | Cell-penetrating | 0.98 | High | 0.521 |
| Rex(4-16) | | | | | | |
| Herpesvirus 8 | 1132 | TRRSKRRSHRKF | Cell-penetrating | 0.99 | Low | 0.582 |
| k8 protein | | | | | | |
| (124-135) | | | | | | |
| BF2d | 1133 | TRSSRAGLQWPVGRVHRLLRKGGC | Cell-penetrating | 0.82 | High | 0.735 |
| Peptide 55 | 1134 | TSHTDAPPARSP | Cell-penetrating | 0.93 | Low | 0.775 |
| HN-1 | 1135 | TSPLNIHNGQKL | Cell-penetrating | 0.9 | Low | 0.64 |
| VP1 BC loop | 1136 | TVDNPASTTNKDKLFAVRK | Cell-penetrating | 0.83 | Low | 0.77 |
| (V) peptides | | | | | | |
| Peptide 1- | 1137 | TWLKYH | Cell-penetrating | 0.64 | Low | 0.534 |
| NTCSΔ | | | | | | |
| Xentry peptides | 1138 | vcvr | Cell-penetrating | 0.63 | High | 0.72 |
| Sweet Arrow | 1139 | VELPPPVELPPPVELPPP | Cell-penetrating | 0.84 | High | 0.84 |
| Protein (SAP) | | | | | | |
| (E) | | | | | | |
| PolyP 4 | 1140 | VHLPPP | Cell-penetrating | 0.8 | Low | 0.96 |
| PolyP 5 | 1141 | VHLPPPVHLPPP | Cell-penetrating | 0.9 | Low | 0.98 |
| PolyP 6 | 1142 | VHLPPPVHLPPPVHLPPP | Cell-penetrating | 0.94 | Low | 0.74 |
| ARF(19-31) scr | 1143 | VIRVHFRLPVRTV | Cell-penetrating | 0.82 | Low | 0.75 |
| PolyP 7 | 1144 | VKLPPP | Cell-penetrating | 0.79 | Low | 0.89 |
| PolyP 8 | 1145 | VKLPPPVKLPPP | Cell-penetrating | 0.89 | Low | 0.84 |
| PolyP 9 | 1146 | VKLPPPVKLPPPVKLPPP | Cell-penetrating | 0.98 | High | 0.89 |
| B1-Lys | 1147 | VKRFKKFFRKLKKKV | Cell-penetrating | 0.97 | High | 0.627 |
| B1-Leu | 1148 | VKRFKKFFRKLKKLV | Cell-penetrating | 0.96 | Low | 0.505 |
| B1 | 1149 | VKRFKKFFRKLKKSV | Cell-penetrating | 0.94 | Low | 0.595 |
| DPV1047 | 1150 | VKRGLKLRHVRPRVTRMDV | Cell-penetrating | 0.93 | Low | 0.86 |
| PV reverse- | 1151 | VKRKKKPALWKTLLKKVLKA | Cell-penetrating | 0.96 | High | 0.5 |
| S4(13) | | | | | | |
| Xentry peptides | 1152 | vlclr | Cell-penetrating | 0.74 | High | 0.78 |
| Peptide 57 | 1153 | VLGQSGYLMPMR | Cell-penetrating | 0.82 | Low | 0.617 |
| Inv1 | 1154 | VNADIKATTVFGGKYVSLTTP | Cell-penetrating | 0.79 | Low | 0.94 |
| Bip6 | 1155 | VPALK | Cell-penetrating | 0.74 | High | 0.96 |
| Bip3 | 1156 | VPALR | Cell-penetrating | 0.75 | High | 0.88 |
| Bip13 | 1157 | VPMIK | Non-cell-penetrating | 0.58 | — | — |
| Bip1 | 1158 | VPMLK | Cell-penetrating | 0.57 | High | 0.96 |
| Bip19 | 1159 | VPTLE | Non-cell-penetrating | 0.59 | — | — |
| Bip2 | 1160 | VPTLK | Cell-penetrating | 0.67 | High | 0.99 |
| Bip16 | 1161 | VPTLQ | Cell-penetrating | 0.6 | High | 0.91 |
| M630 | 1162 | VQAILRRNWNQYKIQ | Cell-penetrating | 0.82 | Low | 0.86 |
| Peptide 10 | 1163 | VQLRRRWC | Cell-penetrating | 0.81 | Low | 0.553 |
| NF-kB | 1164 | VQRKRQKLMP | Cell-penetrating | 0.84 | Low | 0.877 |
| PolyP 1 | 1165 | VRLPPP | Cell-penetrating | 0.8 | Low | 0.92 |
| PolyP 2 | 1166 | VRLPPPVRLPPP | Cell-penetrating | 0.91 | Low | 0.92 |
| PolyP 3 (SAP) | 1167 | VRLPPPVRLPPPVRLPPP | Cell-penetrating | 0.94 | High | 0.85 |
| ARF(2-14) | 1168 | VRRFLVTLRIRRA | Cell-penetrating | 0.95 | High | 0.85 |
| Bip4 | 1169 | VSALK | Cell-penetrating | 0.76 | High | 0.89 |
| Bip8 | 1170 | VSGKK | Cell-penetrating | 0.73 | Low | 0.69 |
| Peptide 47 | 1171 | VSKQPYYMWNGN | Cell-penetrating | 0.73 | Low | 0.74 |
| Bip7 | 1172 | VSLKK | Cell-penetrating | 0.77 | High | 0.62 |
| LMWP | 1173 | VSRRRRRRGGRRRR | Cell-penetrating | 0.98 | Low | 0.501 |
| Protamine | 1174 | VSRRRRRRGGRRRRK | Cell-penetrating | 0.98 | High | 0.614 |
| VG-21 | 1175 | VTPHEIVLVDEYTGEWVDSQFK | Cell-penetrating | 0.65 | Low | 0.755 |
| Xentry peptides | 1176 | VVVR | Cell-penetrating | 0.71 | High | 0.664 |
| GALA | 1177 | WEAALAEALAEALAEHLAEALAEALEALAA | Cell-penetrating | 0.93 | Low | 0.69 |
| KALA | 1178 | WEAKLAKALAKALAKHLAKALAKALKACE | Cell-penetrating | 0.96 | Low | 0.52 |
| | | A | | | | |
| RALA peptide | 1179 | WEARLARALARALARHLARALARA | Cell-penetrating | 0.96 | Low | 0.601 |
| RALA | 1180 | WEARLARALARALARHLARALARALRACEA | Cell-penetrating | 0.96 | Low | 0.604 |
| pAntp (48-58) | 1181 | WFQNRRMKWKK | Cell-penetrating | 0.84 | High | 0.97 |
| TCTP-CPP 25 | 1182 | WIIFKIAASHKK | Cell-penetrating | 0.93 | High | 0.5 |
| TCTP-CPP 18 | 1183 | WIIFRAAASHKK | Cell-penetrating | 0.95 | Low | 0.59 |
| TCTP-CPP 19 | 1184 | WIIFRALISHKK | Cell-penetrating | 0.82 | Low | 0.58 |
| TCTP-CPP 17 | 1185 | WIIFRIAASHKK | Cell-penetrating | 0.91 | Low | 0.53 |
| TCTP-CPP 23 | 1186 | WIIFRIAATHKK | Cell-penetrating | 0.87 | Low | 0.53 |
| TCTP-CPP 21 | 1187 | WIIFRIAAYHKK | Cell-penetrating | 0.83 | High | 0.5 |
| 48 | 1188 | WKARRQCFRVLHHWN | Cell-penetrating | 0.81 | High | 0.7 |
| 47 | 1189 | WKCRRQAFRVLHHWN | Cell-penetrating | 0.8 | High | 0.7 |
| 45 | 1190 | WKCRRQCFRVLHHWN | Cell-penetrating | 0.85 | High | 0.785 |
| NrTP8 | 1191 | WKQSHKKGGKKGSG | Cell-penetrating | 0.95 | Low | 0.82 |
| PF21 | 1192 | WLKLLKKWLKLWKKLLKLW | Cell-penetrating | 1 | Low | 0.52 |
| MK2i | 1193 | WLRRIKAWLRRIKALNRQLGVAA | Cell-penetrating | 0.98 | Low | 0.53 |
| PN291 | 1194 | WRFKAAVALLPAVLLALLAP | Cell-penetrating | 0.8 | Low | 0.597 |
| PN290 | 1195 | WRFKKSKRKV | Cell-penetrating | 0.93 | Low | 0.67 |
| PN287 | 1196 | WRFKWRFK | Cell-penetrating | 1 | High | 0.693 |
| PN288 | 1197 | WRFKWRFKWRFK | Cell-penetrating | 1 | High | 0.73 |
| WR8 | 1198 | WRRRRRRRR | Cell-penetrating | 1 | High | 0.61 |
| cyclic | 1199 | WRWKKKKA | Cell-penetrating | 0.94 | Low | 0.673 |
| [W(RW)4] | | | | | | |
| Unknown | 1200 | WRWRWRWRWRWRWR | Cell-penetrating | 1 | High | 0.715 |
| W2R8 | 1201 | WWRRRRRRRR | Cell-penetrating | 1 | High | 0.58 |
| W3R8 | 1202 | WWWRRRRRRRR | Cell-penetrating | 1 | High | 0.57 |
| W4R8 | 1203 | WWWWRRRRRRRR | Cell-penetrating | 1 | High | 0.576 |
| YARA | 1204 | YARAAARQARA | Cell-penetrating | 0.92 | Low | 0.76 |
| YARA | 1205 | YARAAARQARAKA LARQLGVAA | Cell-penetrating | 0.94 | Low | 0.74 |
| CTP50 | 1206 | YARAARRAARR | Cell-penetrating | 1 | Low | 0.72 |
| CTP505 | 1207 | YAREARRAARR | Cell-penetrating | 0.99 | Low | 0.738 |
| CTP508 | 1208 | YARKARRAARR | Cell-penetrating | 1 | Low | 0.643 |
| Hph-1 | 1209 | YARVRRRGPRR | Cell-penetrating | 0.97 | Low | 0.582 |
| CTP506 | 1210 | YEREARRAARR | Cell-penetrating | 0.97 | Low | 0.69 |
| I-TYR-L-Mca | 1211 | YGDCLPHLKLCKENKDCCSKKCKRRGTNIEK | Cell-penetrating | 0.86 | High | 0.555 |
| | | RCR | | | | |
| CTP504 | 1212 | YGRAARRAARR | Cell-penetrating | 0.99 | Low | 0.7 |
| RTAT-ELPBC | 1213 | YGRGGRRGRRR | Cell-penetrating | 0.99 | Low | 0.679 |
| Tat | 1214 | YGRKKKRRQRRR | Cell-penetrating | 1 | High | 0.517 |
| 1 (TAT) | 1215 | YGRKKRPQRRR | Cell-penetrating | 0.97 | High | 0.568 |
| TAT(47-57) | 1216 | YGRKKRRQRRR | Cell-penetrating | 0.99 | High | 0.555 |
| PEP-2 | 1217 | YGRKKRRQRRRAYFNGCSSPTAPLSPMSP | Cell-penetrating | 0.96 | Low | 0.71 |
| Tat-C-Cy5 | 1218 | YGRKKRRQRRRC | Cell-penetrating | 0.98 | High | 0.709 |
| PEP-1 | 1219 | YGRKKRRQRRRDPYHATSGALSPAKDCGSQ | Cell-penetrating | 0.85 | Low | 0.77 |
| | | KYAYFNGCSSPTLSPMSP | | | | |
| TAT | 1220 | YGRKKRRQRRRGC | Cell-penetrating | 1 | High | 0.617 |
| PN204 | 1221 | YGRKKRRQRRRGCYGRKKRRQRRRG | Cell-penetrating | 0.99 | High | 0.605 |
| TAT-HA2 | 1222 | YGRKKRRQRRRGLFGAIAGFIENGWEGMIDG | Cell-penetrating | 0.89 | Low | 0.57 |
| | | WYG | | | | |
| TAT-NBD | 1223 | YGRKKRRQRRRGTALDWSWLQTE | Cell-penetrating | 0.81 | Low | 0.605 |
| TAT | 1224 | YGRKKRRQRRRPPQG | Cell-penetrating | 0.96 | High | 0.637 |
| PEP-3 | 1225 | YGRKKRRQRRRQRRRPTAPLSPMSP | Cell-penetrating | 0.97 | High | 0.51 |
| Tat-GFP-Tat | 1226 | YGRKKRRQRRRYGRKKRRQRRR | Cell-penetrating | 0.98 | High | 0.54 |
| SP- | 1227 | YGRKKRRQRRRYGRKKRRQRRRYGRKKRR | Cell-penetrating | 0.99 | High | 0.583 |
| Tatm3xCherry | | QRRR | | | | |
| Mutant tat- | 1228 | YGRKKRRQRRTALDASALQTE | Cell-penetrating | 0.77 | High | 0.516 |
| NBD | | | | | | |
| Biotin-labeled | 1229 | YGRKKRRQRRTALDWSWLQTE | Cell-penetrating | 0.77 | Low | 0.588 |
| tat-NBD | | | | | | |
| peptides | | | | | | |
| CTP510 | 1230 | YGRRARRAARR | Cell-penetrating | 0.99 | Low | 0.638 |
| CTP511 | 1231 | YGRRARRRARR | Cell-penetrating | 1 | Low | 0.569 |
| CTP512 | 1232 | YGRRARRRRRR | Cell-penetrating | 1 | Low | 0.567 |
| CTP513 | 1233 | YGRRRRRRRRR | Cell-penetrating | 1 | High | 0.575 |
| M591 | 1234 | YIVLRRRRKRVNTKRS | Cell-penetrating | 1 | High | 0.84 |
| YKA peptide | 1235 | YKALRISRKLAK | Cell-penetrating | 1 | Low | 0.585 |
| Crotamine | 1236 | YKQCHKKGGHCFPKEKICLPPSSDFGKMDCR | Cell-penetrating | 0.8 | High | 0.51 |
| | | WRWKCCKKGSG | | | | |
| NrTP1 | 1237 | YKQCHKKGGKKGSG | Cell-penetrating | 0.96 | Low | 0.79 |
| CTP507 | 1238 | YKRAARRAARR | Cell-penetrating | 1 | Low | 0.652 |
| CTP509 | 1239 | YKRKARRAARR | Cell-penetrating | 0.98 | Low | 0.602 |
| Peptide 3 | 1240 | YNNFAYSVFL | Non-cell-penetrating | 0.62 | — | — |
| CTP502 | 1241 | YPRAARRAARR | Cell-penetrating | 0.99 | Low | 0.718 |
| Peptide 51 | 1242 | YPYDANHTRSPT | Cell-penetrating | 0.9 | Low | 0.828 |
| Peptide 9 | 1243 | YQKQAKIMCS | Non-cell-penetrating | 0.68 | — | — |
| Peptide 7 | 1244 | YRDRFAFQPH | Cell-penetrating | 0.6 | Low | 0.643 |
| PN267 | 1245 | YRFK | Cell-penetrating | 0.86 | High | 0.566 |
| PN282 | 1246 | YRFKYRFKYRLFK | Cell-penetrating | 0.97 | High | 0.56 |
| NrTP7 | 1247 | YRQSHRRGGRRGSG | Cell-penetrating | 1 | Low | 0.755 |
| CTP503 | 1248 | YRRAARRAARA | Cell-penetrating | 1 | Low | 0.727 |
| CTP514 | 1249 | YRRRRRRRRRR | Cell-penetrating | 1 | High | 0.64 |
| ECP(33-40) | 1250 | YRWRCKNQ | Cell-penetrating | 0.77 | High | 0.54 |
| ECP(33-41) | 1251 | YRWRCKNQN | Cell-penetrating | 0.73 | Low | 0.6 |
| Peptide 24 | 1252 | YSHIATLPFTPT | Cell-penetrating | 0.9 | Low | 0.73 |
| NFL-TBS.40- | 1253 | YSSYSAPVSSSLSVRRSYSSSSGS | Cell-penetrating | 0.92 | Low | 0.82 |
| 63 | | | | | | |
| YTA2 | 1254 | YTAIAWVKAFIRKLRK | Cell-penetrating | 0.83 | High | 0.52 |
| Ypep-GFP | 1255 | YTFGLKTSFNVQ | Non-cell-penetrating | 0.51 | — | — |
| Ypep-GFP- | 1256 | YTFGLKTSFNVQYTFGLKTSFNVQ | Cell-penetrating | 0.59 | Low | 0.6 |
| Ypep | | | | | | |
| hCT(12â€“32) | 1257 | YTQDFNKFHTFPQTAIGVGAP | Non-cell-penetrating | 0.56 | — | — |
| Tyr-Oct-6 | 1258 | YYYAAGRKRKKRT | Cell-penetrating | 1 | Low | 0.95 |
| mature CPG2 | 1259 | ALAQKRDNVLFQAATDEQPAVIKTLEKLVNI | | | | |
| | | ETGTGDAEGIAAAGNFLEAELKNLGFTVTRS | | | | |
| | | KSAGLVVGDNIVGKIKGRGGKNLLLMSHMD | | | | |
| | | TVYLKGILAKAPFRVEGDKAYGPGIADDKGG | | | | |
| | | NAVILHTLKLLKEYGVRDYGTITVLFNTDEE | | | | |
| | | KGSFGSRDLIQEEAKLADYVLSFEPTSAGDEK | | | | |
| | | LSLGTSGIAYVQVNITGKASHAGAAPELGVN | | | | |
| | | ALVEASDLVLRTMNIDDKAKNLRFNWTIAK | | | | |
| | | AGNVSNIIPASATLNADVRYARNEDFDAAMK | | | | |
| | | TLEERAQQKKLPEADVKVIVTRGRPAFNAGE | | | | |
| | | GGKKLVDKAVAYYKEAGGTLGVEERTGGG | | | | |
| | | TDAAYAALSGKPVIESLGLPGFGYHSDKAEY | | | | |
| | | VDISAIPRRLYMAARLIMDLGAGK | | | | |

*Prediction confidence of cell penetration
**Prediction confidence of uptake efficiency

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method for loading a lipid vesicle (LV) with a cargo molecule, comprising contacting the LV with a binding complex, wherein the binding complex comprises the cargo molecule and a cell penetrating polypeptide (CPP) covalently or non-covalently coupled to the cargo molecule, and wherein the binding complex becomes internalized by, or associated with, the LV.

2. The method of claim 1, wherein the CPP is non-covalently coupled to the cargo molecule.

3. The method of claim 1, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NETS) ester, a chemical bond formed via Click chemistry, or other covalent linkage.

4. The method of claim 3, wherein the CPP is covalently coupled to the cargo molecule by a cleavable linker.

5. The method of claim 4, wherein the cleavable linker is a photo-cleavable linker.

6. The method of claim 4, further comprising uncoupling the cargo molecule and CPP of the binding complex by cleaving the cleavable linker after the binding complex becomes internalized by, or associated with, the LV.

7. The method of claim 1, wherein the cargo molecule is selected from among a small molecule, macromolecule such as polyimide, proteins, polypeptide (natural or modified), nucleic acid, antibody or antibody-fragment, lipoprotein, carbohydrate, or glycoprotein.

8. The method of claim 1, wherein the LV is a liposome.

9. The method of claim 1, wherein the LV is a lipid nanoparticle, lipid droplet, micelle, reverse micelle, lipid-polymer hybrid nanoparticle, or artificial extracellular vesicle.

10. The method of claim 1, wherein the cargo molecule is a detectable agent or medical imaging agent, or is attached to a detectable or medical imaging agent, such as a fluorescent compound to serve as a marker, dye, tag, or reporter.

11. The method of claim 1, wherein the LV further comprises a targeting agent that targets the LV to a cell type, organ, or tissue.

12. The method of claim 1, wherein the CPP is one listed in Table 2 or Table 11.

13. The method of claim 1, wherein the CPP is selected from among the following: Tat, Antennapedia, VP22, CaP, YopM, Artificial protein B1, 30Kc19, engineered +36 GFP, naturally supercharged human protein, and gamma-AA peptide.

14. The method of claim 1, wherein the method further comprises the step of coupling CPP to the cargo molecule prior to contacting the LV with the binding complex.

15. The loaded LV produced by the method of claim 1.

16. A loaded lipid vesicle (LV), comprising a cargo molecule and a cell penetrating peptide (CPP), wherein the cargo molecule has been internalized by, or associated with, the LV.

17. The loaded LV of claim 16, where the loaded LV comprises a binding complex, wherein the binding complex comprises the cargo molecule and a CPP covalently or non-covalently coupled to the cargo molecule, and wherein the binding complex has been internalized by, or associated with, the LV.

18. The loaded LV of claim 17, wherein the CPP is covalently coupled to the cargo molecule by a disulfide bond, an amide bond, a chemical bond formed between a sulfhydryl group and a maleimide group, a chemical bond formed between a primary amine group and an N-Hydroxysuccinimide (NHS) ester, a chemical bond formed via Click chemistry, or other covalent linkage.

19. A method for delivering a cargo molecule into a cell in vitro or in vivo, comprising administering a loaded lipid vesicle (LV) to the cell in vitro or in vivo, wherein the loaded LV comprises a binding complex, wherein the binding complex comprises the cargo molecule and a cell penetrating polypeptide (CPP) covalently or non-covalently coupled to the cargo molecule, and wherein the loaded LV is internalized into the cell.

20. The method of claim 19, wherein the loaded LV comprises a binding complex, wherein the binding complex comprises the cargo molecule and a CPP covalently or non-covalently coupled to the cargo molecule, and wherein the binding complex has been internalized by, or associated with, the LV.
